(19) United States
(12) Patent Application Publication
(10) Pub. No.: US 2023/0372393 A1
(43) Pub. Date: Nov. 23, 2023

(54) METHODS FOR ALLOGENEIC HEMATOPOIETIC STEM CELL TRANSPLANTATION

(71) Applicant: Orca Biosystems, Inc., Menlo Park, CA (US)

(72) Inventors: Everett Hurteau MEYER, Belmont, CA (US); Robert S. NEGRIN, Stanford, CA (US); Nathaniel FERNHOFF, Menlo Park, CA (US); Scott MCCLELLAN, Menlo Park, CA (US); Scott KILLIAN, Menlo Park, CA (US)

(21) Appl. No.: 18/142,513

(22) Filed: May 2, 2023

Related U.S. Application Data
(63) Continuation of application No. PCT/US2021/058141, filed on Nov. 4, 2021.
(60) Provisional application No. 63/121,742, filed on Dec. 4, 2020.
(60) Provisional application No. 63/121,534, filed on Dec. 4, 2020.
(60) Provisional application No. 63/121,453, filed on Dec. 4, 2020.
(60) Provisional application No. 63/109,811, filed on Nov. 4, 2020.

(51) Int. Cl. A61K 35/15; A61K 35/17; A61K 35/28; A61K 31/436; A61K 31/675; A61K 31/255; A61K 31/7076; A61P 37/06

(52) U.S. Cl. CPC A61K 35/15; A61K 35/17; A61K 35/28; A61K 31/436; A61K 31/675; A61K 31/255; A61K 31/7076; A61P 37/06

(57) ABSTRACT

Various embodiments of the invention provide therapeutic compositions and associated methods for improved hematopoietic stem cell transplantations, including methods to enhance protection from graft versus host disease while maintaining effective immune responses such as graft versus tumor immune responses. Embodiments of the invention are particularly useful for treatment of hematologic cancers (e.g., leukemia, lymphoma, GVHD and other diseases).

### CROSS-REFERENCE

This application is a continuation of International Application PCT/US2021/058141, filed Nov. 4, 2021, which claims the benefit of priority of U.S. Provisional Application No. 63/109,811, filed on Nov. 4, 2020, and U.S. Provisional Application Nos. 63/121,453, 63/121,742, and 63/121,534 each filed on Dec. 4, 2020, all of which are incorporated herein by reference in their entirety for all purposes.

### STATEMENT AS TO FEDERALLY SPONSORED RESEARCH

This invention was made with government support under HL114591 awarded by the National Institutes of Health. The government has certain rights in the invention.

### BACKGROUND

Patients with hematologic malignancies such as leukemia and lymphoma beyond first remission or with refractory relapse are rarely cured with standard chemotherapy. Myeloablative allogeneic hematopoietic cell transplantation (alloHCT) is associated improved survival in these patients, but the morbidity and mortality associated with graft versus host disease (GVHD) is a major factor limiting the success of alloHCT.

### SUMMARY

In a first aspect, embodiments of the invention provide a multi-component pharmaceutical treatment to be administered to a human subject in need thereof. According to one embodiment, the multi-component treatment comprises (a) a solution comprising a first population of CD45+ cells comprising hematopoietic stem and progenitor cells (HSPCs) and granulocytes wherein at most about 10% of the first population of CD45+ cells comprise granulocytes; (b) a solution comprising a population of cells enriched for regulatory T cells (Tregs); (c) a solution comprising a second population of CD45+ cells wherein the second population of CD45+ cells comprise at least about 20% CD3+ conventional T cells (Tcons), at least about 10% monocytes, and at least about 10% granulocytes; and (d) a solution comprising one or more doses of a graft vs host disease (GVHD) prophylactic agent.

In many embodiments, the GVHD prophylactic agent comprises tacrolimus and/or its analogues and derivatives which according to various embodiments can be formulated for oral administration or intravenous administration to a human subject or other administration or delivery method known in the pharmaceutical arts including for example, intramuscular, transdermal, nasal, buccal and vaginal administration.

In various embodiments, the tacrolimus can be administered in an amount to maintain a target blood level in a human subject of at least about 3 ng/ml blood for at least about 20 days after administering the second population of CD45+ cells, in an amount to maintain a target blood level of about 4 ng/ml or more for at least about 40 days after administering the second population of CD45+ cells, and/or in an amount that maintains a target blood level of about 4 ng/ml or more for at least about 40 days after administering the second population of CD45+ cells. In some cases, the tacrolimus is administered in an amount that maintains a target blood level of at most about 10 ng/ml for at least 30 days after administering the second population of CD45+ cells.

In some embodiments, the tacrolimus is administered for at least about 60 days after administering the second population of CD45+ cells, for at least about 90 days after administering the second population of CD45+ cells, for at most about 150 days after administering the second population of CD45+ cells, for at most about 120 days after administering the second population of CD45+ cells.

In some embodiments, the first population of CD45+ cells comprises at least about 0.5% granulocytes, at least about 1% granulocytes, at most about 5% granulocytes, at most about 3% granulocytes, at most about 3% monocytes, at most about 2% monocytes, at most about 0.5% lymphocytes, at most about 2% lymphocytes, at least about 15% granulocytes, at least about 20% granulocytes, at most about 35% granulocytes, at most about 30% granulocytes, at most about 25% granulocytes, at least about 15% monocytes, at least about 20% monocytes, at most about 35% monocytes, at most about 30% monocytes, at most about 25% monocytes, at least about 0.5% NK cells, and or at least about 2% NK cells.

In some embodiments, the second population of CD45+ cells comprises at least about 0.10% CD34+ cells or from about 0.2% to about 20% CD34+ cells and/or at least about 0.10% Tregs.

In various embodiments, the multi-component pharmaceutical treatment further comprises a conditioning regimen, wherein the conditioning regimen is administered before any of components (a) to (d) listed above, e.g., the conditioning regimen is administered from about two days to about ten days before any of (a) to (d). In some embodiments, the conditioning regimen is a myeloablative conditioning regimen. In some cases, the conditioning regimen comprises at least three conditioning reagents, wherein at least one conditioning reagent is thiotepa. In embodiments, the myeloablative conditioning regimen comprises at least one dose of thiotepa, e.g., at least about 5 milligrams thiotepa per kilogram of the human subject's actual or ideal body weight or at least about 10 milligrams thiotepa per kilogram of the human subject's actual or ideal body weight. In various embodiments, the conditioning regimen comprises one or more doses of busulfan, fludarabine and thiotepa. In some embodiments, the one or more doses comprises from about 5 to about 12 mg of thiotepa per kg human subject's actual or ideal body weight, from about 7 to about 11 mg of busulfan per kg human subject actual or ideal body weight, and from about 100 to about 200 mg of fludarabine per meter² body surface area respectively.

In some embodiments, the first population of CD45+ cells comprises less than about 5 EU of endotoxins per ml of the solution, less than about 1 EU of endotoxins per ml of the solution, and/or less than about 0.5 EU of endotoxins per ml of the solution. In some cases, the population of cells enriched for Tregs comprises less than about 5 EU of endotoxins per ml of the solution, less than about 1 EU of endotoxins per ml of the solution, and/or less than about 0.5 EU of endotoxins per ml of the solution. In various embodiments, the second population of CD45+ cells comprises less than about 5 EU of endotoxins per ml of the solution, less than about 1 EU of endotoxins per ml of the solution, and/or less than about 0.5 EU of endotoxins per ml of the solution.

In some embodiments, the HSPCs are CD34+.

In some embodiments, the Tregs are CD4+ CD25+ CD127dim or CD4+ FOXP3+. In some cases, the population of cells enriched for Tregs comprises CD45+ cells, e.g., more than about 90% of the CD45+ cells are Tregs. In various embodiments, the population of cells enriched for Tregs comprises from about 1×10⁵ to about 1×10⁷ Tregs per kilogram of actual or ideal body weight of said human subject or from about 5×10⁵ to about 4×10⁶ Tregs per kilogram of actual or ideal body weight of said human subject.

In some embodiments, the first population of CD45+ cells comprising HSPCs comprises from about 5×10⁵ to about 2×10⁷ HSPCs per kilogram of ideal body of said human subject. In some cases, the second population of CD45+ cells comprises from about 1×10⁵ to about 1×10⁷ Tcons per kilogram of actual or ideal body weight of said human subject or the second population of CD45+ cells comprises from about 5×10⁵ to about 5×10⁶ Tcons per kilogram of actual or ideal body weight of said human subject.

In some embodiments, the first population of CD45+ cells and the population of cells enriched for Tregs are administered before the second population of CD45+ cells.

In various embodiments, a first dose of the one or more doses of the GVHD prophylactic agent is administered after the administration of the second population of CD45+ cells.

Another aspect provides a method of treating a human subject diagnosed with a hematologic malignancy. According to one embodiment, the method comprises administering to the human subject a solution comprising the first population of CD45+ cells, a solution comprising the population of cells enriched for regulatory Tregs, a solution comprising the second population of CD45+ cells, and a solution comprising one or more doses of the GVHD prophylactic agent. In this aspect, the solution comprising the first population of CD45+ cells, the solution comprising the population of cells enriched for regulatory Tregs, the solution comprising the second population of CD45+ cells, and the solution comprising one or more doses of the GVHD prophylactic agent are as defined according to any herein disclosed multi-component pharmaceutical treatment.

The hematologic malignancy may correspond to one or more of acute lymphocytic leukemia, acute myelogenous leukemia, chronic myelogenous leukemia, multiple myeloma, lymphoma, Hodgkin's lymphoma, and non-Hodgkin lymphoma.

In some embodiments, administering comprises infusing into a human subject said first population of CD45+ cells, said population of cells enriched for Tregs, and said second population of CD45+ cells.

In some embodiments, said second population of CD45+ cells can be administered at least about 12 hours after said first population of CD45+ cells, said second population of CD45+ cells is administered from about 24 to about 96 hours after said first population of CD45+ cells, said second population of CD45+ cells is administered from about 36 to about 60 hours after said first population of CD45+ cells, said second population of CD45+ cells is administered at least about 12 hours after said population of cells enriched for Tregs, said second population of CD45+ cells is administered from about 24 to about 96 hours after said population of cells enriched for Tregs, and/or said second population of CD45+ cells is administered from about 36 to about 60 hours after said population of cells enriched for Tregs.

In various embodiments, the human subject does not develop higher than stage 2 GVHD within about 100 days of said administering of said second population of CD45+ cells, said human subject does not develop higher than stage 2 GVHD) within about 180 days or within about 200 days of said administering of said second population of CD45+ cells, said human subject does not develop higher than stage 2 GVHD within about 1 year of said administering of said second population of CD45+ cells.

In some embodiments, said human subject has previously been or is concurrently treated for the hematologic malignancy.

In some embodiments, the GVHD prophylactic agent is tacrolimus (and/or its analogues and derivatives) and is initially administered to the human subject at about 0.03 mg/kg of the human subject's actual or ideal body weight/day or the tacrolimus is initially administered from about 12 hours to about 24 hours after said administering of said second population of CD45+ cells.

In various embodiments, a dose of the tacrolimus can be tapered starting at about 90 days after the first dose is administered to the human subject or a dose of the tacrolimus is tapered starting at about 45 days after the first dose is administered to the human subject.

In some embodiments, the first population of CD45+ cells, the population of cells enriched for Tregs, and the second population of CD45+ cells are obtained from a single donor either on single day or over multiple days.

In some embodiments, at least one mobilized peripheral blood donation is collected from a donor or at most two mobilized peripheral blood donations are collected from the donor.

In various embodiments, at least one of the mobilized peripheral blood donations is processed and sorted to enrich CD34+ cells and Tregs. In some embodiments, the processing and sorting time of the one or more of the mobilized peripheral blood donations is less than about 35 hours, the processing and sorting time of the one or more of the mobilized peripheral blood donations is less than about 30 hours, the processing and sorting time of the one or more of the mobilized peripheral blood donations is less than about 25 hours, the processing and sorting time of the one or more of the mobilized peripheral blood donations is at most about 35 hours, and/or the processing and sorting time of the one or more of the mobilized peripheral blood donations is at most about 25 hours.

In various embodiments, the one or more of the mobilized peripheral blood donations is processed and sorted using one or more immune-separation particles (ISPs), e.g., ISPs comprise affinity reagents such as immuno-magnetic separation particles which may be antibodies each conjugated to an iron-containing particle. In some embodiments, the affinity reagents comprise a plurality of CD34-reagents (e.g., an anti-CD34 antibody) that binds to one or more CD34 receptors on a HSPC. In some cases, an average number of ISP's per HSPC in the HSPC cell population is less than about 20,000, an average number of ISP's per HSPC in the HSPC cell population is equal to or less than about 10,000, and/or an average number of ISP's per HSPC in the HSPC cell population is from about 1500 to about 20,000. In embodiments, the affinity reagents the affinity reagents comprise a plurality of CD25-reagents (e.g., an anti-CD25 antibody) that binds to one or more CD25 receptors on a Treg. In some cases, an average number of ISP's per T-reg cell in the Treg population is equal or less than about 4000 or an average number of ISPs per T-reg cell in the Treg population is from about 1500 to about 2500.

In various embodiments, cells of the mobilized peripheral blood donation are sorted such that the first population of CD45+ cells comprises at most about 10% granulocytes. In some cases, cells of the mobilized peripheral blood donation are sorted such that the first population of CD45+ cells comprises at most about 7% granulocytes.

In some embodiments, cells of the mobilized donor peripheral blood donation are sorted such that the first population of CD45+ cells comprises at most about 4% monocytes. In some cases, cells of the mobilized donor peripheral blood donation are sorted such that the first population of CD45+ cells comprises at least about 0.1% monocytes.

In embodiments, cells of the mobilized donor peripheral blood donation are sorted such that the population enriched for Tregs comprises at most about 10% CD25− cells.

In various embodiments, the first population of CD45+ cells, said population of cells enriched for Tregs, and/or said second population of CD45+ cells is allogeneic relative to said human subject.

In some embodiments, the first population of CD45+ cells, said population of cells enriched for Tregs, and/or said second population of CD45+ cells is obtained from a donor that is HLA-matched relative to said human subject.

In embodiments, the first population of CD45+ cells, said population of cells enriched for Tregs, and/or said second population of CD45+ cells is obtained from a donor that is HLA-mismatched relative to said human subject.

In various embodiments, the first population of CD45+ cells, said population of cells enriched for Tregs, and/or said second population of CD45+ cells is obtained from a donor that is haploidentical relative to said human subject.

In some embodiments, the second population of CD45+ cells comprises a population of invariant natural killer T cells (iNKTs), e.g., iNKTs that are CD3+Vα24Jα18+.

In embodiments, the population of iNKTs comprises more than about 5×10² iNKTs per kilogram of ideal body actual or ideal body weight of said human subject.

In various embodiments, the population of iNKTs comprises from about 5×10² to about 1×10⁷ iNKTs per kilogram of ideal body actual or ideal body weight of said human subject.

In some embodiments, the second population of CD45+ cells comprises a population of memory T cells (Tmems), e.g., Tmems that are CD3+ CD45RA− CD45RO+.

In embodiments, the population of Tmems comprises more than about 3×10⁵ Tmems per kilogram of ideal body actual or ideal body weight of said human subject.

In various embodiments, the population of Tmems comprises from about 3×10⁵ to about 1×10⁹ Tmems per kilogram of ideal body actual or ideal body weight of said human subject.

Yet another aspect provides a multi-component pharmaceutical treatment in which a risk and/or severity of an adverse event associated with the multi-component pharmaceutical treatment is reduced as compared to a similar pharmaceutical treatment in which a human subject receives Tcons but does not receive Tregs or is any herein-disclosed method in which a risk and/or severity of an adverse event associated with the method is reduced as compared to a similar method in which a human subject receives Tcons but does not receive Tregs.

In some embodiments, the adverse event is acute GVHD (aGVHD). which may include stage two or greater aGVHD.

In various embodiments, the adverse event is chronic GVHD (cGVHD) which may be moderate to severe cGVHD.

In some embodiments, the human subject has no cGVHD about one year after being administered the cell populations.

In various embodiments, the adverse event is relapse of the human subject's malignancy.

In some embodiments, the human subject has no relapse of their malignancy about one year after being administered the pharmaceutical dosing regimen.

In embodiments, the human subject has undergone myeloablative conditioning regimen before administration of any cell populations and the adverse event is associated with the myeloablative conditioning.

In various embodiments, the method further comprises providing instructions for use (IFU), the IFU including instructions for administering the cell populations to the patient. In some cases the IFU also include instructions for administering one or more pharmaceutical agents or compositions to the patient.

A further aspect provides a method of transplanting a conventional T cell (Tcons) population as a part of a treatment regimen for a hematologic malignancy in which the method reduces a risk and/or severity of an adverse event associated with the treatment regimen. The method comprises administering to the patient a population of regulatory T cells (Tregs) comprising Tregs and a liquid suspending the Tregs; administering to the patient a heterogenous cell population comprising lymphocytes, granulocytes, monocytes and a liquid suspending said cells. In this aspect, at least about 30% of said lymphocytes comprise Tcons. and after administration of the cell populations, the patient has a reduced risk and/or severity of the adverse event as compared to hematologic malignancy patients who received Tcons but did not receive Tregs.

A yet further aspect provides a method of transplanting cell populations into a human patient as a part of a treatment regimen for a hematologic malignancy in which the method reduces a risk and/or severity of an adverse event associated with the treatment regimen. The method comprises providing a population of hematopoietic stem and progenitor cells (HSPCs) to be administered to the patient; the population of HSPCs comprising HSPCs and a liquid suspending the HSPCs; providing a population of regulatory T cells (Tregs) to be administered to the patient, the population of Tregs comprising Tregs and a liquid suspending the Tregs; and providing a heterogenous cell population to be administered to the patient, the heterogenous cell population comprising lymphocytes, granulocytes, monocytes and a liquid suspending said cells. In this aspect, at least about 30% of said lymphocyte comprise conventional T cells (Tcons) and after administration of the cell populations, the patient has a reduced risk and/or severity of the adverse event as compared to hematologic malignancy patients who received a Tcon cell population but did not receive a T-reg cell population.

In some embodiments, the cell populations are administered to the patient by intravenous infusion.

In embodiments, the respective cell populations are provided as separate cell populations and are derived from a single human blood donor.

In various embodiments, the adverse event is acute graft vs host disease (aGVHD), e.g., stage two or greater aGVHD. In some cases, the patient has no stage two or higher aGVHD about 180 days after being administered the cell populations.

In some embodiments, the adverse event is chronic graft vs host disease (cGVHD). In some cases, the patient has no cGVHD about one year after being administered the cell populations.

In embodiments, the adverse event is moderate to severe cGVHD. In some cases, the patient does not have moderate to severe cGVHD about one year after being administered the cell populations.

In various embodiments, the adverse event is relapse of the patient's malignancy. In some cases, the patient has no relapse of their malignancy about one year after being administered the cell populations.

In some embodiments, the adverse event includes graft versus host disease (GVHD) and relapse of the patient's malignancy. In some cases, the patient has no GHVD or relapse of their malignancy one year after being administered the cell populations.

In embodiments, at least one of the cell populations comprise less than about 5 EU of endotoxins/ml of respective suspension liquid.

In various embodiments, the patient has undergone myeloablative conditioning regimen before administration of the cell populations and the adverse event is associated with the myeloablative conditioning. In some cases, the adverse event includes relapse of the patient's malignancy or infection.

In some embodiments, the heterogenous cell population comprises from about 0.2 to about 2.0 percent hematopoietic stem and progenitor cells.

In embodiments, the hematologic malignancy is acute lymphocytic leukemia, acute myelogenous leukemia, chronic myelogenous leukemia, multiple myeloma, lymphoma, Hodgkin's lymphoma and non-Hodgkin lymphoma.

In various embodiments, a genetic expression level of the T-reg cells correlates to cells that were harvested from the donor within about 60 hours prior to administration to the patient.

In some embodiments, the number of T-reg cells in the T-reg population is about equal to the number of T-con cells in the heterogenous cell population. In some cases, the T-reg cells in the T-reg population inhibit activation of conventional T cells in the heterogenous cell population by the patient's healthy tissue by an amount up to about 20 percent

In embodiments, the peripheral blood of the patient exhibits an elevated ratio of Tregs to CD4+ T cells up to about 100 days after administration of the cell populations as compared to a healthy human subject that was not administered the cells populations.

In various embodiments, at least about 50% of the cells in the HSPC's cell population are colony forming units.

In some embodiments, at least one of the cell populations has an elevated amount of granulocyte colony-stimulating factor as compared to non-mobilized blood. In some cases, the at least one cell populations is the heterogenous cell population.

In embodiments, at least one of the cell populations have a plurality of immuno-separation particles (ISPs) attached to receptors on the cells of the cell population. In some cases, the plurality of ISPs are immuno-magnetic separation particles. In various embodiments, the plurality of ISPs comprise an antibody conjugated to an iron containing particle.

In some embodiments, the population of Tcons is administered at least about 12 hours after the population of HSPCs, e.g., said population of Tcons is administered from about 24 to about 96 hours after the population of HSPCs or said population of Tcons itabs administered from about 36 to about 60 hours after the population of HSPCs.

In some embodiments, the population of Tcons is administered at least about 12 hours after said population of cells comprising Tregs, e.g., the population of Tcons is administered from about 24 to about 96 hours after the population of cells comprising Tregs or said population of Tcons is administered from about 36 to about 60 hours after the population of cells comprising Tregs.

In various embodiments, a herein-disclosed method further comprises administering to the patient over a period of time up to about 180 days a single graft versus host disease (GVHD) prophylactic agent (GVHDPA) comprising tacrolimus (tacrolimus GHVDPA); wherein the tacrolimus GHVDPA is administered to maintain a concentration of tacrolimus in the patient's blood above a threshold level during the period of time; and wherein a risk and/or severity of GHVD is significantly reduced.

In some embodiments, the threshold level is above about 4 ng of tacrolimus per ml of patient blood or the threshold level is above about 5 ng of tacrolimus per ml of patient blood.

In embodiments, the tacrolimus GHVDPA is administered to maintain a concentration of tacrolimus in the patients' blood below an upper threshold level during the period of time. In some cases, the upper threshold level is below about 10 ng of tacrolimus per ml of patient blood.

In various embodiments, the patient has a reduced risk of at least one of malignancy relapse, infection or renal failure.

In some embodiments, the patient does not develop GVHD within about 30 days of administration of the Tcons, does not develop GVHD within about 100 days of administration of the Tcons, does not develop GVHD within about 180 days of administration of the Tcons, and/or does not develop GVHD within about one year of administration of the Tcons.

In various embodiments, the tacrolimus graft versus host disease (GVHD) prophylactic agent (GVHDPA) may be intravenously administered or orally administered to the patient. In various embodiments, administration of the tacrolimus graft versus host disease (GVHD) prophylactic agent (GVHDPA) is started from about 12 to about 24 hours after administration of the T-cons. In some cases, the tacrolimus GHVDPA is administered for a period of time up to about 90 days, is administered for a period of time up to about 60 days. In some embodiments, the tacrolimus GHVDPA is initially administered to the patient at about 0.03 mg/kg patient's actual or ideal body weight/day. In some cases, a dose of the tacrolimus GVHDPA administered to the patient is tapered starting at about 90 days after a first dose is administered to the patient or is tapered starting at about 45 days after a first dose is administered to the patient.

In some embodiments, the method further comprises administering a myeloablative conditioning regimen to the patient prior to the administration of any cell population, the conditioning regimen comprising administration of at least one conditioning agent to the patient.

In embodiments, the patient does not receive any irradiation as part of the myeloablative conditioning regimen.

In various embodiments, the at least one conditioning agent is administered from about two to about ten days prior to the administration of any of the cell populations. In some cases, the at least one conditioning agent is administered about five days prior to the administration of any of the cell populations.

In some embodiments, the at least one conditioning agent comprises thiotepa. In some cases, a dose of thiotepa administered to the patient is in a range of from about 5 to about 10 mg per kilogram of actual or ideal body weight.

In embodiments, the at least one conditioning agent comprises busulfan and fludarabine. In some cases, doses of thiotepa, busulfan, and fludarabine administered to the patient comprise about 10 mg per kilogram of the patient's actual or ideal body weight, about 9.6 mg per kilogram of the patient's actual or ideal body weight, and about 150 mg per meter² body surface area respectively.

In various embodiments, the method further comprises providing instructions for use (IFU), the IFU including instructions for administering the cell populations to the patient. In some cases, the IFU also include instructions for administering one or more pharmaceutical agents or compositions to the patient.

Yet another aspect, provides a method of transplanting cell populations into a human patient as a part of a treatment regimen for a hematologic malignancy. The method comprises administering to the patient a population of hematopoietic stem and progenitor cells (HSPCs; the population of HSPCs comprising HSPCs and a liquid suspending the HSPCs; administering to the patient a population of regulatory T cells (Tregs) to be administered to the patient, the population of Tregs comprising Tregs and a liquid suspending the Tregs; and administering to the patient a heterogenous cell population to be administered to the patient, the heterogenous cell population comprising lymphocytes, granulocytes, monocytes and a liquid suspending said cells, wherein at least about 30% of said lymphocyte comprise conventional T cells (Tcons); and administering to the patient over a period of time up to about 180 days a single graft versus host disease (GVHD) prophylactic agent (GVHDPA) comprising tacrolimus (tacrolimus GHVDPA), wherein the tacrolimus GHVDPA is administered to maintain a concentration of tacrolimus in the patient's blood above a threshold level during the period of time; and wherein a risk and/or severity of GHVD associated with the treatment regimen for the hematologic malignancy is significantly reduced.

Another aspect provides a kit that comprises a solution comprising

a first container comprising a first population of CD45+ cells, a second container comprising a solution comprising a second population of CD45+ cells, and a third container comprising a solution comprising a population of cells enriched for regulatory T cells (Tregs). In this aspect, the solution comprising the first population of CD45+ cells, the solution comprising the second population of CD45+ cells, and the solution comprising the population of cells enriched for Tregs are as defined according to any herein disclosed multi-component pharmaceutical treatment or method. In some embodiments, the kit further comprises a fourth container comprising the GVHD prophylactic agent. In some cases, the further comprising instructions for performing any herein-disclosed method.

Another aspect provides a kit comprising: (a) one or more reagents to sort CD34+ cells from a mobilized peripheral blood composition; (b) one or more reagents to sort regulatory T cells (Tregs) from the mobilized peripheral blood composition; (c) one or more reagents to detect a number of CD3+ conventional T cells in the mobilized peripheral blood; and (d) a solution comprising one or more doses of a graft vs host disease (GVHD) prophylactic agent. In embodiments, the kit further comprises instructions for performing any herein-disclosed method.

An aspect provides a method of transplanting a conventional T cell (Tcon) population into a human subject without eliciting a stage 2 or higher graft versus host disease (GVHD) response up to about 100 days after transplanting. The method comprises (i) administering a heterogenous cell population comprising lymphocytes, granulocytes and monocytes, wherein at least about 30% of said lymphocytes comprises conventional T cells (Tcons); and (ii). administering a population of regulatory T cells (Tregs). In this method, the heterogenous cell component and/or the population of Tregs comprise less than about 5 EU/ml endotoxins.

Another aspect provides a method of treating a human subject comprising a step (a) of administering a plurality of populations of cells, in which the plurality of populations of cells comprises: (i). a population of hematopoietic stem and progenitor cells (HSPCs); (ii) a population of cells comprising regulatory T cells (Tregs); and (iii) a population of conventional T cells (Tcons); and a step (b) of administering no more than one graft versus host disease (GVHD) prophylactic agent for less than about 120 days. In this method, the population of HSPCs comprises less than about 2% CD3+ cells.

A further aspect provides a method treating a human subject in need thereof comprising administering to the human subject at least two pharmaceutical compositions, wherein the pharmaceutical compositions are selected from (a) a pharmaceutical composition comprising a population of hematopoietic stem and progenitor cells (HSPCs); (b) a pharmaceutical composition comprising a population of regulatory T cells (Tregs); and (c) a pharmaceutical composition comprising a population of conventional T cells (Tcons). In this method, the pharmaceutical compositions (a), (b) and (c) comprise less than about 5 EU/ml endotoxins each; and less than 15 human subjects in a group of 100 human subjects administered the two or more pharmaceutical compositions develops a stage 2 or higher graft versus host disease (GVHD) response within about 30 days after being administered the pharmaceutical composition comprising the population of Tcons.

An additional aspect provides a method of transplanting a conventional T cell (Tcon) population into a human subject without eliciting a stage 2 or higher graft versus host disease (GVHD) response up to about 100 days after transplanting. The method comprising: (i). administering a solution comprising a population of conventional T cells (Tcons); and (ii). administering a solution comprising a population of regulatory T cells (Tregs). In this method, the population of Tcons is cryopreserved for at least about 4 hours; and the solution comprising the population of Tcons and the solution comprising the population of Tregs comprise less than about 5 EU of endotoxins per ml of the solution.

An aspect provides a method of treating a hematologic malignancy in a human subject in need thereof, the method comprising administering to the human subject: (a) a population of hematopoietic stem and progenitor cells (HSPCs); (b) a population of regulatory T cells (Tregs); and (c) a population of conventional T cells (Tcons). In the method, the population of HSPCs and the population of Tregs are administered prior to the population of Tcons; and peripheral blood of the human subject exhibits an elevated Treg count until about 100 days after the administering the three populations of cells as compared to a healthy human subject that was not administered the three populations of cells.

A further aspect provides a method of transplanting a conventional T cell (Tcon) population into a human subject without eliciting a stage 2 or higher graft versus host disease (GVHD) response up to about 100 days after transplanting. The method comprising (i). administering a population of conventional T cells (Tcons); and (ii). administering a population of regulatory T cells (Tregs). In this method, the population of Tcons is administered at least about 12 hours after the population of Tregs is administered; and the population of Tcons and the population of Tregs comprise less than about 5 EU/ml endotoxins.

It shall be understood that different aspects and/or embodiments of the invention can be appreciated individually, collectively, or in combination with each other. Any description herein concerning a specific composition, multi-component pharmaceutical treatment, cell population, solution, formulation, kit, and/or method apply to and may be used for any other specific composition, multi-component pharmaceutical treatment, cell population, solution, formulation, kit, and/or method. In other words, any aspect or embodiment described herein can be combined with any other aspect or embodiment as disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A and FIG. 1B illustrate the schematics of the transplant according to the methods described herein (identified as High-Precision Orca-T or OrcaT) and the differences compared to a standard of care (SOC) cohort (identified as Conventional Transplant or SOC).

FIG. 1C illustrates a schematic of graft production and administration.

FIG. 2A illustrates the weight of patients enrolled in the study disclosed in the Examples.

FIG. 2B and FIG. 2C illustrate the HSPC and Treg cell dose administered to the patients enrolled in the study disclosed in the Examples.

FIG. 2D illustrates the purity of Treg cells administered to the patients enrolled in the study disclosed in the Examples.

FIG. 3A shows the time to platelet engraftment in the study group (identified as Orca-T) and the standard of care (SOC) cohort.

FIGS. 3B-3L illustrate engraftment of various cell populations in the patients in the study group disclosed in the Examples. The figures also illustrate the levels of each cell type in the donors before sample collection. Boxplots where shown: boxes show the 75th, 50th, and 25th percentiles; whiskers show the 90th and 10th percentiles. X-axes nomenclature: the leading number (e.g. 01, 02, 025, . . . ) are mentioned for ordering; following the underscore, Dscrn=healthy donor pre-G-CSF mobilization, Rscrn=recipient within 1 month prior to conditioning, apher=healthy donor blood draw at the time of apheresis, d028=recipient day 28 post-transplant, d056-d365=recipient days post-transplant. N's shown indicate the sample sizes for each timepoint. Symbols indicate values for individual measurements. Cell numbers×10⁻³ per uL of blood are equivalent to ×1,000 cells per uL of blood.

FIGS. 3M-3N show the timeline of lymphocyte and monocyte engraftment in a subset of the study group (Orca-T) and the standard of care cohort.

FIG. 30 shows representative flow cytometry data for the frequency of CD3+CD4+ T cells that were Tregs in two subjects compared to a healthy control. In the healthy control, 3.72% of circulating CD3+CD4+ T cells were Tregs (CD25+CD127dim). In the two graft recipients, 28.1% and 23.7% of CD3+CD4+ T cells were Tregs on day +28, 32.3% and 17.8% on day +56, and 19.2% and 20.7% on day +100 post-transplant.

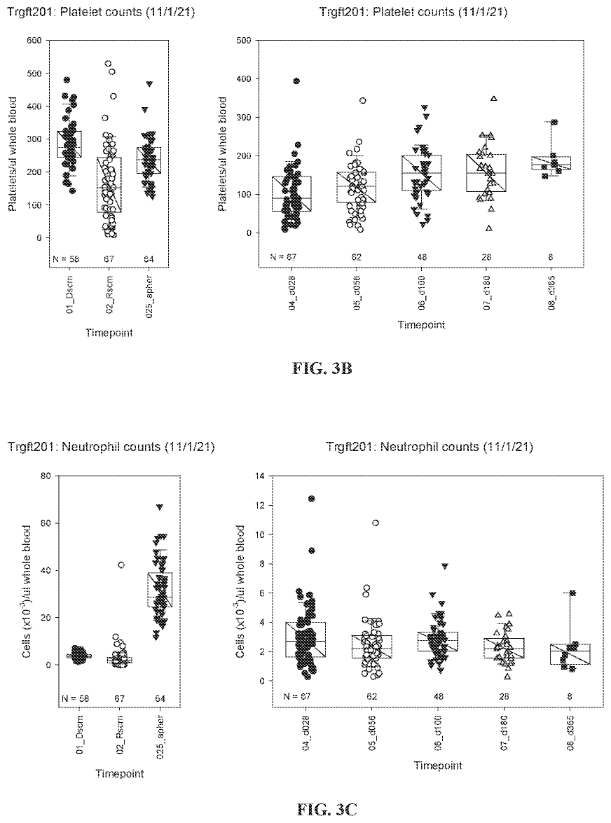

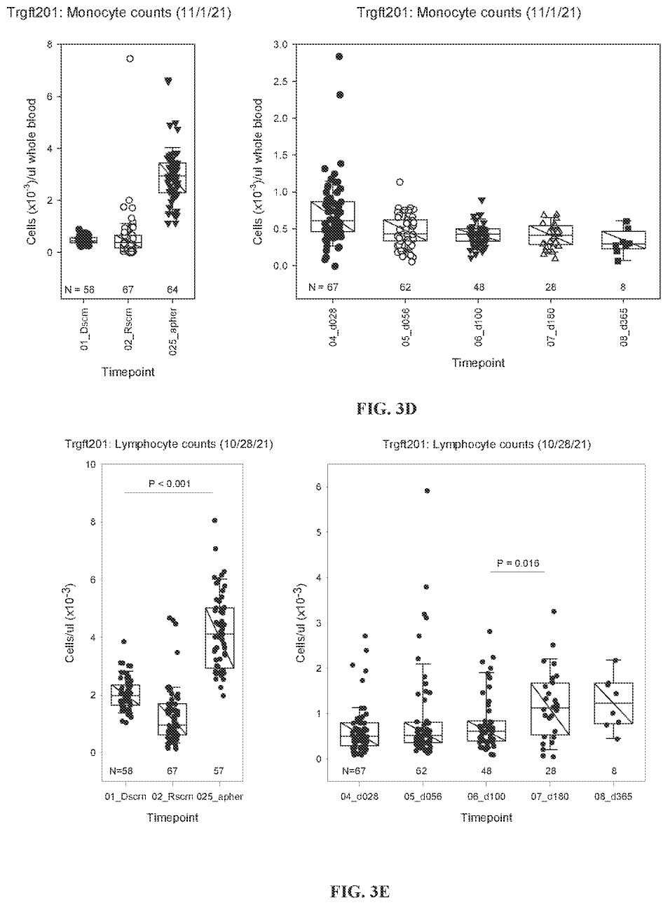

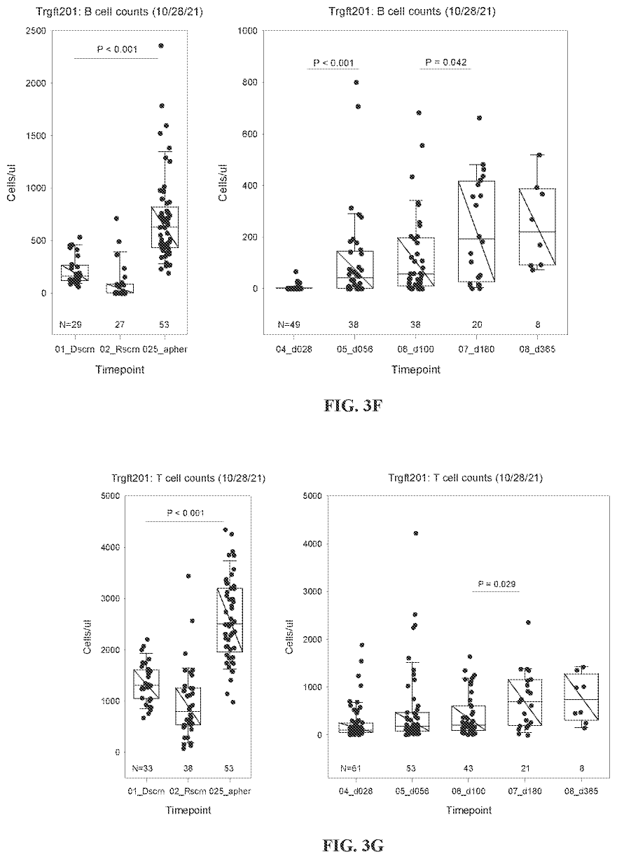

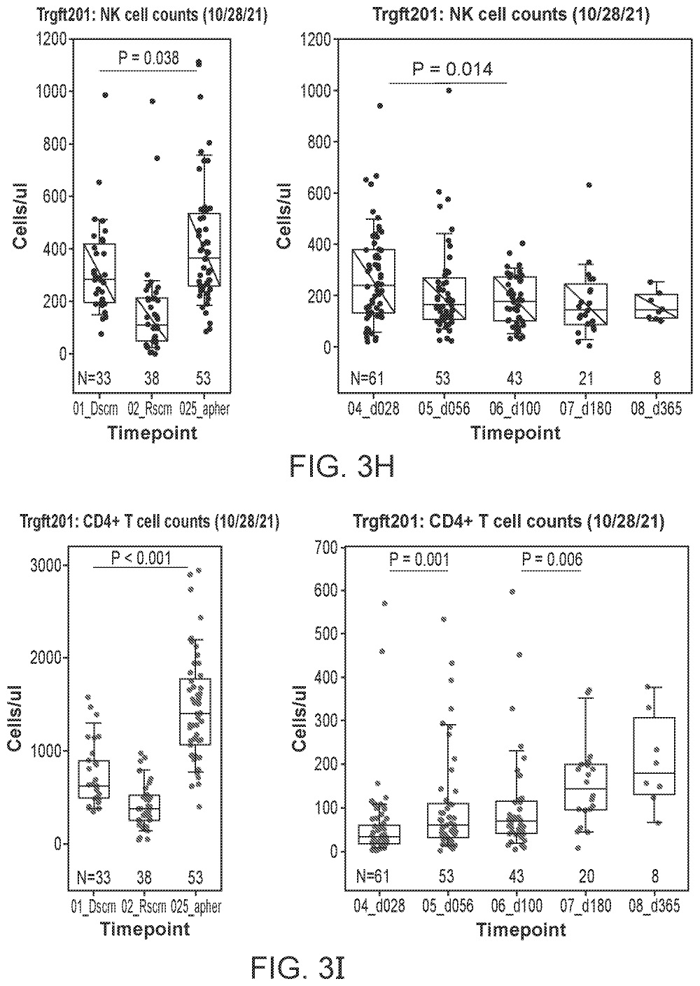

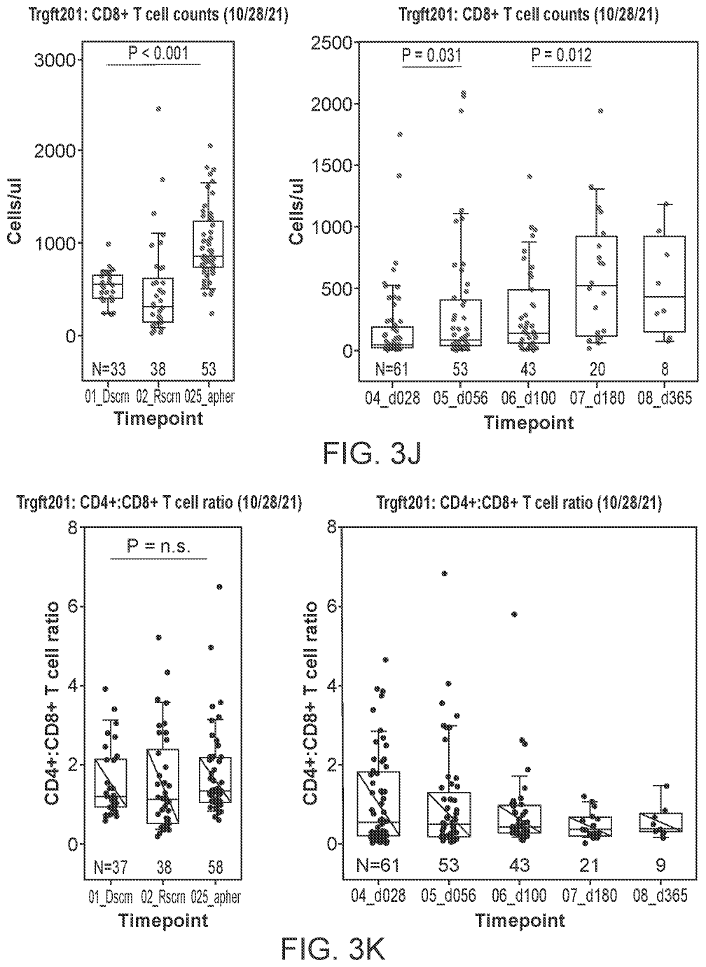

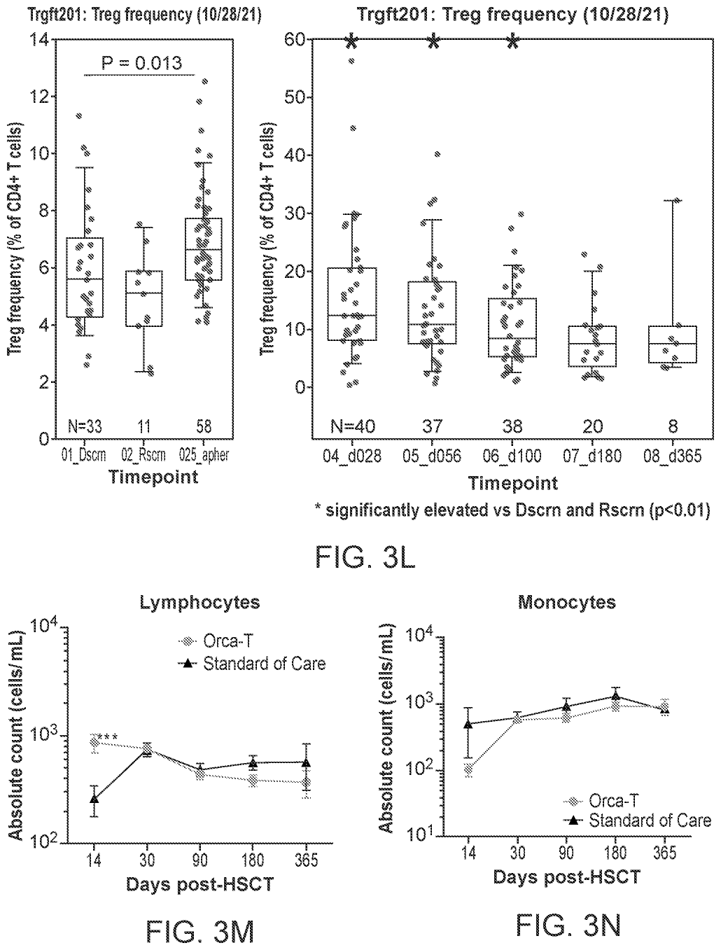

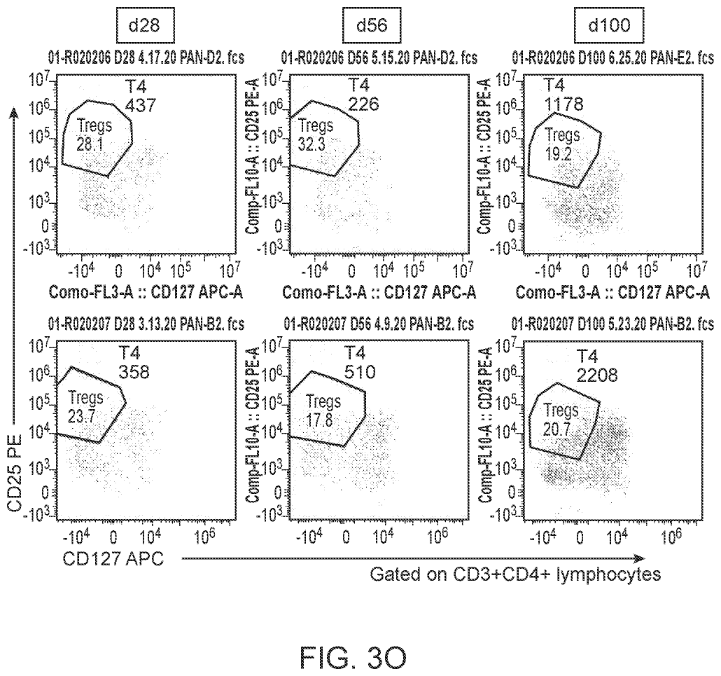

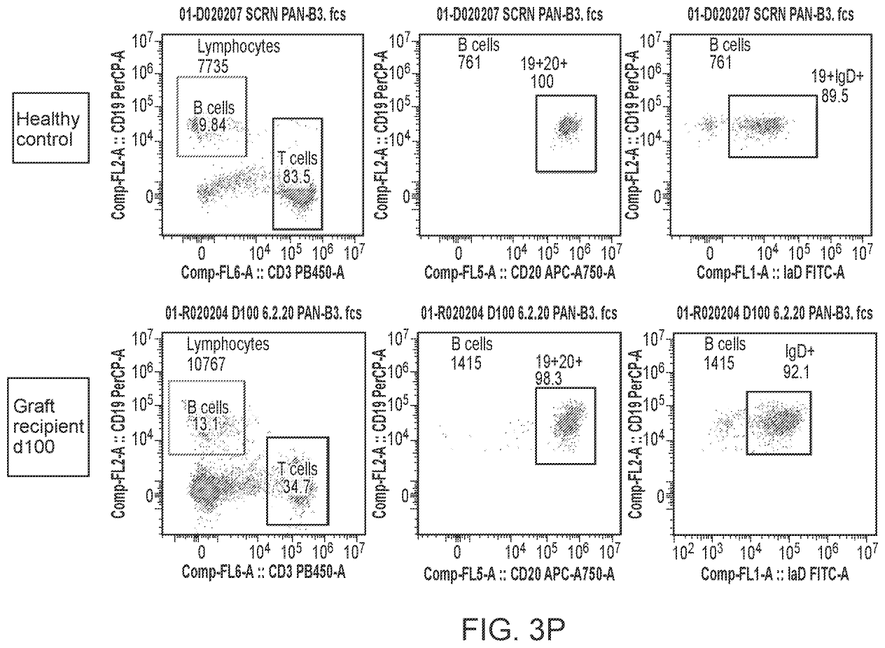

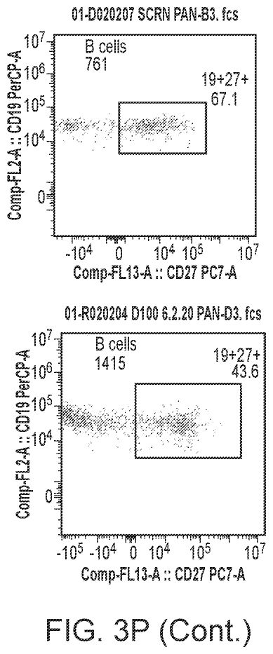

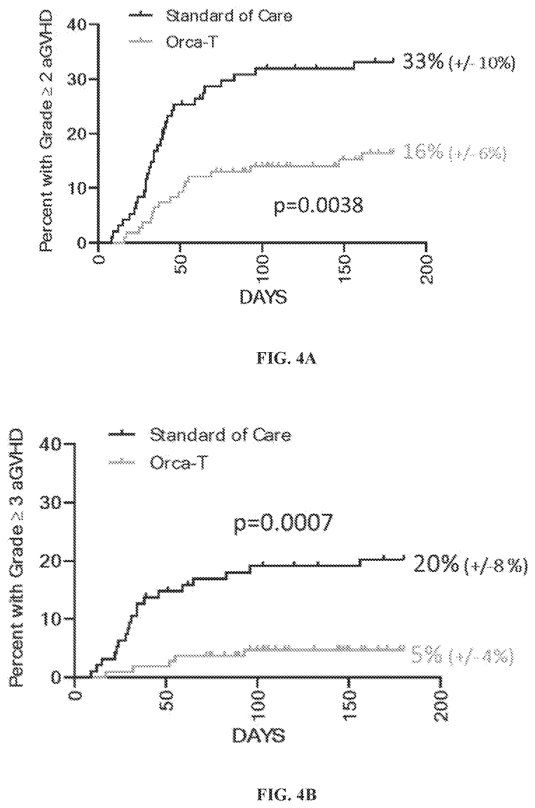

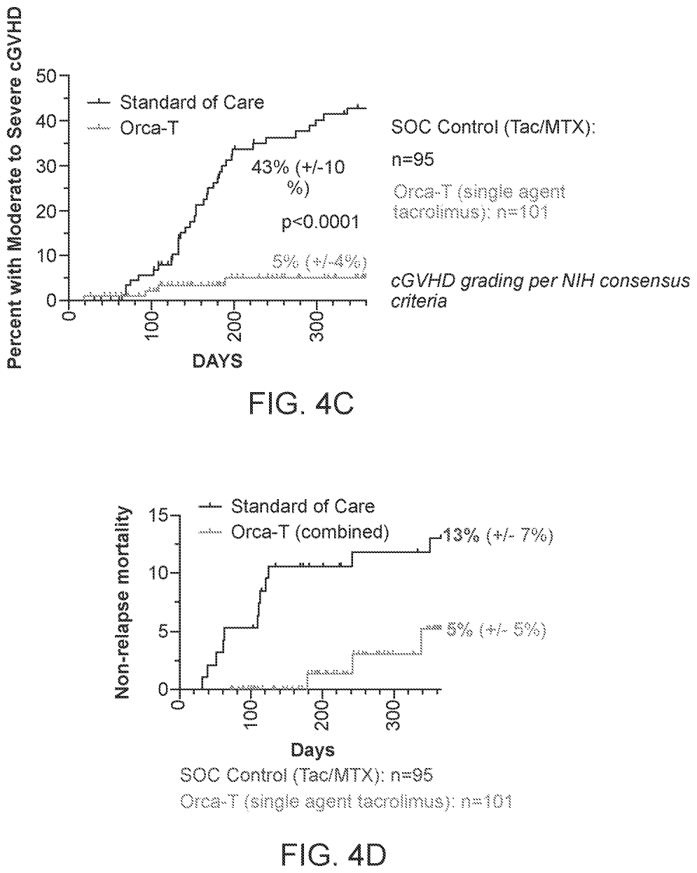

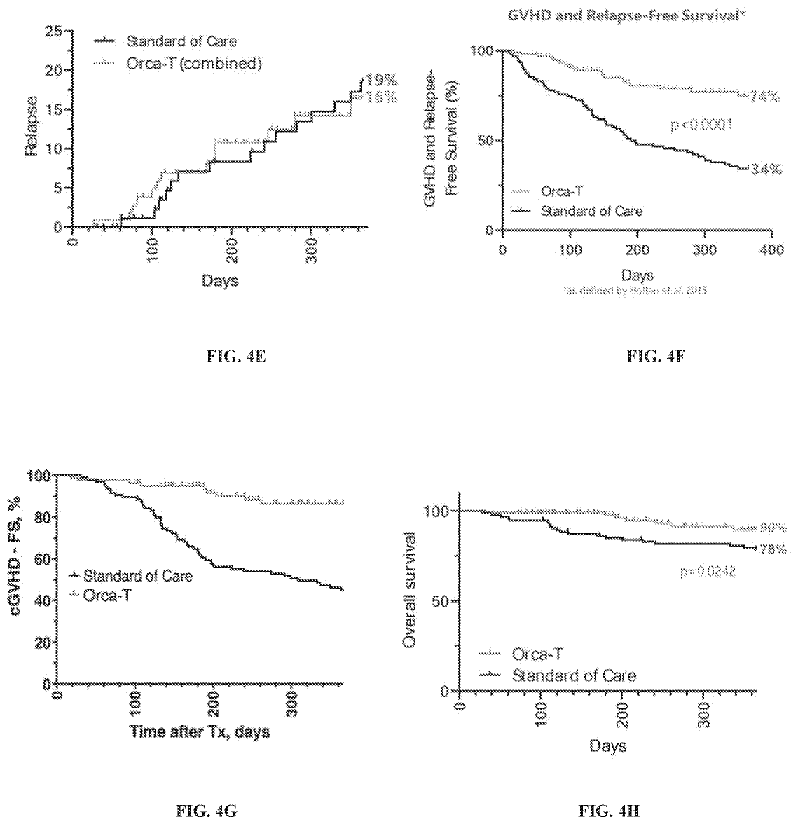

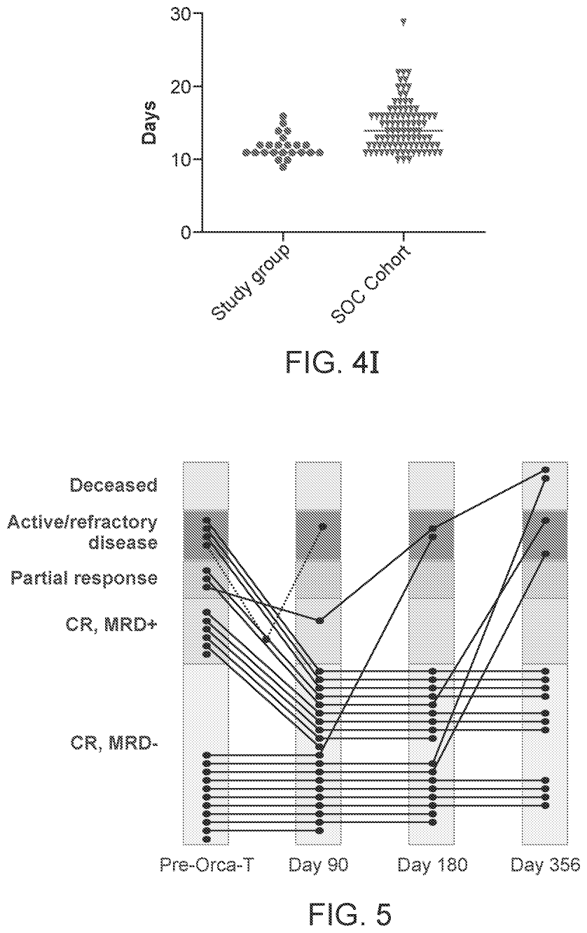

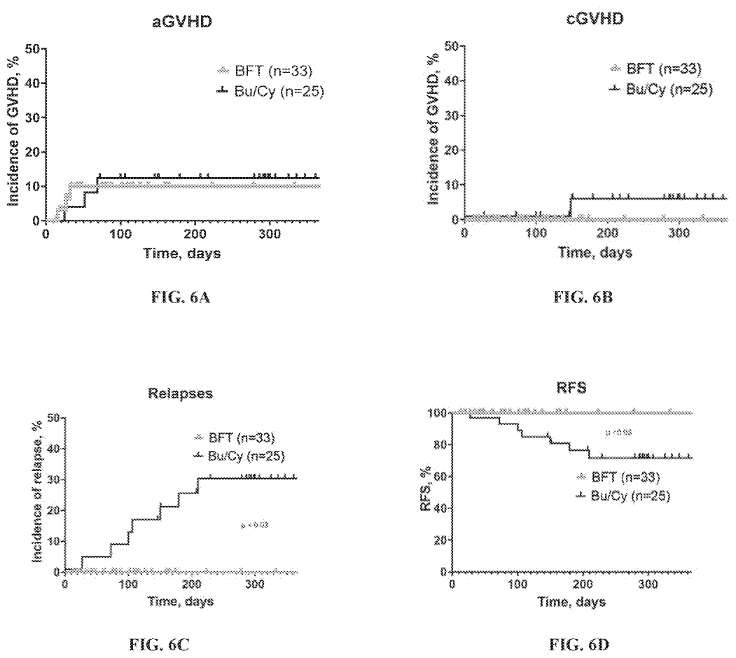

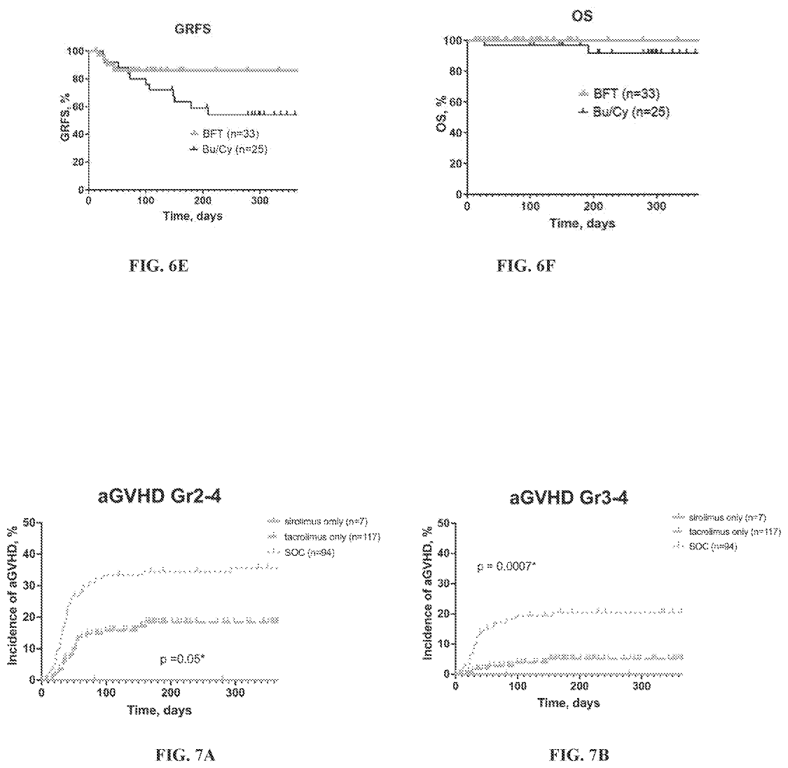

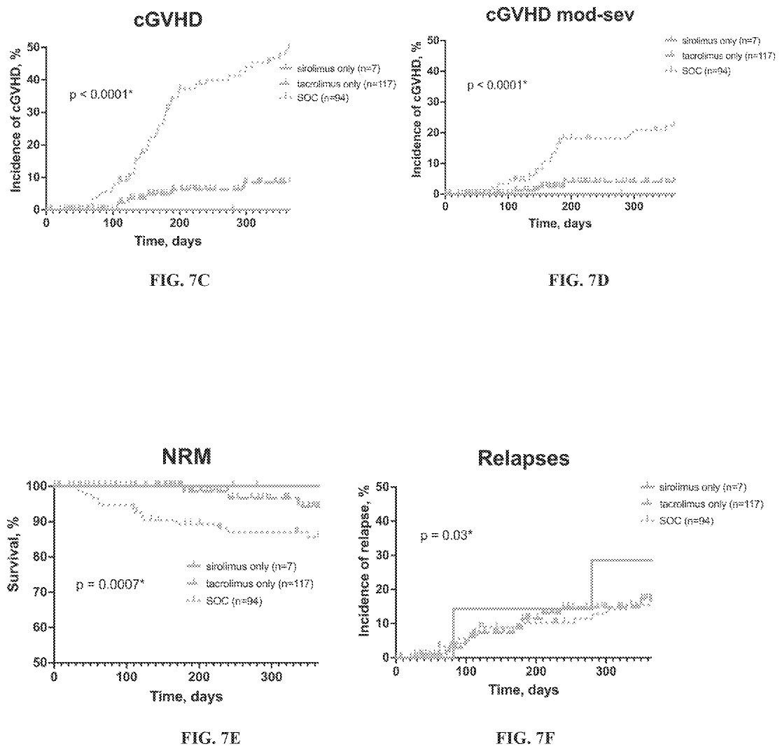

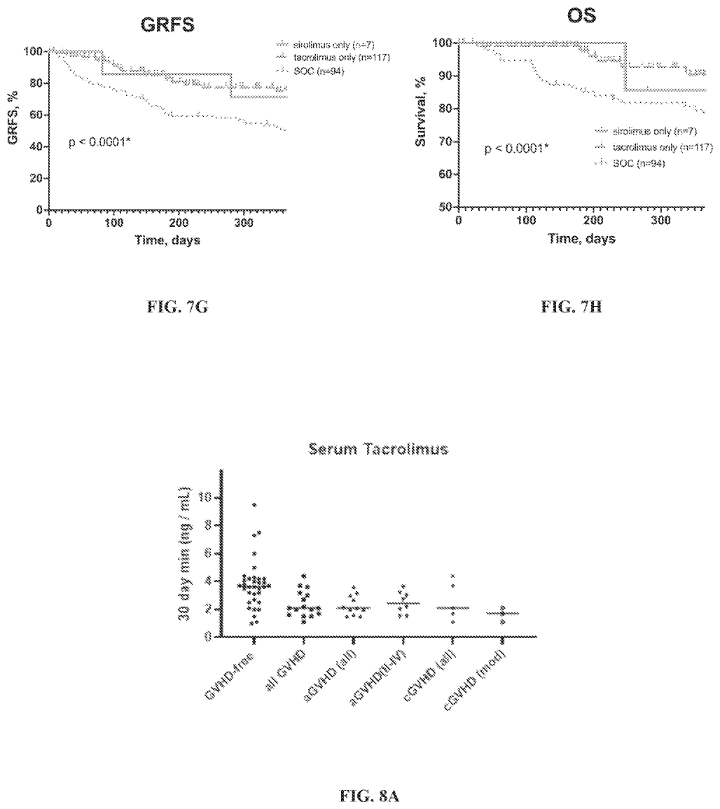

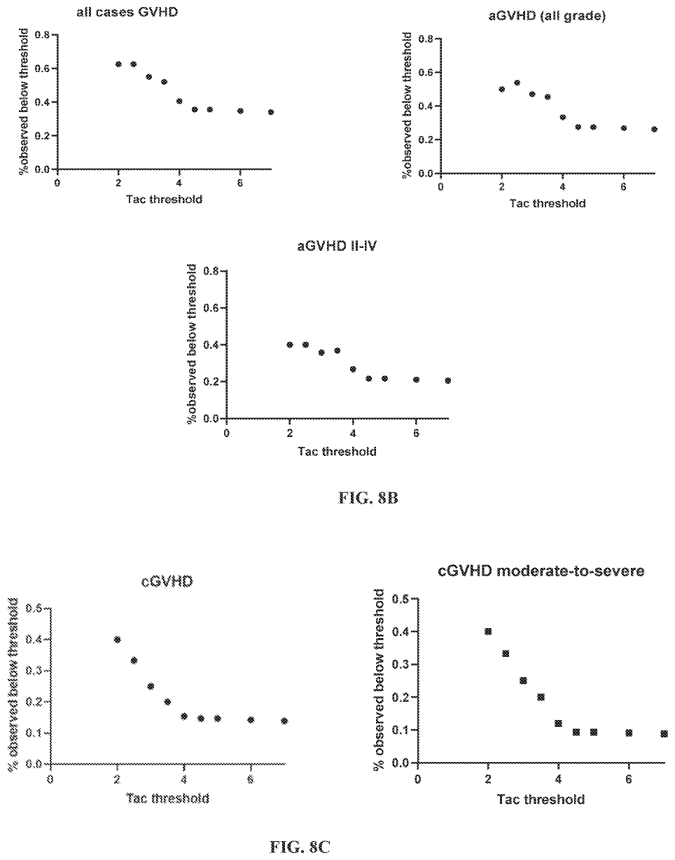

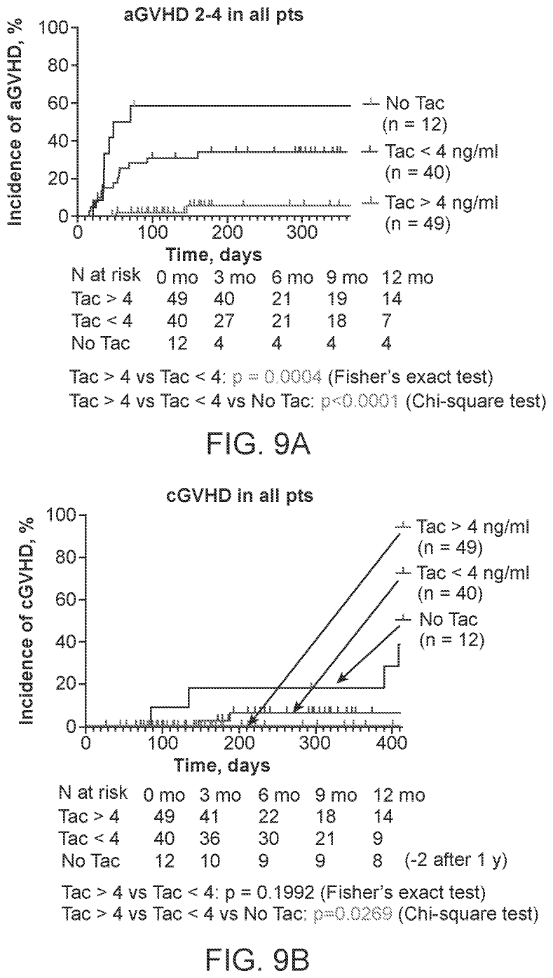

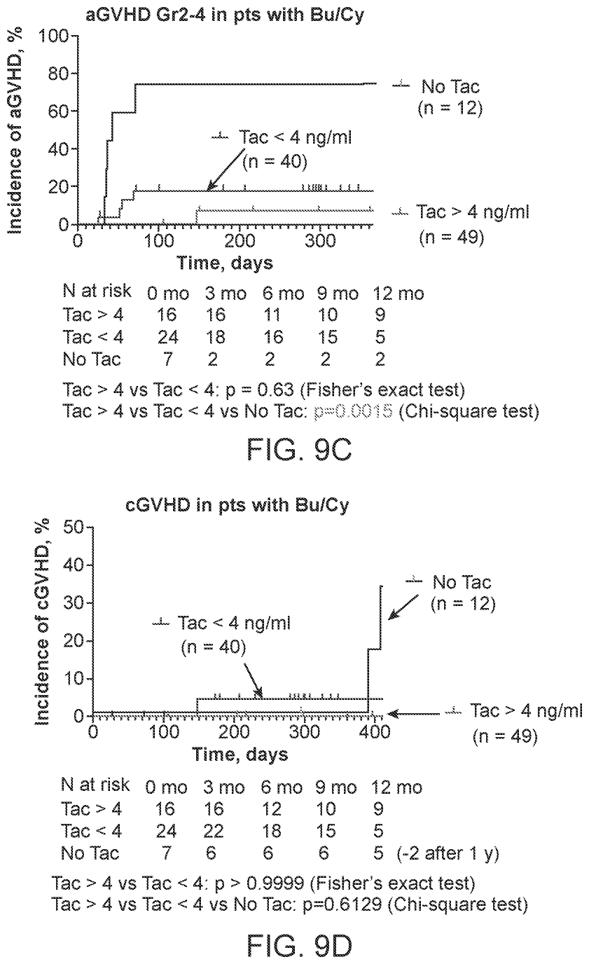

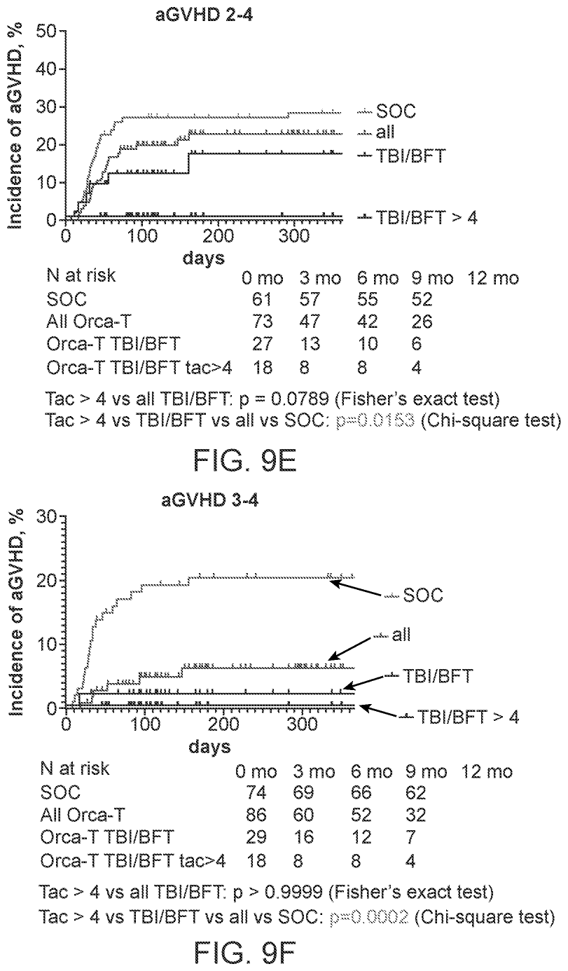

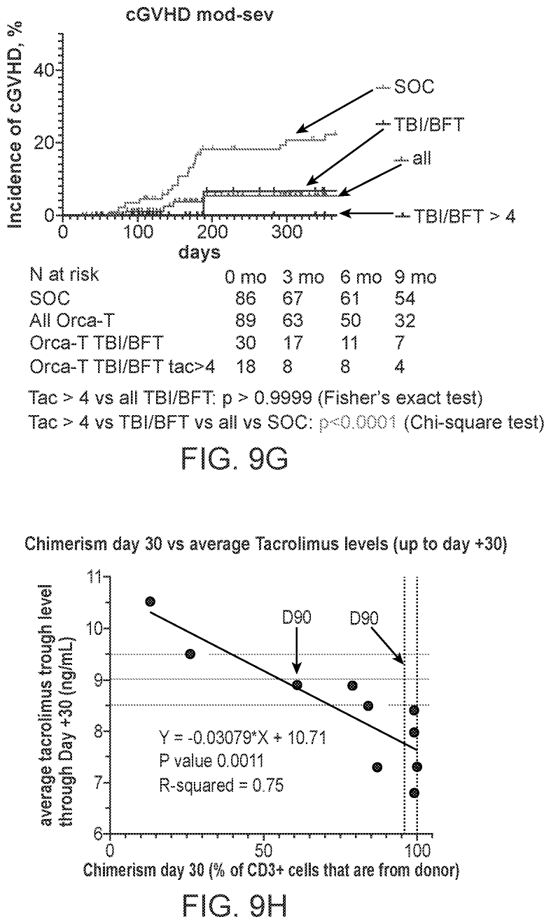

FIG. 3P shows flow cytometry data for B cell markers from a sample from a recipient of a composition of the disclosure compared to a healthy control. In all cases, the Y axis is for CD19+ staining. The left panels show gating of lymphocytes to identify B cells (CD19+) and T cells (CD3+). 13.4% of lymphocytes in the graft recipient were B cells, compared to 9.84% in the healthy control. The second from left panels show that 98.3-100% of cells gates as CD19+ were also CD20+. The panels second from the right show the fraction of B cells that are IgD+, which can be used to identify mature B cells. 92.1% of B cells in the graft recipient were IgD+, and 89.5% in the healthy control. The right-most panels show staining for CD27, which can be used to identify memory B cells, late plasmablasts, and plasma cells, for example. 43.6% of B cells in the graft recipient were CD27+, and 67.1% in the healthy control.

FIG. 4A shows the onset of grade ≥2 aGVHD in the study group (Orca-T) and the standard of care cohort through day +120 post-transplant. At nearly all timepoints, Orca-T data is below the standard of care data.

FIG. 4B shows the onset of grade ≥3 aGVHD in the study group (Orca T) and the standard of care cohort through day +120 post-transplant. At nearly all timepoints, Orca-T data is below the standard of care data.

FIG. 4C shows the onset of moderate to severe cGVHD in the study group (Orca-T) and the standard of care (SOC) cohort through day +365 post-transplant. At nearly all timepoints, Orca-T data is below the standard of care data.

FIG. 4D shows the non-relapse related mortality in the study group (Orca-T) and the standard of care (SOC) cohort through day +365 post-transplant. At nearly all timepoints, Orca-T data is below the standard of care data.

FIG. 4E shows relapse rates in the study group (Orca-T) and the standard of care cohort through day +365 post-transplant. At the final timepoint, Orca-T relapse rate is 16% and the standard of care relapse rate is 19%.

FIG. 4F shows GVHD and relapse-free survival rates in the study group (Orca-T) and the standard of care cohort through day +365 post-transplant. At nearly all timepoints, Orca-T data is above the standard of care data.

FIG. 4G shows cGVHD-free survival rates in the study group and the standard of care cohort through day +365 post-transplant. At nearly all timepoints, Orca-T data is above the standard of care data.

FIG. 4H shows overall survival rates in the study group and the standard of care cohort through day +365 post-transplant. At the final timepoint, Orca-T overall survival rate is 90% and the standard of care overall survival rate is 78%. FIG. 4I shows hospitalization days in a subset of the study group and the standard of care (SOC) cohort through day +365 post-transplant

FIG. 5 summarizes the disease status of a small subset of subjects in the study group before transplant and at day +90, +180, and +356 post-transplant. CR signifies complete remission, MRD signifies minimal residual disease.

FIGS. 6A-6F compare the aGVHD, cGVHD, relapse, relapse-free survival, GVHD and relapse free survival (GRFS) and overall survival rates in a subset of the patients in the study group that received different conditioning regimens.

FIGS. 7A-7H compare the aGVHD, cGVHD, non-relapse related mortality, relapse, relapse-free survival, GVHD and relapse free survival (GRFS) and overall survival rates in a subset of the patients in the study group that received different GVHD prophylactic agents.

FIGS. 8A-8C illustrate aGVHD and cGVHD rates in patients with different serum tacrolimus trough levels.

FIG. 9A and FIG. 9B compare the aGVHD and cGVHD levels in patients that had different serum tacrolimus levels.

FIG. 9C and FIG. 9D compare the aGVHD and cGVHD levels in patients that had different serum tacrolimus levels but were given the same conditioning regimen of busulfan and cyclophosphamide (Bu/Cy).

FIGS. 9E-9G compare the aGVHD and cGVHD levels in patients that had different serum tacrolimus levels but were given the same conditioning regimen of Total Body Irradiation (TBI)/Busulfan, Fludarabine, Thiotepa (TBI/BFT).

FIG. 9H shows the average trough tacrolimus level through day +30 post-transplant, plotted against the proportion of CD3+ cells of donor origin at day +30 (except that chimerism data is from day 90 where indicated by “D90”).

### DETAILED DESCRIPTION

### Introduction

Various embodiments of the invention provide compositions, multi-component pharmaceutical treatments, cell populations, solutions, formulations, kits, and/or methods relating to improved allogeneic hematopoietic stem cell transplantation (alloHCT).

AlloHCT is the transplantation of multipotent hematopoietic stem and progenitor cells (HSPCs), usually derived from donor bone marrow, peripheral blood, or umbilical cord blood, into a recipient. The recipient can be subjected to myeloablative conditioning, which kills hematopoietic cells including tumor cells and host immune cells. The HSPCs transplanted into the recipient then reconstitutes the hematopoietic compartment. HCT can be useful as a treatment for cancer due to the ability of donor T cells to exert anti-tumor effects, referred to as graft versus tumor (GVT). In patients with hematologic malignancies that are refractory to chemotherapy, HCT is associated with improved survival.

Surprisingly, alloHCT recipients who received a single agent graft versus host disease (GVHD) prophylactic consisting of tacrolimus had significantly better clinical outcomes than existing alloHCT regimens and standards of care. These recipients experience improved clinical outcomes including, for example, increased overall survival, increased relapse-free survival, increased GVHD- and relapse-free survival (GRFS), more rapid and/or complete engraftment of various hematopoietic components (e.g., neutrophils, platelets, T cells, B cells), improved donor chimerism (e.g., T cell chimerism), decreased relapse, decreased primary graft failure, decreased secondary graft failure, decreased treatment-associated mortality, reduced acute and/or chronic GVHD, and shorter time to discharge from hospital following the single agent GVHD prophylactic consisting of tacrolimus

Although alloHCT is associated with improved survival in patients with hematologic malignancies that are refractory to chemotherapy, some subjects treated with existing alloHCT regimens exhibit cancer relapse, and a number of complications can limit the efficacy of alloHCT. The effectiveness of alloHCT can be limited by, for example, primary graft failure, secondary graft failure, limited or slow engraftment of various hematopoietic components (e.g., neutrophils, platelets, T cells, or B cells), and limited donor chimerism (e.g., T cell chimerism). Additionally, alloHCT can cause treatment-associated morality or toxicity, for example, However, donor T cells can also attack non-tumor host cells, resulting in graft versus host disease (GVHD). GVHD is a major source of post-HCT complications and can be fatal. Management of GVHD can require immunosuppressive therapy or cytotoxic mediations, which can cause toxicity, increase susceptibility to infection, and/or blunt anti-tumor immunity. The early morbidity and mortality associated with acute graft versus host disease (aGVHD; which occurs within the first 100 days post-transplant) is a major factor limiting the success of HCT, as is the long-term morbidity associated with chronic GVHD (cGVHD). GVHD is a risk for both HLA-matched and HLA-mismatched transplantations. GVHD can occur even if the donor and recipient are HLA-matched, because the immune system can still recognize other differences between in the donor tissues.

Both GVT and GVHD are largely mediated by conventional T cells (Tcons), which mount immune responses upon recognition of cognate antigen by T cell receptors. Depleting T cells from hematopoietic stem cell transplantation (HCT) grafts can reduce GVHD, but can also result in reduced GVT and increased likelihood of cancer relapse. Besides Tcons, Tregs are an additional subset of T cells that negatively regulate inflammation and that promote immune tolerance. Tregs can prevent or reduce GVHD through their negative regulation of inflammation, including, for example, inflammation elicited by donor Tcons when they recognize recipient antigens.

Provided herein are methods for improved alloHCT, comprising administering to a subject certain cell populations that comprise populations of cells, including a first population of CD45+ cells that comprises, at least, HSPCs, a population of cells, at least, enriched for Tregs, and a second population of CD45+ cells that comprises, at least, Tcons. Without wishing to be bound by theory, administering the cell population enriched for Tregs reduces the incidence and/or severity of GVHD, while administering the second population of CD45+ cells, which comprises Tcons, enhances GVT. Thus, embodiments of the invention provide a provides compositions, multi-component pharmaceutical treatments, cell populations, solutions, formulations, kits, and/or methods for administering, at least, both populations of T cells, to enhance GVT while minimizing GVHD. Accordingly, the compositions, multi-component pharmaceutical treatments, cell populations, solutions, formulations, kits, and/or methods disclosed herein can retain the graft-versus-tumor (GVT) effects of alloHCT administered to a subject having a cancer (e.g., a hematologic cancer), while preventing or reducing graft versus host disease (GVHD) in the subject. In some embodiments, two or more populations of cells are administered at different times, for example, first population of CD45+ cells that comprises, at least, HSPCs and the cell population enriched for Tregs can be administered prior to the second population of CD45+ cells that comprises, at least, Tcons.

### I. Cell Populations

Embodiments of the invention provide a multi-component pharmaceutical treatment to be administered to a human subject in need thereof. The multi-component treatment comprises (a) a solution comprising a first population of CD45+ cells comprising hematopoietic stem and progenitor cells (HSPCs) and granulocytes wherein at most about 10% of the first population of CD45+ cells comprise granulocytes; (b) a solution comprising a population of cells enriched for regulatory T cells (Tregs); (c) a solution comprising a second population of CD45+ cells wherein the second population of CD45+ cells comprise at least about 20% CD3+ conventional T cells (Tcons), at least about 10% monocytes, and at least about 10% granulocytes; and (d) a solution comprising one or more doses of a graft vs host disease (GVHD) prophylactic agent, e.g., tacrolimus. In various embodiments, the HSPCs are CD34+.

In some embodiments, the first population of CD45+ cells comprising HSPCs comprises from about 5×10⁵ to about 2×10⁷ HSPCs per kilogram of ideal body of said human subject. In embodiments, the first population of CD45+ cells comprises at least about 0.5% granulocytes, at least about 1% granulocytes, at most about 5% granulocytes, at most about 3% granulocytes, at most about 3% monocytes, at most about 2% monocytes, at most about 0.5% lymphocytes, at most about 2% lymphocytes, at least about 15% granulocytes, at least about 20% granulocytes, at most about 35% granulocytes, at most about 30% granulocytes, at most about 25% granulocytes, at least about 15% monocytes, at least about 20% monocytes, at most about 35% monocytes, at most about 30% monocytes, at most about 25% monocytes, at least about 0.5% NK cells, and or at least about 2% NK cells. In various embodiments, the first population of CD45+ cells, the population of cells enriched for Tregs, and the second population of CD45+ cells are obtained from a single donor. In some embodiments, the first population of CD45+ cells, said population of cells enriched for Tregs, and/or said second population of CD45+ cells is allogeneic relative to said human subject. In embodiments, the first population of CD45+ cells, said population of cells enriched for Tregs, and/or said second population of CD45+ cells is obtained from a donor that is HLA-matched relative to said human subject. In various embodiments, the first population of CD45+ cells, said population of cells enriched for Tregs, and/or said second population of CD45+ cells is obtained from a donor that is HLA-mismatched relative to said human subject. In some embodiments, the first population of CD45+ cells, said population of cells enriched for Tregs, and/or said second population of CD45+ cells is obtained from a donor that is haploidentical relative to said human subject.

In embodiments, the Tregs are CD4+CD25+CD127dim or CD4+FOXP3+. In some cases, the population of cells enriched for Tregs comprises CD45+ cells, e.g., more than about 90% of said CD45+ cells are Tregs. In various embodiments, the population of cells enriched for Tregs comprises from about 1×10⁵ to about 1×10⁷ Tregs per kilogram of actual or ideal body weight of said human subject or from about 5×10⁵ to about 4×10⁶ Tregs per kilogram of actual or ideal body weight of said human subject.

In some embodiments, the second population of CD45+ cells comprises from about 1×10⁵ to about 1×10⁷ Tcons per kilogram of actual or ideal body weight of said human subject or the second population of CD45+ cells comprises from about 5×10⁵ to about 5×10⁶ Tcons per kilogram of actual or ideal body weight of said human subject. In embodiments, the second population of CD45+ cells comprises at least about 0.1% CD34+ cells or from about 0.2% to about 20% CD34+ cells and/or at least about 0.1% Tregs. In various embodiments, the second population of CD45+ cells comprises a population of memory T cells (Tmems), e.g., Tmems that are CD3+CD45RA− CD45RO+. In some embodiments, the population of Tmems comprises more than about 3×10⁵ Tmems per kilogram of ideal body actual or ideal body weight of said human subject. In embodiments, the population of Tmems comprises from about 3×10⁵ to about 1×10⁹ Tmems per kilogram of ideal body actual or ideal body weight of said human subject. In various embodiments, the second population of CD45+ cells comprises a population of invariant natural killer T cells (iNKTs), e.g., iNKTs that are CD3+Vα24Jα18+. In some embodiments, the population of iNKTs comprises more than about 5×10² iNKTs per kilogram of ideal body actual or ideal body weight of said human subject. In embodiments, the population of iNKTs comprises from about 5×10² to about 1×10⁷ iNKTs per kilogram of ideal body actual or ideal body weight of said human subject.

Provided herein are compositions, multi-component pharmaceutical treatments, cell populations, solutions, formulations, kits, and/or methods for improved hematopoietic stem cell transplantation (HCT), for example, allogeneic hematopoietic stem cell transplantation (alloHCT). Compositions, multi-component pharmaceutical treatments, cell populations, solutions, formulations, kits, and/or methods disclosed herein can comprise one or more cell populations that can be administered in combination with a GVHD prophylactic agent to achieve positive clinical outcomes. A cell population can comprise one or more types of cells, for example, hematopoietic stem and progenitor cells (HSPCs), conventional T cells (Tcons), regulatory T cells (Tregs), invariant natural killer T cells (iNKTs), memory T cells (Tmems), and combinations thereof.

The disclosure provides parameters for cell populations and methods of administering cell populations that can contribute to successful clinical outcomes in HCT recipient subjects. Without wishing to be bound by theory, parameters that can contribute to successful clinical outcomes in HCT recipient subjects include, for example, co-administration of a GVHD prophylactic agent as described herein (e.g., tacrolimus), populations administered, order and timing for the administration of different populations, purity standards for populations, methods for obtaining populations, methods of handling or storing populations, dosages of populations administered, methods for obtaining populations, and combinations thereof.

In various embodiments, administering comprises infusing into said human subject said first population of CD45+ cells, said population of cells enriched for Tregs, and said second population of CD45+ cells.

In some embodiments, said second population of CD45+ cells (as disclosed herein) is administered at least about 12 hours after said first population of CD45+ cells (as disclosed herein), said second population of CD45+ cells is administered from about 24 to about 96 hours after said first population of CD45+ cells, said second population of CD45+ cells is administered from about 36 to about 60 hours after said first population of CD45+ cells, said second population of CD45+ cells is administered at least about 12 hours after said population of cells enriched for Tregs (as disclosed herein), said second population of CD45+ cells is administered from about 24 to about 96 hours after said population of cells enriched for Tregs, and/or said second population of CD45+ cells is administered from about 36 to about 60 hours after said population of cells enriched for Tregs.

HSPCs can have extensive self-renewal capacity, and an ability to differentiate into specialized cell types, for example, an ability to reconstitute all hematopoietic cell lineages. HSPCs can undergo asynchronous replication, where two daughter cells are produced with different phenotypes. HSPCs cells can exist in a mitotically quiescent form. HSPCs can be derived from bone marrow, peripheral blood, and/or umbilical cord blood.

Subsets of immune cells, such as conventional T cells (Tcons), regulatory T cells (Tregs), invariant natural killer T cells (iNKTs), and memory T cells (Tmems) can contribute to aspects of GVHD following HCT, and can also contribute to, for example, GVT immune responses, immune reconstitution, infection susceptibility, and patient survival.

GVHD can be mediated in large part by donor T cells, which can elicit inflammatory responses upon recognition of recipient antigens. T cell depletion (TCD) of cell populations for transplantation to a subject can be undertaken to decrease the likelihood of acute and/or chronic GVHD. T cells can be depleted using methods including, but not limited to, physical adsorption of T cells to protein ligands such as lectins, immunodepletion with T cell specific antibodies, and immunoaffinity techniques (for example, use of T cell or lymphocyte-specific antibodies in immunoadsorption columns, magnetic activated cell sorting (MACS), or fluorescent activated cell sorting (FACS)). Applying TCD techniques to donor grafts can result in, for example, 10-fold to 10⁵-fold depletion of T cells, and reduced incidence of GVHD. However, TCD can also result in increased incidence of cancer relapse, as the lack of T cells can reduce a graft-versus-tumor (GVT) immune response. Additionally, TCD can result in impaired immune recovery, and increased susceptibility to infections.

Both GVT and GVHD can be largely mediated by conventional T cells (Tcons), which mount immune responses upon recognition of cognate antigen by T cell receptors (tumor antigens for GVT, non-tumor recipient antigens for GVHD). Tcons can, for example, contribute to GVT, GVHD, or a combination thereof. In some embodiments, administration of Tcons after administration of Tregs can enhance GVT immunity, and/or reduce susceptibility to infection.

Tcons can broadly refer to all CD3+ T cells, cells expressing CD3 and CD4 or cells expressing CD3 and CD8, cells expressing medium to high levels of CD127, cells expressing CD3 and medium to high levels of CD127, cells expressing CD3, cells expressing medium to high levels of CD127, and cells expressing CD4 or CD8. In some embodiments, Tcons do not express Vα24Jα18 TCR. Tcons and Regulatory T cells (“Tregs”) can be non-mutually-exclusive cell populations. In some embodiments, Tcons and Tregs are mutually exclusive cell populations.

Regulatory T cells (“Tregs”) are a specialized subpopulation of T cells that negatively regulate (e.g., suppress) activation of the immune system and thereby promote immune tolerance. Without wishing to be bound by theory, cell populations of the disclosure enriched for Tregs contribute to positive clinical outcomes by, for example, reducing the incidence and/or severity of GVHD in a transplant recipient subject, and/or improving immune reconstitution in a transplant recipient. Administering cell population enriched for Tregs with a population of CD45+ cells that comprises, at least, HSPCs can, for example, facilitate retention of graft versus tumor (GVT) and reduced incidence and/or severity of GVHD. Without wishing to be bound by theory, administering population of cells enriched for Tregs can prevent GVHD, and administering second population of CD45+ cells that comprises, at least, Tcons can promote GVT effects, for example, relative to alternate hematopoietic stem cell transplantation (HCT) methods, i.e., methods that are distinct from the compositions, multi-component pharmaceutical treatments, cell populations, solutions, formulations, kits, and/or methods disclosed and/or claimed herein. In some embodiments, administering a population of cells enriched for Tregs reduces the risk of developing GVHD, and administering second population of CD45+ cells that comprises, at least, Tcons promotes GVT effects relative to alternate HCT methods, i.e., methods that are distinct from the compositions, multi-component pharmaceutical treatments, cell populations, solutions, formulations, kits, and/or methods disclosed and/or claimed herein.

As used herein, an alternate composition lacks one or more cell populations and/or prophylactic agent that are disclosed herein and/or recited in the claims. As examples, an alternate composition lacks one or more of a cell population comprising HSPCs, a cell population comprising Tregs, a cell population comprising Tcons, and a prophylactic agent. In some embodiments, an alternate composition or treatment regimen comprises an additional cell population or agent compared to a composition or treatment regimen of the disclosure, e.g., a an additional or different GVHD prophylactic agent.

There are a number of subsets of Tregs, for example, TCRαβ+CD4+ regulatory T cells, which include natural regulatory T cells (nTregs) and induced regulatory T cells (iTregs). nTregs can be T cells produced in the thymus and delivered to the periphery as a long-lived lineage of self-antigen-specific lymphocytes. iTregs can be recruited from circulating lymphocytes and acquire regulatory properties under particular conditions of stimulation in the periphery. nTregs and iTregs are CD4+CD25+; both can inhibit proliferation of CD4+CD25− T cells in a dose-dependent manner. In some embodiments, Tregs are anergic and do not proliferate upon TCR stimulation. In addition to being positive for CD4 and CD25, Tregs can be positive for the transcription factor FOXP3, an intracellular marker. Tregs can be identified or selected based on various marker expression profiles. Non-limiting examples of marker expression profiles that can be used to select Tregs include (1) CD4+CD25+CD127dim, (2) CD4+FOXP3+, (3) CD3+CD4+CD25+, (5) CD3+CD4+CD25+CD127dim, (6) CD3+CD4+CD25+CD127dim FOXP3+, (7) CD3+FOXP3+, (8) CD3+CD4+FOXP3+, (9) CD3+CD4+CD25+FOXP3+, (10) CD3+CD25+FOXP3+, (11) CD3+CD25+CD127dim, (12) CD4+CD25+, (13) CD4+CD25+CD127dimFOXP3+, (14) FOXP3+, CD4+FOXP3+, (15) CD4+CD25+FOXP3+, (16) CD25+FOXP3+, and (17) CD25+CD127dim.

Selection based on certain expression profiles can be achieved based on extracellular markers and without requiring cell permeabilization, for example, selection based on CD4+CD25+CD127dim.

A cell population that comprises Tregs can, for example, reduce the incidence of graft rejection, reduce the incidence and/or severity of GVHD, promote hematopoietic reconstitution, promote immune reconstitution, promote mixed chimerism, or a combination thereof.

A cell population of the disclosure can comprise invariant natural killer T cells (iNKTs). iNKTs are subclass of CD1d-restricted Natural Killer T (NKT) cells that express a highly conserved αβ-T cell receptor that comprises of Vα24Jα18 TCRα chain in humans (referred to herein as “Vα24Jα18+”). iNKT cells can be identified by binding with CD1d-multimers like that are loaded with u-galactosylceramide (GalCer), PBS-57, PBS-44 or other natural or synthetic glycolipids. Another method of identification is an antibody or combination of antibodies that specifically recognize the Vα24Jα18 region. Examples include a Vα24 antibody, a Jα18 antibody, or the monoclonal antibody clone 6B11 which binds specifically to a unique region of the Vα24Jα18 TCR and can be used to identify iNKT cells. iNKTs can be CD3+Vα24Jα18+.

In some embodiments, iNKTs can promote engraftment, promote GVT, reduce incidence and/or severity of GVHD, decrease susceptibility to cancer relapse, decrease susceptibility to infection, or a combination thereof. In some embodiments, iNKTs promote the activity of Tregs. In some embodiments, iNKTs promote the activity of HSPCs.

A cell population of the disclosure can comprise memory T cells (Tmems). Tmems can refer to antigen-experienced T cells that express, for example, the phenotypic markers CD45RO, TCRα, TCRβ, CD3, CD4, CD95, and IL-2Rβ or the phenotypic markers CD45RO, TCRα, TCRβ, CD3, CD8, CD95, and IL-2Rβ. Tmems provide immunity and are capable of persisting for a long period of time in an inactive state. Tmems are able to rapidly acquire effector functions upon re-challenge with antigen. A population of Tmems can include any combination of the subclasses T central memory cells and T effector memory cells. In some embodiments, Tmems are CD3+CD45RA−CD45RO+. In various methods, Tmems administered to a subject receiving HCT can, for example, promote GVT, reduce GVHD, decrease susceptibility to cancer relapse, decrease susceptibility to infection, or a combination thereof.

### A. Acquisition and Processing of Cells

In some embodiments, at least one mobilized peripheral blood donation is collected from a donor or at most two mobilized peripheral blood donations are collected from the donor.

In embodiments, at least one of the mobilized peripheral blood donations is processed and sorted to enrich CD34+ cells and Tregs. In some embodiments, the processing and sorting time of the one or more of the mobilized peripheral blood donations is less than about 35 hours, the processing and sorting time of the one or more of the mobilized peripheral blood donations is less than about 30 hours, the processing and sorting time of the one or more of the mobilized peripheral blood donations is less than about 25 hours, the processing and sorting time of the one or more of the mobilized peripheral blood donations is at most about 35 hours, and/or the processing and sorting time of the one or more of the mobilized peripheral blood donations is at most about 25 hours.

In various embodiments, the one or more of the mobilized peripheral blood donations is processed and sorted using one or more immune-separation particles (ISPs), e.g., ISPs comprise affinity reagents such as immuno-magnetic separation particles which may be antibodies each conjugated to an iron-containing particle. In some embodiments, the affinity reagents comprise a plurality of CD34-reagents (e.g., an anti-CD34 antibody) that binds to one or more CD34 receptors on a HSPC.

In some cases, at least a portion of the plurality of ISPs are attached to CD34+ receptors on the HPSC's of the HSPC cell population; optionally, an average number of ISP's per HSPC in the HSPC cell population is less than about 20,000, an average number of ISP's per HSPC in the HSPC cell population is equal to or less than about 10,000, and/or an average number of ISP's per HSPC in the HSPC cell population is from about 1000 to about 20,000.

In some embodiments, an average number of ISP's per HSPC in the HSPC cell population may be about 1,500 to about 20,000. In some embodiments, an average number of ISP's per HSPC in the HSPC cell population may be at least about 1,500. In some embodiments, an average number of ISP's per HSPC in the HSPC cell population may be at most about 20,000. In some embodiments, an average number of ISP's per HSPC in the HSPC cell population may be about 1,500 to about 2,000, about 1,500 to about 5,000, about 1,500 to about 6,000, about 1,500 to about 10,000, about 1,500 to about 12,000, about 1,500 to about 15,000, about 1,500 to about 20,000, about 2,000 to about 5,000, about 2,000 to about 6,000, about 2,000 to about 10,000, about 2,000 to about 12,000, about 2,000 to about 15,000, about 2,000 to about 20,000, about 5,000 to about 6,000, about 5,000 to about 10,000, about 5,000 to about 12,000, about 5,000 to about 15,000, about 5,000 to about 20,000, about 6,000 to about 10,000, about 6,000 to about 12,000, about 6,000 to about 15,000, about 6,000 to about 20,000, about 10,000 to about 12,000, about 10,000 to about 15,000, about 10,000 to about 20,000, about 12,000 to about 15,000, about 12,000 to about 20,000, or about 15,000 to about 20,000. In some embodiments, an average number of ISP's per HSPC in the HSPC cell population may be about 1,500, about 2,000, about 5,000, about 6,000, about 10,000, about 12,000, about 15,000, or about 20,000. In some embodiments, an average number of ISP's per HSPC in the HSPC cell population may be at least 1,500, 2,000, 5,000, 6,000, 10,000, 12,000, 15,000, or 20,000. In some embodiments, an average number of ISP's per HSPC in the HSPC cell population may be at most 1,500, 2,000, 5,000, 6,000, 10,000, 12,000, 15,000, or 20,000.

In some cases, at least a portion of the plurality of ISPs are attached to CD25+ receptors on the cells of the Treg cell population; optionally, an average number of ISP's per T-reg cell in the Treg population is equal or less than about 4000 or an average number of ISPs per T-reg cell in the Treg population is from about 1500 to about 2500. In some cases, at least a portion of the plurality of ISPs are attached to CD3+ receptors on the cells of the heterogenous cell population; optionally, an average number of ISPs per cell in population of T heterogenous is less than about 4,000.

In some embodiments, an average number of ISP's per Treg cells in the Treg cell population may be about 500 to about 4,000. In some embodiments, an average number of ISP's per Treg cells in the Treg cell population may be at least about 500. In some embodiments, an average number of ISP's per Treg cells in the Treg cell population may be at most about 4,000. In some embodiments, an average number of ISP's per Treg cells in the Treg cell population may be about 500 to about 1,000, about 500 to about 1,500, about 500 to about 2,000, about 500 to about 2,500, about 500 to about 3,000, about 500 to about 4,000, about 1,000 to about 1,500, about 1,000 to about 2,000, about 1,000 to about 2,500, about 1,000 to about 3,000, about 1,000 to about 4,000, about 1,500 to about 2,000, about 1,500 to about 2,500, about 1,500 to about 3,000, about 1,500 to about 4,000, about 2,000 to about 2,500, about 2,000 to about 3,000, about 2,000 to about 4,000, about 2,500 to about 3,000, about 2,500 to about 4,000, or about 3,000 to about 4,000. In some embodiments, an average number of ISP's per Treg cells in the Treg cell population may be about 500, about 1,000, about 1,500, about 2,000, about 2,500, about 3,000, or about 4,000. In some embodiments, an average number of ISP's per Treg cells in the Treg cell population may be at least 500, 1,000, 1,500, 2,000, 2,500, 3,000, or 4,000. In some embodiments, an average number of ISP's per Treg cells in the Treg cell population may be at most 500, 1,000, 1,500, 2,000, 2,500, 3,000, or 4,000.

In some embodiments, an average number of ISP's per Tcon cell in the Tcon cell population may be about 100 to about 1,000. In some embodiments, an average number of ISP's per Tcon cell in the Tcon cell population may be at least about 100. In some embodiments, an average number of ISP's per Tcon cell in the Tcon cell population may be at most about 1,000. In some embodiments, an average number of ISP's per Tcon cell in the Tcon cell population may be about 100 to about 200, about 100 to about 500, about 100 to about 1,000, about 200 to about 500, about 200 to about 1,000, or about 500 to about 1,000. In some embodiments, an average number of ISP's per Tcon cell in the Tcon cell population may be about 100, about 200, about 500, or about 1,000. In some embodiments, an average number of ISP's per Tcon cell in the Tcon cell population may be at least 100, 200, 500, or 1,000. In some embodiments, an average number of ISP's per Tcon cell in the Tcon cell population may be at most 100, 200, 500, or 1,000.

In various embodiments, cells of the mobilized peripheral blood donation are sorted such that the first population of CD45+ cells comprises at most about 10% granulocytes. In some cases, cells of the mobilized peripheral blood donation are sorted such that the first population of CD45+ cells comprises at most about 7% granulocytes.

In some embodiments, cells of the mobilized donor peripheral blood donation are sorted such that the first population of CD45+ cells comprises at most about 4% monocytes. In some cases, cells of the mobilized donor peripheral blood donation are sorted such that the first population of CD45+ cells comprises at least about 0.1% monocytes.

In embodiments, cells of the mobilized donor peripheral blood donation are sorted such that the population enriched for Tregs comprises at most about 10% CD25− cells.

In some embodiments, a cell population of the disclosure is obtained from whole blood. A cell population of the disclosure can be obtained from a peripheral blood apheresis product, for example, a mobilized peripheral blood apheresis product, e.g., mobilized by administration of GCSF, GM-CSF, mozobil, and combinations thereof, to a donor. A cell population of the disclosure can be obtained from at least one apheresis product, two apheresis products, three apheresis products, four apheresis products, five apheresis products, six apheresis products, or more. In some embodiments, a cell population of the disclosure is obtained from one apheresis product. In some embodiments, a cell population of the disclosure is obtained from two apheresis products. In some embodiments, a cell population of the disclosure is obtained from an apheresis product from one donor and an apheresis product from an at least second donor.

In some embodiments, a cell population of the disclosure is obtained from bone marrow.

In some embodiments, a cell population of the disclosure is obtained from umbilical cord blood.

A cell population of the disclosure can be refined by selection from a population of cells, for example, peripheral blood or a peripheral blood apheresis product. Selection methods for cell populations can comprise methods involving positive or negative selection of a cell population of interest. Selection methods for cell populations can comprise affinity reagents, including but not limited to an antibody, a full-length antibody, a fragment of an antibody, a naturally occurring antibody, a synthetic antibody, an engineered antibody, a full-length affibody, a fragment of an affibody, a full-length affilin, a fragment of an affilin, a full-length anticalin, a fragment of an anticalin, a full-length avimer, a fragment of an avimer, a full-length DARPin, a fragment of a DARPin, a full-length fynomer, a fragment of a fynomer, a full-length kunitz domain peptide, a fragment of a kunitz domain peptide, a full-length monobody, a fragment of a monobody, a peptide, or a polyaminoacid. In some embodiments, the affinity reagent is directly conjugated to a detection reagent and/or purification reagent. In some cases, the detection reagent and purification reagent are the same. In some cases, the detection reagent and purification reagent are different. For example, the detection reagent and/or purification reagent is fluorescent, magnetic, or the like. In some cases, the detection reagent and/or purification reagent is a magnetic particle for column purification. For example, magnetic column purification may be performed using the Miltenyi system (CliniMACs) of columns, antibodies, buffers, preparation materials and reagents.

In various embodiments, at least one of the cell populations have a plurality of immuno-separation particles (ISPs) attached to receptors on the cells of the cell population. In some cases, the plurality of ISPs are immuno-magnetic separation particles. In some embodiments, the plurality of ISPs comprise an antibody conjugated to an iron containing particle. In some cases, at least a portion of the plurality of ISPs are attached to CD34+ receptors on the HPSC's of the HSPC cell population; optionally, an average number of ISP's per HSPC in the HSPC cell population is less than about 6,000, an average number of ISP's per HSPC in the HSPC cell population is equal to or less than about 3,000, and/or an average number of ISP's per HSPC in the HSPC cell population is from about 1700 to about 3,000. In some cases, at least a portion of the plurality of ISPs are attached to CD25+ receptors on the cells of the Treg cell population; optionally, an average number of ISP's per T-reg cell in the Treg population is equal or less than about 1700 or an average number of ISPs per T-reg cell in the Treg population is from about 1400 to about 1700. In some cases, at least a portion of the plurality of ISPs are attached to CD3+ receptors on the cells of the heterogenous cell population; optionally, an average number of ISPs per cell in population of T heterogenous is less than about 1,000.

Affinity reagents can comprise immunoaffinity reagents, utilizing the binding specificity of antibodies or fragments or derivatives thereof to positively or negatively select for a cell population of interest. Selection methods for cell populations can comprise an affinity agent and a column, such as magnetic activated cell sorting (MACS) with specific antibodies and microbeads. Selection methods for cell populations can comprise fluorescent activated cell sorting (FACS), with cell populations sorted based on staining profiles with one or more fluorescently-conjugated antibodies. Selection methods for cell populations can comprise physical adsorption, for example, physical adsorption of T cells to protein ligands such as lectins.

HSPCs can be obtained by harvesting from bone marrow or from peripheral blood. Bone marrow can be aspirated from the posterior iliac crest or the anterior iliac crest while the donor is under either local or general anesthesia. HSPCs can be obtained by harvesting from peripheral blood, for example, by peripheral blood apheresis. The number of stem cells harvested can be increased by treating the donor with a mobilization agent, i.e., an agent that mobilizes stem cells from the bone marrow into peripheral blood. Non-limiting examples of mobilization agents include granulocyte colony-stimulating factor (G-CSF), granulocyte macrophage colony-stimulating factor (GM-CSF), mozobil, and combinations thereof. Techniques to mobilize stem cells into peripheral blood can comprise administering to a donor, for example, 10 to 40 μ/kg/day of a mobilization agent. A mobilization agent can be administered to the donor in, for example, 1, 2, 3, 4, 5, 6, 7, 8, 9, or 10 doses. An apheresis product can be isolated from a donor about, for example, 1, 2, 3, 4, 5, 6, 7, 8, 9, 10, 11, 12, 13, 14, 15, 16, 17, 18, 19, 20, 21, 22, 23, 24, 26, 28, or 30 hour(s) after a dose of mobilization agent.

A population of CD45+ cells of the disclosure can comprise a HSPCs. The HSPCs can be selected based on expression of CD34. For example, the HSPCs of the disclosure can be selected using anti-CD34 antibodies as part of a magnetic activated cell sorting (MACS) or fluorescent activated cell sorting (FACS) system.

The number of HPSCs in a population of CD45+ cells can be determined, for example, by quantifying CD34+ cells via flow cytometry. In some embodiments, dose calculations are adjusted based on measures of cell viability measurements, e.g., viability determined via flow cytometry with propidium iodide or 7-AAD, or via trypan blue exclusion.

A cell population of the disclosure can be enriched for Tregs. Tregs can be selected based on expression of markers including CD3, CD4, CD25, CD127, FOXP3, and combinations thereof.

Tregs can be selected using magnetic activated cell sorting (MACS). Tregs can be selected using fluorescent activated cell sorting (FACS). Tregs can be selected using multiple procedures, for example, multiple MACS selections, multiple FACS selections, or a combination of MACS and FACS selections. For example, a first selection may be performed for expression of CD25, isolating CD25+ cells from a hematopoietic cell sample, for example with MACS. A second selection may be performed by contacting the CD25+ cells with antibodies specific for CD4 and for CD127, where FACS is used to isolate cells that are CD4+CD127dim.

Tregs can be isolated from whole blood. Tregs can be isolated from a peripheral blood apheresis product. Tregs can be isolated from a population of cells previously enriched and/or depleted for one or more other cell types, e.g., isolated from a population of cells depleted of CD34+ cells. In some embodiments, Tregs are isolated from the flow-through fraction of a CD34+ MACS selection.

The number of Tregs in a population of cells can be determined, for example, by flow cytometry, where Tregs can be identified as, for example, CD4+CD25+CD127dim or CD4+FOXP3+. Dose calculations can be adjusted based on measures of cell viability measurements, e.g., viability determined via flow cytometry with propidium iodide or 7-AAD, or via trypan blue exclusion.

A second population of CD45+ cells can comprise a population of Tcons. A second population of CD45+ cells that comprises, at least, Tcons can be sourced from peripheral blood. A second population of CD45+ cells can be sourced from a peripheral blood apheresis product.

In some embodiments, no selection steps are carried out, and a ond population of CD45+ cells that comprises, at least, Tcons is sourced directly from an aliquot of peripheral blood or apheresis product. In some embodiments, a population of cells can be enriched for Tcons, for example, by sorting based on the expression of various markers using MACS, FACS, or a combination thereof. A second population of CD45+ cells can be enriched by sorting for CD3+ cells. A second population of CD45+ cells can be enriched by sorting for CD4+ and CD8+ cells. A second population of CD45+ cells can be enriched by negative selection, where non-Tcon cells are removed, for example, by MACS depletion of cells expressing CD34, CD19, CD25, or a combination thereof.

The number of Tcons present in a second population of CD45+ cells can be quantified, for example, by quantifying CD3+ cells via flow cytometry. The number of CD3+ cells in an aliquot can be determined and a volume comprising an appropriate dose of CD3 cells administered to the recipient. Dose calculations can be adjusted based on measures of cell viability, e.g., viability determined via flow cytometry with propidium iodide or 7-AAD, or via trypan blue exclusion.

An apheresis product of the disclosure can be split into two portions, one portion used to provide the second population of CD45+ cells that comprises, at least, Tcons and the other portion to isolate and purify the population of CD45+ cells that comprises, at least, HSPCs, and the cell population enriched for Tregs. In alternate embodiments, CD34+ cells are isolated and purified from the apheresis product, creating a CD34-negative cell fraction from which the cell Treg are then isolated to help provide the cell population enriched for Tregs.

A cell population of the disclosure can comprise a population of iNKTs. A population of iNKTs can be sourced from peripheral blood. A population of iNKTs can be sourced from a peripheral blood apheresis product.

A population of cells can be enriched for iNKTs, for example, by sorting based on the expression of various markers using MACS, FACS, or a combination thereof. A population of iNKTs can be enriched, for example, by sorting for CD3+Vα24Jα18+ cells.

The number of iNKTs present in a population can be quantified, for example, by quantifying CD3+Vα24Jα18+ cells via flow cytometry. The number of CD3+Vα24Jα18+ cells in an aliquot can be determined and a volume comprising an appropriate dose of iNKTs administered to the recipient. In some embodiments, dose calculations are adjusted based on measures of cell viability measurements, e.g., viability determined via flow cytometry with propidium iodide or 7-AAD, or via trypan blue exclusion.

A cell population of the disclosure can comprise a population of Tmems. A population of Tmems can be sourced from peripheral blood. A population of Tmems can be sourced from a peripheral blood apheresis product.

A population of cells can be enriched for Tmems, for example, by sorting based on the expression of various markers using MACS, FACS, or a combination thereof. A population of Tmems can be enriched, for example, by sorting for CD3+CD45RA−CD45RO+ cells.

The number of Tmems present in a population can be quantified, for example, by quantifying CD3+CD45RA−CD45RO+ cells via flow cytometry. The number of CD3+CD45RA− CD45RO+ cells in an aliquot can be determined and a volume comprising an appropriate dose of Tmems administered to the recipient. Dose calculations can be adjusted based on measures of cell viability measurements, e.g., viability determined via flow cytometry with propidium iodide or 7-AAD, or via trypan blue exclusion.

A cell population of the disclosure or a cell population of the disclosure can be administered freshly after isolation, or after cryopreservation and subsequent thawing.

Cells freshly isolated from a donor (“fresh cells”) can be administered to a recipient subject. Fresh cells can be stored in a buffer, for example, CliniMACS PBS-EDTA Buffer with 0.5% human serum albumin, or Plasma-Lyte-A, pH 7.4 supplemented with 2% human serum albumin. Fresh cells can be stored at a reduced temperature (e.g., 2-8° C.), and without being cryopreserved/frozen.

After acquiring a fresh population of cells from a donor, the fresh cells can be stored for at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 25 hours, at least about 26 hours, at least about 27 hours, at least about 28 hours, at least about 29 hours, at least about 30 hours, at least about 31 hours, at least about 32 hours, at least about 33 hours, at least about 34 hours, at least about 35 hours, at least about 36 hours, at least about 37 hours, at least about 38 hours, at least about 39 hours, at least about 40 hours, at least about 44 hours, at least about 48 hours, at least about 50 hours, at least about 55 hours, at least about 60 hours, at least about 61 hours, at least about 62 hours, at least about 65 hours, at least about 70 hours, at least about 72 hours, at least about 80 hours, at least about 90 hours, at least about 96 hours, at least about 120 hours, at least about 150 hours, at least about 200 hours, at least about 300 hours, or more prior to administration to a subject.

After acquiring a fresh population of cells from a donor, the fresh cells can be stored for at most about 1 hour, at most about 2 hours, at most about 3 hours, at most about 4 hours, at most about 5 hours, at most about 6 hours, at most about 7 hours, at most about 8 hours, at most about 9 hours, at most about 10 hours, at most about 12 hours, at most about 14 hours, at most about 16 hours, at most about 18 hours, at most about 20, at most 22 hours, at most about 24 hours, at most about 30 hours, at most about 36 hours, at most about 40 hours, at most about 48 hours, at most about 60 hours, at most about 70 hours, at most about 72 hours, at most about 80 hours, at most about 90 hours, at most about 96 hours, at most about 120 hours, at most about 150 hours, at most about 200 hours, or at most about 300 hours prior to administration to a subject.

Cells of the disclosure can be cryopreserved. In some embodiments, cryopreservation can be beneficial to the methods disclosed herein. For example, cryopreservation of the second population of CD45+ cells that comprises, at least, Tcons prior to subsequent thawing and administering to a subject may reduce GVHD.

An additional aspect provides a method of transplanting a conventional T cell (Tcon) population into a human subject without eliciting a stage 2 or higher graft versus host disease (GVHD) response up to about 100 days after transplanting. The method comprising: (i). administering a solution comprising a population of conventional T cells (Tcons); and (ii). administering a solution comprising a population of regulatory T cells (Tregs). In this method, the population of Tcons is cryopreserved for at least about 4 hours; and the solution comprising the population of Tcons and the solution comprising the population of Tregs comprise less than about 5 EU of endotoxins per ml of the solution.

Cryopreservation can comprise addition of a preservative agent (e.g., DMSO), and gradual cooling of cells in a controlled-rate freezer to prevent osmotic cellular injury resulting from ice crystal formation. Cryopreservation can comprise commercial cryopreservation reagents and materials, for example, Cryobags and CryoStor® CS10.

Cryopreserved cells can be stored for periods of time ranging from hours to years at low temperatures. Cryopreserved cells can be stored at ultralow temperatures, for example, −50° C., −60° C., −70° C., −80° C., −90° C., −100° C., −110° C., −120° C., −130° C., −140° C., −150° C., −160° C., −170° C., −180° C., −190° C., −196° C., or less. Cryopreserved cells can be stored in storage devices comprising liquid nitrogen.

Cells can be cryopreserved before or after certain steps in the methods of the disclosure, for example, before or after sorting steps, before or after characterization steps, such as determining cell viability or the concentration of cells of a particular type.

In some embodiments, whole blood can be cryopreserved. Whole blood can be cryopreserved without sorting or characterization. Whole blood can be cryopreserved after sorting but without characterization. Whole blood can be cryopreserved after characterization but without sorting. Whole blood can be cryopreserved after characterization and sorting. Whole blood can be cryopreserved after quantifying a cell type of the disclosure Whole blood can be cryopreserved after quantifying conventional T cells (Tcons, e.g., CD3+ cells). Whole blood can be cryopreserved after quantifying viability of all cells or a population of cells of the disclosure (e.g., conventional T cells).

A peripheral blood apheresis product of the disclosure can be cryopreserved. A peripheral blood apheresis product can be cryopreserved without sorting or characterization. A peripheral blood apheresis product can be cryopreserved after sorting but without characterization. A peripheral blood apheresis product can be cryopreserved after characterization but without sorting. A peripheral blood apheresis product can be cryopreserved after characterization and sorting. A peripheral blood apheresis product can be cryopreserved after quantifying a cell type of the disclosure. A peripheral blood apheresis product can be cryopreserved after quantifying conventional T cells (Tcons, e.g., CD3+ cells). A peripheral blood apheresis product can be cryopreserved after quantifying viability of all cells or a population of cells of the disclosure (e.g., conventional T cells).

A population of cells sorted or selected from another population of cells can be cryopreserved, for example, a population of CD45+ cells, HSPCs, Tregs, Tcons, iNKTs, or Tmems can be cryopreserved.

A cell population of the disclosure can be cryopreserved for any amount of time. Cells of the disclosure may be cryopreserved for at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 at least about 14 hours, at least about 16 hours, at least about 18 hours, at least about 20 hours, at least about 22 hours, at least about 24 hours, at least about 30 hours, at least about 36 hours at least about 48 hours, at least about 50 hours, at least about 55 hours, at least about 60 hours, at least about 61 hours, at least about 62 hours, at least about 65 hours, at least about 70 hours, at least about 72 hours, at least about 80 hours, at least about 90 hours, at least about 96 hours, at least about 120 hours, at least about 150 hours, at least about 200 hours, at least about 300 hours, or more prior to thawing and administration to a subject.

In some embodiments, a cell population of the disclosure is cryopreserved for at most about 1 hour, at most about 2 hours, at most about 3 hours, at most about 4 hours, at most about 5 hours, at most about 6 hours, at most about 7 hours, at most about 8 hours, at most about 9 hours, at most about 10 hours, at most about 11 hours, at most about 12 at most about 14 hours, at most about 16 hours, at most about 18 hours, at most about 20 hours, at most about 22 hours, at most about 24 hours, at most about 30 hours, at most about 36 hours at most about 48 hours, at most about 50 hours, at most about 55 hours, at most about 60 hours, at most about 61 hours, at most about 62 hours, at most about 65 hours, at most about 70 hours, at most about 72 hours, at most about 80 hours, at most about 90 hours, at most about 96 hours, at most about 120 hours, at most about 150 hours, at most about 200 hours, or at most about 300 hours prior to thawing and administration to a subject.

In some embodiments, a cell population of the disclosure is cryopreserved for at least about 1 day, at least about 2 days, at least about 3 days, at least about 4 days, at least about 5 days, at least about 6 days, at least about 7 days, at least about 10 days, at least about 14 days, at least about 21 days, at least about 28 days, at least about 50 days, at least about 60 days, or at least about 96 days, or more prior to thawing and administration to a subject.

In some embodiments, a cell population of the disclosure is cryopreserved for at most about 1 day, at most about 2 days, at most about 3 days, at most about 4 days, at most about 5 days, at most about 6 days, at most about 7 days, at most about 10 days, at most about 14 days, at most about 21 days, at most about 28 days, at most about 50 days, at most about 60 days, or at most about 96 days prior to thawing and administration to a subject.

### B. Donors

In embodiments, the respective cell populations are provided as separate cell populations and are derived from a single human blood donor.

A cell population can comprise cells that are from one or more donors that have each been HLA typed, for example, to determine a degree of HLA matching to a subject that will receive the cell population.

Human leukocyte antigens (HLA), also broadly referred to as Major histocompatibility complex (MHC) antigens, can be protein molecules expressed on the surface of a cell that can confer an antigenic identity to that cell. HLA/MHC antigens are target molecules that can be recognized by T cells and natural killer (NK) cells as being derived from the same source of hematopoietic stem cells as the immune effector cells (“self”), or as being derived from another source of hematopoietic cells (“non-self”). HLA class I antigens (A, B, and C in humans) can be expressed by the vast majority of cells, while HLA class II antigens (DR, DP, and DQ in humans) can be expressed primarily on professional antigen presenting cells. Both HLA classes can be implicated in GVHD.

HLA antigens are encoded by highly polymorphic genes; a range of alleles exist for each HLA class I and II gene. Allelic gene products can differ in one or more amino acids in the a and/or R domain(s). Panels of specific antibodies or nucleic acid reagents can be used to determine HLA haplotypes of individuals, for example, using leukocytes that express class I and class II molecules. HLA alleles can be described at various levels of detail. Most designations begin with HLA- and the locus name, then * and some (even) number of digits specifying the allele. The first two digits can specify a group of alleles. The third through fourth digits, when present, can specify a synonymous allele. Digits five through six, when present, can denote any synonymous mutations within the coding frame of the gene. The seventh and eighth digits, when present, can distinguish mutations outside the coding region. Letters such as L, N, Q, or S may follow an allele's designation to specify an expression level or other non-genomic data known about it. Thus, a completely described allele may be up to 9 digits long, not including the HLA-prefix and locus notation.

The set of HLA alleles inherited from one parent forms a haplotype. HLA haploidentical can refer to a donor-recipient pair where one chromosome is matched at least at HLA-A; HLA-B, and HLA-DR between the donor and recipient. The haploidentical pair may or may not be matched at other alleles, e.g., other HLA genes on the other chromosome, or additional histocompatibility loci on either chromosome. Such donors can frequently occur in families, e.g. a parent can be haploidentical to a child; and siblings may be haploidentical.

A cell population can be from a donor that has been HLA-typed at any number of HLA alleles. A donor and a subject can be HLA matched, e.g., matched at all typed HLA alleles. A donor and a subject can be HLA mismatched, e.g., at least one HLA antigen can be mismatched between the donor and recipient.

In some embodiments, a donor and a subject can be HLA-typed at six alleles consisting of HLA-A, HLA-B, and HLA-DR alleles. The donor and subject can be matched at, for example 3/6 4/6, 5/6, or 6/6 of the alleles. In some embodiments, the donor and subject are matched at least at 5/6 alleles. In some embodiments, the donor and subject are matched at 6/6 alleles.

In some embodiments, a donor and a subject can be HLA-typed at eight alleles consisting of HLA-A, HLA-B, HLA-C, and HLA-DR alleles (e.g., HLA-DRB1 alleles). The donor and subject can be matched at, for example 4/8, 5/8, 6/8, 7/8, or 8/8 of the alleles. In some embodiments, the donor and subject are matched at least at 6/8 alleles. In some embodiments, the donor and subject are matched at least at 7/8 alleles. In some embodiments, the donor and subject are matched at 8/8 alleles.

In some embodiments, a donor and a subject can be HLA-typed at ten alleles consisting of HLA-A, HLA-B, HLA-C, and HLA-DR alleles (e.g., HLA-DRB1 alleles). The donor and subject can be matched at, for example 5/10, 6/10, 7/10, 8/10, 9/10, or 10/10 of the alleles. In some embodiments, the donor and subject are matched at least at 7/10 alleles. In some embodiments, the donor and subject are matched at least at 8/10 alleles. In some embodiments, the donor and subject are matched at least at 9/10 alleles. In some embodiments, the donor and subject are matched at 10/10 alleles.

A cell population can be generated from a matched sibling donor that is an 8/8 match for HLA-A, -B, -C, and -DRB1, all typed using DNA-based high-resolution methods. A cell population can be generated from a matched sibling donor that is an 7/8 match for HLA-A, -B, -C, and -DRB1, all typed using DNA-based high-resolution methods. A cell population can be generated from a matched sibling donor that is an 6/8 match for HLA-A, -B, -C, and -DRB1, all typed using DNA-based high-resolution methods. A cell population can be generated from a matched sibling donor that is an 10/10 match for HLA-A, -B, -C, -DQB1 and -DRB1, all typed using DNA-based high-resolution methods. A cell population can be generated from a matched sibling donor that is an 9/10 match for HLA-A, -B, -C, -DQB1 and -DRB1, all typed using DNA-based high-resolution methods. A cell population can be generated from a matched sibling donor that is an 8/10 match for HLA-A, -B, -C, -DQB1 and -DRB1, all typed using DNA-based high-resolution methods. A cell population can be generated from a matched sibling donor that is an 7/10 match for HLA-A, -B, -C, -DQB1 and -DRB1, all typed using DNA-based high-resolution methods.

A cell population can be generated from a matched unrelated donor that is a 8/8 match at HLA-A, -B, -C, and -DRB1, all typed using DNA-based high-resolution methods. A cell population can be generated from a matched unrelated donor that is a 7/8 match at HLA-A, -B, -C, and -DRB1, all typed using DNA-based high-resolution methods. A cell population can be generated from a matched unrelated donor that is a 6/8 match at HLA-A, -B, -C, and -DRB1, all typed using DNA-based high-resolution methods. A cell population can be generated from a matched unrelated donor that is a 10/10 match at HLA-A, -B, -C, -DQB1 and -DRB1, all typed using DNA-based high-resolution methods. A cell population can be generated from a matched unrelated donor that is a 9/10 match at HLA-A, -B, -C, -DQB1 and -DRB1, all typed using DNA-based high-resolution methods. A cell population can be generated from a matched unrelated donor that is a 8/10 match at HLA-A, -B, -C, -DQB1 and -DRB1, all typed using DNA-based high-resolution methods

In various embodiments, the first population of CD45+ cells, said population of cells enriched for Tregs, and/or said second population of CD45+ cells is allogeneic relative to said human subject.

In some embodiments, the first population of CD45+ cells, said population of cells enriched for Tregs, and/or said second population of CD45+ cells is obtained from a donor that is HLA-matched relative to said human subject.

In embodiments, the first population of CD45+ cells, said population of cells enriched for Tregs, and/or said second population of CD45+ cells is obtained from a donor that is HLA-mismatched relative to said human subject.

In various embodiments, the first population of CD45+ cells, said population of cells enriched for Tregs, and/or said second population of CD45+ cells is obtained from a donor that is haploidentical relative to said human subject.

A cell population can be derived from an allogeneic donor. A cell population can be generated from a donor that is a first-degree blood relative of the subject. A cell population can be generated from a donor that is a second-degree blood relative of the subject. A cell population can be generated from a donor that is not related to the subject. A cell population can be generated from a donor that is HLA matched to a recipient subject. A cell population can be generated from a donor that is HLA mismatched to a recipient subject. A cell population can be generated from a donor that is haploidentical to a recipient subject. A cell population can be generated from a donor that is related to a recipient subject, for example, a parent, child, sibling, grandparent, grandchild, aunt, uncle, or cousin. A cell population can be generated from a donor that is at least 16 years old. A cell population can be generated from a donor that is at least 18 years old.

A cell population can be generated from a donor that meets eligibility criteria for donors of viable, leukocyte-rich cells or tissues as defined by 21 CFR § 1271 2018 and relevant FDA Guidance for Industry. For example, a cell population can be generated from a donor that meets eligibility criteria outlined in any one or more of the following: Eligibility Determination for Donors of Human Cells, Tissues, and Cellular and Tissue-Based Products, 2007; Use of Donor Screening Tests to Test Donors of Human Cells, Tissues and Cellular and Tissue-Based Products for Infection with Treponema pallidum (Syphilis), 2015; Use of Nucleic Acid Tests to Reduce the Risk of Transmission of Hepatitis B Virus from Donors of Human Cells, Tissues, and Cellular and Tissue-Based Products, 2016; Use of Nucleic Acid Tests to Reduce the Risk of Transmission of West Nile Virus from Living Donors of Human Cells, Tissues, and Cellular and Tissue-Based Products (HCT/Ps), 2016; and Donor Screening Recommendations to Reduce the Risk of Transmission of Zika Virus by Human Cells, Tissues, and Cellular and Tissue-Based Products, 2018). A cell population can be generated from a donor that meets any criteria for donation as specified by standard NMDP guidelines (NMDP donors).

A cell population can be generated from a donor that does not exhibit evidence of active infection. A cell population can be generated from a donor that is not seropositive for HIV-1 or -2, HTLV-1 or -2. A cell population can be generated from a donor that is not positive for anti-hepatitis C (HCV) antibody or HCV NAT. A cell population can be generated from a donor that tests negative for chronic HBV infection. A cell population can be generated from a donor that does not have high potential for Zika virus infection as defined as any of the following: (i) Medical diagnosis of Zika virus infection in the past 6 months; (ii) Residence in, or travel to, an area with active Zika virus transmission within the past 6 months; (iii) Unprotected sex within the past 6 months with a person who is known to have either of the risk factors (i) or (ii). A cell population can be generated from a donor that does not have signs or symptoms consistent with active Zika virus infection.

One or more cell populations of the disclosure can be obtained from a single donor, for example, obtained from mobilized peripheral blood apheresis of a single donor. HSPCs, Tregs, Tcons, iNKTs, Tmems, or any combination thereof can be obtained from a single donor.

One or more cell populations of the disclosure can be obtained from one donor, and one or more additional cell populations of the disclosure can be obtained from a second donor. One cell population of the disclosure can be obtained from a single donor, and a second cell population of the disclosure can be obtained from multiple donors. Populations of the disclosure can be obtained from multiple donors, for example, obtained from mobilized peripheral blood apheresis of multiple donors. HSPCs can be obtained from multiple donors. Tregs can be obtained from multiple donors. Tcons can be obtained from multiple donors. iNKTs can be obtained from multiple donors. Tmems can be obtained from multiple donors.

### C. Doses of Cell Populations

Doses of cell populations administered to a subject may be based on the subject's body weight. In some cases, a subject's body weight may be used to determine a dose of one or more cell populations to be administered to the subject. In some cases, a cell dose may be based on the ideal body weight of the subject instead of their actual weight, e.g., actual body weight”. Ideal body weight may be a preferable method of dose calculation to avoid erroneous cell doses due to excess body fat and/or muscle mass. A subject's ideal body weight may be calculated using their height and sex. Other methods that calculate a subject's ideal body weight may be used. For instance, other methods which determine a subject's body fat percentage. A dose of cell populations may be based on the subject's adjusted body weigh (ABW) if the subject's actual body weight is greater than 120% of his/her ideal body weight (IBW).

### HSPCs

A first population of CD45+ cells which comprise, at least, HSPCs can comprise at least about 1×10⁴, at least about 1×10⁵, at least about 5×10⁵, at least about 6×10⁵, at least about 7×10⁵, at least about 8×10⁵, at least about 9×10⁵, at least about 1×10⁶, at least about 1.1×10⁶, at least about 1.2×10⁶, at least about 1.3×10⁶, at least about 1.4×10⁶, at least about 1.5×10⁶, at least about 1.6×10⁶, at least about 1.7×10⁶, at least about 1.8×10⁶, at least about 1.9×10⁶, at least about 2×10⁶, at least about 2.1×10⁶, at least about 2.2×10⁶, at least about 2.3×10⁶, at least about 2.4×10⁶, at least about 2.5×10⁶, at least about 2.6×10⁶, at least about 2.7×10⁶, at least about 2.8×10⁶, at least about 2.9×10⁶, at least about 3×10⁶, at least about 3.1×10⁶, at least about 3.2×10⁶, at least about 3.3×10⁶, at least about 3.4×10⁶, at least about 3.5×10⁶, at least about 3.6×10⁶, at least about 3.7×10⁶, at least about 3.8×10⁶, at least about 3.9×10⁶, at least about 4×10⁶, at least about 4.1×10⁶, at least about 4.2×10⁶, at least about 4.3×10⁶, at least about 4.4×10⁶, at least about 4.5×10⁶, at least about 4.6×10⁶, at least about 4.7×10⁶, at least about 4.8×10⁶, at least about 4.9×10⁶, at least about 5×10⁶, at least about 5.1×10⁶, at least about 5.2×10⁶, at least about 5.3×10⁶, at least about 5.4×10⁶, at least about 5.5×10⁶, at least about 5.6×10⁶, at least about 5.7×10⁶, at least about 5.8×10⁶, at least about 5.9×10⁶, at least about 6×10⁶, at least about 6.5×10⁶, at least about 7×10⁶, at least about 7.5×10⁶, at least about 8×10⁶, at least about 8.5×10⁶, at least about 9×10⁶, at least about 9.5×10⁶, at least about 1×10⁷, at least about 1.5×10⁷, at least about 2×10⁷, at least about 2.5×10⁷, at least about 3×10⁷, at least about 3.5×10⁷, at least about 4×10⁷, at least about 4.5×10⁷, at least about 5×10⁷, at least about 5.5×10⁷, at least about 6×10⁷, at least about 6.5×10⁷, at least about 7×10⁷, at least about 7.5×10⁷, at least about 8×10⁷, at least about 8.5×10⁷, at least about 9×10⁷, at least about 9.5×10⁷, at least about 1×10⁸, at least about 1×10⁸, at least about 1.5×10⁸, at least about 2×10⁸, at least about 2.5×10⁸, at least about 3×10⁸, at least about 3.5×10⁸, at least about 4×10⁷, at least about 4.5×10⁸, at least about 5×10⁸, at least about 5.5×10⁸, at least about 6×10⁸, at least about 6.5×10⁸, at least about 7×10⁸, at least about 7.5×10⁸, at least about 8×10⁸, at least about 8.5×10⁸, at least about 9×10⁸, at least about 9.5×10⁸, at least about 1×10⁹, or more cells of the first population of CD45+ cells and/or HSPCs (e.g., CD34+ cells) per kg of recipient subject's actual body weight or ideal body weight.

A first population of CD45+ cells can comprise at most about 1×10⁴, at most about 1×10⁵, at most about 5×10⁵, at most about 6×10⁵, at most about 7×10⁵, at most about 8×10⁵, at most about 9×10⁵, at most about 1×10⁶, at most about 1.1×10⁶, at most about 1.2×10⁶, at most about 1.3×10⁶, at most about 1.4×10⁶, at most about 1.5×10⁶, at most about 1.6×10⁶, at most about 1.7×10⁶, at most about 1.8×10⁶, at most about 1.9×10⁶, at most about 2×10⁶, at most about 2.1×10⁶, at most about 2.2×10⁶, at most about 2.3×10⁶, at most about 2.4×10⁶, at most about 2.5×10⁶, at most about 2.6×10⁶, at most about 2.7×10⁶, at most about 2.8×10⁶, at most about 2.9×10⁶, at most about 3×10⁶, at most about 3.1×10⁶, at most about 3.2×10⁶, at most about 3.3×10⁶, at most about 3.4×10⁶, at most about 3.5×10⁶, at most about 3.6×10⁶, at most about 3.7×10⁶, at most about 3.8×10⁶, at most about 3.9×10⁶, at most about 4×10⁶, at most about 4.1×10⁶, at most about 4.2×10⁶, at most about 4.3×10⁶, at most about 4.4×10⁶, at most about 4.5×10⁶, at most about 4.6×10⁶, at most about 4.7×10⁶, at most about 4.8×10⁶, at most about 4.9×10⁶, at most about 5×10⁶, at most about 5.1×10⁶, at most about 5.2×10⁶, at most about 5.3×10⁶, at most about 5.4×10⁶, at most about 5.5×10⁶, at most about 5.6×10⁶, at most about 5.7×10⁶, at most about 5.8×10⁶, at most about 5.9×10⁶, at most about 6×10⁶, at most about 6.5×10⁶, at most about 7×10⁶, at most about 7.5×10⁶, at most about 8×10⁶, at most about 8.5×10⁶, at most about 9×10⁶, at most about 9.5×10⁶, at most about 1×10⁷, at most about 1.5×10⁷, at most about 2×10⁷, at most about 2.5×10⁷, at most about 3×10⁷, at most about 3.5×10⁷, at most about 4×10⁷, at most about 4.5×10⁷, at most about 5×10⁷, at most about 5.5×10⁷, at most about 6×10⁷, at most about 6.5×10⁷, at most about 7×10⁷, at most about 7.5×10⁷, at most about 8×10⁷, at most about 8.5×10⁷, at most about 9×10⁷, at most about 9.5×10⁷, at most about 1×10⁸, at most about 1×10⁸, at most about 1.5×10⁸, at most about 2×10⁸, at most about 2.5×10⁸, at most about 3×10⁸, at most about 3.5×10⁸, at most about 4×10⁷, at most about 4.5×10⁸, at most about 5×10⁸, at most about 5.5×10⁸, at most about 6×10⁸, at most about 6.5×10⁸, at most about 7×10⁸, at most about 7.5×10⁸, at most about 8×10⁸, at most about 8.5×10⁸, at most about 9×10⁸, at most about 9.5×10⁸, or at most about 1×10⁹ cells of the first population of CD45+ cells and/or HSPCs (e.g., CD34+ cells) per kg of recipient subject's actual body weight or ideal body weight.

For example, a first population of CD45+ cells can comprise 1×10⁴ to 1×10⁹, 1×10⁵ to 1×10⁸, 1×10⁵ to 2×10⁷, 5×10⁵ to 2×10⁷, 5×10⁵ to 1.5×10⁷, 5×10⁵ to 1×10⁷, 5×10⁵ to 9×10⁶, 5×10⁵ to 8×10⁶, 5×10⁵ to 7×10⁶, 5×10⁵ to 6×10⁶, 5×10⁵ to 5×10⁶, 5×10⁵ to 4×10⁶, 5×10⁵ to 3×10⁶, 5×10⁵ to 2×10⁶, 5×10⁵ to 1×10⁶, 1×10⁶ to 1.5×10⁷, 1×10⁶ to 1×10⁷, 1×10⁶ to 9×10⁶, 1×10⁶ to 8×10⁶, 1×10⁶ to 7×10⁶, 1×10⁶ to 6×⁶, 1×10⁶ to 5×10⁶, 1×10⁶ to 4×10⁶, 1×10⁶ to 3×10⁶, 1×10⁶ to 2×10⁶, 1.5×10⁶ to 1.5×10⁷, 1.5×10⁶ to 1×10⁷, 1.5×10⁶ to 9×10⁶, 1.5×10⁶ to 8×10⁶, 1.5×10⁶ to 7×10⁶, 1.5×10⁶ to 6×10⁶, 1.5×10⁶ to 5×10⁶, 1.5×10⁶ to 4×10⁶, 1.5×10⁶ to 3×10⁶, 1.5×10⁶ to 2×10⁶, 2 ×10⁶ to 1.5×10⁷, 2×10⁶ to 1×10⁷, 2×10⁶ to 9×10⁶, 2×10⁶ to 8×10⁶, 2×10⁶ to 7×10⁶, 2×10⁶ to 6×10⁶, 2×10⁶ to 5×10⁶, 2 ×10⁶ to 4×10⁶, 2×10⁶ to 3×10⁶, 2.5×10⁶ to 1.5×10⁷, 2.5×10⁶ to 1×10⁷, 2.5×10⁶ to 9×10⁶, 2.5×10⁶ to 8×10⁶, 2.5×10⁶ to 7×10⁶, 2.5×10⁶ to 6×10⁶, 2.5×10⁶ to 5×10⁶, 2.5×10⁶ to 4×10⁶, or 2.5×10⁶ to 3×10⁶ cells of the first population of CD45+ cells and/or HSPCs (e.g., CD34+ cells) per kg of recipient subject's actual body weight or ideal body weight.

A first population of CD45+ cells can have a defined level of purity for CD34+ cells. For example, a first population of CD45+ cells can comprise at least about 5%, at least about at least about 10%, at least about at least about 15%, at least about 20%, at least about 25%, at least about 30%, at least about 35%, at least about 40%, at least about 45%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, or more CD34+ cells as a percentage of total cells, as a percentage of nucleated cells, or as a percentage of CD45+ cells.

A first population of CD45+ cells can have a defined level of contaminating CD3+ cells. In some embodiments, at most about 1×10², at most about 2×10², at most about 3×10², at most about 4×10², at most about 5×10², at most about 6×10², at most about 7×10², at most about 8×10², at most about 9×10², at most about 1×10³, at most about 2×10³, at most about 3×10³, at most about 4×10³, at most about 5×10³, at most about 6×10³, at most about 7×10³, at most about 8×10³, at most about 9×10³, at most about 1×10⁴, at most about 2×10⁴, at most about 3×10⁴, at most about 4×10⁴, at most about 5×10⁴, at most about 6×10⁴ at most about 7×10⁴, at most about 8×10⁴, at most about 9×10⁴, or at most about 1×10⁵ CD3+ cells per kg of a recipient subject's body weight are present in a first population of CD45+ cells or ideal body weight.

In some embodiments, a first population of CD45+ cells comprises at most about 0.001%, at most about 0.002%, at most about 0.003%, at most about 0.004%, at most about 0.005%, at most about 0.006%, at most about 0.007%, at most about 0.008% 0.009%, at most about 0.01%, at most about 0.02%, at most about 0.03%, at most about 0.04%, at most about 0.05%, at most about 0.06%, at most about 0.07%, at most about 0.08%, at most about 0.09%, at most about 0.1%, at most about 0.2%, at most about 0.3%, at most about 0.4%, at most about 0.5%, at most about 0.6%, at most about 0.7%, at most about 0.8%, at most about 0.9%, at most about 1%, at most about 1.1%, at most about 1.2%, at most about 1.3%, at most about 1.4%, at most about 1.5%, at most about 1.6%, at most about 1.7%, at most about 1.8%, at most about 1.9%, at most about 2%, at most about 2.1%, at most about 2.2%, at most about 2.3%, at most about 2.4%, at most about 2.5%, at most about 2.6%, at most about 2.7%, at most about 2.8%, at most about 2.9%, at most about 3%, at most about 3.1%, at most about 3.2%, at most about 3.3%, at most about 3.4%, at most about 3.5%, at most about 3.6%, at most about 3.7%, at most about 3.8%, at most about 3.9%, at most about 4%, at most about 5%, at most about 6%, at most about 7%, at most about 8%, at most about 9%, or at most about 10% CD3+ cells as a percentage of total cells, as a percentage of nucleated cells, or as a percentage of CD45+ cells.

In some embodiments, the first population of CD45+ cells comprises less than about 5 EU of endotoxins per ml of the solution, less than about 1 EU of endotoxins per ml of the solution, and/or less than about 0.5 EU of endotoxins per ml of the solution.

In embodiments, at least one of the cell populations comprise less than about 5 EU of endotoxins/ml of respective suspension liquid.

A first population of CD45+ cells can comprise 0.5 EU/ml endotoxins to 10 EU/ml endotoxins. A first population of CD45+ cells can comprise at least 0.5 EU/ml endotoxins. A first population of CD45+ cells can comprise at most 10 EU/ml endotoxins. A first population of CD45+ cells can comprise 10 EU/ml endotoxins to 8 EU/ml endotoxins, 10 EU/ml endotoxins to 6 EU/ml endotoxins, 10 EU/ml endotoxins to 5 EU/ml endotoxins, 10 EU/ml endotoxins to 4 EU/ml endotoxins, 10 EU/ml endotoxins to 2 EU/ml endotoxins, 10 EU/ml endotoxins to 1 EU/ml endotoxins, 10 EU/ml endotoxins to 0.5 EU/ml endotoxins, 8 EU/ml endotoxins to 6 EU/ml endotoxins, 8 EU/ml endotoxins to 5 EU/ml endotoxins, 8 EU/ml endotoxins to 4 EU/ml endotoxins, 8 EU/ml endotoxins to 2 EU/ml endotoxins, 8 EU/ml endotoxins to 1 EU/ml endotoxins, 8 EU/ml endotoxins to 0.5 EU/ml endotoxins, 6 EU/ml endotoxins to 5 EU/ml endotoxins, 6 EU/ml endotoxins to 4 EU/ml endotoxins, 6 EU/ml endotoxins to 2 EU/ml endotoxins, 6 EU/ml endotoxins to 1 EU/ml endotoxins, 6 EU/ml endotoxins to 0.5 EU/ml endotoxins, 5 EU/ml endotoxins to 4 EU/ml endotoxins, 5 EU/ml endotoxins to 2 EU/ml endotoxins, 5 EU/ml endotoxins to 1 EU/ml endotoxins, 5 EU/ml endotoxins to 0.5 EU/ml endotoxins, 4 EU/ml endotoxins to 2 EU/ml endotoxins, 4 EU/ml endotoxins to 1 EU/ml endotoxins, 4 EU/ml endotoxins to 0.5 EU/ml endotoxins, 2 EU/ml endotoxins to 1 EU/ml endotoxins, 2 EU/ml endotoxins to 0.5 EU/ml endotoxins, or 1 EU/ml endotoxins to 0.5 EU/ml endotoxins. A first population of CD45+ cells can comprise 10 EU/ml endotoxins, 8 EU/ml endotoxins, 6 EU/ml endotoxins, 5 EU/ml endotoxins, 4 EU/ml endotoxins, 2 EU/ml endotoxins, 1 EU/ml endotoxins, or 0.5 EU/ml endotoxins. A first population of CD45+ cells can comprise at least 8 EU/ml endotoxins, 6 EU/ml endotoxins, 5 EU/ml endotoxins, 4 EU/ml endotoxins, 2 EU/ml endotoxins, 1 EU/ml endotoxins, or 0.5 EU/ml endotoxins. A first population of CD45+ cells can comprise at most 10 EU/ml endotoxins, 8 EU/ml endotoxins, 6 EU/ml endotoxins, 5 EU/ml endotoxins, 4 EU/ml endotoxins, 2 EU/ml endotoxins or 1 EU/ml endotoxins.

A first population of CD45+ cells can comprise 0.5% w/w to 10% w/w unbound reagents. These unbound reagents may include any affinity reagents used for the sorting of HSPCs, for instance, antibodies, or purification particles or magnetic particles. A first population of CD45+ cells can comprise at least 0.5% w/w unbound reagents. A first population of CD45+ cells can comprise at most 10% w/w unbound reagents. A first population of CD45+ cells can comprise 10% w/w to 8% w/w, 10% w/w to 6% w/w, 10% w/w to 5% w/w, 10% w/w to 4% w/w, 10% w/w to 2% w/w, 10% w/w to 1% w/w, 10% w/w to 0.5% w/w, 8% w/w to 6% w/w, 8% w/w to 5% w/w, 8% w/w to 4% w/w, 8% w/w to 2% w/w, 8% w/w to 1% w/w, 8% w/w to 0.5% w/w, 6% w/w to 5% w/w, 6% w/w to 4% w/w, 6% w/w to 2% w/w, 6% w/w to 1% w/w, 6% w/w to 0.5% w/w, 5% w/w to 4% w/w, 5% w/w to 2% w/w, 5% w/w to 1% w/w, 5% w/w to 0.5% w/w, 4% w/w to 2% w/w, 4% w/w to 1% w/w, 4% w/w to 0.5% w/w, 2% w/w to 1% w/w, 2% w/w to 0.5% w/w, or 1% w/w to 0.5% w/w unbound reagents. A first population of CD45+ cells can comprise 10% w/w, 8% w/w, 6% w/w, 5% w/w, 4% w/w, 2% w/w, 1% w/w, or 0.5% w/w unbound reagents. A first population of CD45+ cells can comprise at least 8% w/w, 6% w/w, 5% w/w, 4% w/w, 2% w/w, 1% w/w, or 0.5% w/w unbound reagents. A first population of CD45+ cells can comprise at most 10% w/w, 8% w/w, 6% w/w, 5% w/w, 4% w/w, 2% w/w or 1% w/w unbound reagents.

A first population of CD45+ cells can comprise 5×10³ to 90×10³ microbeads per cell. These microbeads may comprise microbeads used to purify the HSPC population, for instance, an anti-CD34 antibody comprising microbead used to sort the HSPC population. A first population of CD45+ cells can comprise at least 5×10³ microbeads per cell. A first population of CD45+ cells can comprise at most 90×10³ microbeads per cell. A first population of CD45+ cells can comprise 90×10³ to 70×10³, 90×10³ to 50×10³, 90×10³ to 40×10³, 90×10³ to 30×10³, 90×10³ to 20×10³, 90×10³ to 10×10³, 90×10³ to 5×10³, 70×10³ to 50×10³, 70×10³ to 40×10³, 70×10³ to 30×10³, 70×10³ to 20×10³, 70×10³ to 10×10³, 70×10³ to 5×10³, 50×10³ to 40×10³, 50×10³ to 30×10³, 50×10³ to 20×10³, 50×10³ to 10×10³, 50×10³ to 5×10³, 40×10³ to 30×10³, 40×10³ to 20×10³, 40×10³ to 10×10³, 40×10³ to 5×10³, 30×10³ to 20×10³, 30×10³ to 10×10³, 30×10³ to 5×10³, 20×10³ to 10×10³, 20×10³ to 5×10³, or 10×10³ to 5×10³ microbeads per cell. A first population of CD45+ cells can comprise 90×10³, 70×10³, 50×10³, 40×10³, 30×10³, 20×10³, 10×10³, or 5×10³ microbeads per cell. A first population of CD45+ cells can comprise at least 70×10³, 50×10³, 40×10³, 30×10³, 20×10³, 10×10³, or 5×10³ microbeads per cell. A first population of CD45+ cells can comprise at most 90×10³, 70×10³, 50×10³, 40×10³, 30×10³, 20×10³, or 10×10³ microbeads per cell.

### Tregs

A population of cells enriched for Tregs can comprise at least about 1×10⁴, at least about 1×10⁵, at least about 5×10⁵, at least about 6×10⁵, at least about 7×10⁵, at least about 8×10⁵, at least about 9×10⁵, at least about 1×10⁶, at least about 1.1×10⁶, at least about 1.2×10⁶, at least about 1.3×10⁶, at least about 1.4×10⁶, at least about 1.5×10⁶, at least about 1.6×10⁶, at least about 1.7×10⁶, at least about 1.8×10⁶, at least about 1.9×10⁶, at least about 2×10⁶, at least about 2.1×10⁶, at least about 2.2×10⁶, at least about 2.3×10⁶, at least about 2.4×10⁶, at least about 2.5×10⁶, at least about 2.6×10⁶, at least about 2.7×10⁶, at least about 2.8×10⁶, at least about 2.9×10⁶, at least about 3×10⁶, at least about 3.1×10⁶, at least about 3.2×10⁶, at least about 3.3×10⁶, at least about 3.4×10⁶, at least about 3.5×10⁶, at least about 3.6×10⁶, at least about 3.7×10⁶, at least about 3.8×10⁶, at least about 3.9×10⁶, at least about 4×10⁶, at least about 4.1×10⁶, at least about 4.2×10⁶, at least about 4.3×10⁶, at least about 4.4×10⁶, at least about 4.5×10⁶, at least about 4.6×10⁶, at least about 4.7×10⁶, at least about 4.8×10⁶, at least about 4.9×10⁶, at least about 5×10⁶, at least about 5.1×10⁶, at least about 5.2×10⁶, at least about 5.3×10⁶, at least about 5.4×10⁶, at least about 5.5×10⁶, at least about 5.6×10⁶, at least about 5.7×10⁶, at least about 5.8×10⁶, at least about 5.9×10⁶, at least about 6×10⁶, at least about 6.5×10⁶, at least about 7×10⁶, at least about 7.5×10⁶, at least about 8×10⁶, at least about 8.5×10⁶, at least about 9×10⁶, at least about 9.5×10⁶, at least about 1×10⁷, at least about 1.5×10⁷, at least about 2×10⁷, at least about 2.5×10⁷, at least about 3×10⁷, at least about 3.5×10⁷, at least about 4×10⁷, at least about 4.5×10⁷, at least about 5×10⁷, at least about 5.5×10⁷, at least about 6×10⁷, at least about 6.5×10⁷, at least about 7×10⁷, at least about 7.5×10⁷, at least about 8×10⁷, at least about 8.5×10⁷, at least about 9×10⁷, at least about 9.5×10⁷, at least about 1×10⁸, at least about 1×10⁸, at least about 1.5×10⁸, at least about 2×10⁸, at least about 2.5×10⁸, at least about 3×10⁸, at least about 3.5×10⁸, at least about 4×10⁷, at least about 4.5×10⁸, at least about 5×10⁸, at least about 5.5×10⁸, at least about 6×10⁸, at least about 6.5×10⁸, at least about 7×10⁸, at least about 7.5×10⁸, at least about 8×10⁸, at least about 8.5×10⁸, at least about 9×10⁸, at least about 9.5×10⁸, at least about 1×10⁹, or more cells of the cell population enriched for Tregs and/or Tregs per kg of recipient subject's actual body weight or ideal body weight (e.g., where Tregs are CD4+CD25+CD127dim, CD3+CD4+CD25+, CD3+CD4+CD25+CD127dim, CD3+CD4+CD25+CD127dim FOXP3+, CD3+FOXP3+, CD3+CD4+FOXP3+, CD3+CD4+CD25+FOXP3+, CD3+CD25+FOXP3+, CD3+CD25+CD127dim, CD4+CD25+, CD4+CD25+CD127dimFOXP3+, FOXP3+, CD4+FOXP3+, CD4+CD25+FOXP3+, CD25+FOXP3+, or CD25+CD127dim).

A population of cells enriched for Tregs can comprise at most about 1×10⁴, at most about 1×10⁵, at most about 5×10⁵, at most about 6×10⁵, at most about 7×10⁵, at most about 8×10⁵, at most about 9×10⁵ at most about 1×10⁶, at most about 1.1×10⁶, at most about 1.2×10⁶, at most about 1.3×10⁶, at most about 1.4×10⁶, at most about 1.5×10⁶, at most about 1.6×10⁶, at most about 1.7×10⁶, at most about 1.8×10⁶, at most about 1.9×10⁶, at most about 2×10⁶, at most about 2.1×10⁶, at most about 2.2×10⁶, at most about 2.3×10⁶, at most about 2.4×10⁶, at most about 2.5×10⁶, at most about 2.6×10⁶, at most about 2.7×10⁶, at most about 2.8×10⁶, at most about 2.9×10⁶, at most about 3×10⁶, at most about 3.1×10⁶, at most about 3.2×10⁶, at most about 3.3×10⁶, at most about 3.4×10⁶, at most about 3.5×10⁶, at most about 3.6×10⁶, at most about 3.7×10⁶, at most about 3.8×10⁶, at most about 3.9×10⁶, at most about 4×10⁶, at most about 4.1×10⁶, at most about 4.2×10⁶, at most about 4.3×10⁶, at most about 4.4×10⁶, at most about 4.5×10⁶, at most about 4.6×10⁶, at most about 4.7×10⁶, at most about 4.8×10⁶, at most about 4.9×10⁶, at most about 5×10⁶, at most about 5.1×10⁶, at most about 5.2×10⁶, at most about 5.3×10⁶, at most about 5.4×10⁶, at most about 5.5×10⁶, at most about 5.6×10⁶, at most about 5.7×10⁶, at most about 5.8×10⁶, at most about 5.9×10⁶, at most about 6×10⁶, at most about 6.5×10⁶, at most about 7×10⁶, at most about 7.5×10⁶, at most about 8×10⁶, at most about 8.5×10⁶, at most about 9×10⁶, at most about 9.5×10⁶, at most about 1×10⁷, at most about 1.5×10⁷, at most about 2×10⁷, at most about 2.5×10⁷, at most about 3×10⁷, at most about 3.5×10⁷, at most about 4×10⁷, at most about 4.5×10⁷, at most about 5×10⁷, at most about 5.5×10⁷, at most about 6×10⁷, at most about 6.5×10⁷, at most about 7×10⁷, at most about 7.5×10⁷, at most about 8×10⁷, at most about 8.5×10⁷, at most about 9×10⁷, at most about 9.5×10⁷, at most about 1×10⁸, at most about 1×10⁸, at most about 1.5×10⁸, at most about 2×10⁸, at most about 2.5×10⁸, at most about 3×10⁸, at most about 3.5×10⁸, at most about 4×10⁷, at most about 4.5×10⁸, at most about 5×10⁸, at most about 5.5×10⁸, at most about 6×10⁸, at most about 6.5×10⁸, at most about 7×10⁸, at most about 7.5×10⁸, at most about 8×10⁸, at most about 8.5×10⁸, at most about 9×10⁸, at most about 9.5×10⁸, or at most about 1×10⁹ cells of the cell population enriched for Tregs and/or Tregs per kg of recipient subject's actual body weight or ideal body weight (e.g., where Tregs are CD4+CD25+CD127dim, CD3+CD4+CD25+, CD3+CD4+CD25+CD127dim, CD3+CD4+CD25+CD127dim FOXP3+, CD3+FOXP3+, CD3+CD4+FOXP3+, CD3+CD4+CD25+FOXP3+, CD3+CD25+FOXP3+, CD3+CD25+CD127dim, CD4+CD25+, CD4+CD25+CD127dimFOXP3+, FOXP3+, CD4+FOXP3+, CD4+CD25+FOXP3+, CD25+FOXP3+, or CD25+CD127dim).

For example, a population of cells enriched for Tregs can comprise 1×10⁴ to 1×10⁹, 1×10⁵ to 1×10⁸, 1×10⁵ to 2×10⁷, 5×10⁵ to 2×10⁷, 5×10⁵ to 1.5×10⁷, 5×10⁵ to 1×10⁷, 5×10⁵ to 9×10⁶, 5×10⁵ to 8×10⁶, 5×10⁵ to 7×10⁶, 5×10⁵ to 6×10⁶, 5×10⁵ to 5×10⁶, 5×10⁵ to 4×10⁶, 5×10⁵ to 3×10⁶, 5×10⁵ to 2×10⁶, 5×10⁵ to 1×10⁶, 1×10⁶ to 1.5×10⁷, 1×10⁶ to 1×10⁷, 1×10⁶ to 9×10⁶, 1×10⁶ to 8×10⁶, 1×10⁶ to 7×10⁶, 1×10⁶ to 6×⁶, 1×10⁶ to 5×10⁶, 1×10⁶ to 4×10⁶, 1×10⁶ to 3×10⁶, 1×10⁶ to 2×10⁶, 1.5×10⁶ to 1.5×10⁷, 1.5×10⁶ to 1×10⁷, 1.5×10⁶ to 9×10⁶, 1.5×10⁶ to 8×10⁶, 1.5×10⁶ to 7×10⁶, 1.5×10⁶ to 6×10⁶, 1.5×10⁶ to 5×10⁶, 1.5×10⁶ to 4×10⁶, 1.5×10⁶ to 3×10⁶, 1.5×10⁶ to 2×10⁶, 2×10⁶ to 1.5×10⁷, 2×10⁶ to 1×10⁷, 2×10⁶ to 9×10⁶, 2×10⁶ to 8×10⁶, 2×10⁶ to 7×10⁶, 2×10⁶ to 6×10⁶, 2×10⁶ to 5×10⁶, 2×10⁶ to 4×10⁶, 2×10⁶ to 3×10⁶, 2.5×10⁶ to 1.5×10⁷, 2.5×10⁶ to 1×10⁷, 2.5×10⁶ to 9×10⁶, 2.5×10⁶ to 8×10⁶, 2.5×10⁶ to 7×10⁶, 2.5×10⁶ to 6×10⁶, 2.5×10⁶ to 5×10⁶, 2.5×10⁶ to 4×10⁶, or 2.5×10⁶ to 3×10⁶ cells of the cell population enriched for Tregs and/or Tregs per kg of recipient subject's actual body weight or ideal body weight (e.g., where Tregs are CD4+CD25+CD127dim, CD3+CD4+CD25+, CD3+CD4+CD25+CD127dim, CD3+CD4+CD25+CD127dim FOXP3+, CD3+FOXP3+, CD3+CD4+FOXP3+, CD3+CD4+CD25+FOXP3+, CD3+CD25+FOXP3+, CD3+CD25+CD127dim, CD4+CD25+, CD4+CD25+CD127dimFOXP3+, FOXP3+, CD4+FOXP3+, CD4+CD25+FOXP3+, CD25+FOXP3+, or CD25+CD127dim).

A population of cells enriched for Tregs can have a defined level of purity for Treg cells. For example, a population of cells enriched for Tregs of the disclosure can comprise at least about 5%, at least about at least about 10%, at least about at least about 15%, at least about 20%, at least about 25%, at least about 30%, at least about 35%, at least about 40%, at least about 45%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, or more Treg cells as a percentage of total cells, as a percentage of nucleated cells, or as a percentage of CD45+ cells (e.g., where Tregs are CD4+CD25+CD127dim, CD3+CD4+CD25+, CD3+CD4+CD25+CD127dim, CD3+CD4+CD25+CD127dim FOXP3+, CD3+FOXP3+, CD3+CD4+FOXP3+, CD3+CD4+CD25+FOXP3+, CD3+CD25+FOXP3+, CD3+CD25+CD127dim, CD4+CD25+, CD4+CD25+CD127dimFOXP3+, FOXP3+, CD4+FOXP3+, CD4+CD25+FOXP3+, CD25+FOXP3+, or CD25+CD127dim).

A population of cells enriched for Tregs of the disclosure can comprise 50% to 100%, 60% to 100%, 70% to 100%, 75% to 100%, 80% to 100%, 81% to 100%, 82% to 100%, 83% to 100%, 84% to 100%, 84% to 100%, 86% to 100%, 87% to 100%, 88% to 100%, 89% to 100%, 90% to 91%, 92% to 100%, 93% to 100%, 94% to 100%, 95% to 100%, 96% to 100%, 97% to 100%, 98% to 100%, 99% to 100%, 99.5% to 100%, 50% to 99%, 60% to 99%, 70% to 99%, 80% to 99%, 81% to 99%, 82% to 99%, 83% to 99%, 84% to 99%, 85% to 99%, 86% to 99%, 87% to 99%, 88% to 99%, 89% to 99%, 90% to 99%, 91% to 99%, 92% to 99%, 94% to 99%, 95% to 99%, 96% to 97%, 98% to 99%, 50% to 98%, 60% to 98%, 70% to 98%, 80% to 98%, 81% to 98%, 82% to 98%, 83% to 98%, 84% to 98%, 85% to 98%, 86% to 98%, 87% to 98%, 88% to 98%, 89% to 98%, 90% to 98%, 91% to 98%, 92% to 98%, 94% to 98%, 95% to 98%, 96% to 97%, 98% to 98%, 50% to 97%, 60% to 97%, 70% to 97%, 80% to 97%, 81% to 97%, 82% to 97%, 83% to 97%, 84% to 97%, 85% to 97%, 86% to 97%, 87% to 97%, 88% to 97%, 89% to 97%, 90% to 97%, 91% to 97%, 92% to 97%, 94% to 97%, 95% to 97%, 96% to 97%, 50% to 96%, 60% to 96%, 70% to 96%, 80% to 96%, 81% to 96%, 82% to 96%, 83% to 96%, 84% to 96%, 85% to 96%, 86% to 96%, 87% to 96%, 88% to 96%, 89% to 96%, 90% to 96%, 91% to 96%, 92% to 96%, 94% to 96%, 95% to 96%, 50% to 95%, 60% to 95%, 70% to 95%, 80% to 95%, 81% to 95%, 82% to 95%, 83% to 95%, 84% to 95%, 85% to 95%, 86% to 95%, 87% to 95%, 88% to 95%, 89% to 95%, 90% to 95%, 91% to 95%, 92% to 95%, or 94% to 95% Tregs as a percentage of total cells, nucleated cells, or CD45+ cells (e.g., where the Tregs are CD4+CD25+CD127dim, CD3+CD4+CD25+, CD3+CD4+CD25+CD127dim, CD3+CD4+CD25+CD127dim FOXP3+, CD3+FOXP3+, CD3+CD4+FOXP3+, CD3+CD4+CD25+FOXP3+, CD3+CD25+FOXP3+, CD3+CD25+CD127dim, CD4+CD25+, CD4+CD25+CD127dimFOXP3+, FOXP3+, CD4+FOXP3+, CD4+CD25+FOXP3+, CD25+FOXP3+, or CD25+CD127dim).

A population of cells enriched for Tregs of the disclosure can have a defined level of contaminating non-Treg cells. In some embodiments, at most about 1×10², at most about 2×10², at most about 3×10², at most about 4×10², at most about 5×10², at most about 6×10², at most about 7×10², at most about 8×10², at most about 9×10², at most about 1×10³ at most about 2×10³, at most about 3×10³, at most about 4×10³, at most about 5×10³, at most about 6×10³, at most about 7×10³, at most about 8×10³, at most about 9×10³, at most about 1×10⁴, at most about 2×10⁴, at most about 3×10⁴, at most about 4×10⁴, at most about 5×10⁴ at most about 6×10⁴, at most about 7×10⁴, at most about 8×10⁴, at most about 9×10⁴, or at most about 1×10⁵ non-Treg cells per kg of body weight or ideal body weight are present in a population of cells enriched for Tregs of the disclosure, where non-Treg cells are FOXP3− or CD127+/bright.

In some embodiments, a population of cells enriched for Tregs of the disclosure comprises at most about 0.001%, at most about 0.002%, at most about 0.003%, at most about 0.004%, at most about 0.005%, at most about 0.006%, at most about 0.007%, at most about 0.008% 0.009%, at most about 0.01%, at most about 0.02%, at most about 0.03%, at most about 0.04%, at most about 0.05%, at most about 0.06%, at most about 0.07%, at most about 0.08%, at most about 0.09%, at most about 0.1%, at most about 0.2%, at most about 0.3%, at most about 0.4%, at most about 0.5%, at most about 0.6%, at most about 0.7%, at most about 0.8%, at most about 0.9%, at most about 1%, at most about 1.1%, at most about 1.2%, at most about 1.3%, at most about 1.4%, at most about 1.5%, at most about 1.6%, at most about 1.7%, at most about 1.8%, at most about 1.9%, at most about 2%, at most about 2.1%, at most about 2.2%, at most about 2.3%, at most about 2.4%, at most about 2.5%, at most about 2.6%, at most about 2.7%, at most about 2.8%, at most about 2.9%, at most about 3%, at most about 3.1%, at most about 3.2%, at most about 3.3%, at most about 3.4%, at most about 3.5%, at most about 3.6%, at most about 3.7%, at most about 3.8%, at most about 3.9%, at most about 4%, at most about 5%, at most about 6%, at most about 7%, at most about 8%, at most about 9%, or at most about 10% non-Treg cells, where non-Treg cells are FOXP3− or CD127+/bright.

In various embodiments, the population of cells enriched for Tregs comprises less than about 5 EU of endotoxins per ml of the solution, less than about 1 EU of endotoxins per ml of the solution, and/or less than about 0.5 EU of endotoxins per ml of the solution.

In some embodiments, at least one of the cell populations comprise less than about 5 EU of endotoxins/ml of respective suspension liquid.

A population of cells enriched for Tregs can comprise 0.5 EU/ml endotoxins to 10 EU/ml endotoxins. A population of cells enriched for Tregs can comprise at least 0.5 EU/ml endotoxins. A population of cells enriched for Tregs can comprise at most 10 EU/ml endotoxins. A population of cells enriched for Tregs can comprise 10 EU/ml endotoxins to 8 EU/ml endotoxins, 10 EU/ml endotoxins to 6 EU/ml endotoxins, 10 EU/ml endotoxins to 5 EU/ml endotoxins, 10 EU/ml endotoxins to 4 EU/ml endotoxins, 10 EU/ml endotoxins to 2 EU/ml endotoxins, 10 EU/ml endotoxins to 1 EU/ml endotoxins, 10 EU/ml endotoxins to 0.5 EU/ml endotoxins, 8 EU/ml endotoxins to 6 EU/ml endotoxins, 8 EU/ml endotoxins to 5 EU/ml endotoxins, 8 EU/ml endotoxins to 4 EU/ml endotoxins, 8 EU/ml endotoxins to 2 EU/ml endotoxins, 8 EU/ml endotoxins to 1 EU/ml endotoxins, 8 EU/ml endotoxins to 0.5 EU/ml endotoxins, 6 EU/ml endotoxins to 5 EU/ml endotoxins, 6 EU/ml endotoxins to 4 EU/ml endotoxins, 6 EU/ml endotoxins to 2 EU/ml endotoxins, 6 EU/ml endotoxins to 1 EU/ml endotoxins, 6 EU/ml endotoxins to 0.5 EU/ml endotoxins, 5 EU/ml endotoxins to 4 EU/ml endotoxins, 5 EU/ml endotoxins to 2 EU/ml endotoxins, 5 EU/ml endotoxins to 1 EU/ml endotoxins, 5 EU/ml endotoxins to 0.5 EU/ml endotoxins, 4 EU/ml endotoxins to 2 EU/ml endotoxins, 4 EU/ml endotoxins to 1 EU/ml endotoxins, 4 EU/ml endotoxins to 0.5 EU/ml endotoxins, 2 EU/ml endotoxins to 1 EU/ml endotoxins, 2 EU/ml endotoxins to 0.5 EU/ml endotoxins, or 1 EU/ml endotoxins to 0.5 EU/ml endotoxins. A population of cells enriched for Tregs can comprise 10 EU/ml endotoxins, 8 EU/ml endotoxins, 6 EU/ml endotoxins, 5 EU/ml endotoxins, 4 EU/ml endotoxins, 2 EU/ml endotoxins, 1 EU/ml endotoxins, or 0.5 EU/ml endotoxins. A population of cells enriched for Tregs can comprise at least 8 EU/ml endotoxins, 6 EU/ml endotoxins, 5 EU/ml endotoxins, 4 EU/ml endotoxins, 2 EU/ml endotoxins, 1 EU/ml endotoxins, or 0.5 EU/ml endotoxins. A population of cells enriched for Tregs can comprise at most 10 EU/ml endotoxins, 8 EU/ml endotoxins, 6 EU/ml endotoxins, 5 EU/ml endotoxins, 4 EU/ml endotoxins, 2 EU/ml endotoxins or 1 EU/ml endotoxins.

A population of cells enriched for Tregs can comprise 0.5% w/w to 10% w/w unbound reagents. These unbound reagents may include any affinity reagents used for the sorting of Tregs, for instance, antibodies, or purification particles or magnetic particles. A population of cells enriched for Tregs can comprise at least 0.5% w/w unbound reagents. A population of cells enriched for Tregs can comprise at most 10% w/w unbound reagents. A population of cells enriched for Tregs can comprise 10% w/w to 8% w/w, 10% w/w to 6% w/w, 10% w/w to 5% w/w, 10% w/w to 4% w/w, 10% w/w to 2% w/w, 10% w/w to 1% w/w, 10% w/w to 0.5% w/w, 8% w/w to 6% w/w, 8% w/w to 5% w/w, 8% w/w to 4% w/w, 8% w/w to 2% w/w, 8% w/w to 1% w/w, 8% w/w to 0.5% w/w, 6% w/w to 5% w/w, 6% w/w to 4% w/w, 6% w/w to 2% w/w, 6% w/w to 1% w/w, 6% w/w to 0.5% w/w, 5% w/w to 4% w/w, 5% w/w to 2% w/w, 5% w/w to 1% w/w, 5% w/w to 0.5% w/w, 4% w/w to 2% w/w, 4% w/w to 1% w/w, 4% w/w to 0.5% w/w, 2% w/w to 1% w/w, 2% w/w to 0.5% w/w, or 1% w/w to 0.5% w/w unbound reagents. A population of cells enriched for Tregs can comprise 10% w/w, 8% w/w, 6% w/w, 5% w/w, 4% w/w, 2% w/w, 1% w/w, or 0.5% w/w unbound reagents. A population of cells enriched for Tregs can comprise at least 8% w/w, 6% w/w, 5% w/w, 4% w/w, 2% w/w, 1% w/w, or 0.5% w/w unbound reagents. A population of cells enriched for Tregs can comprise at most 10% w/w, 8% w/w, 6% w/w, 5% w/w, 4% w/w, 2% w/w or 1% w/w unbound reagents.

A population of cells enriched for Tregs can comprise 1×10³ to 50×10³ microbeads per cell. These microbeads may comprise microbeads used to purify the Treg population, for instance, an anti-CD25 antibody comprising microbead used to sort the Treg population, or an anti-CD4 antibody comprising microbead used to sort the Treg population, and/or an anti-CD127 antibody comprising microbead used to sort the Treg population. A population of cells enriched for Tregs can comprise at least 1×10³ microbeads per cell. A population of cells enriched for Tregs can comprise at most 50×10³ microbeads per cell. A population of cells enriched for Tregs can comprise 50×10³ to 40×10³, 50×10³ to 30×10³, 50×10³ to 20×10³, 50×10³ to 10×10³, 50×10³ to 5×10³, 50×10³ to 4×10³, 50×10³ to 2×10³, 50×10³ to 1×10³, 40×10³ to 30×10³, 40×10³ to 20×10³, 40×10³ to 10×10³, 40×10³ to 5×10³, 40×10³ to 4×10³, 40×10³ to 2×10³, 40×10³ to 1×10³, 30×10³ to 20×10³, 30×10³ to 10×10³, 30×10³ to 5×10³, 30×10³ to 4×10³, 30×10³ to 2×10³, 30×10³ to 1×10³, 20×10³ to 10×10³, 20×10³ to 5×10³, 20×10³ to 4×10³, 20×10³ to 2×10³, 20×10³ to 1×10³, 10×10³ to 5×10³, 10×10³ to 4×10³, 10 ×10³ to 2×10³, 10 ×10³ to 1×10³, 5×10³ to 4×10³, 5×10³ to 2×10³, 5×10³ to 1×10³, 4×10³ to 2×10³, 4×10³ to 1×10³, or 2×10³ to 1×10³ microbeads per cell. A population of cells enriched for Tregs can comprise 50×10³, 40×10³, 30×10³, 20×10³, 10×10³, 5×10³, 4×10³, 2×10³, or 1×10³ microbeads per cell.

### Tcons

A second population of CD45+ cells that comprises, at least, Tcons can comprise at least about 1×10⁴, at least about 1×10⁵, at least about 5×10⁵, at least about 6×10⁵, at least about 7×10⁵, at least about 8×10⁵, at least about 9×10⁵, at least about 1×10⁶, at least about 1.1×10⁶, at least about 1.2×10⁶, at least about 1.3×10⁶, at least about 1.4×10⁶, at least about 1.5×10⁶, at least about 1.6×10⁶, at least about 1.7×10⁶, at least about 1.8×10⁶, at least about 1.9×10⁶, at least about 2×10⁶, at least about 2.1×10⁶, at least about 2.2×10⁶, at least about 2.3×10⁶, at least about 2.4×10⁶, at least about 2.5×10⁶, at least about 2.6×10⁶, at least about 2.7×10⁶, at least about 2.8×10⁶, at least about 2.9×10⁶, at least about 3×10⁶, at least about 3.1×10⁶, at least about 3.2×10⁶, at least about 3.3×10⁶, at least about 3.4×10⁶, at least about 3.5×10⁶, at least about 3.6×10⁶, at least about 3.7×10⁶, at least about 3.8×10⁶, at least about 3.9×10⁶, at least about 4×10⁶, at least about 4.1×10⁶, at least about 4.2×10⁶, at least about 4.3×10⁶, at least about 4.4×10⁶, at least about 4.5×10⁶, at least about 4.6×10⁶, at least about 4.7×10⁶, at least about 4.8×10⁶, at least about 4.9×10⁶, at least about 5×10⁶, at least about 5.1×10⁶, at least about 5.2×10⁶, at least about 5.3×10⁶, at least about 5.4×10⁶, at least about 5.5×10⁶, at least about 5.6×10⁶, at least about 5.7×10⁶, at least about 5.8×10⁶, at least about 5.9×10⁶, at least about 6×10⁶, at least about 6.5×10⁶, at least about 7×10⁶, at least about 7.5×10⁶, at least about 8×10⁶, at least about 8.5×10⁶, at least about 9×10⁶, at least about 9.5×10⁶, at least about 1×10⁷, at least about 1.5×10⁷, at least about 2×10⁷, at least about 2.5×10⁷, at least about 3×10⁷, at least about 3.5×10⁷, at least about 4×10⁷, at least about 4.5×10⁷, at least about 5×10⁷, at least about 5.5×10⁷, at least about 6×10⁷, at least about 6.5×10⁷, at least about 7×10⁷, at least about 7.5×10⁷, at least about 8×10⁷, at least about 8.5×10⁷, at least about 9×10⁷, at least about 9.5×10⁷, at least about 1×10⁸, at least about 1×10⁸, at least about 1.5×10⁸, at least about 2×10⁸, at least about 2.5×10⁸, at least about 3×10⁸, at least about 3.5×10⁸, at least about 4×10⁷, at least about 4.5×10⁸, at least about 5×10⁸, at least about 5.5×10⁸, at least about 6×10⁸, at least about 6.5×10⁸, at least about 7×10⁸, at least about 7.5×10⁸, at least about 8×10⁸, at least about 8.5×10⁸, at least about 9×10⁸, at least about 9.5×10⁸, at least about 1×10⁹, or more cells of the second population of CD45+ cells and/or Tcons per kg of recipient subject's actual body weight or ideal body weight (e.g., where Tcons are CD3+ or CD3+CD127+/bright).

A second population of CD45+ cells that comprises, at least, Tcons can comprise at most about 1×10⁴, at most about 1×10⁵, at most about 5×10⁵, at most about 6×10⁵, at most about 7×10⁵, at most about 8×10⁵, at most about 9×10⁵, at most about 1×10⁶, at most about 1.1×10⁶, at most about 1.2×10⁶, at most about 1.3×10⁶, at most about 1.4×10⁶, at most about 1.5×10⁶, at most about 1.6×10⁶, at most about 1.7×10⁶, at most about 1.8×10⁶, at most about 1.9×10⁶, at most about 2×10⁶, at most about 2.1×10⁶, at most about 2.2×10⁶, at most about 2.3×10⁶, at most about 2.4×10⁶, at most about 2.5×10⁶, at most about 2.6×10⁶, at most about 2.7×10⁶, at most about 2.8×10⁶, at most about 2.9×10⁶, at most about 3×10⁶, at most about 3.1×10⁶, at most about 3.2×10⁶, at most about 3.3×10⁶, at most about 3.4×10⁶, at most about 3.5×10⁶, at most about 3.6×10⁶, at most about 3.7×10⁶, at most about 3.8×10⁶, at most about 3.9×10⁶, at most about 4×10⁶, at most about 4.1×10⁶, at most about 4.2×10⁶, at most about 4.3×10⁶, at most about 4.4×10⁶, at most about 4.5×10⁶, at most about 4.6×10⁶, at most about 4.7×10⁶, at most about 4.8×10⁶, at most about 4.9×10⁶, at most about 5×10⁶, at most about 5.1×10⁶, at most about 5.2×10⁶, at most about 5.3×10⁶, at most about 5.4×10⁶, at most about 5.5×10⁶, at most about 5.6×10⁶, at most about 5.7×10⁶, at most about 5.8×10⁶, at most about 5.9×10⁶, at most about 6×10⁶, at most about 6.5×10⁶, at most about 7×10⁶, at most about 7.5×10⁶, at most about 8×10⁶, at most about 8.5×10⁶, at most about 9×10⁶, at most about 9.5×10⁶, at most about 1×10⁷, at most about 1.5×10⁷, at most about 2×10⁷, at most about 2.5×10⁷, at most about 3×10⁷, at most about 3.5×10⁷, at most about 4×10⁷, at most about 4.5×10⁷, at most about 5×10⁷, at most about 5.5×10⁷, at most about 6×10⁷, at most about 6.5×10⁷, at most about 7×10⁷, at most about 7.5×10⁷, at most about 8×10⁷, at most about 8.5×10⁷, at most about 9×10⁷, at most about 9.5×10⁷, at most about 1×10⁸, at most about 1×10⁸, at most about 1.5×10⁸, at most about 2×10⁸, at most about 2.5×10⁸, at most about 3×10⁸, at most about 3.5×10⁸, at most about 4×10⁷, at most about 4.5×10⁸, at most about 5×10⁸, at most about 5.5×10⁸, at most about 6×10⁸, at most about 6.5×10⁸, at most about 7×10⁸, at most about 7.5×10⁸, at most about 8×10⁸, at most about 8.5×10⁸, at most about 9×10⁸, at most about 9.5×10⁸, or at most about 1×10⁹ cells of the second population of CD45+ cells and/or Tcons per kg of recipient subject's actual body weight or ideal body weight (e.g., where Tcons are CD3+ or CD3+CD127+/bright).

For example, a second population of CD45+ cells that comprises, at least, Tcons can comprise 1×10⁴ to 1×10¹, 1×10⁵ to 1×10⁸, 1 ×10⁵ to 2×10⁷, 5×10⁵ to 2×10⁷, 5×10⁵ to 1.5×10⁷, 5×10⁵ to 1×10⁷, 5×10⁵ to 9×10⁶, 5×10⁵ to 8×10⁶, 5×10⁵ to 7×10⁶, 5×10⁵ to 6×10⁶, 5×10⁵ to 5×10⁶, 5×10⁵ to 4×10⁶, 5×10⁵ to 3×10⁶, 5×10⁵ to 2×10⁶, 5×10⁵ to 1×10⁶, 1×10⁶ to 1.5×10⁷, 1×10⁶ to 1×10⁷, 1×10⁶ to 9×10⁶, 1×10⁶ to 8×10⁶, 1×10⁶ to 7×10⁶, 1×10⁶ to 6×10⁶, 1×10⁶ to 5×10⁶, 1×10⁶ to 4×10⁶, 1×10⁶ to 3×10⁶, 1×10⁶ to 2×10⁶, 1.5×10⁶ to 1.5×10⁷, 1.5×10⁶ to 1×10⁷, 1.5×10⁶ to 9×10⁶, 1.5×10⁶ to 8×10⁶, 1.5×10⁶ to 7×10⁶, 1.5×10⁶ to 6×10⁶, 1.5×10⁶ to 5×10⁶, 1.5×10⁶ to 4×10⁶, 1.5×10⁶ to 3×10⁶, 1.5×10⁶ to 2×10⁶, 2×10⁶ to 1.5×10⁷, 2×10⁶ to 1×10⁷, 2×10⁶ to 9×10⁶, 2×10⁶ to 8×10⁶, 2×10⁶ to 7×10⁶, 2×10⁶ to 6×10⁶, 2×10⁶ to 5×10⁶, 2×10⁶ to 4×10⁶, 2×10⁶ to 3×10⁶, 2.5×10⁶ to 1.5×10⁷, 2.5×10⁶ to 1×10⁷, 2.5×10⁶ to 9×10⁶, 2.5×10⁶ to 8×10⁶, 2.5×10⁶ to 7×10⁶, 2.5×10⁶ to 6×10⁶, 2.5×10⁶ to 5×10⁶, 2.5×10⁶ to 4×10⁶, or 2.5×10⁶ to 3×10⁶ cells of the second population of CD45+ cells and/or Tcons per kg of recipient subject's actual body weight or ideal body weight (e.g., where Tcons are CD3+ or CD3+CD127+/bright).

A second population of CD45+ cells of the disclosure can have a defined level of purity for Tcon cells. For example, a second population of CD45+ cells of the disclosure can comprise at least about 5%, at least about at least about 10%, at least about at least about 15%, at least about 20%, at least about 25%, at least about 30%, at least about 35%, at least about 40%, at least about 45%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, or more Tcon cells as a percentage of total cells, as a percentage of nucleated cells, or as a percentage of CD45+ cells (e.g., where Tcons are CD3+ or CD3+CD127+/bright).

A second population of CD45+ cells of the disclosure can comprise 50% to 100%, 60% to 100%, 70% to 100%, 75% to 100%, 80% to 100%, 81% to 100%, 82% to 100%, 83% to 100%, 84% to 100%, 84% to 100%, 86% to 100%, 87% to 100%, 88% to 100%, 89% to 100%, 90% to 91%, 92% to 100%, 93% to 100%, 94% to 100%, 95% to 100%, 96% to 100%, 97% to 100%, 98% to 100%, 99% to 100%, 99.5% to 100%, 50% to 99%, 60% to 99%, 70% to 99%, 80% to 99%, 81% to 99%, 82% to 99%, 83% to 99%, 84% to 99%, 85% to 99%, 86% to 99%, 87% to 99%, 88% to 99%, 89% to 99%, 90% to 99%, 91% to 99%, 92% to 99%, 94% to 99%, 95% to 99%, 96% to 97%, 98% to 99%, 50% to 98%, 60% to 98%, 70% to 98%, 80% to 98%, 81% to 98%, 82% to 98%, 83% to 98%, 84% to 98%, 85% to 98%, 86% to 98%, 87% to 98%, 88% to 98%, 89% to 98%, 90% to 98%, 91% to 98%, 92% to 98%, 94% to 98%, 95% to 98%, 96% to 97%, 98% to 98%, 50% to 97%, 60% to 97%, 70% to 97%, 80% to 97%, 81% to 97%, 82% to 97%, 83% to 97%, 84% to 97%, 85% to 97%, 86% to 97%, 87% to 97%, 88% to 97%, 89% to 97%, 90% to 97%, 91% to 97%, 92% to 97%, 94% to 97%, 95% to 97%, 96% to 97%, 50% to 96%, 60% to 96%, 70% to 96%, 80% to 96%, 81% to 96%, 82% to 96%, 83% to 96%, 84% to 96%, 85% to 96%, 86% to 96%, 87% to 96%, 88% to 96%, 89% to 96%, 90% to 96%, 91% to 96%, 92% to 96%, 94% to 96%, 95% to 96%, 50% to 95%, 60% to 95%, 70% to 95%, 80% to 95%, 81% to 95%, 82% to 95%, 83% to 95%, 84% to 95%, 85% to 95%, 86% to 95%, 87% to 95%, 88% to 95%, 89% to 95%, 90% to 95%, 91% to 95%, 92% to 95%, or 94% to 95%, Tcons as a percentage of total cells, nucleated cells, or CD45+ cells (e.g., where Tcons are CD3+ or CD3+CD127+/bright).

A second population of CD45+ cells of the disclosure can have a defined level of contaminating non-Tcon cells. In some embodiments, at most about 1×10², at most about 2×10², at most about 3×10², at most about 4×10², at most about 5×10², at most about 6×10², at most about 7×10², at most about 8×10², at most about 9×10², at most about 1×10³ at most about 2×10³, at most about 3×10³, at most about 4×10³, at most about 5×10³, at most about 6×10³, at most about 7×10³, at most about 8×10³, at most about 9×10³, at most about 1×10⁴, at most about 2×10⁴, at most about 3×10⁴, at most about 4×10⁴, at most about 5×10⁴, at most about 6×10⁴, at most about 7×10⁴, at most about 8×10⁴, at most about 9×10⁴, or at most about 1×10⁵ non-Tcon cells per kg of recipient subject's actual body weight or ideal body weight are present in a second population of CD45+ cells of the disclosure, (e.g., where non-Tcon cells are CD3− or CD3+CD127dim).

In some embodiments, a second population of CD45+ cells of the disclosure comprises at most about 0.001%, at most about 0.002%, at most about 0.003%, at most about 0.004%, at most about 0.005%, at most about 0.006%, at most about 0.007%, at most about 0.008% 0.009%, at most about 0.01%, at most about 0.02%, at most about 0.03%, at most about 0.04%, at most about 0.05%, at most about 0.06%, at most about 0.07%, at most about 0.08%, at most about 0.09%, at most about 0.1%, at most about 0.2%, at most about 0.3%, at most about 0.4%, at most about 0.5%, at most about 0.6%, at most about 0.7%, at most about 0.8%, at most about 0.9%, at most about 1%, at most about 1.1%, at most about 1.2%, at most about 1.3%, at most about 1.4%, at most about 1.5%, at most about 1.6%, at most about 1.7%, at most about 1.8%, at most about 1.9%, at most about 2%, at most about 2.1%, at most about 2.2%, at most about 2.3%, at most about 2.4%, at most about 2.5%, at most about 2.6%, at most about 2.7%, at most about 2.8%, at most about 2.9%, at most about 3%, at most about 3.1%, at most about 3.2%, at most about 3.3%, at most about 3.4%, at most about 3.5%, at most about 3.6%, at most about 3.7%, at most about 3.8%, at most about 3.9%, at most about 4%, at most about 5%, at most about 6%, at most about 7%, at most about 8%, at most about 9%, or at most about 10% non-Tcon cells, (e.g., where non-Tcon cells are CD3− or CD3+CD127dim).

In some cases, the population of cells enriched for Tregs comprises less than about 5 EU of endotoxins per ml of the solution, less than about 1 EU of endotoxins per ml of the solution, and/or less than about 0.5 EU of endotoxins per ml of the solution.

In embodiments, at least one of the cell populations comprise less than about 5 EU of endotoxins/ml of respective suspension liquid.

A second population of CD45+ cells that comprises, at least, Tcons can comprise 0.5 EU/ml endotoxins to 10 EU/ml endotoxins. A second population of CD45+ cells can comprise at least 0.5 EU/ml endotoxins. A second population of CD45+ cells can comprise at most 10 EU/ml endotoxins. A second population of CD45+ cells can comprise 10 EU/ml endotoxins to 8 EU/ml endotoxins, 10 EU/ml endotoxins to 6 EU/ml endotoxins, 10 EU/ml endotoxins to 5 EU/ml endotoxins, 10 EU/ml endotoxins to 4 EU/ml endotoxins, 10 EU/ml endotoxins to 2 EU/ml endotoxins, 10 EU/ml endotoxins to 1 EU/ml endotoxins, 10 EU/ml endotoxins to 0.5 EU/ml endotoxins, 8 EU/ml endotoxins to 6 EU/ml endotoxins, 8 EU/ml endotoxins to 5 EU/ml endotoxins, 8 EU/ml endotoxins to 4 EU/ml endotoxins, 8 EU/ml endotoxins to 2 EU/ml endotoxins, 8 EU/ml endotoxins to 1 EU/ml endotoxins, 8 EU/ml endotoxins to 0.5 EU/ml endotoxins, 6 EU/ml endotoxins to 5 EU/ml endotoxins, 6 EU/ml endotoxins to 4 EU/ml endotoxins, 6 EU/ml endotoxins to 2 EU/ml endotoxins, 6 EU/ml endotoxins to 1 EU/ml endotoxins, 6 EU/ml endotoxins to 0.5 EU/ml endotoxins, 5 EU/ml endotoxins to 4 EU/ml endotoxins, 5 EU/ml endotoxins to 2 EU/ml endotoxins, 5 EU/ml endotoxins to 1 EU/ml endotoxins, 5 EU/ml endotoxins to 0.5 EU/ml endotoxins, 4 EU/ml endotoxins to 2 EU/ml endotoxins, 4 EU/ml endotoxins to 1 EU/ml endotoxins, 4 EU/ml endotoxins to 0.5 EU/ml endotoxins, 2 EU/ml endotoxins to 1 EU/ml endotoxins, 2 EU/ml endotoxins to 0.5 EU/ml endotoxins, or 1 EU/ml endotoxins to 0.5 EU/ml endotoxins. A second population of CD45+ cells can comprise about 10 EU/ml endotoxins, 8 EU/ml endotoxins, 6 EU/ml endotoxins, 5 EU/ml endotoxins, 4 EU/ml endotoxins, 2 EU/ml endotoxins, 1 EU/ml endotoxins, or 0.5 EU/ml endotoxins. A second population of CD45+ cells can comprise at least 8 EU/ml endotoxins, 6 EU/ml endotoxins, 5 EU/ml endotoxins, 4 EU/ml endotoxins, 2 EU/ml endotoxins, 1 EU/ml endotoxins, or 0.5 EU/ml endotoxins. A second population of CD45+ cells can comprise at most 10 EU/ml endotoxins, 8 EU/ml endotoxins, 6 EU/ml endotoxins, 5 EU/ml endotoxins, 4 EU/ml endotoxins, 2 EU/ml endotoxins or 1 EU/ml endotoxins.

A second population of CD45+ cells can comprise less than 0.1% w/w to 3% w/w unbound reagents. These unbound reagents may include any affinity reagents used for the sorting of Tcons or other cell populations, for instance, antibodies, or purification particles or magnetic particles. A second population of CD45+ cells can comprise less than about 0.1% w/w unbound reagents. A second population of CD45+ cells can comprise less than 3% w/w to 2% w/w, 3% w/w to 1% w/w, 3% w/w to 0.5% w/w, 3% w/w to 0.25% w/w, 3% w/w to 0.1% w/w, 2% w/w to 1% w/w, 2% w/w to 0.5% w/w, 2% w/w to 0.25% w/w, 2% w/w to 0.1% w/w, 1% w/w to 0.5% w/w, 1% w/w to 0.25% w/w, 1% w/w to 0.1% w/w, 0.5% w/w to 0.25% w/w, 0.5% w/w to 0.1% w/w, or 0.25% w/w to 0.1% w/w unbound reagents. A second population of CD45+ cells can comprise less than about 3% w/w, 2% w/w, 1% w/w, 0.5% w/w, 0.25% w/w, or 0.1% w/w unbound reagents.

A second population of CD45+ cells can comprise less than 50 to 2,000 microbeads per cell. These microbeads may comprise microbeads used to purify the Tcon population or other cell populations, for instance, a CD25 microbead, or a CD4 microbead, or a CD127 microbead, or a CD34 microbead used to sort a cell population. A second population of CD45+ cells can comprise less than 2,000 microbeads per cell. A second population of CD45+ cells can comprise less than 2,000 to 1,000, 2,000 to 700, 2,000 to 500, 2,000 to 300, 2,000 to 100, 2,000 to 50, 1,000 to 700, 1,000 to 500, 1,000 to 300, 1,000 to 100, 1,000 to 50, 700 to 500, 700 to 300, 700 to 100, 700 to 50, 500 to 300, 500 to 100, 500 to 50, 300 to 100, 300 to 50, or 100 to 50 microbeads per cell. A second population of CD45+ cells can comprise about 2,000, 1,000, 700, 500, 300, 100, or 50 microbeads per cell. A second population of CD45+ cells can comprise no microbeads per cell.

In a second population of CD45+ cells, the ratio of Tcons:Tregs administered to a subject can be, for example, about 1:100, 1:50, 1:25, 1:20, 1:15, 1:10, 1:9, 1:8, 1:7, 1:6, 1:5, 1:4, 1:3, 1:2.5, 1:2, 1.5:2, 1:1.5, 1:1, 1.5:1, 2:1, 2:1.5, 2.5:1, 3:1, 4:1, 5:1, 6:1, 7:1, 8:1, 9:1, 10:1, 15:1, 20:1, 25:1, 50:1, or 100:1.

Cells of the second population of CD45+ cells that comprises, at least, Tcons can be cryopreserved for any amount of time. Cells of the second population of CD45+ cells may be cryopreserved for at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 at least about 14 hours, at least about 16 hours, at least about 18 hours, at least about 20 hours, at least about 22 hours, at least about 24 hours, at least about 30 hours, at least about 36 hours at least about 48 hours, at least about 50 hours, at least about 55 hours, at least about 60 hours, at least about 61 hours, at least about 62 hours, at least about 65 hours, at least about 70 hours, at least about 72 hours, at least about 80 hours, at least about 90 hours, at least about 96 hours, at least about 120 hours, at least about 150 hours, at least about 200 hours, at least about 300 hours, or more prior to thawing and administration to a subject.

In some embodiments, cells of the second population of CD45+ cells that comprises, at least, Tcons are cryopreserved for at most about 1 hour, at most about 2 hours, at most about 3 hours, at most about 4 hours, at most about 5 hours, at most about 6 hours, at most about 7 hours, at most about 8 hours, at most about 9 hours, at most about 10 hours, at most about 11 hours, at most about 12 at most about 14 hours, at most about 16 hours, at most about 18 hours, at most about 20 hours, at most about 22 hours, at most about 24 hours, at most about 30 hours, at most about 36 hours at most about 48 hours, at most about 50 hours, at most about 55 hours, at most about 60 hours, at most about 61 hours, at most about 62 hours, at most about 65 hours, at most about 70 hours, at most about 72 hours, at most about 80 hours, at most about 90 hours, at most about 96 hours, at most about 120 hours, at most about 150 hours, at most about 200 hours, or at most about 300 hours prior to thawing and administration to a subject.

In some embodiments, cells of the second population of CD45+ cells are cryopreserved for at least about 1 day, at least about 2 days, at least about 3 days, at least about 4 days, at least about 5 days, at least about 6 days, at least about 7 days, at least about 10 days, at least about 14 days, at least about 21 days, at least about 28 days, at least about 50 days, at least about 60 days, or at least about 96 days, or more prior to thawing and administration to a subject.

In some embodiments, cells of the second population of CD45+ cells are cryopreserved for at most about 1 day, at most about 2 days, at most about 3 days, at most about 4 days, at most about 5 days, at most about 6 days, at most about 7 days, at most about 10 days, at most about 14 days, at most about 21 days, at most about 28 days, at most about 50 days, at most about 60 days, or at most about 96 days prior to thawing and administration to a subject.

iNKTs

A cell population that comprises a population of iNKTs can comprise at least about 1×10⁴, at least about 1×10⁵, at least about 5×10⁵, at least about 6×10⁵, at least about 7×10⁵, at least about 8×10⁵, at least about 9×10⁵, at least about 1×10⁶, at least about 1.1×10⁶, at least about 1.2×10⁶, at least about 1.3×10⁶, at least about 1.4×10⁶, at least about 1.5×10⁶, at least about 1.6×10⁶, at least about 1.7×10⁶, at least about 1.8×10⁶, at least about 1.9×10⁶, at least about 2×10⁶, at least about 2.1×10⁶, at least about 2.2×10⁶, at least about 2.3×10⁶, at least about 2.4×10⁶, at least about 2.5×10⁶, at least about 2.6×10⁶, at least about 2.7×10⁶, at least about 2.8×10⁶, at least about 2.9×10⁶, at least about 3×10⁶, at least about 3.1×10⁶, at least about 3.2×10⁶, at least about 3.3×10⁶, at least about 3.4×10⁶, at least about 3.5×10⁶, at least about 3.6×10⁶, at least about 3.7×10⁶, at least about 3.8×10⁶, at least about 3.9×10⁶, at least about 4×10⁶, at least about 4.1×10⁶, at least about 4.2×10⁶, at least about 4.3×10⁶, at least about 4.4×10⁶, at least about 4.5×10⁶, at least about 4.6×10⁶, at least about 4.7×10⁶, at least about 4.8×10⁶, at least about 4.9×10⁶, at least about 5×10⁶, at least about 5.1×10⁶, at least about 5.2×10⁶, at least about 5.3×10⁶, at least about 5.4×10⁶, at least about 5.5×10⁶, at least about 5.6×10⁶, at least about 5.7×10⁶, at least about 5.8×10⁶, at least about 5.9×10⁶, at least about 6×10⁶, at least about 6.5×10⁶, at least about 7×10⁶, at least about 7.5×10⁶, at least about 8×10⁶, at least about 8.5×10⁶, at least about 9×10⁶, at least about 9.5×10⁶, at least about 1×10⁷, at least about 1.5×10⁷, at least about 2×10⁷, at least about 2.5×10⁷, at least about 3×10⁷, at least about 3.5×10⁷, at least about 4×10⁷, at least about 4.5×10⁷, at least about 5×10⁷, at least about 5.5×10⁷, at least about 6×10⁷, at least about 6.5×10⁷, at least about 7×10⁷, at least about 7.5×10⁷, at least about 8×10⁷, at least about 8.5×10⁷, at least about 9×10⁷, at least about 9.5×10⁷, at least about 1×10⁸, at least about 1×10⁸, at least about 1.5×10⁸, at least about 2×10⁸, at least about 2.5×10⁸, at least about 3×10⁸, at least about 3.5×10⁸, at least about 4×10⁷, at least about 4.5×10⁸, at least about 5×10⁸, at least about 5.5×10⁸, at least about 6×10⁸, at least about 6.5×10⁸, at least about 7×10⁸, at least about 7.5×10⁸, at least about 8×10⁸, at least about 8.5×10⁸, at least about 9×10⁸, at least about 9.5×10⁸, at least about 1×10⁹, or more iNKTs per kg of recipient subject's actual body weight or ideal body weight (e.g., where iNKTs are CD3+Vα24Jα18+).

A cell population that comprises a population of iNKTs can comprise at most about 1×10⁴, at most about 1×10⁵, at most about 5×10⁵, at most about 6×10⁵, at most about 7×10⁵, at most about 8×10⁵, at most about 9×10⁵, at most about 1×10⁶, at most about 1.1×10⁶, at most about 1.2×10⁶, at most about 1.3×10⁶, at most about 1.4×10⁶, at most about 1.5×10⁶, at most about 1.6×10⁶, at most about 1.7×10⁶, at most about 1.8×10⁶, at most about 1.9×10⁶, at most about 2×10⁶, at most about 2.1×10⁶, at most about 2.2×10⁶, at most about 2.3×10⁶, at most about 2.4×10⁶, at most about 2.5×10⁶, at most about 2.6×10⁶, at most about 2.7×10⁶, at most about 2.8×10⁶, at most about 2.9×10⁶, at most about 3×10⁶, at most about 3.1×10⁶, at most about 3.2×10⁶, at most about 3.3×10⁶, at most about 3.4×10⁶, at most about 3.5×10⁶, at most about 3.6×10⁶, at most about 3.7×10⁶, at most about 3.8×10⁶, at most about 3.9×10⁶, at most about 4×10⁶, at most about 4.1×10⁶, at most about 4.2×10⁶, at most about 4.3×10⁶, at most about 4.4×10⁶, at most about 4.5×10⁶, at most about 4.6×10⁶, at most about 4.7×10⁶, at most about 4.8×10⁶, at most about 4.9×10⁶, at most about 5×10⁶, at most about 5.1×10⁶, at most about 5.2×10⁶, at most about 5.3×10⁶, at most about 5.4×10⁶, at most about 5.5×10⁶, at most about 5.6×10⁶, at most about 5.7×10⁶, at most about 5.8×10⁶, at most about 5.9×10⁶, at most about 6×10⁶, at most about 6.5×10⁶, at most about 7×10⁶, at most about 7.5×10⁶, at most about 8×10⁶, at most about 8.5×10⁶, at most about 9×10⁶, at most about 9.5×10⁶, at most about 1×10⁷, at most about 1.5×10⁷, at most about 2×10⁷, at most about 2.5×10⁷, at most about 3×10⁷, at most about 3.5×10⁷, at most about 4×10⁷, at most about 4.5×10⁷, at most about 5×10⁷, at most about 5.5×10⁷, at most about 6×10⁷, at most about 6.5×10⁷, at most about 7×10⁷, at most about 7.5×10⁷, at most about 8×10⁷, at most about 8.5×10⁷, at most about 9×10⁷, at most about 9.5×10⁷, at most about 1×10⁸, at most about 1×10⁸, at most about 1.5×10⁸, at most about 2×10⁸, at most about 2.5×10⁸, at most about 3×10⁸, at most about 3.5×10⁸, at most about 4×10⁷, at most about 4.5×10⁸, at most about 5×10⁸, at most about 5.5×10⁸, at most about 6×10⁸, at most about 6.5×10⁸, at most about 7×10⁸, at most about 7.5×10⁸, at most about 8×10⁸, at most about 8.5×10⁸, at most about 9×10⁸, at most about 9.5×10⁸, or at most about 1×10⁹ iNKTs per kg of recipient subject's actual body weight or ideal body weight (e.g., where iNKTs are CD3+Vα24Jα18+).

For example, a cell population that comprises a population of iNKTs can comprise 1×10⁴ to 1×10⁹, 1×10⁵ to 1×10⁸, 1×10⁵ to 2×10⁷, 5×10⁵ to 2×10⁷, 5×10⁵ to 1.5×10⁷, 5×10⁵ to 1×10⁷, 5×10⁵ to 9×10⁶, 5×10⁵ to 8×10⁶, 5×10⁵ to 7×10⁶, 5×10⁵ to 6×10⁶, 5×10⁵ to 5×10⁶, 5×10⁵ to 4×10⁶, 5×10⁵ to 3×10⁶, 5×10⁵ to 2×10⁶, 5×10⁵ to 1×10⁶, 1×10⁶ to 1.5×10⁷, 1×10⁶ to 1 ×10⁷, 1×10⁶ to 9×10⁶, 1×10⁶ to 8×10⁶, 1×10⁶ to 7×10⁶, 1×10⁶ to 6×10⁶, 1×10⁶ to 5×10⁶, 1×10⁶ to 4×10⁶, 1×10⁶ to 3×10⁶, 1×10⁶ to 2×10⁶, 1.5×10⁶ to 1.5×10⁷, 1.5×10⁶ to 1×10⁷, 1.5×10⁶ to 9×10⁶, 1.5×10⁶ to 8×10⁶, 1.5×10⁶ to 7×10⁶, 1.5×10⁶ to 6×10⁶, 1.5×10⁶ to 5×10⁶, 1.5×10⁶ to 4×10⁶, 1.5×10⁶ to 3×10⁶, 1.5×10⁶ to 2×10⁶, 2×10⁶ to 1.5×10⁷, 2×10⁶ to 1×10⁷, 2×10⁶ to 9×10⁶, 2×10⁶ to 8×10⁶, 2×10⁶ to 7×10⁶, 2×10⁶ to 6×10⁶, 2×10⁶ to 5×10⁶, 2×10⁶ to 4×10⁶, 2×10⁶ to 3×10⁶, 2.5×10⁶ to 1.5×10⁷, 2.5×10⁶ to 1×10⁷, 2.5×10⁶ to 9×10⁶, 2.5×10⁶ to 8×10⁶, 2.5×10⁶ to 7×10⁶, 2.5×10⁶ to 6×10⁶, 2.5×10⁶ to 5×10⁶, 2.5×10⁶ to 4×10⁶, or 2.5×10⁶ to 3×10⁶ iNKTs per kg of recipient subject's actual body weight or ideal body weight (e.g., where iNKTs are CD3+Vα24Jα18+).

A population of iNKTs of the disclosure can have a defined level of purity for iNKT cells. For example, a population of iNKTs of the disclosure can comprise at least about 5%, at least about at least about 10%, at least about at least about 15%, at least about 20%, at least about 25%, at least about 30%, at least about 35%, at least about 40%, at least about 45%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, or more iNKT cells as a percentage of total cells, as a percentage of nucleated cells, or as a percentage of CD45+ cells (e.g., where iNKTs are CD3+Vα24Jα18+).

A population of iNKTs of the disclosure can comprise 50% to 100%, 60% to 100%, 70% to 100%, 75% to 100%, 80% to 100%, 81% to 100%, 82% to 100%, 83% to 100%, 84% to 100%, 84% to 100%, 86% to 100%, 87% to 100%, 88% to 100%, 89% to 100%, 90% to 91%, 92% to 100%, 93% to 100%, 94% to 100%, 95% to 100%, 96% to 100%, 97% to 100%, 98% to 100%, 99% to 100%, 99.5% to 100%, 50% to 99%, 60% to 99%, 70% to 99%, 80% to 99%, 81% to 99%, 82% to 99%, 83% to 99%, 84% to 99%, 85% to 99%, 86% to 99%, 87% to 99%, 88% to 99%, 89% to 99%, 90% to 99%, 91% to 99%, 92% to 99%, 94% to 99%, 95% to 99%, 96% to 97%, 98% to 99%, 50% to 98%, 60% to 98%, 70% to 98%, 80% to 98%, 81% to 98%, 82% to 98%, 83% to 98%, 84% to 98%, 85% to 98%, 86% to 98%, 87% to 98%, 88% to 98%, 89% to 98%, 90% to 98%, 91% to 98%, 92% to 98%, 94% to 98%, 95% to 98%, 96% to 97%, 98% to 98%, 50% to 97%, 60% to 97%, 70% to 97%, 80% to 97%, 81% to 97%, 82% to 97%, 83% to 97%, 84% to 97%, 85% to 97%, 86% to 97%, 87% to 97%, 88% to 97%, 89% to 97%, 90% to 97%, 91% to 97%, 92% to 97%, 94% to 97%, 95% to 97%, 96% to 97%, 50% to 96%, 60% to 96%, 70% to 96%, 80% to 96%, 81% to 96%, 82% to 96%, 83% to 96%, 84% to 96%, 85% to 96%, 86% to 96%, 87% to 96%, 88% to 96%, 89% to 96%, 90% to 96%, 91% to 96%, 92% to 96%, 94% to 96%, 95% to 96%, 50% to 95%, 60% to 95%, 70% to 95%, 80% to 95%, 81% to 95%, 82% to 95%, 83% to 95%, 84% to 95%, 85% to 95%, 86% to 95%, 87% to 95%, 88% to 95%, 89% to 95%, 90% to 95%, 91% to 95%, 92% to 95%, or 94% to 95%, iNKTs as a percentage of total cells, nucleated cells, or CD45+ cells (e.g., where iNKTs are CD3+Vα24Jα18+).

A population of iNKTs of the disclosure can have a defined level of contaminating non-iNKT cells. In some embodiments, at most about 1×10², at most about 2×10², at most about 3×10², at most about 4×10², at most about 5×10², at most about 6×10², at most about 7×10², at most about 8×10², at most about 9×10², at most about 1×10³, at most about 2×10³, at most about 3×10³, at most about 4×10³, at most about 5×10³, at most about 6×10³, at most about 7×10³, at most about 8×10³, at most about 9×10³, at most about 1×10⁴, at most about 2×10⁴, at most about 3×10⁴, at most about 4×10⁴, at most about 5×10⁴, at most about 6×10⁴, at most about 7×10⁴, at most about 8×10⁴, at most about 9×10⁴, or at most about 1×10⁵ non-iNKT cells per kg of recipient subject's actual body weight or ideal body weight are present in a population of iNKTs of the disclosure, (e.g., where non-iNKT cells are Vα24Jα18−).

In some embodiments, a population of iNKTs of the disclosure comprises at most about 0.001%, at most about 0.002%, at most about 0.003%, at most about 0.004%, at most about 0.005%, at most about 0.006%, at most about 0.007%, at most about 0.008% 0.009%, at most about 0.01%, at most about 0.02%, at most about 0.03%, at most about 0.04%, at most about 0.05%, at most about 0.06%, at most about 0.07%, at most about 0.08%, at most about 0.09%, at most about 0.1%, at most about 0.2%, at most about 0.3%, at most about 0.4%, at most about 0.5%, at most about 0.6%, at most about 0.7%, at most about 0.8%, at most about 0.9%, at most about 1%, at most about 1.1%, at most about 1.2%, at most about 1.3%, at most about 1.4%, at most about 1.5%, at most about 1.6%, at most about 1.7%, at most about 1.8%, at most about 1.9%, at most about 2%, at most about 2.1%, at most about 2.2%, at most about 2.3%, at most about 2.4%, at most about 2.5%, at most about 2.6%, at most about 2.7%, at most about 2.8%, at most about 2.9%, at most about 3%, at most about 3.1%, at most about 3.2%, at most about 3.3%, at most about 3.4%, at most about 3.5%, at most about 3.6%, at most about 3.7%, at most about 3.8%, at most about 3.9%, at most about 4%, at most about 5%, at most about 6%, at most about 7%, at most about 8%, at most about 9%, or at most about 10% non-iNKT cells, (e.g., where non-iNKT cells are Vα24Jα18−).

### Tmems

A cell population that comprises a population of Tmems can comprise at least about 1×10⁴, at least about 1×10⁵, at least about 5×10⁵, at least about 6×10⁵, at least about 7×10⁵, at least about 8×10⁵, at least about 9×10⁵, at least about 1×10⁶, at least about 1.1×10⁶, at least about 1.2×10⁶, at least about 1.3×10⁶, at least about 1.4×10⁶, at least about 1.5×10⁶, at least about 1.6×10⁶, at least about 1.7×10⁶, at least about 1.8×10⁶, at least about 1.9×10⁶, at least about 2×10⁶, at least about 2.1×10⁶, at least about 2.2×10⁶, at least about 2.3×10⁶, at least about 2.4×10⁶, at least about 2.5×10⁶, at least about 2.6×10⁶, at least about 2.7×10⁶, at least about 2.8×10⁶, at least about 2.9×10⁶, at least about 3×10⁶, at least about 3.1×10⁶, at least about 3.2×10⁶, at least about 3.3×10⁶, at least about 3.4×10⁶, at least about 3.5×10⁶, at least about 3.6×10⁶, at least about 3.7×10⁶, at least about 3.8×10⁶, at least about 3.9×10⁶, at least about 4×10⁶, at least about 4.1×10⁶, at least about 4.2×10⁶, at least about 4.3×10⁶, at least about 4.4×10⁶, at least about 4.5×10⁶, at least about 4.6×10⁶, at least about 4.7×10⁶, at least about 4.8×10⁶, at least about 4.9×10⁶, at least about 5×10⁶, at least about 5.1×10⁶, at least about 5.2×10⁶, at least about 5.3×10⁶, at least about 5.4×10⁶, at least about 5.5×10⁶, at least about 5.6×10⁶, at least about 5.7×10⁶, at least about 5.8×10⁶, at least about 5.9×10⁶, at least about 6×10⁶, at least about 6.5×10⁶, at least about 7×10⁶, at least about 7.5×10⁶, at least about 8×10⁶, at least about 8.5×10⁶, at least about 9×10⁶, at least about 9.5×10⁶, at least about 1×10⁷, at least about 1.5×10⁷, at least about 2×10⁷, at least about 2.5×10⁷, at least about 3×10⁷, at least about 3.5×10⁷, at least about 4×10⁷, at least about 4.5×10⁷, at least about 5×10⁷, at least about 5.5×10⁷, at least about 6×10⁷, at least about 6.5×10⁷, at least about 7×10⁷, at least about 7.5×10⁷, at least about 8×10⁷, at least about 8.5×10⁷, at least about 9×10⁷, at least about 9.5×10⁷, at least about 1×10⁸, at least about 1×10⁸, at least about 1.5×10⁸, at least about 2×10⁸, at least about 2.5×10⁸, at least about 3×10⁸, at least about 3.5×10⁸, at least about 4×10⁷, at least about 4.5×10⁸, at least about 5×10⁸, at least about 5.5×10⁸, at least about 6×10⁸, at least about 6.5×10⁸, at least about 7×10⁸, at least about 7.5×10⁸, at least about 8×10⁸, at least about 8.5×10⁸, at least about 9×10⁸, at least about 9.5×10⁸, at least about 1×10⁹, or more Tmems per kg of recipient subject's actual body weight or ideal body weight (e.g., where Tmems are CD3+CD45RA− CD45RO+).

A cell population that comprises a population of Tmems can comprise at most about 1×10⁴, at most about 1×10⁵, at most about 5×10⁵, at most about 6×10⁵, at most about 7×10⁵, at most about 8×10⁵, at most about 9×10⁵, at most about 1×10⁶, at most about 1.1×10⁶, at most about 1.2×10⁶, at most about 1.3×10⁶, at most about 1.4×10⁶, at most about 1.5×10⁶, at most about 1.6×10⁶, at most about 1.7×10⁶, at most about 1.8×10⁶, at most about 1.9×10⁶, at most about 2×10⁶, at most about 2.1×10⁶, at most about 2.2×10⁶, at most about 2.3×10⁶, at most about 2.4×10⁶, at most about 2.5×10⁶, at most about 2.6×10⁶, at most about 2.7×10⁶, at most about 2.8×10⁶, at most about 2.9×10⁶, at most about 3×10⁶, at most about 3.1×10⁶, at most about 3.2×10⁶, at most about 3.3×10⁶, at most about 3.4×10⁶, at most about 3.5×10⁶, at most about 3.6×10⁶, at most about 3.7×10⁶, at most about 3.8×10⁶, at most about 3.9×10⁶, at most about 4×10⁶, at most about 4.1×10⁶, at most about 4.2×10⁶, at most about 4.3×10⁶, at most about 4.4×10⁶, at most about 4.5×10⁶, at most about 4.6×10⁶, at most about 4.7×10⁶, at most about 4.8×10⁶, at most about 4.9×10⁶, at most about 5×10⁶, at most about 5.1×10⁶, at most about 5.2×10⁶, at most about 5.3×10⁶, at most about 5.4×10⁶, at most about 5.5×10⁶, at most about 5.6×10⁶, at most about 5.7×10⁶, at most about 5.8×10⁶, at most about 5.9×10⁶, at most about 6×10⁶, at most about 6.5×10⁶, at most about 7×10⁶, at most about 7.5×10⁶, at most about 8×10⁶, at most about 8.5×10⁶, at most about 9×10⁶, at most about 9.5×10⁶, at most about 1×10⁷, at most about 1.5×10⁷, at most about 2×10⁷, at most about 2.5×10⁷, at most about 3×10⁷, at most about 3.5×10⁷, at most about 4×10⁷, at most about 4.5×10⁷, at most about 5×10⁷, at most about 5.5×10⁷, at most about 6×10⁷, at most about 6.5×10⁷, at most about 7×10⁷, at most about 7.5×10⁷, at most about 8×10⁷, at most about 8.5×10⁷, at most about 9×10⁷, at most about 9.5×10⁷, at most about 1×10⁸, at most about 1×10⁸, at most about 1.5×10⁸, at most about 2×10⁸, at most about 2.5×10⁸, at most about 3×10⁸, at most about 3.5×10⁸, at most about 4×10⁷, at most about 4.5×10⁸, at most about 5×10⁸, at most about 5.5×10⁸, at most about 6×10⁸, at most about 6.5×10⁸, at most about 7×10⁸, at most about 7.5×10⁸, at most about 8×10⁸, at most about 8.5×10⁸, at most about 9×10⁸, at most about 9.5×10⁸, or at most about 1×10⁹ Tmems per kg of recipient subject's actual body weight or ideal body weight (e.g., where Tmems are CD3+CD45RA− CD45RO+).

For example, a cell population that comprises a population of Tmems can comprise 1×10⁴ to 1×10⁹, 1×10⁵ to 1×10⁸, 1×10⁵ to 2×10⁷, 5×10⁵ to 2×10⁷, 5×10⁵ to 1.5×10⁷, 5 ×10⁵ to 1×10⁷, 5×10⁵ to 9×10⁶, 5×10⁵ to 8×10⁶, 5×10⁵ to 7×10⁶, 5×10⁵ to 6×10⁶, 5×10⁵ to 5×10⁶, 5×10⁵ to 4×10⁶, 5×10⁵ to 3×10⁶, 5×10⁵ to 2×10⁶, 5×10⁵ to 1×10⁶, 1×10⁶ to 1.5×10⁷, 1×10⁶ to 1×10⁷, 1×10⁶ to 9×10⁶, 1×10⁶ to 8×10⁶, 1×10⁶ to 7×10⁶, 1×10⁶ to 6×10⁶, 1×10⁶ to 5×10⁶, 1×10⁶ to 4×10⁶, 1×10⁶ to 3×10⁶, 1×10⁶ to 2×10⁶, 1.5×10⁶ to 1.5×10⁷, 1.5×10⁶ to 1×10⁷, 1.5×10⁶ to 9×10⁶, 1.5×10⁶ to 8×10⁶, 1.5×10⁶ to 7×10⁶, 1.5×10⁶ to 6×10⁶, 1.5×10⁶ to 5×10⁶, 1.5×10⁶ to 4×10⁶, 1.5×10⁶ to 3×10⁶, 1.5×10⁶ to 2×10⁶, 2×10⁶ to 1.5×10⁷, 2×10⁶ to 1×10⁷, 2×10⁶ to 9×10⁶, 2×10⁶ to 8×10⁶, 2×10⁶ to 7×10⁶, 2×10⁶ to 6×10⁶, 2×10⁶ to 5×10⁶, 2×10⁶ to 4×10⁶, 2×10⁶ to 3×10⁶, 2.5×10⁶ to 1.5×10⁷, 2.5×10⁶ to 1×10⁷, 2.5×10⁶ to 9×10⁶, 2.5×10⁶ to 8×10⁶, 2.5×10⁶ to 7×10⁶, 2.5×10⁶ to 6×10⁶, 2.5×10⁶ to 5×10⁶, 2.5×10⁶ to 4×10⁶, or 2.5×10⁶ to 3×10⁶ Tmems per kg of recipient subject's actual body weight or ideal body weight (e.g., where Tmems are CD3+CD45RA− CD45RO+).

A population of Tmems of the disclosure can have a defined level of purity for Tmem cells. For example, a population of Tmems of the disclosure can comprise at least about 5%, at least about at least about 10%, at least about at least about 15%, at least about 20%, at least about 25%, at least about 30%, at least about 35%, at least about 40%, at least about 45%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, or more Tmem cells as a percentage of total cells, as a percentage of nucleated cells, or as a percentage of CD45+ cells (e.g., where Tmems are CD3+CD45RA− CD45RO+).

A population of Tmems of the disclosure can comprise 50% to 100%, 60% to 100%, 70% to 100%, 75% to 100%, 80% to 100%, 81% to 100%, 82% to 100%, 83% to 100%, 84% to 100%, 84% to 100%, 86% to 100%, 87% to 100%, 88% to 100%, 89% to 100%, 90% to 91%, 92% to 100%, 93% to 100%, 94% to 100%, 95% to 100%, 96% to 100%, 97% to 100%, 98% to 100%, 99% to 100%, 99.5% to 100%, 50% to 99%, 60% to 99%, 70% to 99%, 80% to 99%, 81% to 99%, 82% to 99%, 83% to 99%, 84% to 99%, 85% to 99%, 86% to 99%, 87% to 99%, 88% to 99%, 89% to 99%, 90% to 99%, 91% to 99%, 92% to 99%, 94% to 99%, 95% to 99%, 96% to 97%, 98% to 99%, 50% to 98%, 60% to 98%, 70% to 98%, 80% to 98%, 81% to 98%, 82% to 98%, 83% to 98%, 84% to 98%, 85% to 98%, 86% to 98%, 87% to 98%, 88% to 98%, 89% to 98%, 90% to 98%, 91% to 98%, 92% to 98%, 94% to 98%, 95% to 98%, 96% to 97%, 98% to 98%, 50% to 97%, 60% to 97%, 70% to 97%, 80% to 97%, 81% to 97%, 82% to 97%, 83% to 97%, 84% to 97%, 85% to 97%, 86% to 97%, 87% to 97%, 88% to 97%, 89% to 97%, 90% to 97%, 91% to 97%, 92% to 97%, 94% to 97%, 95% to 97%, 96% to 97%, 50% to 96%, 60% to 96%, 70% to 96%, 80% to 96%, 81% to 96%, 82% to 96%, 83% to 96%, 84% to 96%, 85% to 96%, 86% to 96%, 87% to 96%, 88% to 96%, 89% to 96%, 90% to 96%, 91% to 96%, 92% to 96%, 94% to 96%, 95% to 96%, 50% to 95%, 60% to 95%, 70% to 95%, 80% to 95%, 81% to 95%, 82% to 95%, 83% to 95%, 84% to 95%, 85% to 95%, 86% to 95%, 87% to 95%, 88% to 95%, 89% to 95%, 90% to 95%, 91% to 95%, 92% to 95%, or 94% to 95%, Tmems as a percentage of total cells, nucleated cells, or CD45+ cells (e.g., where Tmems are CD3+CD45RA− CD45RO+).

A population of Tmems of the disclosure can have a defined level of contaminating non-Tmem cells. In some embodiments, at most about 1×10², at most about 2×10², at most about 3×10², at most about 4×10², at most about 5×10², at most about 6×10², at most about 7×10², at most about 8×10², at most about 9×10², at most about 1×10³, at most about 2×10³, at most about 3×10³, at most about 4×10³, at most about 5×10³, at most about 6×10³, at most about 7×10³, at most about 8×10³, at most about 9×10³, at most about 1×10⁴, at most about 2×10⁴, at most about 3×10⁴, at most about 4×10⁴, at most about 5×10⁴, at most about 6×10⁴, at most about 7×10⁴, at most about 8×10⁴, at most about 9×10⁴, or at most about 1×10⁵ non-Tmem cells per kg of recipient subject's actual body weight or ideal body weight are present in a population of Tmems of the disclosure, (e.g., where the non-Tmem cells are CD45RO−).

In some embodiments, a population of Tmems of the disclosure comprises at most about 0.001%, at most about 0.002%, at most about 0.003%, at most about 0.004%, at most about 0.005%, at most about 0.006%, at most about 0.007%, at most about 0.008% 0.009%, at most about 0.01%, at most about 0.02%, at most about 0.03%, at most about 0.04%, at most about 0.05%, at most about 0.06%, at most about 0.07%, at most about 0.08%, at most about 0.09%, at most about 0.1%, at most about 0.2%, at most about 0.3%, at most about 0.4%, at most about 0.5%, at most about 0.6%, at most about 0.7%, at most about 0.8%, at most about 0.9%, at most about 1%, at most about 1.1%, at most about 1.2%, at most about 1.3%, at most about 1.4%, at most about 1.5%, at most about 1.6%, at most about 1.7%, at most about 1.8%, at most about 1.9%, at most about 2%, at most about 2.1%, at most about 2.2%, at most about 2.3%, at most about 2.4%, at most about 2.5%, at most about 2.6%, at most about 2.7%, at most about 2.8%, at most about 2.9%, at most about 3%, at most about 3.1%, at most about 3.2%, at most about 3.3%, at most about 3.4%, at most about 3.5%, at most about 3.6%, at most about 3.7%, at most about 3.8%, at most about 3.9%, at most about 4%, at most about 5%, at most about 6%, at most about 7%, at most about 8%, at most about 9%, or at most about 10% non-Tmem cells, (e.g., where the non-Tmem cells are CD45RO−).

### D. Sequence and Timing of Administration of Cell Populations

An aspect provides a multi-component pharmaceutical treatment to be administered to a human subject in need thereof. The multi-component treatment comprises (a) a solution comprising a first population of CD45+ cells comprising hematopoietic stem and progenitor cells (HSPCs) and granulocytes wherein at most about 10% of the first population of CD45+ cells comprise granulocytes; (b) a solution comprising a population of cells enriched for regulatory T cells (Tregs); (c) a solution comprising a second population of CD45+ cells wherein the second population of CD45+ cells comprise at least about 20% CD3+ conventional T cells (Tcons), at least about 10% monocytes, and at least about 10% granulocytes; and (d) a solution comprising one or more doses of a graft vs host disease (GVHD) prophylactic agent.

Another aspect provides a method of treating a human subject diagnosed with a hematologic malignancy. The method comprises administering to the human subject a solution comprising the first population of CD45+ cells, a solution comprising the population of cells enriched for regulatory T cells (Tregs), a solution comprising the second population of CD45+ cells, and a solution comprising one or more doses of the GVHD prophylactic agent. In this aspect, the solution comprising the first population of CD45+ cells, the solution comprising the population of cells enriched for regulatory Tregs, the solution comprising the second population of CD45+ cells, and the solution comprising one or more doses of the GVHD prophylactic agent are as defined according to any herein disclosed multi-component pharmaceutical treatment.

Disclosed herein are methods for enhanced allogeneic hematopoietic stem cell transplantation, comprising administering to a subject solutions that comprise populations of cells.

In some embodiments, cell populations that comprise a first population of CD45+ cells which comprises at least HSPCs, a population of cells enriched for regulatory T cells (Tregs), and a second population of CD45+ cells which comprises at least Tcons are administered to a subject.

The first population of CD45+ cells and the population of cells enriched for Tregs can be administered at the same or similar times, or at different times. In some embodiments, first population of CD45+ cells and the population of cells enriched for Tregs are administered on the same day.

In various embodiments, the first population of CD45+ cells and the population of cells enriched for Tregs are administered before the second population of CD45+ cells.

The first population of CD45+ cells and the population of cells enriched for Tregs can administered at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours apart.

The second population of CD45+ cells can be administered to the subject after the first population of CD45+ cells.

The second population of CD45+ cells can be administered to the subject at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the first population of CD45+ cells.

In some embodiments, the second population of CD45+ cells is administered to the subject at most about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, or 120 hours after the first population of CD45+ cells.

The second population of CD45+ cells can be administered to the subject, for example, between about 6-96, 12-84, 12-72, 12-66, 12-60, 12-54, 12-48, 12-42, 12-36, 12-30, 12-24, 12-18, 18-72, 18-66, 18-60, 18-54, 18-48, 18-42, 18-36, 18-30, 18-24, 24-72, 24-66, 24-60, 24-54, 24-48, 24-42, 24-36, 24-30, 30-72, 30-66, 30-60, 30-54, 30-48, 30-42, 30-36, 36-72, 36-66, 36-60, 36-54, 36-48, 36-42, 42-72, 42-66, 42-60, 42-54, 42-48, 48-72, 48-66, 48-60, 48-54, 54-72, 54-66, 54-60, 60-72, 60-66, or 66-72 hours after the first population of CD45+ cells.

The second population of CD45+ cells can be administered to the subject after the population of cells enriched for Tregs.

The population Tcons can be administered to the subject greater than at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of cells enriched for Tregs.

In some embodiments, the second population of CD45+ cells is administered to the subject at most about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of cells enriched for Tregs.

The second population of CD45+ cells can be administered to the subject, for example, between about 6-96, 12-84, 12-72, 12-66, 12-60, 12-54, 12-48, 12-42, 12-36, 12-30, 12-24, 12-18, 18-72, 18-66, 18-60, 18-54, 18-48, 18-42, 18-36, 18-30, 18-24, 24-72, 24-66, 24-60, 24-54, 24-48, 24-42, 24-36, 24-30, 30-72, 30-66, 30-60, 30-54, 30-48, 30-42, 30-36, 36-72, 36-66, 36-60, 36-54, 36-48, 36-42, 42-72, 42-66, 42-60, 42-54, 42-48, 48-72, 48-66, 48-60, 48-54, 54-72, 54-66, 54-60, 60-72, 60-66, or 66-72 hours after the population of cells enriched for Tregs.

The second population of CD45+ cells can be administered to the subject after the first population of CD45+ cells and the population of cells enriched for Tregs.

The second population of CD45+ cells can be administered to the subject, for example, greater than at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the first population of CD45+ cells and the population of cells enriched for Tregs.

In some embodiments, the second population of CD45+ cells is administered to the subject at most about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the first population of CD45+ cells and the population of cells enriched for Tregs.

The second population of CD45+ cells can be administered to the subject, for example, between about 6-96, 12-84, 12-72, 12-66, 12-60, 12-54, 12-48, 12-42, 12-36, 12-30, 12-24, 12-18, 18-72, 18-66, 18-60, 18-54, 18-48, 18-42, 18-36, 18-30, 18-24, 24-72, 24-66, 24-60, 24-54, 24-48, 24-42, 24-36, 24-30, 30-72, 30-66, 30-60, 30-54, 30-48, 30-42, 30-36, 36-72, 36-66, 36-60, 36-54, 36-48, 36-42, 42-72, 42-66, 42-60, 42-54, 42-48, 48-72, 48-66, 48-60, 48-54, 54-72, 54-66, 54-60, 60-72, 60-66, or 66-72 hours after the first population of CD45+ cells and the population of cells enriched for Tregs.

In some embodiments, a population of hematopoietic stem and progenitor cells (HSPCs), a population of cells enriched for regulatory T cells (Tregs), a population of conventional T cells (Tcons), and a population of invariant natural killer T cells (iNKTs) are administered to a subject.

The population of iNKTs can be administered to the subject at the same time or at a similar time as the first population of CD45+ cells. In some embodiments, the population of iNKTs is administered to the subject after the first population of CD45+ cells.

The population of iNKTs can be administered to the subject greater than at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the first population of CD45+ cells.

In some embodiments, the population of iNKTs is administered to the subject at most about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the first population of CD45+ cells.

The population of iNKTs can be administered to the subject, for example, between about 6-96, 12-84, 12-72, 12-66, 12-60, 12-54, 12-48, 12-42, 12-36, 12-30, 12-24, 12-18, 18-72, 18-66, 18-60, 18-54, 18-48, 18-42, 18-36, 18-30, 18-24, 24-72, 24-66, 24-60, 24-54, 24-48, 24-42, 24-36, 24-30, 30-72, 30-66, 30-60, 30-54, 30-48, 30-42, 30-36, 36-72, 36-66, 36-60, 36-54, 36-48, 36-42, 42-72, 42-66, 42-60, 42-54, 42-48, 48-72, 48-66, 48-60, 48-54, 54-72, 54-66, 54-60, 60-72, 60-66, or 66-72 hours after the first population of CD45+ cells.

The population of iNKTs can be administered to the subject at the same time or at a similar time as the population of cells enriched for Tregs. In some embodiments, the population of iNKTs is administered to the subject after the population of cells enriched for Tregs.

A population of iNKTs can be administered to the subject greater than at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of cells enriched for Tregs.

In some embodiments, the population of iNKTs is administered to the subject at most about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of cells enriched for Tregs.

The population of iNKTs can be administered to the subject, for example, between about 6-96, 12-84, 12-72, 12-66, 12-60, 12-54, 12-48, 12-42, 12-36, 12-30, 12-24, 12-18, 18-72, 18-66, 18-60, 18-54, 18-48, 18-42, 18-36, 18-30, 18-24, 24-72, 24-66, 24-60, 24-54, 24-48, 24-42, 24-36, 24-30, 30-72, 30-66, 30-60, 30-54, 30-48, 30-42, 30-36, 36-72, 36-66, 36-60, 36-54, 36-48, 36-42, 42-72, 42-66, 42-60, 42-54, 42-48, 48-72, 48-66, 48-60, 48-54, 54-72, 54-66, 54-60, 60-72, 60-66, or 66-72 hours after the population of cells enriched for Tregs.

In some embodiments, a population of hematopoietic stem and progenitor cells (HSPCs), a population of cells enriched for regulatory T cells (Tregs), a population of conventional T cells (Tcons), and a population of memory T cells (Tmems) are administered to a subject.

A population of Tmems can be administered to the subject at the same time or at a similar time as the first population of CD45+ cells. In some embodiments, the population of Tmems is administered to the subject after the first population of CD45+ cells.

The population of Tmems can be administered to the subject greater than at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the first population of CD45+ cells.

In some embodiments, the population of Tmems is administered to the subject at most about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the first population of CD45+ cells.

The population of Tmems can be administered to the subject, for example, between about 6-96, 12-84, 12-72, 12-66, 12-60, 12-54, 12-48, 12-42, 12-36, 12-30, 12-24, 12-18, 18-72, 18-66, 18-60, 18-54, 18-48, 18-42, 18-36, 18-30, 18-24, 24-72, 24-66, 24-60, 24-54, 24-48, 24-42, 24-36, 24-30, 30-72, 30-66, 30-60, 30-54, 30-48, 30-42, 30-36, 36-72, 36-66, 36-60, 36-54, 36-48, 36-42, 42-72, 42-66, 42-60, 42-54, 42-48, 48-72, 48-66, 48-60, 48-54, 54-72, 54-66, 54-60, 60-72, 60-66, or 66-72 hours after the first population of CD45+ cells.

The population of Tmems can be administered to the subject at the same time or at a similar time as the population of cells enriched for Tregs. In some embodiments, the population of Tmems is administered to the subject after the population of cells enriched for Tregs.

The population of Tmems can be administered to the subject greater than at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of cells enriched for Tregs.

In some embodiments, the population of Tmems is administered to the subject at most about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of cells enriched for Tregs.

The population of Tmems can be administered to the subject, for example, between about 6-96, 12-84, 12-72, 12-66, 12-60, 12-54, 12-48, 12-42, 12-36, 12-30, 12-24, 12-18, 18-72, 18-66, 18-60, 18-54, 18-48, 18-42, 18-36, 18-30, 18-24, 24-72, 24-66, 24-60, 24-54, 24-48, 24-42, 24-36, 24-30, 30-72, 30-66, 30-60, 30-54, 30-48, 30-42, 30-36, 36-72, 36-66, 36-60, 36-54, 36-48, 36-42, 42-72, 42-66, 42-60, 42-54, 42-48, 48-72, 48-66, 48-60, 48-54, 54-72, 54-66, 54-60, 60-72, 60-66, or 66-72 hours after the population of cells enriched for Tregs.

In some embodiments, the population of Tcons is administered at least about 12 hours after the population of HSPCs, e.g., said population of Tcons is administered from about 24 to about 96 hours after the population of HSPCs or said population of Tcons is administered from about 36 to about 60 hours after the population of HSPCs.

In embodiments, the population of Tcons is administered at least about 12 hours after said population of cells comprising Tregs, e.g., the population of Tcons is administered from about 24 to about 96 hours after the population of cells comprising Tregs or said population of Tcons is administered from about 36 to about 60 hours after the population of cells comprising Tregs.

### Heterogenous Cell Populations

A further aspect provides a method of transplanting a conventional T cell (Tcons) population as a part of a treatment regimen for a hematologic malignancy in which the method reduces a risk and/or severity of an adverse event associated with the treatment regimen. The method comprises administering to the patient a population of regulatory T cells (Tregs) comprising Tregs and a liquid suspending the Tregs; administering to the patient a heterogenous cell population comprising lymphocytes, granulocytes, monocytes and a liquid suspending said cells. In this aspect, at least about 30% of said lymphocytes comprise Tcons. and after administration of the cell populations, the patient has a reduced risk and/or severity of the adverse event as compared to hematologic malignancy patients who received Tcons but did not receive Tregs.

A yet further aspect provides a method of transplanting cell populations into a human patient as a part of a treatment regimen for a hematologic malignancy in which the method reduces a risk and/or severity of an adverse event associated with the treatment regimen. The method comprises providing a population of hematopoietic stem and progenitor cells (HSPCs) to be administered to the patient; the population of HSPCs comprising HSPCs and a liquid suspending the HSPCs; providing a population of regulatory T cells (Tregs) to be administered to the patient, the population of Tregs comprising Tregs and a liquid suspending the Tregs; and providing a heterogenous cell population to be administered to the patient, the heterogenous cell population comprising lymphocytes, granulocytes, monocytes and a liquid suspending said cells. In this aspect, at least about 30% of said lymphocyte comprise conventional T cells (Tcons) and after administration of the cell populations, the patient has a reduced risk and/or severity of the adverse event as compared to hematologic malignancy patients who received a Tcon cell population but did not receive a T-reg cell population.

In various embodiments, the heterogenous cell population comprises from about 0.2 to about 2.0 percent hematopoietic stem and progenitor cells.

In some embodiments, the hematologic malignancy is acute lymphocytic leukemia, acute myelogenous leukemia, chronic myelogenous leukemia, multiple myeloma, lymphoma, Hodgkin's lymphoma and non-Hodgkin lymphoma.

In embodiments, a genetic expression level of the T-reg cells correlates to cells that were harvested from the donor within about 60 hours prior to administration to the patient.

In various embodiments, the number of T-reg cells in the T-reg population is about equal to the number of T-con cells in the heterogenous cell population. In some cases, the T-reg cells in the T-reg population inhibit activation of conventional T cells in the heterogenous cell population by the patient's healthy tissue by an amount up to about 20 percent

In some embodiments, the peripheral blood of the patient exhibits an elevated ratio of Tregs to CD4+ T cells up to about 100 days after administration of the cell populations as compared to a healthy human subject that was not administered the cells populations.

In embodiments, at least about 50% of the cells in the HSPC's cell population are colony forming units.

In various embodiments, at least one of the cell populations has an elevated amount of granulocyte colony-stimulating factor as compared to non-mobilized blood. In some cases, the at least one cell populations is the heterogenous cell population.

An aspect provides a method of transplanting cell populations into a human patient as a part of a treatment regimen for a hematologic malignancy. The method comprises administering to the patient a population of hematopoietic stem and progenitor cells (HSPCs; the population of HSPCs comprising HSPCs and a liquid suspending the HSPCs; administering to the patient a population of regulatory T cells (Tregs) to be administered to the patient, the population of Tregs comprising Tregs and a liquid suspending the Tregs; and administering to the patient a heterogenous cell population to be administered to the patient, the heterogenous cell population comprising lymphocytes, granulocytes, monocytes and a liquid suspending said cells, wherein at least about 30% of said lymphocyte comprise conventional T cells (Tcons); and administering to the patient over a period of time up to about 180 days a single graft versus host disease (GVHD) prophylactic agent (GVHDPA) comprising tacrolimus (tacrolimus GHVDPA), wherein the tacrolimus GHVDPA is administered to maintain a concentration of tacrolimus in the patient's blood above a threshold level during the period of time; and wherein a risk and/or severity of GHVD associated with the treatment regimen for the hematologic malignancy is significantly reduced.

In some embodiments, a heterogenous cell population may be administered to a subject. A heterogenous cell population may comprise many different cell types found in the peripheral blood of a human donor. A heterogenous cell population may comprise granulocytes, monocytes and lymphocytes. A heterogenous cell population may comprise T cells (such as Tcons, Tregs, Tmems, naïve T cells, CD4+ T cells, NK-T cells), B cells, NK cells, HSPCs, dendritic cells (such as plasmacytoid dendritic cells and myeloid dendritic cells) and other cell populations found in peripheral blood. A heterogenous cell population may be administered to a subject in addition to the other populations described herein. For instance, a heterogenous cell population may be administered with HSPCs as described herein. In some cases, a heterogenous cell population may be administered with HSPCs and Tregs as described herein. In some cases, a heterogenous cell population may be administered with Tregs as described herein. In some cases, a heterogenous cell population may be administered with Tcons as described herein. In some cases, a heterogenous cell population may be administered instead of the Tcon population as described herein.

In some embodiments, a heterogenous cell population administered to a subject may comprise a combination of granulocytes and monocytes. A combination of granulocytes and monocytes may comprise from 30% to 80% of the heterogenous cell population. At least 30% of the heterogenous cell population may comprise a combination of granulocytes and monocytes. At most 80% of the heterogenous cell population may comprise a combination of granulocytes and monocytes. In some cases, 30% to 40%, 30% to 50%, 30% to 60%, 30% to 70%, 30% to 80%, 40% to 50%, 40% to 60%, 40% to 70%, 40% to 80%, 50% to 60%, 50% to 70%, 50% to 80%, 60% to 70%, 60% to 80%, or 70% to 80% of the heterogenous cell population may comprise a combination of granulocytes and monocytes. In some cases, 30%, 40%, 50%, 60%, 70%, or 80% of the heterogenous cell population may comprise a combination of granulocytes and monocytes. In some cases, at least 30%, 40%, 50%, 60% or 70% of the heterogenous cell population may comprise a combination of granulocytes and monocytes. In some cases, at most 40%, 50%, 60%, 70%, or 80% of the heterogenous cell population may comprise a combination of granulocytes and monocytes.

In some embodiments, a heterogenous cell population administered to the subject may comprise lymphocytes. Lymphocytes comprise CD45+ cells. Lymphocytes may comprise from 8% to 50% of the heterogenous cell population. In some cases, at least 8% of the heterogenous cell population may comprise lymphocytes. In some cases, at most 50% of the heterogenous cell population may comprise lymphocytes. In some cases, 8% to 10%, 8% to 20%, 8% to 25%, 8% to 30%, 8% to 40%, 8% to 45%, 8% to 50%, 10% to 20%, 10% to 25%, 10% to 30%, 10% to 40%, 10% to 45%, 10% to 50%, 20% to 25%, 20% to 30%, 20% to 40%, 20% to 45%, 20% to 50%, 25% to 30%, 25% to 40%, 25% to 45%, 25% to 50%, 30% to 40%, 30% to 45%, 30% to 50%, 40% to 45%, 40% to 50%, or 45% to 50% of the heterogenous cell population may comprise lymphocytes. In some cases, 8%, 10%, 20%, 25%, 30%, 40%, 45%, or 50% of the heterogenous cell population may comprise lymphocytes. In some cases, at least 8%, 10%, 20%, 25%, 30%, 40% or 45% of the heterogenous cell population may comprise lymphocytes. In some cases, at most 10%, 20%, 25%, 30%, 40%, 45%, or 50% of the heterogenous cell population may comprise lymphocytes.

In some embodiments, lymphocytes in the heterogenous cell population may comprise Tcons. Tcons may comprise from 40% to 85% of the lymphocyte subset of the heterogenous cell population. In some cases, at least 40% of the lymphocyte subset may comprise Tcons. In some cases, at most 85% of the lymphocyte subset may comprise Tcons. In some cases, 40% to 50%, 40% to 60%, 40% to 65%, 40% to 70%, 40% to 75%, 40% to 80%, 40% to 85%, 50% to 60%, 50% to 65%, 50% to 70%, 50% to 75%, 50% to 80%, 50% to 85%, 60% to 65%, 60% to 70%, 60% to 75%, 60% to 80%, 60% to 85%, 65% to 70%, 65% to 75%, 65% to 80%, 65% to 85%, 70% to 75%, 70% to 80%, 70% to 85%, 75% to 80%, 75% to 85%, or 80% to 85% of the lymphocyte subset may comprise Tcons. In some cases, 40%, 50%, 60%, 65%, 70%, 75%, 80%, or 85% of the lymphocyte subset may comprise Tcons. In some cases, at least 40%, 50%, 60%, 65%, 70%, 75% or 80% of the lymphocyte subset may comprise Tcons. In some cases, at most 50%, 60%, 65%, 70%, 75%, 80%, or 85% of the lymphocyte subset may comprise Tcons.

In some embodiments, CD3+ lymphocytes in the heterogenous cell population may comprise CD4+ T cells. In some cases, 30% to 70% of the CD3+ lymphocyte subset may comprise CD4+ T cells. In some cases, at least 30% of the CD3+ lymphocyte subset may comprise CD4+ T cells. In some cases, at most 70% of the CD3+ lymphocyte subset may comprise CD4+ T cells. In some cases, 30% to 40%, 30% to 50%, 30% to 60%, 30% to 70%, 40% to 50%, 40% to 60%, 40% to 70%, 50% to 60%, 50% to 70%, or 60% to 70% of the CD3+ lymphocyte subset may comprise CD4+ T cells. In some cases, 30%, 40%, 50%, 60%, or 70% of the CD3+ lymphocyte subset may comprise CD4+ T cells. In some cases, at least 30%, 40%, 50% or 60% of the CD3+ lymphocyte subset may comprise CD4+ T cells. In some cases, at most 40%, 50%, 60%, or 70% of the CD3+ lymphocyte subset may comprise CD4+ T cells.

In some embodiments, CD3+ lymphocytes in the heterogenous cell population may comprise CD8+ T cells. In some cases, 20% to 65% of the CD3+ lymphocyte subset may comprise CD8+ T cells. In some cases, at least 20% of the CD3+ lymphocyte subset may comprise CD8+ T cells. In some cases, at most 65% of the CD3+ lymphocyte subset may comprise CD8+ T cells. In some cases, 20% to 30%, 20% to 40%, 20% to 50%, 20% to 60%, 20% to 65%, 30% to 40%, 30% to 50%, 30% to 60%, 30% to 65%, 40% to 50%, 40% to 60%, 40% to 65%, 50% to 60%, 50% to 65%, or 60% to 65% of the CD3+ lymphocyte subset may comprise CD8+ T cells. In some cases, 20%, 30%, 40%, 50%, 60%, or 65% of the CD3+ lymphocyte subset may comprise CD8+ T cells. In some cases, at least 20%, 30%, 40%, 50% or 60% of the CD3+ lymphocyte subset may comprise CD8+ T cells. In some cases, at most 30%, 40%, 50%, 60%, or 65% of the CD3+ lymphocyte subset may comprise CD8+ T cells.

In some embodiments, lymphocytes in the heterogenous cell population may comprise B cells. In some cases, 4% to 35% of the lymphocyte subset may comprise B cells. In some cases, at least 4% of the lymphocyte subset may comprise B cells. In some cases, at most 35% of the lymphocyte subset may comprise B cells. In some cases, 4% to 5%, 4% to 10%, 4% to 20%, 4% to 30%, 4% to 35, 5% to 10%, 5% to 20%, 5% to 30%, 5% to 35%, 10% to 20%, 10% to 30%, 10% to 35%, 20% to 30%, 20% to 35%, or 30% to 35% of the lymphocyte subset may comprise B cells. In some cases, 4%, 5%, 10%, 20%, 30%, or 35% of the lymphocyte subset may comprise B cells. In some cases, at least 4%, 5%, 10%, 20% or 30% of the lymphocyte subset may comprise B cells. In some cases, at most 5%, 10%, 20%, 30%, or 35% of the lymphocyte subset may comprise B cells. B cells may be CD45+CD19+ or CD45+CD19+CD3− cells.

In some embodiments, lymphocytes in the heterogenous cell population may comprise NK cells. In some cases, 4% to 35% of the lymphocyte subset may comprise NK cells. In some cases, at least 4% of the lymphocyte subset may comprise NK cells. In some cases, at most 35% of the lymphocyte subset may comprise NK cells. In some cases, 4% to 5%, 4% to 10%, 4% to 20%, 4% to 30%, 4% to 35%, 5% to 10%, 5% to 20%, 5% to 30%, 5% to 35%, 10% to 20%, 10% to 30%, 10% to 35%, 20% to 30%, 20% to 35%, or 30% to 35% of the lymphocyte subset may comprise NK cells. In some cases, 4%, 5%, 10%, 20%, 30%, or 35% of the lymphocyte subset may comprise NK cells. In some cases, at least 4%, 5%, 10%, 20% or 30% of the lymphocyte subset may comprise NK cells. In some cases, at most 5%, 10%, 20%, 30%, or 35% of the lymphocyte subset may comprise NK cells. NK cells may be CD45+CD56+ or CD45+CD56+CD3− cells.

In some embodiments, CD3+ lymphocytes in the heterogenous cell population may comprise NK-T cells. In some cases, 3% to 30% of the CD3+ lymphocyte subset may comprise NK-T cells. In some cases, at least 4% of the CD3+ lymphocyte subset may comprise NK-T cells. In some cases, at most 35% of the CD3+ lymphocyte subset may comprise NK-T cells. In some cases, 3% to 5%, 3% to 10%, 3% to 20%, 3% to 30%, 5% to 10%, 5% to 20%, 5% to 30%, 10% to 20%, 10% to 30%, 20% to 30%, of the CD3+ lymphocyte subset may comprise NK-T cells. In some cases, 3%, 5%, 10%, 20% or 30% of the CD3+ lymphocyte subset may comprise NK-T cells. In some cases, at least 3%, 5%, 10% or 20% of the CD3+ lymphocyte subset may comprise NK-T cells. In some cases, at most 10%, 20% or 30% of the CD3+ lymphocyte subset may comprise NK-T cells. NK-T cells may be CD45+CD56+ or CD45+CD56+CD3+ cells.

In some embodiments, lymphocytes in the heterogenous cell population may comprise CD34+ cells. In some cases, 0.1% to 2% of the lymphocyte subset may comprise CD34+ cells. In some cases, at least 0.1% of the lymphocyte subset may comprise CD34+ cells. In some cases, at most 2% of the lymphocyte subset may comprise CD34+ cells. In some cases, 0.1% to 0.5%, 0.10% to 1%, 0.1% to 1.5%, 0.1% to 2%, 0.5% to 1%, 0.5% to 1.5%, 0.5% to 2%, 1% to 1.5%, 1% to 2%, or 1.5% to 2% of the lymphocyte subset may comprise CD34+ cells. In some cases, 0.1%, 0.5%, 1%, 1.5%, or 2% of the lymphocyte subset may comprise CD34+ cells. In some cases, at least 0.1%, 0.5%, 1% or 1.5% of the lymphocyte subset may comprise CD34+ cells. In some cases, at most 0.5%, 1%, 1.5%, or 2% of the lymphocyte subset may comprise CD34+ cells.

### II. GVHD Prophylactic Agents

Subjects administered a composition of the disclosure (e.g., a cell component comprising a populations of cells described herein) and a GVHD prophylactic agent (for example, a single GVHD prophylactic agent) exhibit a low incidence of ≥grade 1 aGVHD, for example, a lower incidence of ≥grade 1 aGVHD than subjects that are administered an alternate composition. A single GVHD prophylactic agent can be a calcineurin inhibitor such as tacrolimus or another agent which acts on the same targets as tacrolimus or comprises an active fragment of tacrolimus. In some embodiments, a low dose of a GVHD prophylactic agent is tacrolimus with a target trough level of about 5 ng/mL to about 10 ng/mL. In some embodiments, a low dose of a GVHD prophylactic agent is tacrolimus with a target trough level of about 4 ng/mL to about 6 ng/mL.

A single GVHD prophylactic agent can be sirolimus. In some embodiments, a low dose of a GVHD prophylactic agent is sirolimus with a target trough level of about 3 ng/mL to about 8 ng/mL. In some embodiments, a low dose of a GVHD prophylactic agent is sirolimus with a target trough level of about 4 ng/mL to about 8 ng/mL.

Methods for alloHCT of the disclosure utilize tacrolimus. Combining tacrolimus with one or more cell populations in an alloHCT regimen as disclosed herein is shown to result in surprising improvements in clinical outcomes.

An aspect provides a multi-component pharmaceutical treatment to be administered to a human subject in need thereof. The multi-component treatment comprises (a) a solution comprising a first population of CD45+ cells comprising hematopoietic stem and progenitor cells (HSPCs) and granulocytes wherein at most about 10% of the first population of CD45+ cells comprise granulocytes; (b) a solution comprising a population of cells enriched for regulatory T cells (Tregs); (c) a solution comprising a second population of CD45+ cells wherein the second population of CD45+ cells comprise at least about 20% CD3+ conventional T cells (Tcons), at least about 10% monocytes, and at least about 10% granulocytes; and (d) a solution comprising one or more doses of a graft vs host disease (GVHD) prophylactic agent, wherein the GVHD prophylactic agent is tacrolimus.

In various embodiments, the tacrolimus is administered in an amount to maintain a target blood level of at least about 3 ng/ml for at least about 20 days after administering the second population of CD45+ cells, in an amount to maintain a target blood level of about 4 ng/ml or more for at least about 40 days after administering the second population of CD45+ cells, and/or in an amount that maintains a target blood level of about 4 ng/ml or more for at least about 40 days after administering the second population of CD45+ cells. In some cases, the tacrolimus is administered in an amount that maintains a target blood level of at most about 10 ng/ml for at least 30 days after administering the second population of CD45+ cells.

In some embodiments, the tacrolimus is administered for at least about 60 days after administering the second population of CD45+ cells, for at least about 90 days after administering the second population of CD45+ cells, for at most about 150 days after administering the second population of CD45+ cells, for at most about 120 days after administering the second population of CD45+ cells.

In some embodiments, the tacrolimus is formulated for oral administration or for intravenous administration.

In embodiments, a herein-disclosed method further comprises administering to the patient over a period of time up to about 180 days a single graft versus host disease (GVHD) prophylactic agent (GVHDPA) comprising tacrolimus (tacrolimus GHVDPA); wherein the tacrolimus GHVDPA is administered to maintain a concentration of tacrolimus in the patient's blood above a threshold level during the period of time; and wherein a risk and/or severity of GHVD is significantly reduced. In various embodiments, the threshold level is above about 4 ng of tacrolimus per ml of patient blood or the threshold level is above about 5 ng of tacrolimus per ml of patient blood. In some embodiments, the tacrolimus GHVDPA is administered to maintain a concentration of tacrolimus in the patients' blood below an upper threshold level during the period of time. In some cases, the upper threshold level is below about 10 ng of tacrolimus per ml of patient blood.

In embodiments, the tacrolimus graft versus host disease (GVHD) prophylactic agent (GVHDPA) is intravenously administered or orally administered. In various embodiments, administration of the tacrolimus graft versus host disease (GVHD) prophylactic agent (GVHDPA) is started from about 12 to about 24 hours after administration of the T-cons. In some cases, the tacrolimus GHVDPA is administered for a period of time up to about 90 days, is administered for a period of time up to about 60 days. In some embodiments, the tacrolimus GHVDPA is initially administered to the patient at about 0.03 mg/kg patient's actual or ideal body weight/day. In some cases, a dose of the tacrolimus GVHDPA administered to the patient is tapered starting at about 90 days after a first dose is administered to the patient or is tapered starting at about 45 days after a first dose is administered to the patient.

Tacrolimus is a macrolide that can exhibit immunosuppressive activity in vivo, and can prevent or reduce the activation of T-lymphocytes in response to antigenic or mitogenic stimulation. Tacrolimus can therefore be used to reduce the risk of GVHD in alloHCT recipient subjects. However, methods disclosed herein—that utilize tacrolimus as a single-agent prophylactic—achieve superior clinical outcomes to those observed in other alloHCT methods that utilize tacrolimus GVHD prophylactic. For example, in some embodiments alloHCT methods of the disclosure that utilize tacrolimus achieve superior relapse-free survival compared to a standard of care regimen that comprises more potent GVHD prophylaxis with methotrexate plus tacrolimus, and even compared to an alloHCT method that utilizes a drug with a similar target and mechanism of action (sirolimus). Thus, although tacrolimus can be referred to as a GVHD prophylactic herein, it can also contribute to additional therapeutic effects beyond or not directly related to GVHD prophylaxis. Therefore, as used herein, the term “tacrolimus as a single-agent GVHD prophylactic” also “includes tacrolimus as a single-agent prophylactic for additional therapeutics effects.” In other words, the term “tacrolimus as a single-agent prophylactic” and “tacrolimus as a single-agent GVHD prophylactic” are synonyms.

Treatment with tacrolimus as a single-agent prophylactic can result in, for example, decreased cytokine production and decreased T cell signal transduction. Tacrolimus can bind to the FKBP-12 protein and form a complex with calcium-dependent proteins, thereby inhibiting calcineurin phosphatase activity. This prevents or reduces the dephosphorylation and translocation of nuclear factor of activated T-cells (NFAT), a nuclear component thought to initiate gene transcription for the expression of lymphokines. Tacrolimus also inhibits the transcription for genes which encode IL-3, IL-4, IL-5, GM-CSF, and TNF, all of which are involved in the early stages of T-cell activation.

In some embodiments, tacrolimus as a single-agent prophylactic is administered to a subject orally. Absorption of tacrolimus from the gastrointestinal tract after oral administration can be incomplete and variable. The absolute bioavailability of tacrolimus in healthy subjects after oral administration can be 18±5%. The rate and extent of absorption can vary based on whether tacrolimus is given with food. In some embodiments, tacrolimus is administered parenterally, for example, intravenously or subcutaneously. In some embodiments, tacrolimus is administered by a topical, intramuscular, intradermal, intraperitoneal, intraspinal, or epidural route. Tacrolimus can be administered as an extended release formulation. In some embodiments, tacrolimus is used as a free base. In some embodiments, tacrolimus is used as a pharmaceutically-acceptable salt.

In some embodiments, methods of the disclosure can allow low doses of tacrolimus to be used. In some embodiments, a low dose of tacrolimus can improve donor T cell chimerism in a subject. In some embodiments, a low dose of tacrolimus can improve alloHCT engraftment as disclosed herein. In some embodiments, a low dose of tacrolimus can reduce the incidence or relative risk of adverse effects that can be associated with high doses of tacrolimus, such as blurred vision, liver and kidney toxicity, seizures, tremors, hypertension, hypomagnesemia, diabetes mellitus, hyperkalemia, itching, insomnia, and confusion.

In some embodiments, the tacrolimus is initially administered to the human subject at about 0.03 mg/kg human subject's actual or ideal body weight/day or the tacrolimus is initially administered from about 12 hours to about 24 hours after said administering of said second population of CD45+ cells, as disclosed herein.

A circulating level of tacrolimus can be monitored, and doses adjusted accordingly to achieve a target concentration. For example, a whole blood concentration of tacrolimus can be monitored, and doses adjusted and administered to achieve a target trough level. A target trough level of tacrolimus can be, for example, less than about 25 ng/mL, less than about 20 ng/mL, less than about 15 ng/mL, less than about 14 ng/mL, less than about 13 ng/mL, less than about 12 ng/mL, less than about 11 ng/mL, less than about 10 ng/mL, less than about 9 ng/mL, less than about 8 ng/mL, less than about 7 ng/mL, less than about 6 ng/mL, less than about 5 ng/mL, less than about 4 ng/mL, less than about 3 ng/mL, less than about 2 ng/mL, or less than about 1 ng/mL.

In some embodiments, a target trough level of tacrolimus is about 1-25 ng/mL, about 1-20 ng/mL, about 1-15 ng/mL, about 1-12 ng/mL, about 1-11 ng/mL, about 1-10 ng/mL, about 1-9 ng/mL, about 1-8 ng/mL, about 1-7 ng/mL, about 1-6 ng/mL, about 1-5 ng/mL, about 1-4 ng/mL, about 1-3 ng/mL, about 1-2 ng/mL, about 2-25 ng/mL, about 2-20 ng/mL, about 2-15 ng/mL, about 2-12 ng/mL, about 2-11 ng/mL, about 2-10 ng/mL, about 2-9 ng/mL, about 2-8 ng/mL, about 2-7 ng/mL, about 2-6 ng/mL, about 2-5 ng/mL, about 2-4 ng/mL, about 2-3 ng/mL, about 3-25 ng/mL, about 3-20 ng/mL, about 3-15 ng/mL, about 3-12 ng/mL, about 3-11 ng/mL, about 3-10 ng/mL, about 3-9 ng/mL, about 3-8 ng/mL, about 3-7 ng/mL, about 3-6 ng/mL, about 3-5 ng/mL, about 3-4 ng/mL, about 4-25 ng/mL, about 4-20 ng/mL, about 4-15 ng/mL, about 4-12 ng/mL, about 4-11 ng/mL, about 4-10 ng/mL, about 4-9 ng/mL, about 4-8 ng/mL, about 4-7 ng/mL, about 4-6 ng/mL, about 4-5 ng/mL, about 5-25 ng/mL, about 5-20 ng/mL, about 5-15 ng/mL, about 5-12 ng/mL, about 5-11 ng/mL, about 5-10 ng/mL, about 5-9 ng/mL, about 5-8 ng/mL, about 5-7 ng/mL, about 5-6 ng/mL, about 6-25 g/mL, about 6-20 ng/mL, about 6-15 ng/mL, about 6-12 ng/mL, about 6-11 ng/mL, about 6-10 ng/mL, about 6-9 ng/mL, about 6-8 ng/mL, about 6-7 ng/mL, about 8-25 ng/mL, about 8-20 ng/mL, about 8-15 ng/mL, about 8-12 ng/mL, about 8-11 ng/mL, about 8-10 ng/mL, about 8-9 ng/mL, about 10-25 ng/mL, about 10-20 ng/mL, about 10-15 ng/mL, about 10-12 ng/mL, or about 10-11 ng/mL.

In some embodiments, a target trough level of tacrolimus is about 6 ng/mL to about 10 ng/mL. In some embodiments, a target trough level of tacrolimus is about 6 ng/mL to about 9 ng/mL. In some embodiments, a target trough level of tacrolimus is about 6 ng/mL to about 8 ng/mL. In some embodiments, a target trough level of tacrolimus is about 5 ng/mL to about 10 ng/mL. In some embodiments, a target trough level of tacrolimus is about 5 ng/mL to about 9 ng/mL. In some embodiments, a target trough level of tacrolimus is about 5 ng/mL to about 8 ng/mL. In some embodiments, a target trough level of tacrolimus is about 4 ng/mL to about 10 ng/mL. In some embodiments, a target trough level of tacrolimus is about 4 ng/mL to about 9 ng/mL. In some embodiments, a target trough level of tacrolimus is about 4 ng/mL to about 8 ng/mL.

In some embodiments, a dose of tacrolimus as a single-agent prophylactic or a target trough level of tacrolimus can be adjusted based on a clinical parameter disclosed herein. For example, in some cases, a dose or a target trough level of tacrolimus can be reduced if a subject exhibits lower donor T cell chimerism than desired, e.g., a percent of peripheral blood donor-derived CD3+ cells that is less than 95%, less than 90%, less than 85%, less than 80%, less than 75%, less than 70%, less than 65%, less than 60%, less than 55%, less than 50%, or less than 45% when evaluated after a suitable amount of time after administration of a cell population of the disclosure, for example, at about 14 days, 15 days, 20 days, 21 days, 25 days, 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100 days, 110 days, 120 days, 130 days, 140 days, 150 days, 160 days, 170 days, 180 days, or 1 year after administration of a cell population of the disclosure (e.g., a first population of CD45+ cells). In some embodiments, a target trough level of tacrolimus can be increased if a subject exhibits signs of GVHD as disclosed herein.

In some embodiments, a subject achieves at least about 80% chimerism at about day 30. In some embodiments, a subject achieves at least about 80% chimerism at about day 30 and has a target trough level of tacrolimus of between about 6.5 ng/mL and about 9 ng/mL.

Administration of tacrolimus to a subject can commence before or after administration of a cell population disclosed herein (e.g., a first population of CD45+ cells). In some embodiments, administration of tacrolimus to a subject commences before administration of a cell population disclosed herein (e.g., a first population of CD45+ cells). In some embodiments, administration of tacrolimus to a subject commences after administration of a cell population disclosed herein (e.g., a first population of CD45+ cells). In some embodiments, administration of tacrolimus to a subject commences at about the same time as administration of a cell population disclosed herein (e.g., a first population of CD45+ cells).

In some embodiments, administration of tacrolimus to a subject commences at least 1 day, at least 2 days, at least 3 days, at least 4 days, at least 5 days, at least 6 days, at least 7 days, at least 10 days, at least 14 days, or at least 20 days before administration of a cell population disclosed herein (e.g., a population of CD45+ cells). In some embodiments, administration of tacrolimus to a subject commences at most 1 day, at most 2 days, at most 3 days, at most 4 days, at most 5 days, at most 6 days, at most 7 days, at most 10 days, at most 14 days, or at most 20 days before administration of a cell population disclosed herein (e.g., a population of CD45+ cells). In some embodiments, administration of tacrolimus to a subject commences 1 day, 2 days, 3 days, 4 days, 5 days, 6 days, 7 days, 10 days, 14 days, or 20 days before administration of a cell population disclosed herein (e.g., a population of CD45+ cells). In some embodiments, administration of tacrolimus to a subject commences 1 day before administration of a first population of CD45+ cells. In some embodiments, administration of tacrolimus to a subject commences 1 day before administration of a second population of CD45+ cells.

In embodiments, the tacrolimus is initially administered to the human subject at about 0.03 mg/kg human subject's actual or ideal body weight/day or the tacrolimus is initially administered from about 12 hours to about 24 hours after said administering of said second population of CD45+ cells, as disclosed herein.

In some embodiments, administration of tacrolimus to a subject commences at least 1 day, at least 2 days, at least 3 days, at least 4 days, at least 5 days, at least 6 days, at least 7 days, at least 10 days, at least 14 days, or at least 20 days after administration of a cell population disclosed herein (e.g., a population of CD45+ cells). In some embodiments, administration of tacrolimus to a subject commences at most 1 day, at most 2 days, at most 3 days, at most 4 days, at most 5 days, at most 6 days, at most 7 days, at most 10 days, at most 14 days, or at most 20 days after administration of a cell population disclosed herein (e.g., a population of CD45+ cells). In some embodiments, administration of tacrolimus to a subject commences 1 day, 2 days, 3 days, 4 days, 5 days, 6 days, 7 days, 10 days, 14 days, or 20 days after administration of a cell population disclosed herein (e.g., a population of CD45+ cells). In some embodiments, administration of tacrolimus to a subject commences 1 day after administration of a population of CD45+ cells. In some embodiments, administration of tacrolimus to a subject commences 1 day after administration of a second population of CD45+ cells. In some embodiments, administration of tacrolimus to a subject commences the same day as a cell population of the disclosure is administered.

Tacrolimus can be administered to a subject for any amount of time after administration of a cell population of the disclosure (e.g., a population of CD45+ cells). In some embodiments, tacrolimus is administered to a subject for the first 7 days, 14 days, first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years after administration of a cell population of the disclosure (e.g., a population of CD45+ cells).

In some embodiments, tacrolimus is administered to a subject for less than about 20 days, less than about 30 days, less than about 40 days, less than about 50 days, less than about 60 days, less than about 70 days, less than about 80 days, less than about 90 days, less than about 100 days, less than about 110 days, less than about 120 days, less than about 150 days, less than about 200 days, less than about 365 days, less than about 13 months, less than about 14 months, less than about 15 months, less than about 16 months, less than about 17 months, less than about 18 months, less than about 19 months, less than about 20 months, less than about 21 months, less than about 22 months, less than about 23 months, less than about 2 years, less than about 2.5 years, less than about 3 years, less than about 3.5 years, less than about 4 years, less than about 4.5 years, or less than about or 5 years after administration of a cell population of the disclosure (e.g., a population of CD45+ cells).

In various embodiments, a dose of the tacrolimus is tapered starting at about 90 days after the first dose is administered to the human subject or a dose of the tacrolimus is tapered starting at about 45 days after the first dose is administered to the human subject.

In some embodiments, a subject achieves at least about 80% chimerism at about day 30. In some embodiments, a subject achieves at least about 80% chimerism at about day 30 and has a target trough level is of about 6.5 ng/mL and about 9 ng/mL.

### III. Subjects

Provided herein are compositions for administration to a recipient subject having a cancer, and methods of administering the same. The compositions and methods can be useful for treating or reducing cancer in the subject. In some embodiments, a second population of CD45+ cells that comprises, at least, Tcons is administered to the subject in order to elicit graft-versus-tumor (GVT) immune responses and with reduced graft versus host disease (GVHD).

In some embodiments, a subject is at least 13, at least 14, at least 15, at least 16, at least 17, at least 18, at least 19, at least 20, at least 21, at least 22, at least 23, at least 24, or at least 25 years of age. In some embodiments, a subject is at least 18 years of age. In some embodiments, a subject is at least 16 years of age. In some embodiments, a subject is at least 13 years of age.

In some embodiments, a subject is at most 50, at most 55, at most 60, at most 65, at most 70, at most 75, or at most 80 years of age. In some embodiments, a subject is at most 65 years of age. In some embodiments, a subject is at most 70 years of age.

### A. Conditions

Another aspect provides a method of treating a human subject diagnosed with a hematologic malignancy. The method comprises administering to the human subject a solution comprising the first population of CD45+ cells, a solution comprising the population of cells enriched for regulatory T cells (Tregs), a solution comprising the second population of CD45+ cells, and a solution comprising one or more doses of the GVHD prophylactic agent (e.g., tacrolimus). In this aspect, the solution comprising the first population of CD45+ cells, the solution comprising the population of cells enriched for regulatory Tregs, the solution comprising the second population of CD45+ cells, and the solution comprising one or more doses of the GVHD prophylactic agent are as defined according to any herein disclosed multi-component pharmaceutical treatment.

A further aspect provides a method of transplanting a conventional T cell (Tcons) population as a part of a treatment regimen for a hematologic malignancy in which the method reduces a risk and/or severity of an adverse event associated with the treatment regimen. The method comprises administering to the patient a population of regulatory T cells (Tregs) comprising Tregs and a liquid suspending the Tregs; administering to the patient a heterogenous cell population comprising lymphocytes, granulocytes, monocytes and a liquid suspending said cells. In this aspect, at least about 30% of said lymphocytes comprise Tcons. and after administration of the cell populations, the patient has a reduced risk and/or severity of the adverse event as compared to hematologic malignancy patients who received Tcons but did not receive Tregs.

A yet further aspect provides a method of transplanting cell populations into a human patient as a part of a treatment regimen for a hematologic malignancy in which the method reduces a risk and/or severity of an adverse event associated with the treatment regimen. The method comprises providing a population of hematopoietic stem and progenitor cells (HSPCs) to be administered to the patient; the population of HSPCs comprising HSPCs and a liquid suspending the HSPCs; providing a population of regulatory T cells (Tregs) to be administered to the patient, the population of Tregs comprising Tregs and a liquid suspending the Tregs; and providing a heterogenous cell population to be administered to the patient, the heterogenous cell population comprising lymphocytes, granulocytes, monocytes and a liquid suspending said cells. In this aspect, at least about 30% of said lymphocyte comprise conventional T cells (Tcons) and after administration of the cell populations, the patient has a reduced risk and/or severity of the adverse event as compared to hematologic malignancy patients who received a Tcon cell population but did not receive a T-reg cell population.

Another aspect provides a method of transplanting cell populations into a human patient as a part of a treatment regimen for a hematologic malignancy. The method comprises administering to the patient a population of hematopoietic stem and progenitor cells (HSPCs; the population of HSPCs comprising HSPCs and a liquid suspending the HSPCs; administering to the patient a population of regulatory T cells (Tregs) to be administered to the patient, the population of Tregs comprising Tregs and a liquid suspending the Tregs; and administering to the patient a heterogenous cell population to be administered to the patient, the heterogenous cell population comprising lymphocytes, granulocytes, monocytes and a liquid suspending said cells, wherein at least about 30% of said lymphocyte comprise conventional T cells (Tcons); and administering to the patient over a period of time up to about 180 days a single graft versus host disease (GVHD) prophylactic agent (GVHDPA) comprising tacrolimus (tacrolimus GHVDPA), wherein the tacrolimus GHVDPA is administered to maintain a concentration of tacrolimus in the patient's blood above a threshold level during the period of time; and wherein a risk and/or severity of GHVD associated with the treatment regimen for the hematologic malignancy is significantly reduced.

The methods of the disclosure can be used for treating a subject (e.g., a human subject) with a cancer. In some embodiments, the subject has been treated for cancer, e.g. by treatment with a chemotherapeutic drug or with radiation. The methods of the disclosure can be useful for treating a hematologic malignancy, for example, leukemia or lymphoma. Examples of hematologic malignancies that can be treated by the methods of the disclosure include, but are not limited to, acute lymphocytic leukemia (ALL), acute myelogenous leukemia (AML), chronic myelogenous leukemia (CML), multiple myeloma, and lymphomas such as Hodgkin and non-Hodgkin lymphomas. A cancer can be a solid tumor. In some embodiments, the cancer is a primary or metastatic tumor.

The types of cancer that can be treated using the methods of the present invention include but are not limited to leukemia, lymphoma, adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's lymphoma, Kaposi's sarcoma, kidney cancer, laryngeal and pharyngeal cancer, acute lymphocytic leukemia, acute myeloid leukemia, children's leukemia, chronic lymphocytic leukemia, chronic myeloid leukemia, liver cancer, lung cancer, lung carcinoid tumors, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, nonmelanoma skin cancers, stomach cancer, testicular cancer; thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, and Waldenstrom's macroglobulinemia.

Patients with high-risk hematologic malignancies are rarely cured with standard chemotherapy. High-risk malignancies include, for example, leukemia or lymphoma that has progressed beyond first remission, or leukemia or lymphoma with refractory relapse.

In some embodiments, the human subject or patient has previously been or is concurrently treated for the hematologic malignancy.

A subject that receives a composition of the disclosure can have, for example, acute myeloid leukemia, acute lymphoid leukemia, mixed phenotype leukemia, myelofibrosis, high-risk myelodysplastic syndrome, very high-risk myelodysplastic syndrome, myelofibrosis (MF) that is eligible for transplant per National Comprehensive Cancer Network Guidelines, intermediate-2- or high-risk MF according to the IPSS, DIPSS or DIPSS-plus scoring systems, intermediate-1-risk MF associated with high-risk features such as high symptoms burden, low platelet counts, or complex cytogenetics, primary myelofibrosis, myelofibrosis evolved from another myeloproliferative neoplasm, myelodysplastic syndrome, non-Hodgkin lymphoma, a non-malignant indication for allogeneic hematopoietic stem cell transplantation (alloHCT).

In some embodiments, a subject has acute myeloid leukemia. In some embodiments, a subject has acute lymphoid leukemia. In some embodiments, a subject has mixed phenotype leukemia. In some embodiments, a subject has high-risk myelodysplastic syndrome. In some embodiments, a subject has very high-risk myelodysplastic syndrome. In some embodiments, a subject has myelofibrosis (MF) that is eligible for transplant per National Comprehensive Cancer Network Guidelines. In some embodiments, a subject has intermediate-2- or high-risk myelofibrosis according to the IPSS, DIPSS or DIPSS-plus scoring systems. In some embodiments, a subject has intermediate-1-risk myelofibrosis associated with high-risk features such as high symptoms burden, low platelet counts, or complex cytogenetics. In some embodiments, a subject has primary myelofibrosis. In some embodiments, a subject has myelofibrosis. In some embodiments, a subject has myelofibrosis evolved from another myeloproliferative neoplasm. In some embodiments, a subject has myelodysplastic syndrome. In some embodiments, a subject has non-Hodgkin lymphoma. In some embodiments, a subject has a non-malignant indication for alloHCT.

A subject can be in complete remission (CR). A subject can be in complete remission with incomplete hematologic recovery (CRi), e.g., without the presence of known minimal residual disease. A subject can have minimal residual disease. A subject can have no evidence of minimal residual disease. A subject can have active disease. A subject can have a leukemia (e.g., acute myeloid, acute lymphoid, or mixed phenotype) that is not in morphologic CR with bone marrow infiltration by leukemic blasts of ≤10%. A subject can have a leukemia (e.g., acute myeloid, acute lymphoid, or mixed phenotype) that is in morphologic CR with evidence of minimal residual positivity by either multiparameter flow cytometric analysis or by a nucleic acid-based technique.

Complete remission (CR) for acute myeloid, lymphoid or mixed phenotype leukemia can be indicated by meeting all of the following criteria: (i) Bone marrow blasts <5%; (ii) Absence of circulating blasts and blasts with Auer rods; (ii) Absence of extramedullary disease 4. ANC ≥1.0×10⁹/L (1,000/μL); (iii) Platelet count ≥100×10⁹/L (100,000/μL); and (iv) Independence of red cell transfusions. Complete Response with Incomplete Hematologic Recovery (CRi) can be indicated by meeting all the CR criteria except for residual neutropenia (<1.0×10⁹/L) or thrombocytopenia (<100×10⁹/L).

### B. Sensitivities

In some embodiments, a subject does not have a known allergy or hypersensitivity to, or intolerance of, tacrolimus. In some embodiments, a subject does not have a known allergy or hypersensitivity to, or intolerance of, sirolimus.

In some embodiments, subjects are not sensitive to iron dextran (e.g., subjects with sensitivity to iron dextran are not eligible to receive a composition of the disclosure. In some cases, this may be because of the magnetic beads used in some embodiments to isolate, deplete, and/or purify cell types).

In some embodiments, subjects are not sensitive to products derived from cyanine dyes (e.g., subjects with sensitivity to products derived from cyanine dyes are not eligible to receive a composition of the disclosure).

In some embodiments, subjects are not sensitive to proteins products derived from murine sources (e.g., subjects with sensitivity to proteins products derived from murine sources are not eligible to receive a composition of the disclosure).

In some embodiments, subjects are not sensitive to proteins products derived from bovine sources (e.g., subjects with sensitivity to proteins products derived from bovine sources are not eligible to receive a composition of the disclosure).

In some embodiments, subjects are not sensitive to proteins products derived from algal sources (e.g., subjects with sensitivity to proteins products derived from algal sources are not eligible to receive a composition of the disclosure).

In some embodiments, subjects are not sensitive to proteins products derived from Streptomyces avidinii (e.g., subjects with sensitivity to proteins products derived from Streptomyces avidinii are not eligible to receive a composition of the disclosure).

### C. Organ Function and Biomarkers

A subject can have an estimated glomerular filtration rate (eGFR) >30 mL/minute. A subject can have an estimated glomerular filtration rate (eGFR) >40 mL/minute. A subject can have an eGCF of >50 mL/minute. A subject can have an estimated glomerular filtration rate (eGFR) >60 mL/minute.

A subject can have a cardiac ejection fraction at rest ≥45%, or shortening fraction of ≥27% by echocardiogram or radionuclide scan (MUGA).

A subject can have a diffusing capacity of the lung for carbon monoxide (DLCO) (adjusted for hemoglobin) of ≥50%.

A subject can have a negative serum or urine beta-HCG test, e.g., in females of childbearing potential within 3 weeks of registration.

A subject can have total bilirubin <2 times upper limit of normal (ULN).

A subject can have Gilbert's syndrome, wherein hemolysis has been excluded.

A subject can have an ALT reading within 3 times upper limit of normal (ULN). A subject can have an AST reading within 3 times upper limit of normal (ULN).

### D. Additional Therapies and Other Subject Characteristics

In some embodiments, a subject has not received a prior alloHCT. In some embodiments, a subject is not a candidate for autologous transplant. In some embodiments, a subject is not receiving corticosteroids or other immunosuppressive therapy. In some embodiments, a subject is receiving topical corticosteroids or oral systemic corticosteroid doses less than or equal to 10 mg/day. In some embodiments, a subject does not receive donor lymphocyte infusion (DLI). In some embodiments, a subject does not receive a T cell depleting pharmaceutical, e.g., post-transplant cyclophosphamide (Cy), peri-transplant anti-thymocyte globulin (ATG), or alemtuzumab. In some embodiments, a subject that has previously been exposed to a T cell-depleting agent has a 5 half-life washout of the agent prior to planned transplant day 0 (day of infusion of the Treg and HSPC components of the graft). In some embodiments, a subject is not positive for anti-donor HLA antibodies against a mismatched allele in the selected donor as determined by either: (a) A positive crossmatch test of any titer; or (b) The presence of anti-donor HLA antibody to any HLA locus. In some embodiments, the subject has a Karnofsky performance score ≥70%. In some embodiments, a subject does not have a hematopoietic cell transplantation-specific Comorbidity Index (HCT-CI) of >4. In some embodiments, a subject does not have an uncontrolled bacterial, viral or fungal infection. In some embodiments, a subject is not taking antimicrobial therapy and with progression or no clinical improvement in infection. In some embodiments, a subject is not seropositive for HIV-1 or -2, HTLV-1 or -2, Hepatitis B sAg, or Hepatitis C antibody. In some embodiments, a subject does not have an uncontrolled autoimmune disease that requires active immunosuppressive treatment. In some embodiments, a subject does has not had concurrent malignancies or active disease within 1 year, for example, excluding non-melanoma skin cancers that have been curatively resected. In some embodiments, a subject does not exhibit psychosocial circumstances that preclude the patient being able to go through transplant or participate responsibly in follow up care. In some embodiments, a subject is not pregnant or breastfeeding. In some embodiments, a subject does not have a serious medical condition or abnormality in clinical laboratory tests that, in the medical professional's judgment, precludes the subject's safety upon receipt of a composition of the disclosure. In some embodiments, a subject is eligible for myeloablative alloHCT.

In some embodiments, a subject receives a prophylactic agent to reduce the risk of bacterial, fungal, and/or viral infection, e.g., during the peri-transplant period.

In some embodiments, a subject receives a supportive therapy for HCT-related toxicity. In some embodiments, a subject does not receive a supportive therapy for HCT-related toxicity. In some embodiments, a subject receives a growth factor. In some embodiments, a subject does not receive a growth factor. In some embodiments, a subject receives intravenous immunoglobulin. In some embodiments, a subject does not receive intravenous immunoglobulin. In some embodiments, a subject receives an analgesic. In some embodiments, a subject does not receive an analgesic. In some embodiments, a subject receives an anti-emetic. In some embodiments, a subject does not receive an anti-emetic. In some embodiments, a subject receives electrolyte replacement. In some embodiments, a subject does not receive electrolyte replacement. In some embodiments, a subject receives a tyrosine kinase inhibitor (e.g., a FLT3 inhibitor). In some embodiments, a subject does not receive a tyrosine kinase inhibitor (e.g., a FLT3 inhibitor). In some embodiments, a subject receives prednisone or an equivalent thereof, e.g., at a dose of ≤10 mg/day. In some embodiments, a subject does not receive prednisone or an equivalent thereof. In some embodiments, a subject receives corticosteroid treatment to manage GVHD. In some embodiments, a subject does not receive corticosteroid treatment to manage GVHD. In some embodiments, a subject receives high-dose corticosteroid treatment to manage GVHD. In some embodiments, a subject does not receive high-dose corticosteroid treatment to manage GVHD. In some embodiments, a subject receives corticosteroid treatment to manage, for example, adrenal insufficiency, hypersensitivity reactions, or other non-cancer-related symptoms including premedication for known hypersensitivity reactions to contrast for scans. In some embodiments, a subject does not receive corticosteroid treatment. In some embodiments, a subject receives an immunosuppressive medication. In some embodiments, a subject does not receive an immunosuppressive medication. In some embodiments, a subject receives a donor lymphocyte infusion. In some embodiments, a subject does not receive a donor lymphocyte infusion.

### E. Conditioning Regimen

Conditioning regimens can be used as part of an alloHCT regimen of the disclosure. Chemotherapy and/or irradiation given soon before a transplant is called a conditioning regimen. Conditioning regimens can help eradicate a patient's disease prior to the infusion of HSPCs, suppress immune reactions, and allow a donor HSPCs to reconstitute the vacant hematopoietic compartment that results from the conditioning regimen. In some embodiments of the methods of the disclosure, a subject can be treated with myeloablative conditioning prior to infusion of cell populations described herein. In some embodiments of the methods of the disclosure, a subject can be treated with myeloreductive conditioning prior to infusion of cell populations described herein. In some embodiments of the methods of the disclosure, a subject can be treated with a reduced intensity myeloablative conditioning prior to infusion of cell populations described herein. In some embodiments of the methods of the disclosure, a subject can be treated with non-myeloablative conditioning prior to administering a cell population or cell populations described herein.

As used herein, the term conditioning regimen and the like applies to myeloablative conditioning, myeloreductive conditioning, reduced intensity myeloablative therapy/conditioning, and/or non-myeloablative conditioning. As used herein, the term myeloablative therapy/conditioning also includes myeloreductive conditioning and reduced intensity myeloablative conditioning.

In aspects and embodiments, a treatment and/or method further comprises a conditioning regimen, wherein the conditioning regimen is administered before administration of one of (a) a solution comprising a first population of CD45+ cells comprising hematopoietic stem and progenitor cells (HSPCs) and granulocytes wherein at most about 10% of the first population of CD45+ cells comprise granulocytes; (b) a solution comprising a population of cells enriched for regulatory T cells (Tregs); and (c) a solution comprising a second population of CD45+ cells wherein the second population of CD45+ cells comprise at least about 20% CD3+ conventional T cells (Tcons), at least about 10% monocytes, and at least about 10% granulocytes; and (d) a solution comprising one or more doses of a graft vs host disease (GVHD) prophylactic agent. In embodiments, the conditioning regimen is a myeloablative conditioning regimen. In some cases, the conditioning regimen comprises at least three conditioning reagents, wherein at least one conditioning reagent is thiotepa. In various embodiments, the myeloablative conditioning regimen comprises at least one dose of thiotepa, e.g., at least about 5 milligrams thiotepa per kilogram of the human subject's actual or ideal body weight or at least about 10 milligrams thiotepa per kilogram of the human subject's actual or ideal body weight. In some embodiments, the conditioning regimen comprises one or more doses of busulfan, fludarabine and thiotepa. In embodiments, the one or more doses comprises from about 5 to about 12 mg of thiotepa per kg human subject's actual or ideal body weight, from about 7 to about 11 mg of busulfan per kg human subject actual or ideal body weight, and from about 100 to about 200 mg of fludarabine per meter² body surface area respectively.

In various embodiments, the method further comprises administering a myeloablative conditioning regimen to the patient prior to the administration of any cell population, the conditioning regimen comprising administration of at least one conditioning agent to the patient.

In some embodiments, the patient does not receive any irradiation as part of the myeloablative conditioning regimen.

In embodiments, the at least one conditioning agent is administered from about two to about ten days prior to the administration of any of the cell populations. In some cases, the at least one conditioning agent is administered about five days prior to the administration of any of the cell populations.

In various embodiments, the human subject has undergone myeloablative conditioning regimen before administration of any cell populations and the adverse event is associated with the myeloablative conditioning.

In some embodiments, the at least one conditioning agent comprises thiotepa. In some cases, a dose of thiotepa administered to the patient is in a range of from about 5 to about 10 mg per kilogram of actual or ideal body weight.

In embodiments, the at least one conditioning agent comprises busulfan and fludarabine. In some cases, doses of thiotepa, busulfan, and fludarabine administered to the patient comprise about 10 mg per kilogram of the patient's actual or ideal body weight, about 9.6 mg per kilogram of the patient's actual or ideal body weight, and about 150 mg per meter² body surface area respectively.

In some embodiments, the subject has been conditioned with radiation, chemotherapy, recombinant proteins, antibodies, or toxin-conjugated antibodies, or any combination thereof prior to administering a cell population or cell populations described herein. In some embodiments, the subject is conditioned for cellular graft therapy by first treating the subject with myeloablative therapy. Exemplary myeloablative therapies include chemotherapy or radiotherapy. Myleoablative therapies are thought to provide therapeutic benefit by debulking a tumor and/or reducing the number of cancer cells. Myeloablative regimens eradicate a sufficient number of HSCs that the patient would otherwise increase the chances of a patient developing GVHD. When HSPCs are subsequently administered to the myeloablated subject, the donor cells can further attack the cancer and/or and reconstitute the blood and the immune system of the subject.

In some embodiments, the myeloablative therapy comprises administration of thiotepa (TTP), busulfan, cyclophosphamide, Total Body Irradiation (TBI), fludarabine, etoposide, or any combination thereof. In some embodiments, the myeloablative therapy comprises administration an anti-cKIT antibody. In some embodiments, the myeloablative therapy comprises administration an antibody drug conjugate. The antibody drug conjugate can be, for example, anti-CD45-saporin or anti-cKit-saporin therapeutic antibodies. In some embodiments, the myeloablative therapy is a reduced intensity conditioning therapy. Exemplary conditioning regimens are described in Table 15.

A conditioning regimen of this disclosure may comprise one or more doses of busulfan. A conditioning regimen of this disclosure may comprise fludarabine. A conditioning regimen of this disclosure may comprise one or more doses of Cyclophosphamide. A conditioning regimen of this disclosure may comprise one or more doses of Melphalan. A conditioning regimen of this disclosure may comprise one or more doses of Etoposide.

The methods of the disclosure can comprise administration of a combination of conditioning reagents prior to the administration of the cells. A conditioning regimen as described herein may comprise administering 1, 2, 3 or 4 different conditioning reagents. The conditioning reagents used herein may be alkylating agents. The conditioning reagents used herein may be myeloablative. The conditioning reagents used herein may be non-myeloablative. The conditioning reagents used herein may be myeloreductive. The conditioning reagents used herein may be a form of chemotherapy.

The conditioning regimen described herein may comprise administration of an alkylating agent such as thiotepa (TTP). A conditioning regimen of this disclosure comprising TTP may comprise at least one more conditioning reagent. The conditioning reagents administered to a subject in addition to TTP may comprise one or more reagents selected from busulfan, dimethyl myleran, prednisone, methyl prednisolone, azathioprine, cyclophosphamide, cyclosparine, monoclonal antibodies against T cells, antilymphocyte globulin and anti-thymocyte globulin, fludarabine, etoposide, radiation, total body irradiation (TBI). Aspects and embodiments include any combination of TTP with the one or more conditioning reagents. In some embodiments, a subject is administered a conditioning regimen comprising thiotepa, busulfan, and fludarabine. In some embodiments, a subject is administered a conditioning regimen comprising thiotepa, fludarabine, and TBI (e.g., HFTBI).

The conditioning regimen described herein may comprise administration of an alkylating agent such as TTP. In some cases, a conditioning regimen of the disclosure may comprise TTP administration on more than one day. A conditioning regimen of the disclosure may comprise administering 2 mg/kg to 14 mg/kg TTP to a subject. A conditioning regimen of this disclosure may comprise administering at least 3 mg/kg TTP to a subject. A conditioning regimen of this disclosure may comprise administering at most 14 mg/kg TTP to a subject. A conditioning regimen of this disclosure may comprise administering 2 mg/kg to 5 mg/kg, 2 mg/kg to 6 mg/kg, 2 mg/kg to 8 mg/kg, 2 mg/kg to 10 mg/kg, 2 mg/kg to 12 mg/kg, 2 mg/kg to 14 mg/kg, 5 mg/kg to 6 mg/kg, 5 mg/kg to 8 mg/kg, 5 mg/kg to 10 mg/kg, 5 mg/kg to 12 mg/kg, 5 mg/kg to 14 mg/kg, 6 mg/kg to 8 mg/kg, 6 mg/kg to 10 mg/kg, 6 mg/kg to 12 mg/kg, 6 mg/kg to 14 mg/kg, 8 mg/kg to 10 mg/kg, 8 mg/kg to 12 mg/kg, 8 mg/kg to 14 mg/kg, 10 mg/kg to 12 mg/kg, 10 mg/kg to 14 mg/kg, or 12 mg/kg to 14 mg/kg TTP to a subject. A conditioning regimen of this disclosure may comprise administering 2 mg/kg, 3 mg/kg, 4 mg/kg, 5 mg/kg, 6 mg/kg, 8 mg/kg, 10 mg/kg, 12 mg/kg, or 14 mg/kg TTP to a subject. A conditioning regimen of this disclosure may comprise administering at most 2 mg/kg, 3 mg/kg, 4 mg/kg, 5 mg/kg, 6 mg/kg, 8 mg/kg, 10 mg/kg or 12 mg/kg TTP to a subject. A conditioning regimen of this disclosure may comprise administering at least 2 mg/kg, 3 mg/kg, 4 mg/kg, 5 mg/kg, 6 mg/kg, 8 mg/kg, 10 mg/kg, 12 mg/kg, or 14 mg/kg TTP to a subject.

As used herein, a recited dose, e.g., # mg/kg, may be relative to a subject's actual body weight (in kg) or relative to the subject's ideal body weight (in kg). Or the recited dose may be relative to a subject's adjusted body weight (ABW) if the subject's actual body weight is greater than 120% of the ideal body weight (IBW).

A subject administered one or more cell populations described herein may be administered one or more doses of TTP prior to the cell transplant. A subject receiving one or more cell populations described herein may be administered 1, 2, 3, 4, 5, 6, 7, 8, 9 or 10 doses of TTP prior to the cell transplant. In some cases, each dose of TTP has the same concentration. In some cases, one or more doses of TTP have different concentrations. A subject may be administered 1 mg/kg to 10 mg/kg TTP in a single dose. A subject may be administered at least 1 mg/kg TTP in a single dose. A subject may be administered at most 10 mg/kg TTP in a single dose. A subject may be administered 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 1 mg/kg to 10 mg/kg, 2 mg/kg to 3 mg/kg, 2 mg/kg to 4 mg/kg, 2 mg/kg to 5 mg/kg, 2 mg/kg to 6 mg/kg, 2 mg/kg to 7 mg/kg, 2 mg/kg to 8 mg/kg, 2 mg/kg to 9 mg/kg, 2 mg/kg to 10 mg/kg, 3 mg/kg to 4 mg/kg, 3 mg/kg to 5 mg/kg, 3 mg/kg to 6 mg/kg, 3 mg/kg to 7 mg/kg, 3 mg/kg to 8 mg/kg, 3 mg/kg to 9 mg/kg, 3 mg/kg to 10 mg/kg, 4 mg/kg to 5 mg/kg, 4 mg/kg to 6 mg/kg, 4 mg/kg to 7 mg/kg, 4 mg/kg to 8 mg/kg, 4 mg/kg to 9 mg/kg, 4 mg/kg to 10 mg/kg, 5 mg/kg to 6 mg/kg, 5 mg/kg to 7 mg/kg, 5 mg/kg to 8 mg/kg, 5 mg/kg to 9 mg/kg, 5 mg/kg to 10 mg/kg, 6 mg/kg to 7 mg/kg, 6 mg/kg to 8 mg/kg, 6 mg/kg to 9 mg/kg, 6 mg/kg to 10 mg/kg, 7 mg/kg to 8 mg/kg, 7 mg/kg to 9 mg/kg, 7 mg/kg to 10 mg/kg, 8 mg/kg to 9 mg/kg, 8 mg/kg to 10 mg/kg, or 9 mg/kg to 10 mg/kg TTP in a single dose. A subject may be administered 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg TTP in a single dose. A subject may be administered at most 1 mg/kg, 2 mg/kg, 3 mg/kg, 4 mg/kg, 5 mg/kg, 6 mg/kg, 7 mg/kg, 8 mg/kg, 9 mg/kg or 10 mg/kg TTP in a single dose. A subject may be administered at least 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg TTP in a single dose.

The methods of the disclosure can comprise administration of a combination of conditioning reagents prior to the administration of the cells. A conditioning regimen as described herein may comprise administering 1, 2, 3 or 4 different conditioning reagents. The conditioning reagents used herein may be alkylating agents. The conditioning reagents used herein may be myeloablative. The conditioning reagents used herein may be non-myeloablative. The conditioning reagents used herein may be myeloreductive. The conditioning reagents used herein may be a form of chemotherapy.

A conditioning regimen of this disclosure may comprise one or more doses of busulfan. One or more doses of busulfan may be administered to a subject before the administration of one or more doses of another conditioning reagent such as TTP. One or more doses of busulfan may be administered to a subject after the administration of one or more doses of another conditioning reagent such as TTP. One or more doses of busulfan may be administered to a subject along with the administration of one or more doses of another conditioning reagent such as TTP.

A conditioning regimen of this disclosure may comprise administering about 6 mg/kg to about 12 mg/kg busulfan to a subject. A conditioning regimen of this disclosure may comprise administering at least about 6 mg/kg busulfan to a subject. A conditioning regimen of this disclosure may comprise administering at most about 12 mg/kg busulfan to a subject. A conditioning regimen of this disclosure may comprise administering about 6 mg/kg to about 7 mg/kg, about 6 mg/kg to about 8 mg/kg, about 6 mg/kg to about 9 mg/kg, about 6 mg/kg to about 10 mg/kg, about 6 mg/kg to about 11 mg/kg, about 6 mg/kg to about 12 mg/kg, about 7 mg/kg to about 8 mg/kg, about 7 mg/kg to about 9 mg/kg, about 7 mg/kg to about 10 mg/kg, about 7 mg/kg to about 11 mg/kg, about 7 mg/kg to about 12 mg/kg, about 8 mg/kg to about 9 mg/kg, about 8 mg/kg to about 10 mg/kg, about 8 mg/kg to about 11 mg/kg, about 8 mg/kg to about 12 mg/kg, about 9 mg/kg to about 10 mg/kg, about 9 mg/kg to about 11 mg/kg, about 9 mg/kg to about 12 mg/kg, about 10 mg/kg to about 11 mg/kg, about 10 mg/kg to about 12 mg/kg, or about 11 mg/kg to about 12 mg/kg busulfan to a subject. A conditioning regimen of this disclosure may comprise administering 6 mg/kg, 7 mg/kg, 8 mg/kg, 9 mg/kg, 10 mg/kg, 11 mg/kg, or 12 mg/kg busulfan to a subject. A conditioning regimen of this disclosure may comprise administering at least 6 mg/kg, 7 mg/kg, 8 mg/kg, 9 mg/kg, 10 mg/kg or 11 mg/kg busulfan to a subject. A conditioning regimen of this disclosure may comprise administering at most 7 mg/kg, 8 mg/kg, 9 mg/kg, 10 mg/kg, 11 mg/kg, or 12 mg/kg busulfan to a subject.

A subject receiving one or more cell populations described herein may be administered one or more doses of busulfan prior to the cell transplant. A subject receiving one or more cell components described herein may be administered 1, 2, 3, 4, 5, 6, 7, 8, 9 or 10 doses of busulfan prior to the cell transplant. In some cases, each dose of busulfan has the same concentration. In some cases, one or more doses of busulfan have different concentrations. A subject may be administered 1 mg/kg to 10 mg/kg busulfan in a single dose. A subject may be administered at least 1 mg/kg busulfan in a single dose. A subject may be administered at least 2 mg/kg busulfan in a single dose. A subject may be administered at least 3 mg/kg busulfan in a single dose.

A conditioning regimen of this disclosure may comprise one or more doses of fludarabine. One or more doses of fludarabine may be administered to a subject before the administration of one or more doses of another conditioning reagent such as TTP. One or more doses of fludarabine may be administered to a subject after the administration of one or more doses of another conditioning reagent such as TTP. One or more doses of fludarabine may be administered to a subject along with the administration of one or more doses of another conditioning reagent such as TTP.

A conditioning regimen of this disclosure may comprise administering 20 mg/m² to 180 mg/m² fludarabine to a subject based on the surface area of the subject. A conditioning regimen of this disclosure may comprise administering at least 20 mg/m² fludarabine to a subject. A conditioning regimen of this disclosure may comprise administering at most 180 mg/m² fludarabine to a subject. A conditioning regimen of this disclosure may comprise administering 20 mg/m² to 30 mg/m², 20 mg/m² to 40 mg/m², 20 mg/m² to 50 mg/m², 20 mg/m² to 60 mg/m², 20 mg/m² to 80 mg/m², 20 mg/m² to 100 mg/m², 20 mg/m² to 120 mg/m², 20 mg/m² to 150 mg/m², 20 mg/m² to 180 mg/m², 30 mg/m² to 40 mg/m², 30 mg/m² to 50 mg/m², 30 mg/m² to 60 mg/m², 30 mg/m² to 80 mg/m², 30 mg/m² to 100 mg/m², 30 mg/m² to 120 mg/m², 30 mg/m² to 150 mg/m², 30 mg/m² to 180 mg/m², 40 mg/m² to 50 mg/m², 40 mg/m² to 60 mg/m², 40 mg/m² to 80 mg/m², 40 mg/m² to 100 mg/m², 40 mg/m² to 120 mg/m², 40 mg/m² to 150 mg/m², 40 mg/m² to 180 mg/m², 50 mg/m² to 60 mg/m², 50 mg/m² to 80 mg/m², 50 mg/m² to 100 mg/m², 50 mg/m² to 120 mg/m², 50 mg/m² to 150 mg/m², 50 mg/m² to 180 mg/m², 60 mg/m² to 80 mg/m², 60 mg/m² to 100 mg/m², 60 mg/m² to 120 mg/m², 60 mg/m² to 150 mg/m², 60 mg/m² to 180 mg/m², 80 mg/m² to 100 mg/m², 80 mg/m² to 120 mg/m², 80 mg/m² to 150 mg/m², 80 mg/m² to 180 mg/m², 100 mg/m² to 120 mg/m², 100 mg/m² to 150 mg/m², 100 mg/m² to 180 mg/m², 120 mg/m² to 150 mg/m², 120 mg/m² to 180 mg/m², or 150 mg/m² to 180 mg/m² fludarabine to a subject. A conditioning regimen of this disclosure may comprise administering 20 mg/m², 30 mg/m², 40 mg/m², 50 mg/m², 60 mg/m², 80 mg/m², 100 mg/m², 120 mg/m², 150 mg/m², or 180 mg/m² fludarabine to a subject. A conditioning regimen of this disclosure may comprise administering at least 20 mg/m², 30 mg/m², 40 mg/m², 50 mg/m², 60 mg/m², 80 mg/m², 100 mg/m², 120 mg/m² or 150 mg/m² fludarabine to a subject. A conditioning regimen of this disclosure may comprise administering at most 30 mg/m², 40 mg/m², 50 mg/m², 60 mg/m², 80 mg/m², 100 mg/m², 120 mg/m², 150 mg/m², or 180 mg/m² fludarabine to a subject.

A subject receiving one or more cell components described herein may be administered one or more doses of fludarabine prior to the cell transplant. A subject receiving one or more cell components described herein may be administered 1, 2, 3, 4, 5, 6, 7, 8, 9 or 10 doses of fludarabine prior to the cell transplant. In some cases, each dose of fludarabine has the same concentration. In some cases, one or more doses of fludarabine have different concentrations. A subject may be administered 20 to 60 mg/m² dose of fludarabine in a single dose. A subject may be administered at least 30 mg/m² fludarabine in a single dose. A subject may be administered at least 40 mg/m² fludarabine in a single dose. A subject may be administered at least 50 mg/m² fludarabine in a single dose.

A conditioning regimen of this disclosure may comprise administering 20 mg/m² to 180 mg/m² melphalan to a subject based on the surface area of the subject. A conditioning regimen of this disclosure may comprise administering at least 20 mg/m² melphalan to a subject. A conditioning regimen of this disclosure may comprise administering at most 180 mg/m² melphalan to a subject. A conditioning regimen of this disclosure may comprise administering 20 mg/m² to 30 mg/m², 20 mg/m² to 40 mg/m², 20 mg/m² to 50 mg/m², 20 mg/m² to 60 mg/m², 20 mg/m² to 80 mg/m², 20 mg/m² to 100 mg/m², 20 mg/m² to 120 mg/m², 20 mg/m² to 150 mg/m², 20 mg/m² to 180 mg/m², 30 mg/m² to 40 mg/m², 30 mg/m² to 50 mg/m², 30 mg/m² to 60 mg/m², 30 mg/m² to 80 mg/m², 30 mg/m² to 100 mg/m², 30 mg/m² to 120 mg/m², 30 mg/m² to 150 mg/m², 30 mg/m² to 180 mg/m², 40 mg/m² to 50 mg/m², 40 mg/m² to 60 mg/m², 40 mg/m² to 80 mg/m², 40 mg/m² to 100 mg/m², 40 mg/m² to 120 mg/m², 40 mg/m² to 150 mg/m², 40 mg/m² to 180 mg/m², 50 mg/m² to 60 mg/m², 50 mg/m² to 80 mg/m², 50 mg/m² to 100 mg/m², 50 mg/m² to 120 mg/m², 50 mg/m² to 150 mg/m², 50 mg/m² to 180 mg/m², 60 mg/m² to 80 mg/m², 60 mg/m² to 100 mg/m², 60 mg/m² to 120 mg/m², 60 mg/m² to 150 mg/m², 60 mg/m² to 180 mg/m², 80 mg/m² to 100 mg/m², 80 mg/m² to 120 mg/m², 80 mg/m² to 150 mg/m², 80 mg/m² to 180 mg/m², 100 mg/m² to 120 mg/m², 100 mg/m² to 150 mg/m², 100 mg/m² to 180 mg/m², 120 mg/m² to 150 mg/m², 120 mg/m² to 180 mg/m², or 150 mg/m² to 180 mg/m² melphalan to a subject. A conditioning regimen of this disclosure may comprise administering 20 mg/m², 30 mg/m², 40 mg/m², 50 mg/m², 60 mg/m², 80 mg/m², 100 mg/m², 120 mg/m², 150 mg/m², or 180 mg/m² melphalan to a subject. A conditioning regimen of this disclosure may comprise administering at least 20 mg/m², 30 mg/m², 40 mg/m², 50 mg/m², 60 mg/m², 80 mg/m², 100 mg/m², 120 mg/m² or 150 mg/m² melphalan to a subject. A conditioning regimen of this disclosure may comprise administering at most 30 mg/m², 40 mg/m², 50 mg/m², 60 mg/m², 80 mg/m², 100 mg/m², 120 mg/m², 150 mg/m², or 180 mg/m² melphalan to a subject.

A subject receiving one or more cell components described herein may be administered one or more doses of melphalan prior to the cell transplant. A subject receiving one or more cell components described herein may be administered 1, 2, 3, 4, 5, 6, 7, 8, 9 or 10 doses of melphalan prior to the cell transplant. In some cases, each dose of melphalan has the same concentration. In some cases, one or more doses of melphalan have different concentrations.

A conditioning regimen of this disclosure may comprise administering 100 mg/kg to 140 mg/kg cyclophosphamide. A conditioning regimen of this disclosure may comprise administering at least 100 mg/kg cyclophosphamide. A conditioning regimen of this disclosure may comprise administering at most 140 mg/kg cyclophosphamide. A conditioning regimen of this disclosure may comprise administering 100 mg/kg to 110 mg/kg, 100 mg/kg to 120 mg/kg, 100 mg/kg to 130 mg/kg, 100 mg/kg to 140 mg/kg, 110 mg/kg to 120 mg/kg, 110 mg/kg to 130 mg/kg, 110 mg/kg to 140 mg/kg, 120 mg/kg to 130 mg/kg, 120 mg/kg to 140 mg/kg, or 130 mg/kg to 140 mg/kg cyclophosphamide. A conditioning regimen of this disclosure may comprise administering about 100 mg/kg, 110 mg/kg, 120 mg/kg, 130 mg/kg, or 140 mg/kg cyclophosphamide. A conditioning regimen of this disclosure may comprise administering at least 100 mg/kg, 110 mg/kg, 120 mg/kg or 130 mg/kg cyclophosphamide. A conditioning regimen of this disclosure may comprise administering at most 110 mg/kg, 120 mg/kg, 130 mg/kg, or 140 mg/kg cyclophosphamide.

A subject receiving one or more cell components described herein may be administered one or more doses of cyclophosphamide prior to the cell transplant. A subject receiving one or more cell components described herein may be administered 1, 2, 3, 4, 5, 6, 7, 8, 9 or 10 doses of cyclophosphamide prior to the cell transplant. In some cases, each dose of cyclophosphamide has the same concentration. In some cases, one or more doses of cyclophosphamide have different concentrations.

A conditioning regimen of this disclosure may comprise administering 40 mg/kg to 80 mg/kg etoposide. A conditioning regimen of this disclosure may comprise administering at least 40 mg/kg etoposide. A conditioning regimen of this disclosure may comprise administering at most 80 mg/kg etoposide. A conditioning regimen of this disclosure may comprise administering 40 mg/kg to 50 mg/kg, 40 mg/kg to 60 mg/kg, 40 mg/kg to 70 mg/kg, 40 mg/kg to 80 mg/kg, 50 mg/kg to 60 mg/kg, 50 mg/kg to 70 mg/kg, 50 mg/kg to 80 mg/kg, 60 mg/kg to 70 mg/kg, 60 mg/kg to 80 mg/kg, or 70 mg/kg to 80 mg/kg etoposide. A conditioning regimen of this disclosure may comprise administering about 40 mg/kg, 50 mg/kg, 60 mg/kg, 70 mg/kg, or 80 mg/kg etoposide. A conditioning regimen of this disclosure may comprise administering at least 40 mg/kg, 50 mg/kg, 60 mg/kg or 70 mg/kg etoposide. A conditioning regimen of this disclosure may comprise administering at most 50 mg/kg, 60 mg/kg, 70 mg/kg, or 80 mg/kg etoposide.

A subject receiving one or more cell components described herein may be administered one or more doses of etoposide prior to the cell transplant. A subject receiving one or more cell components described herein may be administered 1, 2, 3, 4, 5, 6, 7, 8, 9 or 10 doses of etoposide prior to the cell transplant. In some cases, each dose of etoposide has the same concentration. In some cases, one or more doses of etoposide have different concentrations.

A conditioning regimen of this disclosure comprising TTP may comprise one or more doses of total body irradiation (TBI) such as hyperfractionated TBI (HFTBI). One or more doses of HFTBI may be administered to a subject before the administration of one or more doses of another conditioning reagent such as TTP. One or more doses of HFTBI may be administered to a subject after the administration of one or more doses of another conditioning reagent such as TTP. One or more doses of HFTBI may be administered to a subject along with the administration of one or more doses of another conditioning reagent such as TTP.

A conditioning regimen of this disclosure may comprise administering 800 cGy to 1,500 cGy HFTBI to a subject. A conditioning regimen of this disclosure may comprise administering at least 800 cGy HFTBI to a subject. A conditioning regimen of this disclosure may comprise administering at most 1,500 cGy HFTBI to a subject. A conditioning regimen of this disclosure may comprise administering 800 cGy to 900 cGy, 800 cGy to 1,000 cGy, 800 cGy to 1,100 cGy, 800 cGy to 1,200 cGy, 800 cGy to 1,300 cGy, 800 cGy to 1,375 cGy, 800 cGy to 1,400 cGy, 800 cGy to 1,500 cGy, 900 cGy to 1,000 cGy, 900 cGy to 1,100 cGy, 900 cGy to 1,200 cGy, 900 cGy to 1,300 cGy, 900 cGy to 1,375 cGy, 900 cGy to 1,400 cGy, 900 cGy to 1,500 cGy, 1,000 cGy to 1,100 cGy, 1,000 cGy to 1,200 cGy, 1,000 cGy to 1,300 cGy, 1,000 cGy to 1,375 cGy, 1,000 cGy to 1,400 cGy, 1,000 cGy to 1,500 cGy, 1,100 cGy to 1,200 cGy, 1,100 cGy to 1,300 cGy, 1,100 cGy to 1,375 cGy, 1,100 cGy to 1,400 cGy, 1,100 cGy to 1,500 cGy, 1,200 cGy to 1,300 cGy, 1,200 cGy to 1,375 cGy, 1,200 cGy to 1,400 cGy, 1,200 cGy to 1,500 cGy, 1,300 cGy to 1,375 cGy, 1,300 cGy to 1,400 cGy, 1,300 cGy to 1,500 cGy, 1,375 cGy to 1,400 cGy, 1,375 cGy to 1,500 cGy, or 1,400 cGy to 1,500 cGy HFTBI to a subject. A conditioning regimen of this disclosure may comprise administering about 800 cGy, 900 cGy, 1,000 cGy, 1,100 cGy, 1,200 cGy, 1,300 cGy, 1,375 cGy, 1,400 cGy, or 1,500 cGy HFTBI to a subject. A conditioning regimen of this disclosure may comprise administering at least 800 cGy, 900 cGy, 1,000 cGy, 1,100 cGy, 1,200 cGy, 1,300 cGy, 1,375 cGy or 1,400 cGy HFTBI to a subject. A conditioning regimen of this disclosure may comprise administering at most 900 cGy, 1,000 cGy, 1,100 cGy, 1,200 cGy, 1,300 cGy, 1,375 cGy, 1,400 cGy, or 1,500 cGy HFTBI to a subject.

A subject receiving one or more cell components described herein may be administered one or more doses of HFTBI prior to the cell transplant. A subject receiving one or more cell components described herein may be administered 1, 2, 3, 4, 5, 6, 7, 8, 9 or 10 doses of HFTBI prior to the cell transplant. In some cases, each dose of HFTBI has the same concentration. In some cases, one or more doses of HFTBI have different concentrations. A subject may be administered a 75 to 150 cGys of HFTBI in a single dose. A subject may be administered at least 75 cGys HFTBI in a single dose. A subject may be administered at least 100 cGys HFTBI in a single dose. A subject may be administered at least 125 cGys HFTBI in a single dose.

Exemplary Conditioning Regimen 1: In some embodiments, a subject is administered a conditioning regimen comprising thiotepa, busulfan, and fludarabine. In some embodiments, a subject is administered thiotepa at 5 mg/kg actual body weight or ideal body weight. In some embodiments, a subject is administered thiotepa at about 5 mg/kg for two days (e.g., consecutive days). In some embodiments, a subject is administered busulfan at about 3.2 mg/kg actual body weight or ideal body weight. In some embodiments, a subject is administered busulfan at about 3.2 mg/kg daily for three days (e.g., consecutive days). In some embodiments, a subject is administered fludarabine at about 50 mg/m². (meters squared−body surface area). In some embodiments, a subject is administered fludarabine at about 50 mg/m² for three days (e.g., consecutive days). In some embodiments, a subject is administered thiotepa at 5 mg/kg actual body weight or ideal body weight, is administered busulfan at about 3.2 mg/kg actual body weight or ideal body weight, and is administered fludarabine at about 50 mg/m2. (meters squared−body surface area). In some embodiments, a subject is administered thiotepa at about 5 mg/kg for two days (e.g., consecutive days), is administered busulfan at about 3.2 mg/kg daily for three days (e.g., consecutive days), and is administered fludarabine at about 50 mg/m² for three days (e.g., consecutive days). In some embodiments, a subject is administered thiotepa at about 5 mg/kg on days −7 and −6, is administered busulfan at about 3.2 mg/kg daily on days −5 to −3, and is administered fludarabine at about 50 mg/m² on days −5 to −3.

Exemplary Conditioning Regimen 2: In some embodiments, a subject is administered a conditioning regimen comprising thiotepa, fludarabine, and TBI (e.g., HFTBI). In some embodiments, a subject is administered thiotepa at 5 mg/kg actual body weight or ideal body weight. In some embodiments, a subject is administered thiotepa at about 5 mg/kg for two days (e.g., consecutive days). In some embodiments, a subject is administered fludarabine at about 25 mg/m². (meters squared−body surface area). In some embodiments, a subject is administered fludarabine at about 25 mg/m² for three days (e.g., consecutive days). In some embodiments, a subject is administered HFTBI of 125 cGy (centigray). In some embodiments, a subject is administered HFTBI in 11 fractions of 125 cGy. In some embodiments, a subject is administered HFTBI in 11 fractions of 125 cGy over 4 days. In some embodiments, a subject is administered thiotepa at 5 mg/kg actual body weight or ideal body weight, is administered fludarabine at about 25 mg/m². (meters squared−body surface area), and is administered HFTBI of 125 cGy. In some embodiments, a subject is administered thiotepa at about 5 mg/kg for two days (e.g., consecutive days), is administered fludarabine at about 50 mg/m² for three days (e.g., consecutive days), and is administered HFTBI in 11 fractions of 125 cGy. In some embodiments, a subject is administered thiotepa at about 5 mg/kg on days −7 and −6, is administered fludarabine at about 50 mg/m² on days −5 to -3, and is administered HFTBI in 11 fractions of 125 cGy over 4 days.

A subject may receive the one or more cell populations described herein 1 day after completing a conditioning regimen. A subject may receive the one or more cell components described herein 2, 3, 4, 5, 6, 7, 8, 9, 10 days after completing a conditioning regimen. A subject may receive the one or more cell components described herein 1 day after receiving a final dose of a conditioning reagent such as TTP. A subject may receive the one or more cell components described herein 2, 3, 4, 5, 6, 7, 8, 9 or 10 days after receiving a final dose of a conditioning reagent such as TTP. A subject may receive the one or more cell components described herein 1 day after receiving a first dose of a conditioning reagent such as TTP. A subject may receive the one or more cell components described herein 2, 3, 4, 5, 6, 7, 8, 9 or 10 days after receiving a first dose of a conditioning reagent such as TTP.

### IV. Graft Versus Host Disease (GVHD

Graft versus host disease (GVHD) is a significant cause of morbidity and mortality in hematopoietic stem cell transplantation (HCT) recipients. Aspects and embodiments herein provide compositions and methods that reduce the incidence of GVHD, reduce the severity of GVHD, reduce the relative risk of GVHD, prevent GVHD, or a combination thereof in HCT recipients.

Graft-versus-host disease (GVHD) is an inflammatory disease that can occur in the allogeneic transplant setting. GVHD involves donor cells (graft) attacking recipient cells (host). GVHD can be life-threatening and can involve, for example, the skin, the intestines, and/or the liver. The morbidity and mortality associated GVHD can be a major factor limiting the success of HCT. GVHD can occur despite use of a HLA-matched sibling donor, and the use of various GVHD prophylactic/immunosuppressive agents, for example, use of two or more of tacrolimus, sirolimus, cyclosporine, methotrexate, mycophenolate, anti-thymocyte globulin and corticosteroids.

### A. GVHD Classification and Grading

GVHD can be classified into acute GVHD (aGVHD) and chronic GVHD (cGVHD). In some embodiments, GVHD that occurs within the first 100 days post-transplant can be referred to as aGVHD, and GVHD after the first 100 days can be referred to as chronic GVHD (cGVHD). cGVHD is a major source of late treatment-related complications, and can be life-threatening. In addition to inflammation, cGVHD can lead to the development of fibrosis, which can result in functional disability.

Yet another aspect provides any herein-disclosed multi-component pharmaceutical treatment in which a risk and/or severity of an adverse event associated with the multi-component pharmaceutical treatment is reduced as compared to a similar pharmaceutical treatment in which a human subject receives Tcons but does not receive Tregs or is any herein-disclosed method in which a risk and/or severity of an adverse event associated with the method is reduced as compared to a similar method in which a human subject receives Tcons but does not receive Tregs.

In various embodiments, the adverse event is acute GVHD (aGVHD).

In some embodiments, the adverse event is stage two or greater aGVHD.

In embodiments, the adverse event is chronic GVHD (cGVHD).

In various embodiments, the human subject has no cGVHD about one year after being administered the cell populations.

In some embodiments, the adverse event is moderate to severe cGVHD.

In embodiments, the adverse event is acute graft vs host disease (aGVHD), e.g., stage two or greater aGVHD. In some cases, the patient has no stage two or higher aGVHD about 180 days after being administered the cell populations.

In various embodiments, the adverse event is chronic graft vs host disease (cGVHD). In some cases, the patient has no cGVHD about one year after being administered the cell populations.

In some embodiments, the adverse event is moderate to severe cGVHD. In some cases, the patient does not have moderate to severe cGVHD about one year after being administered the cell populations.

In embodiments, a patient does not develop GVHD within about 30 days of administration of the Tcons, does not develop GVHD within about 100 days of administration of the Tcons, does not develop GVHD within about 180 days of administration of the Tcons, and/or does not develop GVHD within about one year of administration of the Tcons.

In various embodiments, a human subject does not develop higher than stage 2 GVHD within about 100 days of said administering of said second population of CD45+ cells, said human subject does not develop higher than stage 2 GVHD) within about 180 days or within about 200 days of said administering of said second population of CD45+ cells, said human subject does not develop higher than stage 2 GVHD within about 1 year of said administering of said second population of GCD45+ cells.

aGVHD and cGVHD can be graded using a system that first evaluates GVHD stages for the skin, liver, and gut, and then combines scoring from the organ staging to determine an overall GVHD grade. An example of a GVHD staging criteria that can be used for individual organs are provided in TABLE 1:

TABLE 1

| Organ | Stage | Description |
|---|---|---|
| Skin | 0 | No active (erythematous) GVHD rash |
| | 1 | Maculo-papular eruption over <25% of body area |
| | 2 | Maculo-papular eruption over >25 to 50% of body area |
| | 3 | Generalized erythroderma/maculo-papular eruption |
| | | over >50% of body area |
| | 4 | Generalized erythroderma/maculo-papular eruption |
| | | over >50% of body area plus bullous formation and |
| | | desquamation over >5% of body area |
| Liver | 0 | Bilirubin <2 mg/dL |
| | 1 | Bilirubin 2.0 to 3.0 mg/dL; |
| | | serum glutamic oxaloacetic transaminase (SGOT) SGOT |
| | | 150 to 750 IU |
| | 2 | Bilirubin 3.1 to 6.0 mg/dL |
| | 3 | Bilirubin 6.1 to 15.0 mg/dL |
| | 4 | Bilirubin >15.0 mg/dL |
| Gut | 0 | <500 mL/day or <3 episodes/day |
| | 1 | Diarrhea >30 mL/kg or >500 mL/day |
| | 2 | Diarrhea >60 mL/kg or >1000 mL/day |
| | 3 | Diarrhea >90 mL/kg or >1500 mL/day |
| | 4 | Diarrhea >90 mL/kg or >2000 mL/day; or severe |
| | | abdominal pain with or without ileus |

Table 2 provides one set of criteria for assessing overall GVHD grade.

TABLE 2

| | | | | | ECOG |
|---|---|---|---|---|---|
| Grade | Skin | Liver | | Gut | performance |
| 1 | 1 to 2 | 0 | | 0 | 0 |
| 2 | 1 to 3 | 1 | and/or | 1 | 0-1 |
| 3 | 2 to 3 | 2 to 4 | and/or | 2 to 3 | 2-3 |
| 4 | 2 to 4 | 2 to 4 | and/or | 2 to 3 | 3-4 |

aGVHD grade can also be determined based on most severe target organ involvement as defined in the MAGIC standardization criteria described by Harris et al., “International, multicenter standardization of acute graft-versus-host disease clinical data collection: a report from the Mount Sinai Acute GVHD International Consortium.” Biology of Blood and Marrow Transplantation 22.1 (2016): 4-10. For example, aGVHD organ staging can be evaluated according to TABLE 3, and overall aGVHD grade can be assessed as follows:

Grade 0: No Stage 1-4 of any organ.

Grade I: Stage 1-2 skin without liver, upper GI, or lower GI involvement.

Grade II: Stage 3 rash and/or Stage 1 liver and/or Stage 1 upper GI and/or Stage 1 lower GI.

Grade III: Stage 2-3 liver and/or Stage 2-3 lower GI, with Stage 0-3 skin and/or Stage 0-1 upper GI. Grade IV: Stage 4 skin, liver, or lower GI involvement, with Stage 0-1 upper GI.

TABLE 3

| | Skin (Active | Liver | | | |
|---|---|---|---|---|---|
| Stage | Erythema Only) | (Bilirubin) | | Upper GI | Lower GI (stool output/day) |
| 0 | No active | <2 | mg/dL | No or intermittent | Adult: <500 mL/day or <3 |
| | (erythematous) | | | nausea, vomiting | episodes/day |
| | GvHD rash | | | or anorexia | Child: <10 mL/kg/day or <4 |
| | | | | | episodes/day |
| 1 | Maculopapular | 2-3 | mg/dL | Persistent nausea, | Adult: 500-999 mL/day or |
| | rash <25% BSA | | | vomiting or | 3-4 episodes/day |
| | | | | anorexia | Child: 10-19.9 mL/kg/day or |
| | | | | | 5-7 episodes/day |
| 2 | Maculopapular | 3.1-6 | mg/dL | | Adult: 1000-1500 mL/day or |
| | rash 25-50% BSA | | | | 57 episodes/day |
| | | | | | Child: 20-30 mL/kg/day or |
| | | | | | 7-10 episodes/day |
| 3 | Maculopapular | 6.1-15 | mg/dL | | Adult: >1500 mL/day or >7 |
| | rash >50% BSA | | | | episodes/day |
| | | | | | Child: >30 mL/kg/day or >10 |
| | | | | | episodes/day |
| 4 | Generalized | >15 | mg/dL | | Severe abdominal pain with or |
| | erythroderma | | | | without ileus or grossly bloody |
| | (>50% BSA) | | | | stool (regardless of stool |
| | plus bullous | | | | volume) |

formation and
desquamation >5%
BSA

In some embodiments, GVHD stage and GVHD grade are synonyms.

cGVHD can also be assessed by the method described by Jagasia et al., “National Institutes of Health consensus development project on criteria for clinical trials in chronic graft-versus-host disease: I. The 2014 diagnosis and staging working group report.” Biology of Blood and Marrow Transplantation 21.3 (2015): 389-401. To establish a diagnosis of cGVHD, these criteria can require, for example, at least one diagnostic manifestation of chronic GVHD or at least one distinctive manifestation plus a pertinent biopsy, laboratory or other test (e.g. PFTs, Schirmer's test), evaluation by a specialist (ophthalmologist, gynecologist) or radiographic imaging showing chronic GVHD in the same or another organ.

Organ systems can be scored as described in Jagasia et al., and Mild cGVHD can be present when one or two organs are involved with no more than score 1, plus a lung score of zero; moderate cGVHD can be present when three or more organs are involved with no more than score 1, or when at least one non-lung organ has a score of 2, or when the lungs have a score of 1; and severe cGVHD can be present when at least one organ has a score of 4, or the lungs have a score of D2 or 3.

TABLE 4 provides diagnostic and distinctive manifestations of cGVHD. infection, drug effect, malignancy, or other causes are excluded for distinctive manifestations. Bronchiolitis obliterans syndrome can be diagnostic for lung chronic GVHD only if distinctive sign or symptom present in another organ. Diagnosis of chronic GVHD based on myositis or polymyositis can require a biopsy.

TABLE 4

| Organ or | Diagnostic (Sufficient to Establish | Distinctive¹ (Seen in chronic GVHD, but |
|---|---|---|
| Site | the Diagnosis of chronic GVHD) | Insufficient Alone to Establish a Diagnosis) |
| Skin | Poikiloderma Lichen planus-like | Depigmentation Papulosquamous |
| | features | lesions |
| | Sclerotic features Morphea- | |
| | like features | |
| | Lichen sclerosus-like features | |
| Nails | | Dystrophy |
| | | Longitudinal ridging, splitting or brittle features |
| | | Onycholysis |
| | | Pterygium unguis |
| | | Nail loss (usually symmetric, affects most nails) |
| Scalp and | | New onset of scarring or nonscarring scalp |
| body hair | | alopecia (after recovery from |
| | | chemoradiotherapy) |
| | | Loss of body hair |
| Mouth | Lichen planus-like changes | Xerostomia Mucoceles Mucosal |
| | | atrophy Ulcers |
| | | Pseudomembranes |
| Eyes | | New onset dry, gritty, or painful eyes |
| | | Cicatricial conjunctivitis |
| | | Keratoconjunctivitis sicca |
| | | Confluent areas of punctate keratopathy |
| Genitalia | Lichen planus-like features Lichen | Erosions Fissures |
| | sclerosus-like features | |
| Females | Vaginal scarring or clitoral/labial | Ulcers |
| | agglutination | |
| Males | Phimosis or urethral/meatus scarring | |
| | or stenosis | |
| GI Tract | Esophageal web Strictures or stenosis | |
| | in the upper to | |
| | mid third of the esophagus | |
| Lung | Bronchiolitis obliterans diagnosed | Air trapping and bronchiectasis on chest CT |
| | with lung biopsy | |
| | Bronchiolitis obliterans syndrome² | |
| Muscles, | Joint stiffness or contractures | Myositis or polymyositis³ |
| fascia, joints | secondary to fasciitis or sclerosis | |

In some embodiments, GVHD severity can be graded using the Glucksberg grade (I-IV) or the International Bone Marrow Transplant Registry (IBMTR) grading system (A-D). The severity of acute GVHD can be determined by an assessment of the degree of involvement of the skin, liver, and gastrointestinal tract. The stages of individual organ involvement are combined with (Glucksberg) or without (IBMTR) the patient's performance status to produce an overall grade.

### B. GVHD Prophylaxis and Treatment

Immunosuppressive agents can be used to reduce the likelihood of GVHD (GVHD prophylactic agents), or to treat GVHD once it occurs (GVHD therapeutic agents). However, in some cases, the use of GVHD prophylactic agents, GVHD therapeutic agents, or both can be insufficient to effectively prevent or treat GVHD. For example, the incidence of GVHD in graft recipients can be high despite use of use of tacrolimus, sirolimus, cyclosporine, methotrexate, mycophenolate, anti-thymocyte globulin, corticosteroids, or a combination thereof (e.g., two or more of the agents).

Administering multiple GVHD prophylactic and/or therapeutic agents, high doses of GVHD prophylactic and/or therapeutic agents, or both can fail to effectively treat GVHD in many alloHCT settings or can result in increased susceptibility to infection and decreased graft versus tumor therapeutic effects.

Non-limiting examples of GVHD prophylactic and/or GVHD therapeutic agents that can be used include calcineurin inhibitors (e.g., tacrolimus, cyclosporine A), sirolimus, monoclonal antibodies, methotrexate, mycophenolate, anti-thymocyte globulin, corticosteroids, azathioprine, and mycophenolate mofetil. Monoclonal antibodies useful as immunosuppressive agents include, for example, antagonist antibodies, (e.g., antibodies that antagonize IL-2R such as basiliximab and daclizumab), and antibodies that deplete an immune cell population by antibody dependent cellular cytotoxicity (e.g., anti-CD3 antibodies for T cell depletion such as muromonab-CD3).

Compositions and methods described herein may comprise administering one or more GVHD prophylactic agents to an HCT recipient. GVHD prophylaxis in such cases should be considered different from GVHD treatment such that the GVHD prophylactic agent will be administered to the HCT recipient before an incidence of GVHD is assessed. In some cases, an HCT recipient may be administered one or more GVHD prophylactic agents but not a GVHD therapeutic agent. In some cases, a HCT recipient does not require treatment for GVHD and/or does not receive treatment for GVHD.

Compositions and methods disclosed herein can reduce the incidence of GVHD, reduce the severity of GVHD, reduce the relative risk of GVHD, prevent GVHD, or a combination thereof in HCT recipients. In some embodiments, such benefits are achieved despite administering no GVHD prophylactic agents as disclosed herein. In some embodiments, such benefits are achieved despite administering a reduced number of GVHD prophylactic agents (e.g., a single GVHD prophylactic agents), a low dose of GVHD prophylactic agent(s), or a combination thereof as disclosed herein. In some embodiments, 1 GVHD prophylactic agent is administered to a subject. In some embodiments, no more than GVHD prophylactic agent is administered to a subject. In some embodiments, 2 GVHD prophylactic agents are administered to a subject. In some embodiments, no more than 2 GVHD prophylactic agents are administered to a subject.

In some embodiments, one or more GVHD prophylactic agents may be administered to a HCT recipient for a duration of 2 days, 3 days, 4 days, 5 days, 6 days, 7 days, 2 weeks, 3 weeks, 4 weeks, 2 months, 3 months, 4 months, 5 months, 6 months, 7 months, 8 months, 9 months, 10 months, 11 months, 12 months, 18 months, 24 months, 36 months post-transplant of one or more cell populations. One or more GVHD prophylactic agents may be administered to an HCT recipient starting the day of transplant of one or more cell populations. For instance, a GVHD prophylactic regimen may begin the day an HSPC cell population and/or a Treg cell population is administered to the recipient. Alternatively, a GVHD prophylactic regimen may begin the day a Tcon cell population is administered to the patient.

In some embodiments, tacrolimus is not administered to a subject. In some embodiments, sirolimus is not administered to a subject. In some embodiments, cyclosporine is not administered to a subject. In some embodiments, methotrexate is not administered to a subject. In some embodiments, mycophenolate is not administered to a subject. In some embodiments, anti-thymocyte globulin is not administered to a subject. In some embodiments, corticosteroids are not administered to a subject.

In some embodiments, the GVHD prophylactic agent is tacrolimus.

In embodiments, the tacrolimus graft versus host disease (GVHD) prophylactic agent (GVHDPA) is intravenously administered or orally administered. In various embodiments, administration of the tacrolimus graft versus host disease (GVHD) prophylactic agent (GVHDPA) is started from about 12 to about 24 hours after administration of the T-cons. In some cases, the tacrolimus GHVDPA is administered for a period of time up to about 90 days, is administered for a period of time up to about 60 days. In some embodiments, the tacrolimus GHVDPA is initially administered to the patient at about 0.03 mg/kg patient's actual or ideal body weight/day. In some cases, a dose of the tacrolimus GVHDPA administered to the patient is tapered starting at about 90 days after a first dose is administered to the patient or is tapered starting at about 45 days after a first dose is administered to the patient.

Aspects and embodiments herein provide a method of transplanting cell populations into a human patient as a part of a treatment regimen for a hematologic malignancy. The method comprises administering to the patient a population of hematopoietic stem and progenitor cells (HSPCs; the population of HSPCs comprising HSPCs and a liquid suspending the HSPCs; administering to the patient a population of regulatory T cells (Tregs) to be administered to the patient, the population of Tregs comprising Tregs and a liquid suspending the Tregs; and administering to the patient a heterogenous cell population to be administered to the patient, the heterogenous cell population comprising lymphocytes, granulocytes, monocytes and a liquid suspending said cells, wherein at least about 30% of said lymphocyte comprise conventional T cells (Tcons); and administering to the patient over a period of time up to about 180 days a single graft versus host disease (GVHD) prophylactic agent (GVHDPA) comprising tacrolimus (tacrolimus GHVDPA), wherein the tacrolimus GHVDPA is administered to maintain a concentration of tacrolimus in the patient's blood above a threshold level during the period of time; and wherein a risk and/or severity of GHVD associated with the treatment regimen for the hematologic malignancy is significantly reduced.

In cases where aGVHD occurs, responsiveness of aGVHD to GVHD therapeutic agents (e.g., corticosteroids) can be assessed by the criteria of TABLE 5.

TABLE 5

| | |
|---|---|
| Complete | Complete resolution of acute GvHD symptoms in all organs, |
| response (CR) | without secondary GvHD therapy |
| Partial response (PR) | Improvement in GvHD stage in all initial GvHD target organs |
| | without complete resolution and without worsening in any other |
| | GvHD target organs, without secondary GvHD therapy |
| Very good partial | Improvement in GvHD in all initial GvHD target organs, with maximum |
| response (VGPR) | Stage I involvement in one or more organs (except upper |
| | gastrointestinal tract), without secondary GvHD therapy |
| No response (NR) | Same grade of GvHD or progression of GvHD in any organ or death, or |
| | the addition of secondary GvHD therapy |
| Progression | Worsening GvHD in at least 1 organ with or without amelioration in |
| | any organ |

In some embodiments, in cases where aGVHD occurs in subjects that receive a composition(s) of the disclosure, at least about 40%, at least about 50%, at least about 55%, at least about 60%, at least about 65%, at least about 70%, at least about 75%, at least about 80%, at least about 85%, at least about 90%, or at least about 95% of cases exhibit a complete response to GVHD therapeutic agents.

In some embodiments, in cases where aGVHD occurs in subjects that receive a composition(s) of the disclosure, at least about 40%, at least about 50%, at least about 55%, at least about 60%, at least about 65%, at least about 70%, at least about 75%, at least about 80%, at least about 85%, at least about 90%, or at least about 95% of cases exhibit a partial response to GVHD therapeutic agents.

In some embodiments, in cases where aGVHD occurs in subjects that receive a composition(s) of the disclosure, at least about 40%, at least about 50%, at least about 55%, at least about 60%, at least about 65%, at least about 70%, at least about 75%, at least about 80%, at least about 85%, at least about 90%, or at least about 95% of cases exhibit a very good partial response to GVHD therapeutic agents.

In cases where cGVHD occurs, responsiveness of cGVHD to GVHD therapeutic agents (e.g., corticosteroids) can be assessed by the criteria of TABLE 6. Abbreviations used: ALT, alanine transaminase; FEV1, forced expiratory volume in the first second; OMRS, Oral Mucosa Rating Scale; PFTs, pulmonary function tests; P-ROM, photographic range of motion; ULN, upper limit of normal.

TABLE 6

| Organ | Complete Response | Partial Response | Progression |
|---|---|---|---|
| Skin | NIH Skin Score 0 after | Decrease in NIH Skin | Increase in NIH Skin Score by 1 |
| | previous involvement | Score by 1 or more points | or more points, except 0 to 1 |
| Eyes | NIH Eye Score 0 after | Decrease in NIH Eye Score | Increase in NIH Eye Score by 1 |
| | previous involvement | by 1 or more points | or more points, except 0 to 1 |
| Mouth | NIH Modified OMRS 0 | Decrease in NIH Modified | Increase in NIH Modified |
| | after previous involvement | OMRS of 2 or more points | OMRS of 2 or more points |
| Esophagus | NIH Esophagus Score 0 after | Decrease in NIH | Increase in NIH Esophagus |
| | previous involvement | Esophagus Score by 1 or | Score by 1 or more points, |
| | | more points | except 0 to 1 |
| Upper GI | NIH Upper GI Score 0 after | Decrease in NIH Upper GI | Increase in NIH Upper GI |
| | previous involvement | Score by 1 or more points | Score by 1 or more points, |
| | | | except 0 to 1 |
| Lower GI | NIH Lower GI Score 0 after | Decrease in NIH Lower GI | Increase in NIH Lower GI |
| | previous involvement | Score by 1 or more points | Score by 1 or more points, |
| | | | except from 0 to 1 |
| Liver | Normal ALT, alkaline | Decrease by 50% | Increase by 2 × ULN |
| | phosphatase, and Total | | |
| | bilirubin after previous | | |
| | elevation of 1 or more | | |
| Lungs | Normal % FEV1 after | Increase by 10% | Decrease by 10% predicted |
| | previous involvement | predicted absolute value | absolute value of % FEV1 |
| | If PFTs not available, | of % FEV1 | If PFTs not available, |
| | NIH Lung Symptom | If PFTs not available, | increase in NIH Lung - |
| | Score 0 after previous | decrease in NIH Lung | Symptom Score by 1 or |
| | involvement | Symptom Score by 1 or | more points, except 0 to 1 |
| | | more points | |
| Joints and | Both NIH Joint and Fascia | Decrease in NIH Joint and | Increase in NIH Joint and Fascia |
| fascia | Score 0 and P-ROM score 25 | Fascia Score by 1 or more | Score by 1 or more points or |
| | after previous involvement by | points or increase in P-ROM | decrease in P-ROM score by 1 |
| | at least 1 measure | score by 1 point for any site | point for any site |
| Global | Clinician overall severity | Clinician overall severity | Clinician overall severity score |
| | score 0 | score decreases by 2 or more | increases by 2 or more points on a |
| | | points on a 0-10 scale | 0-10 scale |

In some embodiments, in cases where cGVHD occurs in subjects that receive a composition of the disclosure, at least about 40%, at least about 50%, at least about 55%, at least about 60%, at least about 65%, at least about 70%, at least about 75%, at least about 80%, at least about 85%, at least about 90%, or at least about 95% of cases exhibit a complete response to GVHD therapeutic agents.

In some embodiments, in cases where cGVHD occurs in subjects that receive a composition of the disclosure, at least about 40%, at least about 50%, at least about 55%, at least about 60%, at least about 65%, at least about 70%, at least about 75%, at least about 80%, at least about 85%, at least about 90%, or at least about 95% of cases exhibit a partial response to GVHD therapeutic agents.

### C. Acute GVHD Incidence

Subjects administered a composition of the disclosure (e.g., a cell population as described herein) exhibit a low incidence of ≥grade 1 aGVHD, for example, a lower incidence of ≥grade 1 aGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure develop ≥grade 1 aGVHD, for example, within 30 days post-transplant, 100 days post-transplant, or within another suitable amount of time post-transplant as disclosed herein.

As used herein, an alternate composition lacks one or more cell populations and/or prophylactic agent that are disclosed herein and/or recited in the claims. As examples, an alternate composition lacks one or more of a first population of CD45+ cells that comprises, at least, HSPCs, a cell population enriched for Tregs, a second population of CD45+ cells that comprises, at least, Tcons, and a prophylactic agent.

Subjects administered a composition of the disclosure (e.g., a cell population as described herein) exhibit a low incidence of ≥grade 2 aGVHD, for example, a lower incidence of ≥grade 2 aGVHD than subjects that are administered an alternate composition. In some embodiments, less than about 10%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop ≥grade 2 aGVHD, for example, within 30 days post-transplant, 100 days post-transplant, or within another suitable amount of time post-transplant as disclosed herein.

In some embodiments, less than about 20% of subjects that are administered a composition of the disclosure develop ≥grade 2 aGVHD. In some embodiments, less than about 15% of subjects that are administered a composition of the disclosure develop ≥grade 2 aGVHD. In some embodiments, less than about 10% of subjects that are administered a composition of the disclosure develop ≥grade 2 aGVHD. In some embodiments, less than about 8% of subjects that are administered a composition of the disclosure develop ≥grade 2 aGVHD. In some embodiments, less than about 7% of subjects that are administered a composition of the disclosure develop ≥grade 2 aGVHD.

Subjects administered a composition of the disclosure (e.g., a cell population as described herein) exhibit a low incidence of ≥grade 3 aGVHD, for example, a lower incidence of ≥grade 3 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop ≥grade 3 aGVHD, for example, within 30 days post-transplant, 100 days post-transplant, or within another suitable amount of time post-transplant as disclosed herein.

In some embodiments, less than about 20% of subjects that are administered a composition of the disclosure develop ≥grade 3 aGVHD. In some embodiments, less than about 15% of subjects that are administered a composition of the disclosure develop ≥grade 3 aGVHD. In some embodiments, less than about 10% of subjects that are administered a composition of the disclosure develop ≥grade 3 aGVHD. In some embodiments, less than about 5% of subjects that are administered a composition of the disclosure develop ≥grade 3 aGVHD. In some embodiments, less than about 3% of subjects that are administered a composition of the disclosure develop ≥grade 3 aGVHD. In some embodiments, less than about 2% of subjects that are administered a composition of the disclosure develop ≥grade 3 aGVHD. In some embodiments, less than about 1% of subjects that are administered a composition of the disclosure develop ≥grade 3 aGVHD.

Subjects administered a composition of the disclosure (e.g., a cell population as described herein) exhibit a low incidence of ≥grade 4 aGVHD, for example, a lower incidence of ≥grade 4 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure develop ≥grade 4 aGVHD, for example, within 30 days post-transplant, 100 days post-transplant, or within another suitable amount of time post-transplant as disclosed herein.

In some embodiments, less than about 10% of subjects that are administered a composition of the disclosure develop ≥grade 4 aGVHD. In some embodiments, less than about 5% of subjects that are administered a composition of the disclosure develop ≥grade 4 aGVHD. In some embodiments, less than about 3% of subjects that are administered a composition of the disclosure develop ≥grade 4 aGVHD. In some embodiments, less than about 2% of subjects that are administered a composition of the disclosure develop ≥grade 4 aGVHD. In some embodiments, less than about 1% of subjects that are administered a composition of the disclosure develop ≥grade 4 aGVHD.

The incidence of aGVHD can be assessed after a suitable amount of time elapses post-transplant, for example, about 20 days, about 21 days, about 25 days, about 28 days, about 30 days, about 35 days, about 40 days, about 42 days, about 45 days, about 49 days, about 50 days, about 55 days, about 56 days, about 60 days, about 63 days, about 65 days, about 70 days, about 75 days, about 77 days, about 80 days, about 84 days, about 85 days, about 90 days, about 91 days, about 95 days, about 98 days, about 100 days, about 105 days, about 110 days, about 112 days, about 115 days, about 119 days, about 120 days post-transplant.

The incidence of aGVHD can be calculated based on a population of at least 10, at least at least 11, at least at least 12, at least 13, at least 14, at least 15, at least 16, at least 17, at least 18, at least 19, at least 20, at least 21, at least 22, at least 23, at least 24, at least 25, at least 25, at least 30, at least 35, at least 40, at least 45, at least 50, at least 60, at least 70, at least 80, at least 90, at least 100, at least 120, at least 140, at least 160, at least 180, at least 200, at least 250, at least 300, at least 350, at least 400, at least 450, or at least 500 subjects.

1. No GVHD Prophylaxis

Subjects administered a composition of the disclosure (e.g., a cell population as described herein) in the absence of GVHD prophylactic agents exhibit a low incidence of ≥grade 1 aGVHD, for example, a lower incidence of ≥grade 1 aGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure in the absence of GVHD prophylactic agents develop ≥grade 1 aGVHD, for example, within 30 days post-transplant, 100 days post-transplant, or within another suitable amount of time post-transplant as disclosed herein.

Subjects administered a composition of the disclosure (e.g., a cell population as described herein) in the absence of GVHD prophylactic agents exhibit a low incidence of ≥grade 2 aGVHD, for example, a lower incidence of ≥grade 2 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop ≥grade 2 aGVHD, for example, within 30 days post-transplant, 100 days post-transplant, or within another suitable amount of time post-transplant as disclosed herein.

In some embodiments, less than about 50% of subjects that are administered a composition of the disclosure in the absence of GVHD prophylactic agents develop ≥grade 2 aGVHD. In some embodiments, less than about 40% of subjects that are administered a composition of the disclosure in the absence of GVHD prophylactic agents develop ≥grade 2 aGVHD. In some embodiments, less than about 30% of subjects that are administered a composition of the disclosure in the absence of GVHD prophylactic agents develop ≥grade 2 aGVHD. In some embodiments, less than about 25% of subjects that are administered a composition of the disclosure in the absence of GVHD prophylactic agents develop ≥grade 2 aGVHD. In some embodiments, less than about 20% of subjects that are administered a composition of the disclosure in the absence of GVHD prophylactic agents develop ≥grade 2 aGVHD. In some embodiments, less than about 10% of subjects that are administered a composition of the disclosure in the absence of GVHD prophylactic agents develop ≥grade 2 aGVHD.

Subjects administered a composition of the disclosure (e.g., a cell population as described herein) in the absence of GVHD prophylactic agents exhibit a low incidence of ≥grade 3 aGVHD, for example, a lower incidence of ≥grade 3 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop ≥grade 3 aGVHD, for example, within 30 days post-transplant, 100 days post-transplant, or within another suitable amount of time post-transplant as disclosed herein.

In some embodiments, less than about 40% of subjects that are administered a composition of the disclosure in the absence of GVHD prophylactic agents develop ≥grade 3 aGVHD. In some embodiments, less than about 30% of subjects that are administered a composition of the disclosure in the absence of GVHD prophylactic agents develop ≥grade 3 aGVHD. In some embodiments, less than about 20% of subjects that are administered a composition of the disclosure in the absence of GVHD prophylactic agents develop ≥grade 3 aGVHD. In some embodiments, less than about 10% of subjects that are administered a composition of the disclosure in the absence of GVHD prophylactic agents develop ≥grade 3 aGVHD. In some embodiments, less than about 5% of subjects that are administered a composition of the disclosure in the absence of GVHD prophylactic agents develop ≥grade 3 aGVHD. In some embodiments, less than about 3% of subjects that are administered a composition of the disclosure in the absence of GVHD prophylactic agents develop ≥grade 3 aGVHD. In some embodiments, less than about 2% of subjects that are administered a composition of the disclosure in the absence of GVHD prophylactic agents develop ≥grade 3 aGVHD. In some embodiments, less than about 1% of subjects that are administered a composition of the disclosure in the absence of GVHD prophylactic agents develop ≥grade 3 aGVHD.

Subjects administered a composition of the disclosure (e.g., a cell population as described herein) in the absence of GVHD prophylactic agents exhibit a low incidence of ≥grade 4 aGVHD, for example, a lower incidence of ≥grade 4 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure in the absence of GVHD prophylactic agents develop ≥grade 4 aGVHD, for example, within 30 days post-transplant, 100 days post-transplant, or within another suitable amount of time post-transplant as disclosed herein.

In some embodiments, less than about 20% of subjects that are administered a composition of the disclosure in the absence of GVHD prophylactic agents develop ≥grade 4 aGVHD. In some embodiments, less than about 15% of subjects that are administered a composition of the disclosure in the absence of GVHD prophylactic agents develop ≥grade 4 aGVHD. In some embodiments, less than about 10% of subjects that are administered a composition of the disclosure in the absence of GVHD prophylactic agents develop ≥grade 4 aGVHD. In some embodiments, less than about 5% of subjects that are administered a composition of the disclosure in the absence of GVHD prophylactic agents develop ≥grade 4 aGVHD. In some embodiments, less than about 3% of subjects that are administered a composition of the disclosure in the absence of GVHD prophylactic agents develop ≥grade 4 aGVHD. In some embodiments, less than about 2% of subjects that are administered a composition of the disclosure in the absence of GVHD prophylactic agents develop ≥grade 4 aGVHD. In some embodiments, less than about 1% of subjects that are administered a composition of the disclosure in the absence of GVHD prophylactic agents develop ≥grade 4 aGVHD.

The absence of GVHD prophylactic agents can refer to cases where no GVHD prophylactic agents are administered to the subject for the first 20 days, 21 days, 25 days, 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100 days, 105 days, 110 days, 112 days, 115 days, 119 days, or 120 days post-transplant.

2. Single Agent GVHD Prophylaxis

Subjects administered a composition of the disclosure (e.g., a cell population as described herein) and no more than one GVHD prophylactic agent (for example, a single GVHD prophylactic agent) exhibit a low incidence of ≥grade 1 aGVHD, for example, a lower incidence of ≥grade 1 aGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and no more than one GVHD prophylactic agent (for example, a single GVHD prophylactic agent) develop ≥grade 1 aGVHD, for example, within 30 days post-transplant, 100 days post-transplant, or within another suitable amount of time post-transplant as disclosed herein.

Subjects administered a composition of the disclosure (e.g., a cell population as described herein) and no more than one GVHD prophylactic agent (for example, a single GVHD prophylactic agent) exhibit a low incidence of ≥grade 2 aGVHD, for example, a lower incidence of ≥grade 2 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop ≥grade 2 aGVHD, for example, within 30 days post-transplant, 100 days post-transplant, or within another suitable amount of time post-transplant as disclosed herein.

In some embodiments, less than about 20% of subjects that are administered a composition of the disclosure and no more than one GVHD prophylactic agent (for example, a single GVHD prophylactic agent) develop ≥grade 2 aGVHD. In some embodiments, less than about 15% of subjects that are administered a composition of the disclosure and no more than one GVHD prophylactic agent (for example, a single GVHD prophylactic agent) develop ≥grade 2 aGVHD. In some embodiments, less than about 10% of subjects that are administered a composition of the disclosure and no more than one GVHD prophylactic agent (for example, a single GVHD prophylactic agent) develop ≥grade 2 aGVHD. In some embodiments, less than about 8% of subjects that are administered a composition of the disclosure and no more than one GVHD prophylactic agent (for example, a single GVHD prophylactic agent) develop ≥grade 2 aGVHD. In some embodiments, less than about 7% of subjects that are administered a composition of the disclosure and no more than one GVHD prophylactic agent (for example, a single GVHD prophylactic agent) develop ≥grade 2 aGVHD.

Subjects administered a composition of the disclosure (e.g., a cell population as described herein) and no more than one GVHD prophylactic agent (for example, a single GVHD prophylactic agent) exhibit a low incidence of ≥grade 3 aGVHD, for example, a lower incidence of ≥grade 3 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop ≥grade 3 aGVHD, for example, within 30 days post-transplant, 100 days post-transplant, or within another suitable amount of time post-transplant as disclosed herein.

In some embodiments, less than about 20% of subjects that are administered a composition of the disclosure and no more than one GVHD prophylactic agent (for example, a single GVHD prophylactic agent) develop ≥grade 3 aGVHD. In some embodiments, less than about 15% of subjects that are administered a composition of the disclosure and no more than one GVHD prophylactic agent (for example, a single GVHD prophylactic agent) develop ≥grade 3 aGVHD. In some embodiments, less than about 10% of subjects that are administered a composition of the disclosure and no more than one GVHD prophylactic agent (for example, a single GVHD prophylactic agent) develop ≥grade 3 aGVHD. In some embodiments, less than about 5% of subjects that are administered a composition of the disclosure and no more than one GVHD prophylactic agent (for example, a single GVHD prophylactic agent) develop ≥grade 3 aGVHD. In some embodiments, less than about 3% of subjects that are administered a composition of the disclosure and no more than one GVHD prophylactic agent (for example, a single GVHD prophylactic agent) develop ≥grade 3 aGVHD. In some embodiments, less than about 2% of subjects that are administered a composition of the disclosure and no more than one GVHD prophylactic agent (for example, a single GVHD prophylactic agent) develop ≥grade 3 aGVHD. In some embodiments, less than about 1% of subjects that are administered a composition of the disclosure and no more than one GVHD prophylactic agent (for example, a single GVHD prophylactic agent) develop ≥grade 3 aGVHD.

Subjects administered a composition of the disclosure (e.g., a cell population as described herein) and no more than one GVHD prophylactic agent (for example, a single GVHD prophylactic agent) exhibit a low incidence of ≥grade 4 aGVHD, for example, a lower incidence of ≥grade 4 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure and no more than one GVHD prophylactic agent (for example, a single GVHD prophylactic agent) develop ≥grade 4 aGVHD, for example, within 30 days post-transplant, 100 days post-transplant, or within another suitable amount of time post-transplant as disclosed herein.

In some embodiments, less than about 10% of subjects that are administered a composition of the disclosure and no more than one GVHD prophylactic agent (for example, a single GVHD prophylactic agent) develop ≥grade 4 aGVHD. In some embodiments, less than about 5% of subjects that are administered a composition of the disclosure and no more than one GVHD prophylactic agent (for example, a single GVHD prophylactic agent) develop ≥grade 4 aGVHD. In some embodiments, less than about 3% of subjects that are administered a composition of the disclosure and no more than one GVHD prophylactic agent (for example, a single GVHD prophylactic agent) develop ≥grade 4 aGVHD. In some embodiments, less than about 2% of subjects that are administered a composition of the disclosure and no more than one GVHD prophylactic agent (for example, a single GVHD prophylactic agent) develop ≥grade 4 aGVHD. In some embodiments, less than about 1% of subjects that are administered a composition of the disclosure and no more than one GVHD prophylactic agent (for example, a single GVHD prophylactic agent) develop ≥grade 4 aGVHD.

A single GVHD prophylactic agent can be tacrolimus.

A single GVHD prophylactic agent can be sirolimus.

The no more than one GVHD prophylactic agent (for example, a single GVHD prophylactic agent) can be administered to the subject for the first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years post-transplant. In some embodiments, the no more than one GVHD prophylactic agent (for example, a single GVHD prophylactic agent) is administered to the subject for less than about 20 days, less than about 30 days, less than about 40 days, less than about 50 days, less than about 60 days, less than about 70 days, less than about 80 days, less than about 90 days, less than about 100 days, less than about 110 days, less than about 120 days, less than about 150 days, less than about 200 days, less than about 365 days, less than about 13 months, less than about 14 months, less than about 15 months, less than about 16 months, less than about 17 months, less than about 18 months, less than about 19 months, less than about 20 months, less than about 21 months, less than about 22 months, less than about 23 months, less than about 2 years, less than about 2.5 years, less than about 3 years, less than about 3.5 years, less than about 4 years, less than about 4.5 years, or less than about or 5 years post-transplant.

3. Low Dose GVHD Prophylaxis

Subjects administered a composition of the disclosure (e.g., a cell population as described herein) and a low dose of a GVHD prophylactic agent (for example, a single GVHD prophylactic agent at a low dose) exhibit a low incidence of ≥grade 1 aGVHD, for example, a lower incidence of ≥grade 1 aGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and a low dose of a GVHD prophylactic agent (for example, a single GVHD prophylactic agent at a low dose) develop ≥grade 1 aGVHD, for example, within 30 days post-transplant, 100 days post-transplant, or within another suitable amount of time post-transplant as disclosed herein.

Subjects administered a composition of the disclosure (e.g., a cell population as described herein) and a low dose of a GVHD prophylactic agent (for example, a single GVHD prophylactic agent at a low dose) exhibit a low incidence of ≥grade 2 aGVHD, for example, a lower incidence of ≥grade 2 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop ≥grade 2 aGVHD, for example, within 30 days post-transplant, 100 days post-transplant, or within another suitable amount of time post-transplant as disclosed herein.

In some embodiments, less than about 20% of subjects that are administered a composition of the disclosure and a low dose of a GVHD prophylactic agent (for example, a single GVHD prophylactic agent at a low dose) develop ≥grade 2 aGVHD. In some embodiments, less than about 15% of subjects that are administered a composition of the disclosure and a low dose of a GVHD prophylactic agent (for example, a single GVHD prophylactic agent at a low dose) develop ≥grade 2 aGVHD. In some embodiments, less than about 10% of subjects that are administered a composition of the disclosure and a low dose of a GVHD prophylactic agent (for example, a single GVHD prophylactic agent at a low dose) develop ≥grade 2 aGVHD. In some embodiments, less than about 8% of subjects that are administered a composition of the disclosure and a low dose of a GVHD prophylactic agent (for example, a single GVHD prophylactic agent at a low dose) develop ≥grade 2 aGVHD. In some embodiments, less than about 7% of subjects that are administered a composition of the disclosure and a low dose of a GVHD prophylactic agent (for example, a single GVHD prophylactic agent at a low dose) develop ≥grade 2 aGVHD.

Subjects administered a composition of the disclosure (e.g., a cell population as described herein) and a low dose of a GVHD prophylactic agent (for example, a single GVHD prophylactic agent at a low dose) exhibit a low incidence of ≥grade 3 aGVHD, for example, a lower incidence of ≥grade 3 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop ≥grade 3 aGVHD, for example, within 30 days post-transplant, 100 days post-transplant, or within another suitable amount of time post-transplant as disclosed herein.

In some embodiments, less than about 20% of subjects that are administered a composition of the disclosure and a low dose of a GVHD prophylactic agent (for example, a single GVHD prophylactic agent at a low dose) develop ≥grade 3 aGVHD. In some embodiments, less than about 15% of subjects that are administered a composition of the disclosure and a low dose of a GVHD prophylactic agent (for example, a single GVHD prophylactic agent at a low dose) develop ≥grade 3 aGVHD. In some embodiments, less than about 10% of subjects that are administered a composition of the disclosure and a low dose of a GVHD prophylactic agent (for example, a single GVHD prophylactic agent at a low dose) develop ≥grade 3 aGVHD. In some embodiments, less than about 5% of subjects that are administered a composition of the disclosure and a low dose of a GVHD prophylactic agent (for example, a single GVHD prophylactic agent at a low dose) develop ≥grade 3 aGVHD. In some embodiments, less than about 3% of subjects that are administered a composition of the disclosure and a low dose of a GVHD prophylactic agent (for example, a single GVHD prophylactic agent at a low dose) develop ≥grade 3 aGVHD. In some embodiments, less than about 2% of subjects that are administered a composition of the disclosure and a low dose of a GVHD prophylactic agent (for example, a single GVHD prophylactic agent at a low dose) develop ≥grade 3 aGVHD. In some embodiments, less than about 1% of subjects that are administered a composition of the disclosure and a low dose of a GVHD prophylactic agent (for example, a single GVHD prophylactic agent at a low dose) develop ≥grade 3 aGVHD.

Subjects administered a composition of the disclosure (e.g., a cell population as described herein) and a low dose of a GVHD prophylactic agent (for example, a single GVHD prophylactic agent at a low dose) exhibit a low incidence of ≥grade 4 aGVHD, for example, a lower incidence of ≥grade 4 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure and a low dose of a GVHD prophylactic agent (for example, a single GVHD prophylactic agent at a low dose) develop ≥grade 4 aGVHD, for example, within 30 days post-transplant, 100 days post-transplant, or within another suitable amount of time post-transplant as disclosed herein.

In some embodiments, less than about 10% of subjects that are administered a composition of the disclosure and a low dose of a GVHD prophylactic agent (for example, a single GVHD prophylactic agent at a low dose) develop ≥grade 4 aGVHD. In some embodiments, less than about 5% of subjects that are administered a composition of the disclosure and a low dose of a GVHD prophylactic agent (for example, a single GVHD prophylactic agent at a low dose) develop ≥grade 4 aGVHD. In some embodiments, less than about 3% of subjects that are administered a composition of the disclosure and a low dose of a GVHD prophylactic agent (for example, a single GVHD prophylactic agent at a low dose) develop ≥grade 4 aGVHD. In some embodiments, less than about 2% of subjects that are administered a composition of the disclosure and a low dose of a GVHD prophylactic agent (for example, a single GVHD prophylactic agent at a low dose) develop ≥grade 4 aGVHD. In some embodiments, less than about 1% of subjects that are administered a composition of the disclosure and a low dose of a GVHD prophylactic agent (for example, a single GVHD prophylactic agent at a low dose) develop ≥grade 4 aGVHD.

A low dose of a GVHD prophylactic agent can be, for example a target trough level of less than about 25 ng/mL, less than about 20 ng/mL, less than about 15 ng/mL, less than about 12 ng/mL, less than about 11 ng/mL, less than about 10 ng/mL, less than about 9 ng/mL, less than about 8 ng/mL, less than about 7 ng/mL, less than about 6 ng/mL, less than about 5 ng/mL, less than about 4 ng/mL, less than about 3 ng/mL, less than about 2 ng/mL, or less than about 1 ng/mL.

In some embodiments, a low dose of a GVHD prophylactic is a target trough level of about 1-25 ng/mL, about 1-20 ng/mL, about 1-15 ng/mL, about 1-12 ng/mL, about 1-11 ng/mL, about 1-10 ng/mL, about 1-9 ng/mL, about 1-8 ng/mL, about 1-7 ng/mL, about 1-6 ng/mL, about 1-5 ng/mL, about 1-4 ng/mL, about 1-3 ng/mL, about 1-2 ng/mL, about 2-25 ng/mL, about 2-20 ng/mL, about 2-15 ng/mL, about 2-12 ng/mL, about 2-11 ng/mL, about 2-10 ng/mL, about 2-9 ng/mL, about 2-8 ng/mL, about 2-7 ng/mL, about 2-6 ng/mL, about 2-5 ng/mL, about 2-4 ng/mL, about 2-3 ng/mL, about 3-25 ng/mL, about 3-20 ng/mL, about 3-15 ng/mL, about 3-12 ng/mL, about 3-11 ng/mL, about 3-10 ng/mL, about 3-9 ng/mL, about 3-8 ng/mL, about 3-7 ng/mL, about 3-6 ng/mL, about 3-5 ng/mL, about 3-4 ng/mL, about 4-25 ng/mL, about 4-20 ng/mL, about 4-15 ng/mL, about 4-12 ng/mL, about 4-11 ng/mL, about 4-10 ng/mL, about 4-9 ng/mL, about 4-8 ng/mL, about 4-7 ng/mL, about 4-6 ng/mL, about 4-5 ng/mL, about 5-25 ng/mL, about 5-20 ng/mL, about 5-15 ng/mL, about 5-12 ng/mL, about 5-11 ng/mL, about 5-10 ng/mL, about 5-9 ng/mL, about 5-8 ng/mL, about 5-7 ng/mL, about 5-6 ng/mL, about 6-25 g/mL, about 6-20 ng/mL, about 6-15 ng/mL, about 6-12 ng/mL, about 6-11 ng/mL, about 6-10 ng/mL, about 6-9 ng/mL, about 6-8 ng/mL, about 6-7 ng/mL, about 8-25 ng/mL, about 8-20 ng/mL, about 8-15 ng/mL, about 8-12 ng/mL, about 8-11 ng/mL, about 8-10 ng/mL, about 8-9 ng/mL, about 10-25 ng/mL, about 10-20 ng/mL, about 10-15 ng/mL, about 10-12 ng/mL, or about 10-11 ng/mL.

In some embodiments, a low dose of a GVHD prophylactic agent is tacrolimus with a target trough level of about 5 ng/mL to about 10 ng/mL. In some embodiments, a low dose of a GVHD prophylactic agent is tacrolimus with a target trough level of about 4 ng/mL to about 6 ng/mL.

In some embodiments, a low dose of a GVHD prophylactic agent is sirolimus with a target trough level of about 3 ng/mL to about 8 ng/mL. In some embodiments, a low dose of a GVHD prophylactic agent is sirolimus with a target trough level of about 4 ng/mL to about 8 ng/mL.

The low dose GVHD prophylactic agent (for example, single GVHD prophylactic agent at a low dose) can be administered to the subject for the first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years post-transplant. In some embodiments, the low dose GVHD prophylactic agent (for example, single GVHD prophylactic agent at a low dose) is administered to the subject for less than about 20 days, less than about 30 days, less than about 40 days, less than about 50 days, less than about 60 days, less than about 70 days, less than about 80 days, less than about 90 days, less than about 100 days, less than about 110 days, less than about 120 days, less than about 150 days, less than about 200 days, less than about 365 days, less than about 13 months, less than about 14 months, less than about 15 months, less than about 16 months, less than about 17 months, less than about 18 months, less than about 19 months, less than about 20 months, less than about 21 months, less than about 22 months, less than about 23 months, less than about 2 years, less than about 2.5 years, less than about 3 years, less than about 3.5 years, less than about 4 years, less than about 4.5 years, or less than about or 5 years post-transplant.

### D. Chronic GVHD Incidence

Subjects administered a composition of the disclosure (e.g., a cell population as described herein) exhibit a low incidence of ≥grade 1 cGVHD, for example, a lower incidence of ≥grade 1 cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure develop ≥grade 1 cGVHD, for example, within 365 days post-transplant, two years post-transplant, or within another suitable amount of time post-transplant as disclosed herein.

Subjects administered a composition of the disclosure (e.g., a cell population as described herein) exhibit a low incidence of ≥grade 2 cGVHD, for example, a lower incidence of ≥grade 2 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop ≥grade 2 cGVHD, for example, within 365 days post-transplant, two years post-transplant, or within another suitable amount of time post-transplant as disclosed herein.

In some embodiments, less than about 50% of subjects that are administered a composition of the disclosure develop ≥grade 2 cGVHD. In some embodiments, less than about 40% of subjects that are administered a composition of the disclosure develop ≥grade 2 cGVHD. In some embodiments, less than about 30% of subjects that are administered a composition of the disclosure develop ≥grade 2 cGVHD. In some embodiments, less than about 20% of subjects that are administered a composition of the disclosure develop ≥grade 2 cGVHD. In some embodiments, less than about 10% of subjects that are administered a composition of the disclosure develop ≥grade 2 cGVHD. In some embodiments, less than about 5% of subjects that are administered a composition of the disclosure develop ≥grade 2 cGVHD.

Subjects administered a composition of the disclosure (e.g., a cell population as described herein) exhibit a low incidence of ≥grade 3 cGVHD, for example, a lower incidence of ≥grade 3 cGVHD than subjects that are administered an alternate composition. In some embodiments, less than about 10%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop ≥grade 3 cGVHD, for example, within 365 days post-transplant, two years post-transplant, or within another suitable amount of time post-transplant as disclosed herein.

In some embodiments, less than about 50% of subjects that are administered a composition of the disclosure develop ≥grade 3 cGVHD. In some embodiments, less than about 40% of subjects that are administered a composition of the disclosure develop ≥grade 3 cGVHD. In some embodiments, less than about 30% of subjects that are administered a composition of the disclosure develop ≥grade 3 cGVHD. In some embodiments, less than about 20% of subjects that are administered a composition of the disclosure develop ≥grade 3 cGVHD. In some embodiments, less than about 15% of subjects that are administered a composition of the disclosure develop ≥grade 3 cGVHD. In some embodiments, less than about 10% of subjects that are administered a composition of the disclosure develop ≥grade 3 cGVHD. In some embodiments, less than about 5% of subjects that are administered a composition of the disclosure develop ≥grade 3 cGVHD. In some embodiments, less than about 3% of subjects that are administered a composition of the disclosure develop ≥grade 3 cGVHD. In some embodiments, less than about 2% of subjects that are administered a composition of the disclosure develop ≥grade 3 cGVHD. In some embodiments, less than about 1% of subjects that are administered a composition of the disclosure develop ≥grade 3 cGVHD.

Subjects administered a composition of the disclosure (e.g., a cell population as described herein) exhibit a low incidence of ≥grade 4 cGVHD, for example, a lower incidence of ≥grade 4 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure develop ≥grade 4 cGVHD, for example, within 365 days post-transplant, two years post-transplant, or within another suitable amount of time post-transplant as disclosed herein.

In some embodiments, less than about 40% of subjects that are administered a composition of the disclosure develop ≥grade 4 cGVHD. In some embodiments, less than about 30% of subjects that are administered a composition of the disclosure develop ≥grade 4 cGVHD. In some embodiments, less than about 20% of subjects that are administered a composition of the disclosure develop ≥grade 4 cGVHD. In some embodiments, less than about 10% of subjects that are administered a composition of the disclosure develop ≥grade 4 cGVHD. In some embodiments, less than about 5% of subjects that are administered a composition of the disclosure develop ≥grade 4 cGVHD. In some embodiments, less than about 3% of subjects that are administered a composition of the disclosure develop ≥grade 4 cGVHD. In some embodiments, less than about 2% of subjects that are administered a composition of the disclosure develop ≥grade 4 cGVHD. In some embodiments, less than about 1% of subjects that are administered a composition of the disclosure develop ≥grade 4 cGVHD.

Subjects administered a composition of the disclosure (e.g., a cell population as described herein) exhibit a low incidence of mild to severe cGVHD, for example, a lower incidence of mild to severe cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure develop mild to severe cGVHD, for example, within 365 days post-transplant, two years post-transplant, or within another suitable amount of time post-transplant as disclosed herein.

Subjects administered a composition of the disclosure (e.g., a cell population as described herein) exhibit a low incidence of moderate to severe cGVHD, for example, a lower incidence of moderate to severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 110, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop moderate to severe cGVHD, for example, within 365 days post-transplant, two years post-transplant, or within another suitable amount of time post-transplant as disclosed herein.

In some embodiments, less than about 50% of subjects that are administered a composition of the disclosure develop moderate to severe cGVHD. In some embodiments, less than about 40% of subjects that are administered a composition of the disclosure develop moderate to severe cGVHD. In some embodiments, less than about 30% of subjects that are administered a composition of the disclosure develop moderate to severe cGVHD. In some embodiments, less than about 20% of subjects that are administered a composition of the disclosure develop moderate to severe cGVHD. In some embodiments, less than about 10% of subjects that are administered a composition of the disclosure develop moderate to severe cGVHD. In some embodiments, less than about 5% of subjects that are administered a composition of the disclosure develop moderate to severe cGVHD.

Subjects administered a composition of the disclosure (e.g., a cell population as described herein) exhibit a low incidence of severe cGVHD, for example, a lower incidence of severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop severe cGVHD, for example, within 365 days post-transplant, two years post-transplant, or within another suitable amount of time post-transplant as disclosed herein.

In some embodiments, less than about 50% of subjects that are administered a composition of the disclosure develop severe cGVHD. In some embodiments, less than about 40% of subjects that are administered a composition of the disclosure develop severe cGVHD. In some embodiments, less than about 30% of subjects that are administered a composition of the disclosure develop severe cGVHD. In some embodiments, less than about 20% of subjects that are administered a composition of the disclosure develop severe cGVHD. In some embodiments, less than about 15% of subjects that are administered a composition of the disclosure develop severe cGVHD. In some embodiments, less than about 10% of subjects that are administered a composition of the disclosure develop severe cGVHD. In some embodiments, less than about 5% of subjects that are administered a composition of the disclosure develop severe cGVHD. In some embodiments, less than about 3% of subjects that are administered a composition of the disclosure develop severe cGVHD. In some embodiments, less than about 2% of subjects that are administered a composition of the disclosure develop severe cGVHD. In some embodiments, less than about 1% of subjects that are administered a composition of the disclosure develop severe cGVHD.

The incidence of cGVHD can be assessed after a suitable amount of time elapses post-transplant, for example, about 150 days, about 200 days, about 365 days, about 1.5 years, about 2 years, about 2.5 years, about 3 years, about 3.5 years, about 4 years, about 4.5 years, or about 5 years post-transplant.

The incidence of cGVHD can be calculated based on a population of at least 10, at least at least 11, at least at least 12, at least 13, at least 14, at least 15, at least 16, at least 17, at least 18, at least 19, at least 20, at least 21, at least 22, at least 23, at least 24, at least 25, at least 25, at least 30, at least 35, at least 40, at least 45, at least 50, at least 60, at least 70, at least 80, at least 90, at least 100, at least 120, at least 140, at least 160, at least 180, at least 200, at least 250, at least 300, at least 350, at least 400, at least 450, or at least 500 subjects.

4. No GVHD Prophylaxis

Subjects administered a composition of the disclosure (e.g., a cell population as described herein) in the absence of GVHD prophylactic agents exhibit a low incidence of ≥grade 1 cGVHD, for example, a lower incidence of ≥grade 1 cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure in the absence of GVHD prophylactic agents develop ≥grade 1 cGVHD, for example, within 365 days post-transplant, two years post-transplant, or within another suitable amount of time post-transplant as disclosed herein.

Subjects administered a composition of the disclosure (e.g., a cell population as described herein) in the absence of GVHD prophylactic agents exhibit a low incidence of ≥grade 2 cGVHD, for example, a lower incidence of ≥grade 2 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop ≥grade 2 cGVHD, for example, within 365 days post-transplant, two years post-transplant, or within another suitable amount of time post-transplant as disclosed herein.

In some embodiments, less than about 50% of subjects that are administered a composition of the disclosure in the absence of GVHD prophylactic agents develop ≥grade 2 cGVHD. In some embodiments, less than about 40% of subjects that are administered a composition of the disclosure in the absence of GVHD prophylactic agents develop ≥grade 2 cGVHD. In some embodiments, less than about 30% of subjects that are administered a composition of the disclosure in the absence of GVHD prophylactic agents develop ≥grade 2 cGVHD. In some embodiments, less than about 25% of subjects that are administered a composition of the disclosure in the absence of GVHD prophylactic agents develop ≥grade 2 cGVHD. In some embodiments, less than about 20% of subjects that are administered a composition of the disclosure in the absence of GVHD prophylactic agents develop ≥grade 2 cGVHD. In some embodiments, less than about 10% of subjects that are administered a composition of the disclosure in the absence of GVHD prophylactic agents develop ≥grade 2 cGVHD. In some embodiments, less than about 5% of subjects that are administered a composition of the disclosure in the absence of GVHD prophylactic agents develop ≥grade 2 cGVHD.

Subjects administered a composition of the disclosure (e.g., a cell population as described herein) in the absence of GVHD prophylactic agents exhibit a low incidence of ≥grade 3 cGVHD, for example, a lower incidence of ≥grade 3 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop ≥grade 3 cGVHD, for example, within 365 days post-transplant, two years post-transplant, or within another suitable amount of time post-transplant as disclosed herein.

In some embodiments, less than about 50% of subjects that are administered a composition of the disclosure in the absence of GVHD prophylactic agents develop ≥grade 3 cGVHD. In some embodiments, less than about 40% of subjects that are administered a composition of the disclosure in the absence of GVHD prophylactic agents develop ≥grade 3 cGVHD. In some embodiments, less than about 30% of subjects that are administered a composition of the disclosure in the absence of GVHD prophylactic agents develop ≥grade 3 cGVHD. In some embodiments, less than about 20% of subjects that are administered a composition of the disclosure in the absence of GVHD prophylactic agents develop ≥grade 3 cGVHD. In some embodiments, less than about 10% of subjects that are administered a composition of the disclosure in the absence of GVHD prophylactic agents develop ≥grade 3 cGVHD. In some embodiments, less than about 5% of subjects that are administered a composition of the disclosure in the absence of GVHD prophylactic agents develop ≥grade 3 cGVHD. In some embodiments, less than about 3% of subjects that are administered a composition of the disclosure in the absence of GVHD prophylactic agents develop ≥grade 3 cGVHD. In some embodiments, less than about 2% of subjects that are administered a composition of the disclosure in the absence of GVHD prophylactic agents develop ≥grade 3 cGVHD. In some embodiments, less than about 1% of subjects that are administered a composition of the disclosure in the absence of GVHD prophylactic agents develop ≥grade 3 cGVHD.

Subjects administered a composition of the disclosure (e.g., a cell population as described herein) in the absence of GVHD prophylactic agents exhibit a low incidence of ≥grade 4 cGVHD, for example, a lower incidence of ≥grade 4 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 40%, or less than about 50% of subjects that are administered a composition of the disclosure in the absence of GVHD prophylactic agents develop ≥grade 4 cGVHD, for example, within 365 days post-transplant, two years post-transplant, or within another suitable amount of time post-transplant as disclosed herein.

In some embodiments, less than about 30% of subjects that are administered a composition of the disclosure in the absence of GVHD prophylactic agents develop ≥grade 4 cGVHD. In some embodiments, less than about 20% of subjects that are administered a composition of the disclosure in the absence of GVHD prophylactic agents develop ≥grade 4 cGVHD. In some embodiments, less than about 15% of subjects that are administered a composition of the disclosure in the absence of GVHD prophylactic agents develop ≥grade 4 cGVHD. In some embodiments, less than about 10% of subjects that are administered a composition of the disclosure in the absence of GVHD prophylactic agents develop ≥grade 4 cGVHD. In some embodiments, less than about 5% of subjects that are administered a composition of the disclosure in the absence of GVHD prophylactic agents develop ≥grade 4 cGVHD. In some embodiments, less than about 3% of subjects that are administered a composition of the disclosure in the absence of GVHD prophylactic agents develop ≥grade 4 cGVHD. In some embodiments, less than about 2% of subjects that are administered a composition of the disclosure in the absence of GVHD prophylactic agents develop ≥grade 4 cGVHD. In some embodiments, less than about 1% of subjects that are administered a composition of the disclosure in the absence of GVHD prophylactic agents develop ≥grade 4 cGVHD.

Subjects administered a composition of the disclosure (e.g., a cell population as described herein) in the absence of GVHD prophylactic agents exhibit a low incidence of mild to severe cGVHD, for example, a lower incidence of mild to severe cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure in the absence of GVHD prophylactic agents develop mild to severe cGVHD, for example, within 365 days post-transplant, two years post-transplant, or within another suitable amount of time post-transplant as disclosed herein.

Subjects administered a composition of the disclosure (e.g., a cell population as described herein) in the absence of GVHD prophylactic agents exhibit a low incidence of moderate to severe cGVHD, for example, a lower incidence of moderate to severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop moderate to severe cGVHD, for example, within 365 days post-transplant, two years post-transplant, or within another suitable amount of time post-transplant as disclosed herein.

In some embodiments, less than about 50% of subjects that are administered a composition of the disclosure in the absence of GVHD prophylactic agents develop moderate to severe cGVHD. In some embodiments, less than about 40% of subjects that are administered a composition of the disclosure in the absence of GVHD prophylactic agents develop moderate to severe cGVHD. In some embodiments, less than about 30% of subjects that are administered a composition of the disclosure in the absence of GVHD prophylactic agents develop moderate to severe cGVHD. In some embodiments, less than about 25% of subjects that are administered a composition of the disclosure in the absence of GVHD prophylactic agents develop moderate to severe cGVHD. In some embodiments, less than about 20% of subjects that are administered a composition of the disclosure in the absence of GVHD prophylactic agents develop moderate to severe cGVHD. In some embodiments, less than about 10% of subjects that are administered a composition of the disclosure in the absence of GVHD prophylactic agents develop moderate to severe cGVHD. In some embodiments, less than about 5% of subjects that are administered a composition of the disclosure in the absence of GVHD prophylactic agents develop moderate to severe cGVHD.

Subjects administered a composition of the disclosure (e.g., a cell population as described herein) in the absence of GVHD prophylactic agents exhibit a low incidence of severe cGVHD, for example, a lower incidence of severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop severe cGVHD, for example, within 365 days post-transplant, two years post-transplant, or within another suitable amount of time post-transplant as disclosed herein.

In some embodiments, less than about 50% of subjects that are administered a composition of the disclosure in the absence of GVHD prophylactic agents develop severe cGVHD. In some embodiments, less than about 40% of subjects that are administered a composition of the disclosure in the absence of GVHD prophylactic agents develop severe cGVHD. In some embodiments, less than about 30% of subjects that are administered a composition of the disclosure in the absence of GVHD prophylactic agents develop severe cGVHD. In some embodiments, less than about 20% of subjects that are administered a composition of the disclosure in the absence of GVHD prophylactic agents develop severe cGVHD. In some embodiments, less than about 10% of subjects that are administered a composition of the disclosure in the absence of GVHD prophylactic agents develop severe cGVHD. In some embodiments, less than about 5% of subjects that are administered a composition of the disclosure in the absence of GVHD prophylactic agents develop severe cGVHD. In some embodiments, less than about 3% of subjects that are administered a composition of the disclosure in the absence of GVHD prophylactic agents develop severe cGVHD. In some embodiments, less than about 2% of subjects that are administered a composition of the disclosure in the absence of GVHD prophylactic agents develop severe cGVHD. In some embodiments, less than about 1% of subjects that are administered a composition of the disclosure in the absence of GVHD prophylactic agents develop severe cGVHD.

The absence of GVHD prophylactic agents can refer to cases where no GVHD prophylactic agents are administered to the subject for the first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years post-transplant.

5. Single Agent GVHD Prophylaxis

Subjects administered a composition of the disclosure (e.g., a cell population as described herein) and no more than one GVHD prophylactic agent (for example, a single GVHD prophylactic agent) exhibit a low incidence of ≥grade 1 cGVHD, for example, a lower incidence of ≥grade 1 cGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and no more than one GVHD prophylactic agent (for example, a single GVHD prophylactic agent) develop ≥grade 1 cGVHD, for example, within 365 days post-transplant, two years post-transplant, or within another suitable amount of time post-transplant as disclosed herein.

Subjects administered a composition of the disclosure (e.g., a cell population as described herein) and no more than one GVHD prophylactic agent (for example, a single GVHD prophylactic agent) exhibit a low incidence of ≥grade 2 cGVHD, for example, a lower incidence of ≥grade 2 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop ≥grade 2 cGVHD, for example, within 365 days post-transplant, two years post-transplant, or within another suitable amount of time post-transplant as disclosed herein.

In some embodiments, less than about 50% of subjects that are administered a composition of the disclosure and no more than one GVHD prophylactic agent (for example, a single GVHD prophylactic agent) develop ≥grade 2 cGVHD. In some embodiments, less than about 40% of subjects that are administered a composition of the disclosure and no more than one GVHD prophylactic agent (for example, a single GVHD prophylactic agent) develop ≥grade 2 cGVHD. In some embodiments, less than about 30% of subjects that are administered a composition of the disclosure and no more than one GVHD prophylactic agent (for example, a single GVHD prophylactic agent) develop ≥grade 2 cGVHD. In some embodiments, less than about 20% of subjects that are administered a composition of the disclosure and no more than one GVHD prophylactic agent (for example, a single GVHD prophylactic agent) develop ≥grade 2 cGVHD. In some embodiments, less than about 10% of subjects that are administered a composition of the disclosure and no more than one GVHD prophylactic agent (for example, a single GVHD prophylactic agent) develop ≥grade 2 cGVHD. In some embodiments, less than about 5% of subjects that are administered a composition of the disclosure and no more than one GVHD prophylactic agent (for example, a single GVHD prophylactic agent) develop ≥grade 2 cGVHD.

Subjects administered a composition of the disclosure (e.g., a cell population as described herein) and no more than one GVHD prophylactic agent (for example, a single GVHD prophylactic agent) exhibit a low incidence of ≥grade 3 cGVHD, for example, a lower incidence of ≥grade 3 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop ≥grade 3 cGVHD, for example, within 365 days post-transplant, two years post-transplant, or within another suitable amount of time post-transplant as disclosed herein.

In some embodiments, less than about 50% of subjects that are administered a composition of the disclosure and no more than one GVHD prophylactic agent (for example, a single GVHD prophylactic agent) develop ≥grade 3 cGVHD. In some embodiments, less than about 40% of subjects that are administered a composition of the disclosure and no more than one GVHD prophylactic agent (for example, a single GVHD prophylactic agent) develop ≥grade 3 cGVHD. In some embodiments, less than about 30% of subjects that are administered a composition of the disclosure and no more than one GVHD prophylactic agent (for example, a single GVHD prophylactic agent) develop ≥grade 3 cGVHD. In some embodiments, less than about 20% of subjects that are administered a composition of the disclosure and no more than one GVHD prophylactic agent (for example, a single GVHD prophylactic agent) develop ≥grade 3 cGVHD. In some embodiments, less than about 15% of subjects that are administered a composition of the disclosure and no more than one GVHD prophylactic agent (for example, a single GVHD prophylactic agent) develop ≥grade 3 cGVHD. In some embodiments, less than about 10% of subjects that are administered a composition of the disclosure and no more than one GVHD prophylactic agent (for example, a single GVHD prophylactic agent) develop ≥grade 3 cGVHD. In some embodiments, less than about 5% of subjects that are administered a composition of the disclosure and no more than one GVHD prophylactic agent (for example, a single GVHD prophylactic agent) develop ≥grade 3 cGVHD. In some embodiments, less than about 3% of subjects that are administered a composition of the disclosure and no more than one GVHD prophylactic agent (for example, a single GVHD prophylactic agent) develop ≥grade 3 cGVHD. In some embodiments, less than about 2% of subjects that are administered a composition of the disclosure and no more than one GVHD prophylactic agent (for example, a single GVHD prophylactic agent) develop ≥grade 3 cGVHD. In some embodiments, less than about 1% of subjects that are administered a composition of the disclosure and no more than one GVHD prophylactic agent (for example, a single GVHD prophylactic agent) develop ≥grade 3 cGVHD.

Subjects administered a composition of the disclosure (e.g., a cell population as described herein) and no more than one GVHD prophylactic agent (for example, a single GVHD prophylactic agent) exhibit a low incidence of ≥grade 4 cGVHD, for example, a lower incidence of ≥grade 4 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure and no more than one GVHD prophylactic agent (for example, a single GVHD prophylactic agent) develop ≥grade 4 cGVHD, for example, within 365 days post-transplant, two years post-transplant, or within another suitable amount of time post-transplant as disclosed herein.

In some embodiments, less than about 40% of subjects that are administered a composition of the disclosure and no more than one GVHD prophylactic agent (for example, a single GVHD prophylactic agent) develop ≥grade 4 cGVHD. In some embodiments, less than about 30% of subjects that are administered a composition of the disclosure and no more than one GVHD prophylactic agent (for example, a single GVHD prophylactic agent) develop ≥grade 4 cGVHD. In some embodiments, less than about 20% of subjects that are administered a composition of the disclosure and no more than one GVHD prophylactic agent (for example, a single GVHD prophylactic agent) develop ≥grade 4 cGVHD. In some embodiments, less than about 10% of subjects that are administered a composition of the disclosure and no more than one GVHD prophylactic agent (for example, a single GVHD prophylactic agent) develop ≥grade 4 cGVHD. In some embodiments, less than about 5% of subjects that are administered a composition of the disclosure and no more than one GVHD prophylactic agent (for example, a single GVHD prophylactic agent) develop ≥grade 4 cGVHD. In some embodiments, less than about 3% of subjects that are administered a composition of the disclosure and no more than one GVHD prophylactic agent (for example, a single GVHD prophylactic agent) develop ≥grade 4 cGVHD. In some embodiments, less than about 2% of subjects that are administered a composition of the disclosure and no more than one GVHD prophylactic agent (for example, a single GVHD prophylactic agent) develop ≥grade 4 cGVHD. In some embodiments, less than about 1% of subjects that are administered a composition of the disclosure and no more than one GVHD prophylactic agent (for example, a single GVHD prophylactic agent) develop ≥grade 4 cGVHD.

Subjects administered a composition of the disclosure (e.g., a cell population as described herein) and no more than one GVHD prophylactic agent (for example, a single GVHD prophylactic agent) exhibit a low incidence of mild to severe cGVHD, for example, a lower incidence of mild to severe cGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and no more than one GVHD prophylactic agent (for example, a single GVHD prophylactic agent) develop mild to severe cGVHD, for example, within 365 days post-transplant, two years post-transplant, or within another suitable amount of time post-transplant as disclosed herein.

Subjects administered a composition of the disclosure (e.g., a cell population as described herein) and no more than one GVHD prophylactic agent (for example, a single GVHD prophylactic agent) exhibit a low incidence of moderate to severe cGVHD, for example, a lower incidence of moderate to severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop moderate to severe cGVHD, for example, within 365 days post-transplant, two years post-transplant, or within another suitable amount of time post-transplant as disclosed herein.

In some embodiments, less than about 50% of subjects that are administered a composition of the disclosure and no more than one GVHD prophylactic agent (for example, a single GVHD prophylactic agent) develop moderate to severe cGVHD. In some embodiments, less than about 40% of subjects that are administered a composition of the disclosure and no more than one GVHD prophylactic agent (for example, a single GVHD prophylactic agent) develop moderate to severe cGVHD. In some embodiments, less than about 30% of subjects that are administered a composition of the disclosure and no more than one GVHD prophylactic agent (for example, a single GVHD prophylactic agent) develop moderate to severe cGVHD. In some embodiments, less than about 20% of subjects that are administered a composition of the disclosure and no more than one GVHD prophylactic agent (for example, a single GVHD prophylactic agent) develop moderate to severe cGVHD. In some embodiments, less than about 10% of subjects that are administered a composition of the disclosure and no more than one GVHD prophylactic agent (for example, a single GVHD prophylactic agent) develop moderate to severe cGVHD. In some embodiments, less than about 5% of subjects that are administered a composition of the disclosure and no more than one GVHD prophylactic agent (for example, a single GVHD prophylactic agent) develop moderate to severe cGVHD.

Subjects administered a composition of the disclosure (e.g., a cell population as described herein) and no more than one GVHD prophylactic agent (for example, a single GVHD prophylactic agent) exhibit a low incidence of severe cGVHD, for example, a lower incidence of severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop severe cGVHD, for example, within 365 days post-transplant, two years post-transplant, or within another suitable amount of time post-transplant as disclosed herein.

In some embodiments, less than about 50% of subjects that are administered a composition of the disclosure and no more than one GVHD prophylactic agent (for example, a single GVHD prophylactic agent) develop severe cGVHD. In some embodiments, less than about 40% of subjects that are administered a composition of the disclosure and no more than one GVHD prophylactic agent (for example, a single GVHD prophylactic agent) develop severe cGVHD. In some embodiments, less than about 30% of subjects that are administered a composition of the disclosure and no more than one GVHD prophylactic agent (for example, a single GVHD prophylactic agent) develop severe cGVHD. In some embodiments, less than about 20% of subjects that are administered a composition of the disclosure and no more than one GVHD prophylactic agent (for example, a single GVHD prophylactic agent) develop severe cGVHD. In some embodiments, less than about 15% of subjects that are administered a composition of the disclosure and no more than one GVHD prophylactic agent (for example, a single GVHD prophylactic agent) develop severe cGVHD. In some embodiments, less than about 10% of subjects that are administered a composition of the disclosure and no more than one GVHD prophylactic agent (for example, a single GVHD prophylactic agent) develop severe cGVHD. In some embodiments, less than about 5% of subjects that are administered a composition of the disclosure and no more than one GVHD prophylactic agent (for example, a single GVHD prophylactic agent) develop severe cGVHD. In some embodiments, less than about 3% of subjects that are administered a composition of the disclosure and no more than one GVHD prophylactic agent (for example, a single GVHD prophylactic agent) develop severe cGVHD. In some embodiments, less than about 2% of subjects that are administered a composition of the disclosure and no more than one GVHD prophylactic agent (for example, a single GVHD prophylactic agent) develop severe cGVHD. In some embodiments, less than about 1% of subjects that are administered a composition of the disclosure and no more than one GVHD prophylactic agent (for example, a single GVHD prophylactic agent) develop severe cGVHD.

A single GVHD prophylactic agent can be tacrolimus.

A single GVHD prophylactic agent can be sirolimus.

The no more than one GVHD prophylactic agent (for example, a single GVHD prophylactic agent) can be administered to the subject for the first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years post-transplant. In some embodiments, the no more than one GVHD prophylactic agent (for example, a single GVHD prophylactic agent) is administered to the subject for less than about 20 days, less than about 30 days, less than about 40 days, less than about 50 days, less than about 60 days, less than about 70 days, less than about 80 days, less than about 90 days, less than about 100 days, less than about 110 days, less than about 120 days, less than about 150 days, less than about 200 days, less than about 365 days, less than about 13 months, less than about 14 months, less than about 15 months, less than about 16 months, less than about 17 months, less than about 18 months, less than about 19 months, less than about 20 months, less than about 21 months, less than about 22 months, less than about 23 months, less than about 2 years, less than about 2.5 years, less than about 3 years, less than about 3.5 years, less than about 4 years, less than about 4.5 years, or less than about or 5 years post-transplant.

6. Low Dose GVHD Prophylaxis

Subjects administered a composition of the disclosure (e.g., a cell population as described herein) and a low dose of a GVHD prophylactic agent (for example, a single GVHD prophylactic agent at a low dose) exhibit a low incidence of ≥grade 1 cGVHD, for example, a lower incidence of ≥grade 1 cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and a low dose of a GVHD prophylactic agent (for example, a single GVHD prophylactic agent at a low dose) develop ≥grade 1 cGVHD, for example, within 365 days post-transplant, two years post-transplant, or within another suitable amount of time post-transplant as disclosed herein.

Subjects administered a composition of the disclosure (e.g., a cell population as described herein) and a low dose of a GVHD prophylactic agent (for example, a single GVHD prophylactic agent at a low dose) exhibit a low incidence of ≥grade 2 cGVHD, for example, a lower incidence of ≥grade 2 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop ≥grade 2 cGVHD, for example, within 365 days post-transplant, two years post-transplant, or within another suitable amount of time post-transplant as disclosed herein.

In some embodiments, less than about 50% of subjects that are administered a composition of the disclosure and a low dose of a GVHD prophylactic agent (for example, a single GVHD prophylactic agent at a low dose) develop ≥grade 2 cGVHD. In some embodiments, less than about 40% of subjects that are administered a composition of the disclosure and a low dose of a GVHD prophylactic agent (for example, a single GVHD prophylactic agent at a low dose) develop ≥grade 2 cGVHD. In some embodiments, less than about 30% of subjects that are administered a composition of the disclosure and a low dose of a GVHD prophylactic agent (for example, a single GVHD prophylactic agent at a low dose) develop ≥grade 2 cGVHD. In some embodiments, less than about 20% of subjects that are administered a composition of the disclosure and a low dose of a GVHD prophylactic agent (for example, a single GVHD prophylactic agent at a low dose) develop ≥grade 2 cGVHD. In some embodiments, less than about 15% of subjects that are administered a composition of the disclosure and a low dose of a GVHD prophylactic agent (for example, a single GVHD prophylactic agent at a low dose) develop ≥grade 2 cGVHD. In some embodiments, less than about 10% of subjects that are administered a composition of the disclosure and a low dose of a GVHD prophylactic agent (for example, a single GVHD prophylactic agent at a low dose) develop ≥grade 2 cGVHD. In some embodiments, less than about 5% of subjects that are administered a composition of the disclosure and a low dose of a GVHD prophylactic agent (for example, a single GVHD prophylactic agent at a low dose) develop ≥grade 2 cGVHD.

Subjects administered a composition of the disclosure (e.g., a cell population as described herein) and a low dose of a GVHD prophylactic agent (for example, a single GVHD prophylactic agent at a low dose) exhibit a low incidence of ≥grade 3 cGVHD, for example, a lower incidence of ≥grade 3 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop ≥grade 3 cGVHD, for example, within 365 days post-transplant, two years post-transplant, or within another suitable amount of time post-transplant as disclosed herein.

In some embodiments, less than about 50% of subjects that are administered a composition of the disclosure and a low dose of a GVHD prophylactic agent (for example, a single GVHD prophylactic agent at a low dose) develop ≥grade 3 cGVHD. In some embodiments, less than about 40% of subjects that are administered a composition of the disclosure and a low dose of a GVHD prophylactic agent (for example, a single GVHD prophylactic agent at a low dose) develop ≥grade 3 cGVHD. In some embodiments, less than about 30% of subjects that are administered a composition of the disclosure and a low dose of a GVHD prophylactic agent (for example, a single GVHD prophylactic agent at a low dose) develop ≥grade 3 cGVHD. In some embodiments, less than about 20% of subjects that are administered a composition of the disclosure and a low dose of a GVHD prophylactic agent (for example, a single GVHD prophylactic agent at a low dose) develop ≥grade 3 cGVHD. In some embodiments, less than about 15% of subjects that are administered a composition of the disclosure and a low dose of a GVHD prophylactic agent (for example, a single GVHD prophylactic agent at a low dose) develop ≥grade 3 cGVHD. In some embodiments, less than about 10% of subjects that are administered a composition of the disclosure and a low dose of a GVHD prophylactic agent (for example, a single GVHD prophylactic agent at a low dose) develop ≥grade 3 cGVHD. In some embodiments, less than about 5% of subjects that are administered a composition of the disclosure and a low dose of a GVHD prophylactic agent (for example, a single GVHD prophylactic agent at a low dose) develop ≥grade 3 cGVHD. In some embodiments, less than about 3% of subjects that are administered a composition of the disclosure and a low dose of a GVHD prophylactic agent (for example, a single GVHD prophylactic agent at a low dose) develop ≥grade 3 cGVHD. In some embodiments, less than about 2% of subjects that are administered a composition of the disclosure and a low dose of a GVHD prophylactic agent (for example, a single GVHD prophylactic agent at a low dose) develop ≥grade 3 cGVHD. In some embodiments, less than about 1% of subjects that are administered a composition of the disclosure and a low dose of a GVHD prophylactic agent (for example, a single GVHD prophylactic agent at a low dose) develop ≥grade 3 cGVHD.

Subjects administered a composition of the disclosure (e.g., a cell population as described herein) and a low dose of a GVHD prophylactic agent (for example, a single GVHD prophylactic agent at a low dose) exhibit a low incidence of ≥grade 4 cGVHD, for example, a lower incidence of ≥grade 4 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure and a low dose of a GVHD prophylactic agent (for example, a single GVHD prophylactic agent at a low dose) develop ≥grade 4 cGVHD, for example, within 365 days post-transplant, two years post-transplant, or within another suitable amount of time post-transplant as disclosed herein.

In some embodiments, less than about 40% of subjects that are administered a composition of the disclosure and a low dose of a GVHD prophylactic agent (for example, a single GVHD prophylactic agent at a low dose) develop ≥grade 4 cGVHD. In some embodiments, less than about 30% of subjects that are administered a composition of the disclosure and a low dose of a GVHD prophylactic agent (for example, a single GVHD prophylactic agent at a low dose) develop ≥grade 4 cGVHD. In some embodiments, less than about 20% of subjects that are administered a composition of the disclosure and a low dose of a GVHD prophylactic agent (for example, a single GVHD prophylactic agent at a low dose) develop ≥grade 4 cGVHD. In some embodiments, less than about 10% of subjects that are administered a composition of the disclosure and a low dose of a GVHD prophylactic agent (for example, a single GVHD prophylactic agent at a low dose) develop ≥grade 4 cGVHD. In some embodiments, less than about 5% of subjects that are administered a composition of the disclosure and a low dose of a GVHD prophylactic agent (for example, a single GVHD prophylactic agent at a low dose) develop ≥grade 4 cGVHD. In some embodiments, less than about 3% of subjects that are administered a composition of the disclosure and a low dose of a GVHD prophylactic agent (for example, a single GVHD prophylactic agent at a low dose) develop ≥grade 4 cGVHD. In some embodiments, less than about 2% of subjects that are administered a composition of the disclosure and a low dose of a GVHD prophylactic agent (for example, a single GVHD prophylactic agent at a low dose) develop ≥grade 4 cGVHD. In some embodiments, less than about 1% of subjects that are administered a composition of the disclosure and a low dose of a GVHD prophylactic agent (for example, a single GVHD prophylactic agent at a low dose) develop ≥grade 4 cGVHD.

Subjects administered a composition of the disclosure (e.g., a cell population as described herein) and a low dose of a GVHD prophylactic agent (for example, a single GVHD prophylactic agent at a low dose) exhibit a low incidence of mild to severe cGVHD, for example, a lower incidence of mild to severe cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and a low dose of a GVHD prophylactic agent (for example, a single GVHD prophylactic agent at a low dose) develop mild to severe cGVHD, for example, within 365 days post-transplant, two years post-transplant, or within another suitable amount of time post-transplant as disclosed herein.

Subjects administered a composition of the disclosure (e.g., a cell population as described herein) and a low dose of a GVHD prophylactic agent (for example, a single GVHD prophylactic agent at a low dose) exhibit a low incidence of moderate to severe cGVHD, for example, a lower incidence of moderate to severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop moderate to severe cGVHD, for example, within 365 days post-transplant, two years post-transplant, or within another suitable amount of time post-transplant as disclosed herein.

In some embodiments, less than about 50% of subjects that are administered a composition of the disclosure and a low dose of a GVHD prophylactic agent (for example, a single GVHD prophylactic agent at a low dose) develop moderate to severe cGVHD. In some embodiments, less than about 40% of subjects that are administered a composition of the disclosure and a low dose of a GVHD prophylactic agent (for example, a single GVHD prophylactic agent at a low dose) develop moderate to severe cGVHD. In some embodiments, less than about 30% of subjects that are administered a composition of the disclosure and a low dose of a GVHD prophylactic agent (for example, a single GVHD prophylactic agent at a low dose) develop moderate to severe cGVHD. In some embodiments, less than about 20% of subjects that are administered a composition of the disclosure and a low dose of a GVHD prophylactic agent (for example, a single GVHD prophylactic agent at a low dose) develop moderate to severe cGVHD. In some embodiments, less than about 15% of subjects that are administered a composition of the disclosure and a low dose of a GVHD prophylactic agent (for example, a single GVHD prophylactic agent at a low dose) develop moderate to severe cGVHD. In some embodiments, less than about 10% of subjects that are administered a composition of the disclosure and a low dose of a GVHD prophylactic agent (for example, a single GVHD prophylactic agent at a low dose) develop moderate to severe cGVHD. In some embodiments, less than about 5% of subjects that are administered a composition of the disclosure and a low dose of a GVHD prophylactic agent (for example, a single GVHD prophylactic agent at a low dose) develop moderate to severe cGVHD.

Subjects administered a composition of the disclosure (e.g., a cell population as described herein) and a low dose of a GVHD prophylactic agent (for example, a single GVHD prophylactic agent at a low dose) exhibit a low incidence of severe cGVHD, for example, a lower incidence of severe cGVHD than subjects that are administered an alternate composition. In some embodiments, less than about 10%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop severe cGVHD, for example, within 365 days post-transplant, two years post-transplant, or within another suitable amount of time post-transplant as disclosed herein.

In some embodiments, less than about 50% of subjects that are administered a composition of the disclosure and a low dose of a GVHD prophylactic agent (for example, a single GVHD prophylactic agent at a low dose) develop severe cGVHD. In some embodiments, less than about 40% of subjects that are administered a composition of the disclosure and a low dose of a GVHD prophylactic agent (for example, a single GVHD prophylactic agent at a low dose) develop severe cGVHD. In some embodiments, less than about 30% of subjects that are administered a composition of the disclosure and a low dose of a GVHD prophylactic agent (for example, a single GVHD prophylactic agent at a low dose) develop severe cGVHD. In some embodiments, less than about 20% of subjects that are administered a composition of the disclosure and a low dose of a GVHD prophylactic agent (for example, a single GVHD prophylactic agent at a low dose) develop severe cGVHD. In some embodiments, less than about 15% of subjects that are administered a composition of the disclosure and a low dose of a GVHD prophylactic agent (for example, a single GVHD prophylactic agent at a low dose) develop severe cGVHD. In some embodiments, less than about 10% of subjects that are administered a composition of the disclosure and a low dose of a GVHD prophylactic agent (for example, a single GVHD prophylactic agent at a low dose) develop severe cGVHD. In some embodiments, less than about 5% of subjects that are administered a composition of the disclosure and a low dose of a GVHD prophylactic agent (for example, a single GVHD prophylactic agent at a low dose) develop severe cGVHD. In some embodiments, less than about 3% of subjects that are administered a composition of the disclosure and a low dose of a GVHD prophylactic agent (for example, a single GVHD prophylactic agent at a low dose) develop severe cGVHD. In some embodiments, less than about 2% of subjects that are administered a composition of the disclosure and a low dose of a GVHD prophylactic agent (for example, a single GVHD prophylactic agent at a low dose) develop severe cGVHD. In some embodiments, less than about 1% of subjects that are administered a composition of the disclosure and a low dose of a GVHD prophylactic agent (for example, a single GVHD prophylactic agent at a low dose) develop severe cGVHD.

A low dose of a GVHD prophylactic agent can be, for example a target trough level of less than about 25 ng/mL, less than about 20 ng/mL, less than about 15 ng/mL, less than about 12 ng/mL, less than about 11 ng/mL, less than about 10 ng/mL, less than about 9 ng/mL, less than about 8 ng/mL, less than about 7 ng/mL, less than about 6 ng/mL, less than about 5 ng/mL, less than about 4 ng/mL, less than about 3 ng/mL, less than about 2 ng/mL, or less than about 1 ng/mL.

In some embodiments, a low dose of a GVHD prophylactic is a target trough level of about 1-25 ng/mL, about 1-20 ng/mL, about 1-15 ng/mL, about 1-12 ng/mL, about 1-11 ng/mL, about 1-10 ng/mL, about 1-9 ng/mL, about 1-8 ng/mL, about 1-7 ng/mL, about 1-6 ng/mL, about 1-5 ng/mL, about 1-4 ng/mL, about 1-3 ng/mL, about 1-2 ng/mL, about 2-25 ng/mL, about 2-20 ng/mL, about 2-15 ng/mL, about 2-12 ng/mL, about 2-11 ng/mL, about 2-10 ng/mL, about 2-9 ng/mL, about 2-8 ng/mL, about 2-7 ng/mL, about 2-6 ng/mL, about 2-5 ng/mL, about 2-4 ng/mL, about 2-3 ng/mL, about 3-25 ng/mL, about 3-20 ng/mL, about 3-15 ng/mL, about 3-12 ng/mL, about 3-11 ng/mL, about 3-10 ng/mL, about 3-9 ng/mL, about 3-8 ng/mL, about 3-7 ng/mL, about 3-6 ng/mL, about 3-5 ng/mL, about 3-4 ng/mL, about 4-25 ng/mL, about 4-20 ng/mL, about 4-15 ng/mL, about 4-12 ng/mL, about 4-11 ng/mL, about 4-10 ng/mL, about 4-9 ng/mL, about 4-8 ng/mL, about 4-7 ng/mL, about 4-6 ng/mL, about 4-5 ng/mL, about 5-25 ng/mL, about 5-20 ng/mL, about 5-15 ng/mL, about 5-12 ng/mL, about 5-11 ng/mL, about 5-10 ng/mL, about 5-9 ng/mL, about 5-8 ng/mL, about 5-7 ng/mL, about 5-6 ng/mL, about 6-25 g/mL, about 6-20 ng/mL, about 6-15 ng/mL, about 6-12 ng/mL, about 6-11 ng/mL, about 6-10 ng/mL, about 6-9 ng/mL, about 6-8 ng/mL, about 6-7 ng/mL, about 8-25 ng/mL, about 8-20 ng/mL, about 8-15 ng/mL, about 8-12 ng/mL, about 8-11 ng/mL, about 8-10 ng/mL, about 8-9 ng/mL, about 10-25 ng/mL, about 10-20 ng/mL, about 10-15 ng/mL, about 10-12 ng/mL, or about 10-11 ng/mL.

In some embodiments, a low dose of a GVHD prophylactic agent is tacrolimus with a target trough level of about 5 ng/mL to about 10 ng/mL. In some embodiments, a low dose of a GVHD prophylactic agent is tacrolimus with a target trough level of about 4 ng/mL to about 6 ng/mL.

In some embodiments, a low dose of a GVHD prophylactic agent is sirolimus with a target trough level of about 3 ng/mL to about 8 ng/mL. In some embodiments, a low dose of a GVHD prophylactic agent is sirolimus with a target trough level of about 4 ng/mL to about 8 ng/mL.

The low dose GVHD prophylactic agent (for example, single GVHD prophylactic agent at a low dose) can be administered to the subject for the first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years post-transplant. In some embodiments, the low dose GVHD prophylactic agent (for example, single GVHD prophylactic agent at a low dose) is administered to the subject for less than about 20 days, less than about 30 days, less than about 40 days, less than about 50 days, less than about 60 days, less than about 70 days, less than about 80 days, less than about 90 days, less than about 100 days, less than about 110 days, less than about 120 days, less than about 150 days, less than about 200 days, less than about 365 days, less than about 13 months, less than about 14 months, less than about 15 months, less than about 16 months, less than about 17 months, less than about 18 months, less than about 19 months, less than about 20 months, less than about 21 months, less than about 22 months, less than about 23 months, less than about 2 years, less than about 2.5 years, less than about 3 years, less than about 3.5 years, less than about 4 years, less than about 4.5 years, or less than about or 5 years post-transplant.

### E. Organ-Specific GVHD Stage Incidence

Subjects administered a composition of the disclosure (e.g., a cell population as described herein) exhibit a low incidence of ≥stage 1 GVHD signs for the skin, liver, gut, or a combination thereof, for example, a lower incidence compared to subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure exhibit ≥stage 1 GVHD signs for the skin, liver, gut, or a combination thereof.

Subjects administered a composition of the disclosure (e.g., a cell population as described herein) exhibit a low incidence of ≥stage 2 GVHD signs for the skin, liver, gut, or a combination thereof, for example, a lower incidence compared to subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure exhibit ≥stage 1 GVHD signs for the skin, liver, gut, or a combination thereof.

Subjects administered a composition of the disclosure (e.g., a cell population as described herein) exhibit a low incidence of ≥stage 3 GVHD signs for the skin, liver, gut, or a combination thereof, for example, a lower incidence compared to subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure exhibit ≥stage 3 GVHD signs for the skin, liver, gut, or a combination thereof.

Subjects administered a composition of the disclosure (e.g., a cell population as described herein) exhibit a low incidence of ≥stage 4 GVHD signs for the skin, liver, gut, or a combination thereof, for example, a lower incidence compared to subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure exhibit ≥stage 4 GVHD signs for the skin, liver, gut, or a combination thereof.

The incidence of the organ-specific GVHD signs can be assessed after a suitable amount of time elapses post-transplant, for example, about 20 days, about 21 days, about 25 days, about 28 days, about 30 days, about 35 days, about 40 days, about 42 days, about 45 days, about 49 days, about 50 days, about 55 days, about 56 days, about 60 days, about 63 days, about 65 days, about 70 days, about 75 days, about 77 days, about 80 days, about 84 days, about 85 days, about 90 days, about 91 days, about 95 days, about 98 days, about 100 days, about 105 days, about 110 days, about 112 days, about 115 days, about 119 days, about 120 days, about 150 days, about 200 days, about 365 days, about 1.5 years, about 2 years, about 2.5 years, about 3 years, about 3.5 years, about 4 years, about 4.5 years, or about 5 years post-transplant.

The incidence of the organ-specific GVHD signs can be calculated based on a population of, for example, at least 10, at least at least 11, at least at least 12, at least 13, at least 14, at least 15, at least 16, at least 17, at least 18, at least 19, at least 20, at least 21, at least 22, at least 23, at least 24, at least 25, at least 25, at least 30, at least 35, at least 40, at least 45, at least 50, at least 60, at least 70, at least 80, at least 90, at least 100, at least 120, at least 140, at least 160, at least 180, at least 200, at least 250, at least 300, at least 350, at least 400, at least 450, or at least 500 subjects.

In some embodiments, at least about 50%, at least about 55%, at least about 60%, at least about 65%, at least about 70%, at least about 75%, at least about 80%, at least about 85%, at least about 90%, or at least about 95% of subjects have a CD4:CD8 T cell ratio of ≥1.5 when evaluated after a suitable amount of time post-transplant, for example, at about 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

### V. Other Clinical Outcomes

### A. Survival, Hospitalization, and Relapse

In some embodiments, patient treated or provided with compositions, multi-component pharmaceutical treatments, cell populations, solutions, formulations, kits, and/or methods has a reduced risk of at least one of malignancy relapse, infection or renal failure.

A population of subjects treated or provided with compositions, multi-component pharmaceutical treatments, cell populations, solutions, formulations, kits, and/or methods of the disclosure (e.g., a cell population comprising a population of cells described herein) exhibit a high overall survival rate, for example, a higher overall survival rate compared to subjects that are administered an alternate composition. In some embodiments, at least about 40%, at least about 50%, at least about 55%, at least about 60%, at least about 65%, at least about 70%, at least about 75%, at least about 80%, at least about 85%, at least about 90%, or at least about 95% of subjects that are administered a composition of the disclosure are alive after about 1 year, about 1.5 years, about 2 years, about 2.5 years, about 3 years, about 3.5 years, about 4 years, about 4.5 years, about 5 years, about 7 years, about 10 years, about 15 years, about 20 years, about 30 years post-transplant.

A population of subjects treated or provided with compositions, multi-component pharmaceutical treatments, cell populations, solutions, formulations, kits, and/or methods of the disclosure (e.g., a cell population comprising a population of cells described herein) exhibit a low treatment-associated mortality rate, for example, a lower treatment-associated mortality rate compared to subjects that are administered an alternate composition. In some embodiments, a treatment-associated mortality rate of a population of subjects administered a composition of the disclosure is less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 25%, less than about 30%, less than about 35%, less than about 40%, or less than about 50% when evaluated after a suitable amount of time post-transplant, for example at about 6 months, about 1 year, about 1.5 years, about 2 years, about 2.5 years, about 3 years, about 3.5 years, about 4 years, about 4.5 years, or about 5 years post-transplant. In some embodiments a treatment-associated mortality rate of a population of subjects administered a composition of the disclosure is less than about 5% when evaluated at 1-year post-transplant. In some embodiments a treatment-associated mortality rate of a population of subjects administered a composition of the disclosure is less than about 1% when evaluated at 1-year post-transplant.

A population of subjects treated or provided with compositions, multi-component pharmaceutical treatments, cell populations, solutions, formulations, kits, and/or methods of the disclosure (e.g., a cell population comprising a population of cells described herein) exhibit GVHD free and relapse free survival (GRFS) rate, for example, a higher GRFS rate compared to subjects that are administered an alternate composition. In some embodiments, GRFS can refer to survival without relapse or Grade ≥3 aGVHD or extensive (e.g., severe) cGVHD. In some embodiments, GRFS can refer to survival without relapse or Grade ≥2 aGVHD or extensive (e.g., moderate to severe) cGVHD. In some embodiments, GRFS can refer to survival with no GVHD symptoms. In some embodiments, a GRFS rate of a population of subjects administered a composition of the disclosure is at least about 20%, at least about 30%, at least about 40%, at least about 50%, at least about 55%, at least about 60%, at least about 65%, at least about 70%, at least about 75%, at least about 80%, at least about 85%, at least about 90%, or at least about 95% when evaluated after a suitable amount of time post-transplant, for example at about 6 months, about 1 year, about 1.5 years, about 2 years, about 2.5 years, about 3 years, about 3.5 years, about 4 years, about 4.5 years, or about 5 years post-transplant. In some embodiments, a GRFS rate of a population of subjects administered a composition of the disclosure is at least about 60% when evaluated at 1-year post-transplant. In some embodiments, a GRFS rate of a population of subjects administered a composition of the disclosure is at least about 75% when evaluated at 1-year post-transplant. In some embodiments, a GRFS rate of a population of subjects administered a composition of the disclosure is more than 75% when evaluated at 3 years post-transplant. In some embodiments, a GRFS rate of a population of subjects administered a composition of the disclosure is more than 75% when evaluated at 5 years post-transplant. In some embodiments, a GRFS rate of a population of subjects administered a composition of the disclosure is more than 75% when evaluated at 7 years post-transplant. In some embodiments, a GRFS rate of a population of subjects administered a composition of the disclosure is more than 75% when evaluated at 10 years post-transplant.

A population of subjects treated or provided with compositions, multi-component pharmaceutical treatments, cell populations, solutions, formulations, kits, and/or methods of the disclosure (e.g., a cell population comprising a population of cells described herein) exhibit a short time to discharge from hospital, for example, a shorter time to discharge from hospital compared to subjects that are administered an alternate composition. In some embodiments, the average time to discharge after day 0 of a transplantation regimen is less than about 20 days, less than about 19 days, less than about 18 days, less than about 17 days, less than about 16 days, less than about 15 days, less than about 14 days, less than about 13 days, less than about 12 days, less than about 11 days, less than about 10 days, less than about 9 days, or less than about 8 days. In some embodiments, the average time to discharge after day 0 of a transplantation regimen is less than about 17 days. In some embodiments, the average time to discharge after day 0 of a transplantation regimen is less than about 18 days. In embodiments, the human subject treated or provided with compositions, multi-component pharmaceutical treatments, cell populations, solutions, formulations, kits, and/or methods as disclosed herein have no relapse of their malignancy about one year after being administered the pharmaceutical dosing regimen.

A population of subjects treated or provided with compositions, multi-component pharmaceutical treatments, cell populations, solutions, formulations, kits, and/or methods of the disclosure (e.g., a cell population comprising a population of cells described herein) exhibit a low relapse rate, for example, a lower relapse rate compared to subjects that are administered an alternate composition. In some embodiments, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 20%, less than about 25%, less than about 30%, less than about 35%, less than about 40%, less than about 45%, less than about 50%, less than about 55%, or less than about 60% of subjects that are administered a composition of the disclosure relapse within a suitable amount of time post-transplant, for example at about 90 days, about 100 days, about 120 days, about 6 months, about 1 year, about 1.5 years, about 2 years, about 2.5 years, about 3 years, about 3.5 years, about 4 years, about 4.5 years, or about 5 years post-transplant. In some embodiments, less than about 35% of subjects that are administered a composition of the disclosure relapse within 1-year post-transplant. In some embodiments, less than about 25% of subjects that are administered a composition of the disclosure relapse within 1-year post-transplant.

A population of subjects that do not have active disease when treated or provided with compositions, multi-component pharmaceutical treatments, cell populations, solutions, formulations, kits, and/or methods of the disclosure (e.g., a cell population as described herein) exhibit a low relapse rate, for example, a lower relapse rate compared to subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 20%, less than about 25%, less than about 30%, less than about 35%, less than about 40%, less than about 45%, less than about 50%, less than about 55%, or less than about 60% of subjects that do not have active disease when administered a composition of the disclosure relapse within a suitable amount of time post-transplant, for example at about 90 days, about 100 days, about 120 days, about 6 months, about 1 year, about 1.5 years, about 2 years, about 2.5 years, about 3 years, about 3.5 years, about 4 years, about 4.5 years, or about 5 years post-transplant.

In some cases, the patient treated or provided with compositions, multi-component pharmaceutical treatments, cell populations, solutions, formulations, kits, and/or methods has no relapse of their malignancy about one year after being administered the cell populations.

A population of subjects that are in complete remission when administered a composition of the disclosure (e.g., a cell population as described herein) exhibit a low relapse rate, for example, a lower relapse rate compared to subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 20%, less than about 25%, less than about 30%, less than about 35%, or less than about 40% of subjects that are in complete remission when administered a composition of the disclosure relapse within a suitable amount of time post-transplant, for example at about 90 days, about 100 days, about 120 days, about 6 months, about 1 year, about 1.5 years, about 2 years, about 2.5 years, about 3 years, about 3.5 years, about 4 years, about 4.5 years, or about 5 years post-transplant.

In some cases, the patient has no GHVD or relapse of their malignancy one year after being administered the cell populations.

### B. Engraftment and Immune Reconstitution

7. Graft Failure

A population of subjects that are administered a composition of the disclosure (e.g., a cell population as described herein) exhibit a low rate of primary graft failure, for example, a lower rate of primary graft failure compared to subjects that are administered an alternate composition. Primary graft failure can be a failure to achieve an absolute neutrophil count of >500 cells/μL after Day 30 post-transplant. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 20%, less than about 30%, or less than about 40% of subjects administered a composition of the disclosure exhibit primary graft failure. In some embodiments, less than about 1% of subjects administered a composition of the disclosure exhibit primary graft failure. In some embodiments, less than about 3% of subjects administered a composition of the disclosure exhibit primary graft failure.

A population of subjects that are administered a composition of the disclosure (e.g., a cell population as described herein) exhibit a low rate of secondary graft failure, for example, a lower rate of secondary graft failure compared to subjects that are administered an alternate composition. Secondary graft failure can be a sustained loss of hematopoiesis after engraftment. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 20%, less than about 30%, or less than about 40% of subjects administered a composition of the disclosure exhibit secondary graft failure when evaluated a suitable amount of time post-transplant, for example at about 90 days, about 100 days, about 120 days, about 6 months, about 1 year, about 1.5 years, about 2 years, about 2.5 years, about 3 years, about 3.5 years, about 4 years, about 4.5 years, or about 5 years post-transplant. In some embodiments, less than about 1% of subjects administered a composition of the disclosure exhibit secondary graft failure within 1-year post-transplant. In some embodiments, less than about 5% of subjects administered a composition of the disclosure exhibit secondary graft failure within 1-year post-transplant.

8. Neutrophil Engraftment

A population of subjects that are administered a composition of the disclosure (e.g., a cell population as described herein) exhibit fast neutrophil engraftment, for example, faster neutrophil engraftment compared to subjects that are administered an alternate composition. Neutrophil engraftment can be indicated by a sustained neutrophil count of >500 cells/μL in the peripheral blood of the recipient. In some embodiments, a population of subjects administered a composition of the disclosure achieve neutrophil engraftment by a median of about 7 days, about 8 days, about 9 days, about 10 days, about 11 days, about 12 days, about 12 days, about 14 days, or about 15 days post-transplant. In some embodiments, a population of subjects administered a composition of the disclosure achieve neutrophil engraftment by a median of 13 days post-transplant. In some embodiments, a population of subjects administered a composition of the disclosure achieve neutrophil engraftment by a median of 12 days post-transplant. In some embodiments, a population of subjects administered a composition of the disclosure achieve neutrophil engraftment by a median of 11 days post-transplant.

9. Platelet Engraftment

A population of subjects that are administered a composition of the disclosure (e.g., a cell population comprising a population of cells described herein) exhibit fast platelet engraftment, for example, faster platelet engraftment compared to subjects that are administered an alternate composition. Platelet engraftment can be indicated by a platelet count >20,000/mm³ for 3 consecutive days without platelet transfusion in the peripheral blood of the recipient. In some embodiments, a population of subjects administered a composition of the disclosure achieve platelet engraftment by a median of about 7 days, about 8 days, about 9 days, about 10 days, about 11 days, about 12 days, about 12 days, about 14 days, or about 15 days post-transplant. In some embodiments, a population of subjects administered a composition of the disclosure achieve platelet engraftment by a median of 13 days post-transplant. In some embodiments, a population of subjects administered a composition of the disclosure achieve platelet engraftment by a median of 12 days post-transplant. In some embodiments, a population of subjects administered a composition of the disclosure achieve platelet engraftment by a median of 11 days post-transplant.

10. T Cell Engraftment

A population of subjects that are administered a composition of the disclosure (e.g., a cell population as described herein) exhibit a high proportion of circulating Tregs, for example, a higher proportion of circulating Tregs compared to subjects that are administered an alternate composition. In some embodiments, an average of at least about 5%, at least about 7.5%, at least about 10%, at least about 12.5%, at least about 15%, at least about 16%, at least about 17%, at least about 18%, at least about 19%, at least about 20%, at least about 21%, at least about 22%, at least about 23%, at least about 24%, or at least about 25% of circulating CD4+ T cells are Tregs when subjects are evaluated a suitable amount of time post-transplant, for example, at about 14 days, 15 days, 20 days, 21 days, 25 days, 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100 days, 110 days, 120 days, 130 days, 140 days, 150 days, 160 days, 170 days, or 180 days post-transplant. In some embodiments, an average of at least about 15% of circulating CD4+ T cells are Tregs when subjects are evaluated 28 days post-transplant.

A population of subjects that are administered a composition of the disclosure (e.g., a cell population as described herein) exhibit a normal CD4:CD8 T cell ratio, for example, a higher CD4:CD8 T cell ratio compared to subjects that are administered an alternate composition or normal healthy control subjects.

In some embodiments, at least about 50%, at least about 55%, at least about 60%, at least about 65%, at least about 70%, at least about 75%, at least about 80%, at least about 85%, at least about 90%, or at least about 95% of subjects have a CD4:CD8 T cell ratio of ≥0.8 when evaluated after a suitable amount of time post-transplant, for example, at about 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

In some embodiments, at least about 50%, at least about 55%, at least about 60%, at least about 65%, at least about 70%, at least about 75%, at least about 80%, at least about 85%, at least about 90%, or at least about 95% of subjects have a CD4:CD8 T cell ratio of ≥1 when evaluated after a suitable amount of time post-transplant, for example, at about 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant. In some embodiments, at least 50% of subjects have a CD4:CD8 T cell ratio of ≥1 when evaluated 28 days post-transplant.

In some embodiments, at least about 50%, at least about 55%, at least about 60%, at least about 65%, at least about 70%, at least about 75%, at least about 80%, at least about 85%, at least about 90%, or at least about 95% of subjects have a CD4:CD8 T cell ratio of ≥1.2 when evaluated after a suitable amount of time post-transplant, for example, at about 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

In some embodiments, at least about 50%, at least about 55%, at least about 60%, at least about 65%, at least about 70%, at least about 75%, at least about 80%, at least about 85%, at least about 90%, or at least about 95% of subjects have a CD4:CD8 T cell ratio of ≥1.5 when evaluated after a suitable amount of time post-transplant, for example, at about 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

A population of subjects that are administered a composition of the disclosure (e.g., a cell population as described herein) exhibit a high proportion of donor-derived circulating T cells at an early timepoint after transplant, for example, a higher proportion of donor-derived circulating T cells compared to subjects that are administered an alternate composition.

In some embodiments, more than 50% of circulating T cells are donor-derived in at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

In some embodiments, more than 60% of circulating T cells are donor-derived in at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

In some embodiments, more than 70% of circulating T cells are donor-derived in at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

In some embodiments, more than 80% of circulating T cells are donor-derived in at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

In some embodiments, more than 90% of circulating T cells are donor-derived in at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

In some embodiments, more than 50% of circulating T cells are donor-derived in at least about at least about 70% of subjects evaluated 30 days post-transplant. In some embodiments, more than 70% of circulating T cells are donor-derived in at least about at least about 60% of subjects evaluated 30 days post-transplant.

11. B Cell Engraftment

A population of subjects that are administered a composition of the disclosure (e.g., a cell population as described herein) exhibit a high concentration of circulating B cells at an early timepoint after transplant, for example, a higher concentration of circulating B cells compared to subjects that are administered an alternate composition. Achieving a high concentration of circulating B cells at an early timepoint after transplant can be important for immunocompetence, and can allow vaccines to elicit protective immune responses at an earlier timepoint post-transplant.

In some embodiments, more than about 50 B cells per μL are present in the blood of at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, more than 50 B cells per μL are present in the blood of at least 75% of subjects evaluated about 60 days post-transplant.

In some embodiments, more than about 60 B cells per μL are present in the blood of at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, more than 60 B cells per μL are present in the blood of at least 75% of subjects evaluated about 60 days post-transplant.

In some embodiments, more than about 70 B cells per μL are present in the blood of at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, more than 70 B cells per μL are present in the blood of at least 75% of subjects evaluated about 60 days post-transplant.

In some embodiments, more than about 80 B cells per μL are present in the blood of at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant.

In some embodiments, more than about 90 B cells per μL are present in the blood of at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, more than 90 B cells per μL are present in the blood of at least 75% of subjects evaluated about 180 days post-transplant.

In some embodiments, more than about 100 B cells per μL are present in the blood of at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, more than 100 B cells per μL are present in the blood of at least 75% of subjects evaluated about 180 days post-transplant.

In some embodiments, more than about 110 B cells per μL are present in the blood of at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, more than 110 B cells per μL are present in the blood of at least 75% of subjects evaluated about 180 days post-transplant.

A population of subjects that are administered a composition of the disclosure (e.g., a cell population as described herein) exhibit a high proportion of mature B cells at an early timepoint after transplant, for example, a higher proportion of circulating B cells that are mature B cells (e.g., IgD+ and/or CD27+) compared to subjects that are administered an alternate composition. Achieving a high proportion of mature B cells at an early timepoint after transplant can be important for immunocompetence, and can allow vaccines to elicit protective immune responses at an earlier timepoint post-transplant.

In some embodiments, at least about 50% of circulating CD19+ B cells are IgD+ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant.

In some embodiments, at least about 60% of circulating CD19+ B cells are IgD+ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant.

In some embodiments, at least about 70% of circulating CD19+ B cells are IgD+ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant.

In some embodiments, at least about 80% of circulating CD19+ B cells are IgD+ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, at least about 80% of circulating CD19+ B cells are IgD+ in at least about 75% of subjects evaluated about 100 days post-transplant.

In some embodiments, at least about 90% of circulating CD19+ B cells are IgD+ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, at least about 90% of circulating CD19+ B cells are IgD+ in at least about 75% of subjects evaluated about 100 days post-transplant.

In some embodiments, at least about 25% of circulating CD19+ B cells are CD27+ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant.

In some embodiments, at least about 30% of circulating CD19+ B cells are CD27+ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, at least about 30% of circulating CD19+ B cells are CD27+ in at least about 75% of subjects evaluated at 100 days post-transplant.

In some embodiments, at least about 35% of circulating CD19+ B cells are CD27+ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, at least about 35% of circulating CD19+ B cells are CD27+ in at least about 75% of subjects evaluated at 100 days post-transplant.

In some embodiments, at least about 40% of circulating CD19+ B cells are CD27+ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, at least about 40% of circulating CD19+ B cells are CD27+ in at least about 75% of subjects evaluated at 100 days post-transplant.

Clinical outcomes disclosed herein can be calculated based on a population of, for example, at least 10, at least at least 11, at least at least 12, at least 13, at least 14, at least 15, at least 16, at least 17, at least 18, at least 19, at least 20, at least 21, at least 22, at least 23, at least 24, at least 25, at least 25, at least 30, at least 35, at least 40, at least 45, at least 50, at least 60, at least 70, at least 80, at least 90, at least 100, at least 120, at least 140, at least 160, at least 180, at least 200, at least 250, at least 300, at least 350, at least 400, at least 450, or at least 500 subjects. VI. KITS

Another aspect provides a kit that comprises a solution comprising a first container comprising a first population of CD45+ cells, a second container comprising a solution comprising a second population of CD45+ cells, and a third container comprising a solution comprising a population of cells enriched for regulatory T cells (Tregs). In this aspect, the solution comprising the first population of CD45+ cells, the solution comprising the second population of CD45+ cells, and the solution comprising the population of cells enriched for Tregs are as defined according to any herein disclosed multi-component pharmaceutical treatment or method. In various embodiments, the kit further comprises a fourth container comprising the GVHD prophylactic agent. In some cases, the further comprising instructions for performing any herein-disclosed method.

A further aspect provides a kit comprising: (a) one or more reagents to sort CD34+ cells from a mobilized peripheral blood composition; (b) one or more reagents to sort regulatory T cells (Tregs) from the mobilized peripheral blood composition; (c) one or more reagents to detect a number of CD3+ conventional T cells in the mobilized peripheral blood; and (d) a solution comprising one or more doses of a graft vs host disease (GVHD) prophylactic agent. In some embodiments, the kit further comprises instructions for performing any herein-disclosed method.

Various embodiments provide a kit comprising one or more reagents for sorting HSPCs, for instance, a kit may comprise reagents to enrich a CD34+ cell population from a mobilized peripheral blood donation.

Some embodiments provides a kit comprising one or more reagents for sorting Tregs, for instance, a kit may comprise reagents to enrich a Treg cell population (using markers as described elsewhere herein) from a mobilized peripheral blood donation.

Embodiments provides a kit comprising one or more conditioning reagents for a conditioning regimen, for instance, a kit may comprise reagents for myeloablation or myeloreduction of a recipient.

Some embodiments provides a kit comprising one or more GVHD prophylactic agents.

In any herein-disclosed method, the method further comprises providing instructions for use (IFU), the IFU including instructions for administering the cell populations to the patient. In some cases, the IFU also include instructions for administering one or more pharmaceutical agents or compositions to the patient.

### Definitions

The terminology used herein is for the purpose of describing particular cases only and is not intended to be limiting.

As used herein, unless otherwise indicated, the terms “a”, “an” and “the” are intended to include the plural forms as well as the single forms, unless the context clearly indicates otherwise.

The terms “comprise”, “comprising”, “contain,” “containing,” “including”, “includes”, “having”, “has”, “with”, or variants thereof as used in either the present Specification and/or in the claims, are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean 10% greater than or less than the stated value. In another example, “about” can mean within 1 or more than 1 standard deviation, per the practice in the given value. Where particular values are described in the application and claims, unless otherwise stated the term “about” should be assumed to mean an acceptable error range for the particular value.

The term “substantially” is meant to be a significant extent, for the most part; or essentially. In other words, the term substantially may mean nearly exact to the desired attribute or slightly different from the exact attribute. Substantially may be indistinguishable from the desired attribute. Substantially may be distinguishable from the desired attribute but the difference is unimportant or negligible.

By “one or more” or “at least one” is meant at least one, e.g., one, two, three, four, five, six, seven, eight, nine, ten or more.

The term “similar” is understood to be resembling up to and including identical. Therefore two (or more) items may be identical, substantially identical, comprise equivalent components, comprise substitutable components, comprise analogous components, comprise comparable components, comprise complementary components, comprise related components, comprise like components, and/or the differences between the two (or more items) are insubstantial and/or result in a composition having equivalent properties, identical properties, substantially identical properties, and the like.

The terms “patient” and “subject” are synonyms.

Any aspect or embodiment described herein can be combined with any other aspect or embodiment as disclosed herein.

### VII. Additional Aspects

Aspects provide a method of transplanting a conventional T cell (Tcon) population into a human subject without eliciting a stage 2 or higher graft versus host disease (GVHD) response up to about 100 days after transplanting. The method comprises (i) administering a heterogenous cell population comprising lymphocytes, granulocytes and monocytes, wherein at least about 30% of said lymphocytes comprises conventional T cells (Tcons); and (ii). administering a population of regulatory T cells (Tregs). In this method, the heterogenous cell component and/or the population of Tregs comprise less than about 5 EU/ml endotoxins.

A further aspect provide a method of treating a human subject comprising a step (a) of administering a plurality of populations of cells, in which the plurality of populations of cells comprises: (i). a population of hematopoietic stem and progenitor cells (HSPCs); (ii) a population of cells comprising regulatory T cells (Tregs); and (iii) a population of conventional T cells (Tcons); and a step (b) of administering no more than one graft versus host disease (GVHD) prophylactic agent for less than about 120 days. In this method, the population of HSPCs comprises less than about 2% CD3+ cells.

An aspects is a method treating a human subject in need thereof comprising administering to the human subject at least two pharmaceutical compositions, wherein the pharmaceutical compositions are selected from (a) a pharmaceutical composition comprising a population of hematopoietic stem and progenitor cells (HSPCs); (b) a pharmaceutical composition comprising a population of regulatory T cells (Tregs); and (c) a pharmaceutical composition comprising a population of conventional T cells (Tcons). In this method, the pharmaceutical compositions (a), (b) and (c) comprise less than about 5 EU/ml endotoxins each; and less than 15 human subjects in a group of 100 human subjects administered the two or more pharmaceutical compositions develops a stage 2 or higher graft versus host disease (GVHD) response within about 30 days after being administered the pharmaceutical composition comprising the population of Tcons.

An additional aspect is a method of transplanting a conventional T cell (Tcon) population into a human subject without eliciting a stage 2 or higher graft versus host disease (GVHD) response up to about 100 days after transplanting. The method comprising: (i). administering a solution comprising a population of conventional T cells (Tcons); and (ii). administering a solution comprising a population of regulatory T cells (Tregs). In this method, the population of Tcons is cryopreserved for at least about 4 hours; and the solution comprising the population of Tcons and the solution comprising the population of Tregs comprise less than about 5 EU of endotoxins per ml of the solution.

A yet another aspect provides a method of treating a hematologic malignancy in a human subject in need thereof, the method comprising administering to the human subject: (a) a population of hematopoietic stem and progenitor cells (HSPCs); (b) a population of regulatory T cells (Tregs); and (c) a population of conventional T cells (Tcons). In the method, the population of HSPCs and the population of Tregs are administered prior to the population of Tcons; and peripheral blood of the human subject exhibits an elevated Treg count until about 100 days after the administering the three populations of cells as compared to a healthy human subject that was not administered the three populations of cells.

A further aspect provides a method of transplanting a conventional T cell (Tcon) population into a human subject without eliciting a stage 2 or higher graft versus host disease (GVHD) response up to about 100 days after transplanting. The method comprising (i). administering a population of conventional T cells (Tcons); and (ii). administering a population of regulatory T cells (Tregs). In this method, the population of Tcons is administered at least about 12 hours after the population of Tregs is administered; and the population of Tcons and the population of Tregs comprise less than about 5 EU/ml endotoxins.

### VII. EXAMPLES

The following examples are included for illustrative purposes only and are not intended to limit the scope of the disclosure.

### Example 1: Clinical Study

C. Study Design

A clinical study was conducted in subjects with advanced hematologic malignancies undergoing myeloablative allogeneic hematopoietic cell transplantation (alloHCT).

The graft composition and manufacturing processes are detailed below.

Primary endpoints of the study include the incidence of primary graft failure; and the incidence, severity, and timing of Grade III-V acute GVHD.

Secondary endpoints for all groups include neutrophil engraftment, platelet engraftment, incidence of secondary graft failure, incidence and severity of treatment-emergent adverse events (TEAEs), incidence and severity of steroid-refractory acute GVHD, for example, grade 3-4 steroid-refractory acute GVHD, incidence and severity of chronic GVHD, incidence of post-transplant lymphoproliferative disorder (PTLD), non-relapse mortality (NRM), disease relapse (Arms I & III), relapse free survival, GVHD and relapse free survival (GRFS), overall survival, incidence of serious infections, and T cell immunity reconstitution parameters.

D. Study Populations

Subjects were eligible to receive a composition of the disclosure if they met all the following criteria:
(1) Age ≥18 and ≤65 years at the time of enrollment.
(2) Diagnosed with the any of the following histopathologically-confirmed diseases: (a) Acute myeloid, lymphoid or mixed phenotype leukemia in complete remission (CR) or CR with incomplete hematologic recovery (CRi) without the presence of known minimal residual disease; or (b) Acute myeloid, lymphoid or mixed phenotype leukemia that is either: (i) not in morphologic CR with bone marrow infiltration by leukemic blasts of ≤10%, or (ii) in morphologic CR with evidence of minimal residual positivity by either multiparameter flow cytometric analysis or by a nucleic acid-based technique; (c) High or Very High-risk Myelodysplastic syndromes; (d) Myelofibrosis (MF) that is eligible for transplant per National Comprehensive Cancer Network Guidelines. Specifically, patients should be diagnosed with MF that is either: (i) intermediate-2- or high-risk according to the IPSS, DIPSS or DIPSS-plus scoring systems; or (ii) intermediate-1-risk disease associated with high-risk features such as high symptoms burden, low platelet counts, or complex cytogenetics; per NCCN guidelines and Investigator judgement (e) myeloproliferative syndromes; (f) Non-Hodgkin lymphoma with poor risk features not suitable for autologous HCT.
(3) Planning to undergo myeloablative allogeneic hematopoietic cell transplantation (MA-alloHCT) including a suitable myeloablative conditioning regimen.
(4) Matched to a donor that is a: (a) Matched sibling donor who is an 8/8 match for HLA-A, -B, -C, and -DRB1, all typed using DNA-based high resolution methods; or (b) Matched unrelated donor who is a 8/8 match at HLA-A, -B, -C, and -DRB1, all typed using DNA-based high resolution methods.
(5) Estimated glomerular filtration rate (eGFR) >30 mL/minute; or >50 mL/minute for patients for whom GVHD prophylaxis with tacrolimus is planned.
(6) Cardiac ejection fraction at rest ≥45% or shortening fraction of ≥27% by echocardiogram or radionuclide scan (MUGA).
(7) Diffusing capacity of the lung for carbon monoxide (DLCO) (adjusted for hemoglobin) ≥50%.
(8) Negative serum or urine beta-HCG test in females of childbearing potential within 3 weeks of registration.
(9) Total bilirubin <2 times upper limit of normal (ULN) (patients with Gilbert's syndrome may be included where hemolysis has been excluded and with approval of the medical monitor).
(10) ALT/AST <3 times upper limit of normal (ULN).

Subjects were ineligible to receive the composition of the disclosure if they met any of the following exclusion criteria:
(1) Received a prior allogeneic HCT.
(2) Candidate for autologous transplant.
(3) Currently receiving corticosteroids or other immunosuppressive therapy. Topical corticosteroids or oral systemic corticosteroid doses less than or equal to 10 mg/day are allowed.
(4) Planned donor lymphocyte infusion (DLI) recipient.
(5) Planned recipient of pharmaceutical in vivo or ex vivo T cell depletion, e.g., post-transplant cyclophosphamide (Cy), peri-transplant anti-thymocyte globulin (ATG), or alemtuzumab. For patients that have previously been exposed to a T cell-depleting agent, a 5 half-life washout of the agent must occur prior to planned Day 0 (day of infusion of the Treg and HSPC components of the graft).
(6) Positive for anti-donor HLA antibodies against a mismatched allele in the selected donor as determined by either: (a) A positive crossmatch test of any titer; or (b) The presence of anti-donor HLA antibody to any HLA locus.
(7) Karnofsky performance score <70%.
(8) Hematopoietic cell transplantation-specific Comorbidity Index (HCT-CI) >4.
(9) Uncontrolled bacterial, viral or fungal infections (currently taking antimicrobial therapy and with progression or no clinical improvement) at time of enrollment.
(10) Seropositive for HIV-1 or -2, HTLV-1 or -2, Hepatitis B sAg, or Hepatitis C antibody,
(11) Known allergy or hypersensitivity to, or intolerance of, tacrolimus (or tacrolimus and sirolimus if eligible for single-agent prophylaxis with either)
(12) Documented allergy or hypersensitivity to iron dextran or bovine, murine, algal or Streptomyces avidinii proteins.
(13) Any uncontrolled autoimmune disease requiring active immunosuppressive treatment.
(14) Concurrent malignancies or active disease within 1 year, except non-melanoma skin cancers that have been curatively resected.
(15) Psychosocial circumstances that preclude the patient being able to go through transplant or participate responsibly in follow up care.
(16) Women who are pregnant or breastfeeding.
(17) Women of childbearing potential (WOCBP) or men who have sexual contact with WOCBP unwilling to use effective forms of birth control or abstinence for one year after transplantation.
(18) Any serious medical condition or abnormality in clinical laboratory tests that, in the investigator's or Medical Monitor's judgment, precludes the recipient's safe participation in and completion of the study, or which could affect compliance with the protocol or interpretation of results.

Subjects were part of the following groups:

Arm 1: subjects planning to undergo myeloablative allogeneic hematopoietic cell transplantation (MA-alloHCT) for the treatment of either acute myeloid, lymphoid or mixed phenotype leukemia in complete remission (CR) or CR with incomplete hematologic recovery (CRi) with no known minimal residual disease positivity, planning to MA-alloHCT.

Arm 2: subjects planning to undergo MA-alloHCT for acute myeloid, lymphoid or mixed phenotype leukemia that is either: (i) not in morphologic CR with bone marrow infiltration by leukemic blasts of ≤10%, or (ii) in morphologic CR with evidence of minimal residual positivity by either multiparameter flow cytometric analysis or by a nucleic acid-based technique.

Arm 3: subjects planning to undergo MA-alloHCT for high or very high-risk myelodysplastic syndrome (MDS) myelodysplastic syndromes or for myelofibrosis (primary myelofibrosis or myelofibrosis evolved from other myeloproliferative neoplasms).

Subjects with sensitivity to iron dextran, chemical products derived from cyanine dyes, and proteins products derived from murine, bovine, algal, and Streptomyces avidinii sources are excluded.

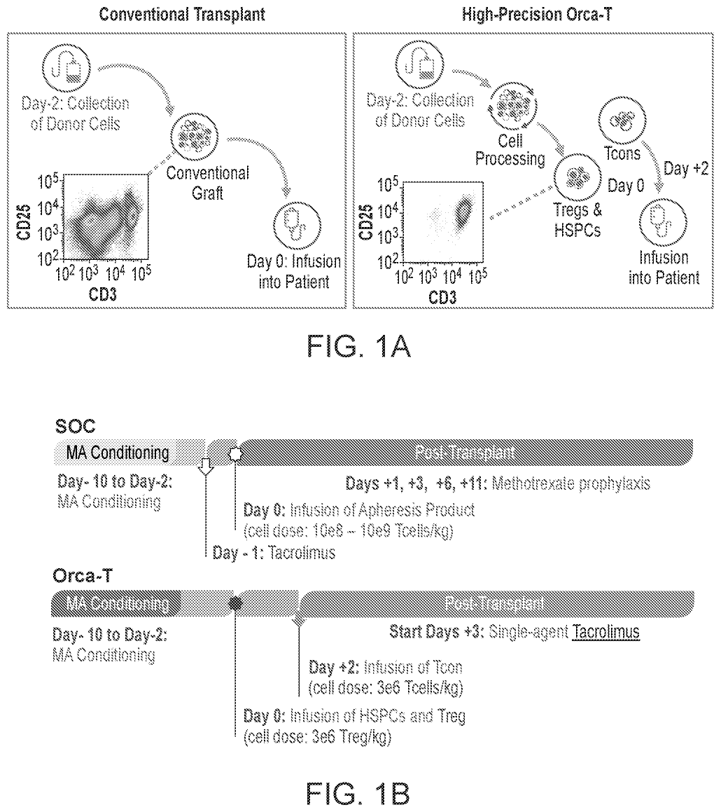

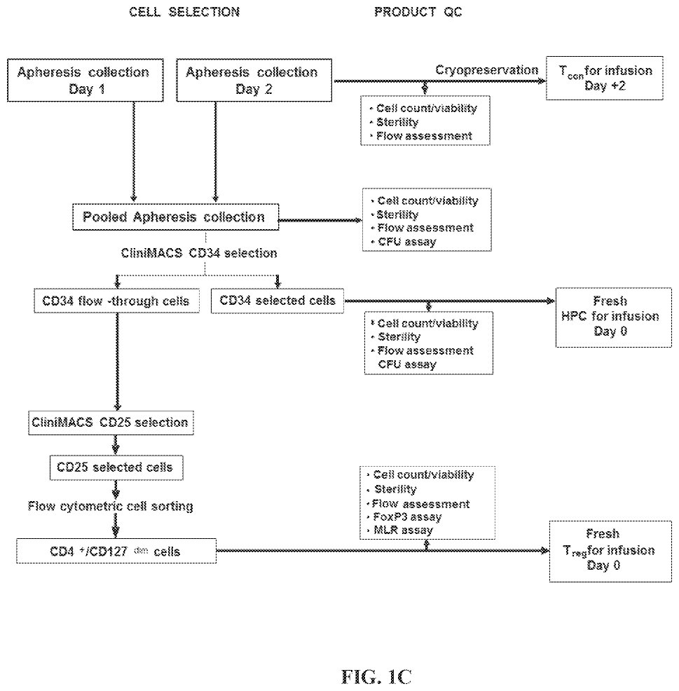

FIGS. 1A-B illustrate the schematics of the transplant according to the present study (identified as High-Precision Orca-T or OrcaT) and the differences compared to a standard of care (SOC) cohort (identified as Conventional Transplant or SOC). FIG. 1C illustrates a schematic of graft production and administration.

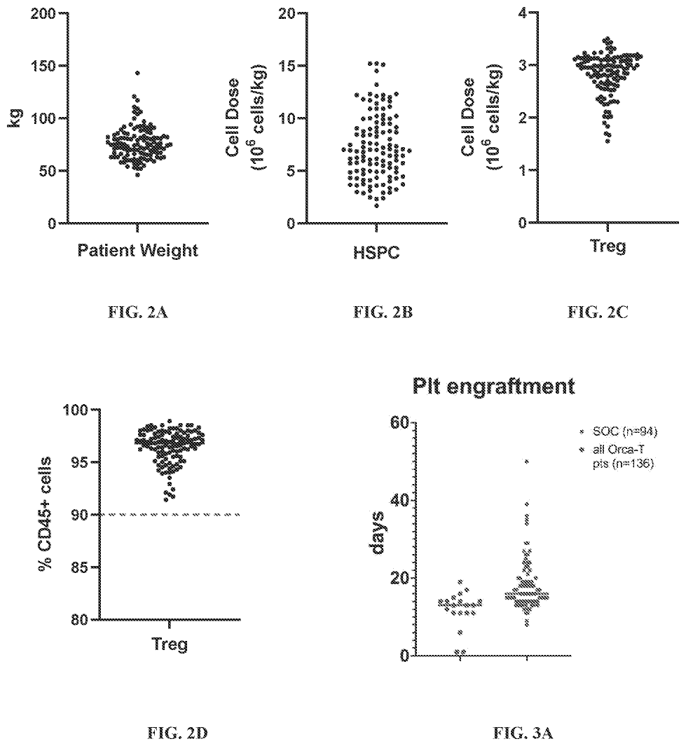

FIG. 2A illustrates the weights of patients enrolled in the study. Table 7 shows the shows the demographics, primary disease, features determining disease risk status, disease status at transplant, and transplant details for a representative subset of the subjects. Abbreviations: DLBCL, diffuse large B cell lymphoma; AML, acute myeloid leukemia; ALL, acute lymphocytic leukemia; MDS, myelodysplastic syndrome; MF, myelofibrosis; MPAL, mixed phenotype acute leukemia; CML, chronic myeloid leukemia; CR1, 1st complete remission; CR2, 2nd complete remission; active, active disease (not in CR as defined for disease entity); MRD+, minimal residual disease positive; MRD, matched related donor; URD, unrelated donor; MF, myelofibrosis. FTBI, fractionated total body irradiation; Cy, cyclophosphamide; VP-16, etoposide; Bu, busulfan; FLT3+, FMS-like tyrosine kinase 3; c-kit, ASXL1, additional sex combs like 1. Days of follow up denotes the number of days since transplant Day 0 for each patient at the time of reporting.

TABLE 7

| | | | | Disease | | | | Days of |
|---|---|---|---|---|---|---|---|---|
| | | | High-risk | status at | Donor | Conditioning | GVHD | follow |
| Age | Sex | Disease | Features | transplant | type | Regimen | prophylaxis | up |
| 49 | M | BLBC | refractory | active | MRD | BCNU/VP16/Cy | Sirolimus | 1219 |
| | | L | | | | | | |
| 42 | M | AML | FLT3+ | CR2 | MRD | BU/Cy | Sirolimus to | 1177 |
| | | | | | | | Tacrolimus | |
| 34 | M | ALL | refractory | active | MRD | TBI/Cy/VP16 | Sirolimus | 1107 |
| 20 | M | ALL | MRD+ | CR2 | MRD | BU/Cy | Sirolimus | 1079 |
| 54 | M | CML | refractory | active | MRD | BU/Cy | Sirolimus | 1058 |
| 53 | M | AML | AML CR2 | CR2 | MRD | BU/Cy | Sirolimus | 981 |
| 56 | M | MF | refractory | active | MRD | BU/Cy | Tacrolimus | 932 |
| 40 | F | AML | refractory | CR1 | MRD | BU/Cy | Tacrolimus | 260 |
| 59 | M | PTCL | refractory | active | MRD | BCNU/ | Sirolimus | 225 |
| | | | | | | VP16/Cy | | |
| 42 | F | AML | refractory | CR1 | MRD | BU/Cy | None | 211 |
| 40 | M | AML | refractory | CR1 | MRD | BU/Cy | None | 204 |
| 48 | M | MDS | Monosomy | active | MRD | BU/Cy | None | 190 |
| | | | 7 | | | | | |
| 35 | M | ALL | MRD+ | CR1 | MRD | FTBI/VP16/Cy | Sirolimus | 176 |
| 28 | M | ALL | CR2 | CR2 | MRD | BU/Cy | None | 127 |
| 56 | F | MF | n/a | active | URD | BU/Cy | Tacrolimus | 119 |
| 39 | M | MPAL | MPAL | CR1 | MRD | FTBI/VP16/Cy | Sirolimus | 120 |
| 41 | F | AML | TP53 and | CR1 | MRD | BU/Cy | Tacrolimus | 99 |
| | | | c-Kit | | | | | |
| | | | mutated | | | | | |
| 39 | M | ALL | Ph-like | CR1 | MRD | FTBI/VP16/Cy | None | 43 |
| 34 | M | MPAL | Complex | CR1 | MRD | FTBI/VP16/Cy | Sirolimus | 36 |
| | | | karyotype, | | | | | |
| | | | MPAL | | | | | |
| 26 | F | AML | AML | CR1 | MRD | BU/Cy | Tacrolimus | 34 |
| | | | t(6:9) | | | | | |
| 52 | M | AML | ASXL1 | CR1 | MRD | BU/Cy | Tacrolimus | 22 |
| | | | mutation | | | | | |

Standard of Care Control Cohort

For comparison of clinical outcomes, a standard of care (SOC) comparator cohort was identified retrospectively from subjects who received a SOC myeloablative alloHCT at one of the same clinical sites during the same period; a schematic of the protocol followed for SOC patients is also shown in FIGS. 1A-B. To be included in the SOC cohort, subjects had to meet all the following criteria: (a) they were diagnosed with a hematologic malignancy eligible for treatment with an alloHCT; (b) they received an allograft of mobilized peripheral blood (i.e., not a bone marrow-derived graft); (c) their donor was a fully HLA-matched related donor (unrelated donors were not included due to the limited number in the treatment cohort to date); (d) they received a myeloablative conditioning regimen; and (e) they were not enrolled on an investigative protocol. Personnel who identified subjects for the SOC cohort were blinded as to their clinical outcomes.

All patients in the SOC cohort received myeloablative conditioning regimens and dual-agent GVHD prophylaxis with tacrolimus and methotrexate.

Clinical characteristics of subjects in the SOC cohort are provided in TABLE 8.

TABLE 8: characteristics of a representative subset of subjects in the standard of care (SOC) cohort. Abbreviations used: DLBCL, diffuse large B cell lymphoma; AML, acute myeloid leukemia; ALL, acute myeloid leukemia; MDS, myelodysplastic syndrome; MF, myelofibrosis; MPAL, mixed phenotype acute leukemia; CML, chronic myeloid leukemia; CR1, 1st complete remission; CR2, 2nd complete remission; “active”, active disease (not CR as defined for each disease entity); MRD, minimal residual disease; MRD, matched related donor; MF, myelofibrosis. FTBI, fractionated total body irradiation; Cy, cyclophosphamide; VP-16, etoposide; Bu, busulfan; FLT3+, FMS-like tyrosine kinase 3; Tac, tacrolimus; MTX, methotrexate.

TABLE 8

| | | | Disease | | Con- | GVHD |
|---|---|---|---|---|---|---|
| | | | Status at | Donor | ditioning | Pro- |
| Age | Sex | Disease | Transplant | Type | regimen | phylaxis |
| 46 | F | ALL | CR 1 | MRD | FTBI/VP16 | FK/MTX |
| 49 | F | ALL | CR 1 | MRD | FTBI/VP16 | FK/MTX |
| 56 | F | AML | CR 1 | MRD | BU/CY | FK/MTX |
| 58 | M | AML | CR 1 | MRD | BU/CY | FK/MTX |
| 44 | M | ALL | CR 1 | MRD | FTBI/VP16 | FK/MTX |
| 45 | M | AML | CR 2 | MRD | FTBI/VP16 | FK/MTX |
| 52 | M | ALL | CR 1 | MRD | BU/CY | FK/MTX |
| 56 | M | AML | CR 1 | MRD | BU/CY | FK/MTX |
| 32 | F | ALL | CR 1 | MRD | FTBI/VP16 | FK/MTX |
| 23 | M | AML | CR 2 | MRD | BU/CY | FK/MTX |
| 59 | F | ALL | CR 1 | MRD | BU/CY | FK/MTX |
| 50 | F | AML | CR 2 | MRD | BU/CY | FK/MTX |
| 50 | M | ALL | CR 1 | MRD | FTBI/VP16 | FK/MTX |
| 31 | F | AML | CR 1 | MRD | BU/CY | FK/MTX |
| 57 | F | AML | CR 1 | MRD | BU/CY | FK/MTX |
| 21 | M | AML | CR 1 | MRD | BU/CY | FK/MTX |
| 27 | M | OAL | CR 2 | MRD | BU/CY | FK/MTX |
| 51 | F | ALL | CR 1 | MRD | FTBI/VP16 | FK/MTX |
| 38 | M | ALL | CR 1 | MRD | FTBI/VP16 | FK/MTX |
| 44 | F | CML | CP1 | MRD | BU/CY | FK/MTX |
| 19 | M | ALL | CR 1 | MRD | FTBI/VP16 | FK/MTX |
| 58 | M | ALL | CR 1 | MRD | BU/CY | FK/MTX |
| 55 | M | ALL | CR 1 | MRD | BU/CY | FK/MTX |
| 39 | M | AML | active | MRD | BU/CY | FK/MTX |
| 25 | M | AML | CR 2 | MRD | BU/CY | FK/MTX |
| 33 | M | NHL | CR 1 | MRD | BU/CY | FK/MTX |
| 32 | M | AML | CR 2 | MRD | BU/CY | FK/MTX |
| 59 | M | AML | CR 1 | MRD | BU/CY | FK/MTX |
| 47 | M | AML | CR 1 | MRD | BU/CY | FK/MTX |
| 47 | M | ALL | CR 1 | MRD | FTBI/VP16 | FK/MTX |
| 43 | M | AML | CR 1 | MRD | BU/CY | FK/MTX |
| 59 | F | ALL | CR 2 | MRD | BU/CY | FK/MTX |
| 27 | M | ALL | CR 1 | MRD | FTBI/VP16 | FK/MTX |
| 40 | M | AML | CR 1 | MRD | BU/CY | FK/MTX |
| 24 | F | ALL | CR 1 | MRD | FTBI/VP16 | FK/MTX |
| 27 | F | NHL | active | MRD | BU/CY | FK/MTX |
| 34 | M | ALL | CR 1 | MRD | FTBI/VP16 | FK/MTX |
| 58 | M | AML | CR 1 | MRD | BU/CY | FK/MTX |
| 41 | M | CML | active | MRD | BU/CY | FK/MTX |
| 58 | M | ALL | CR 1 | MRD | BU/CY | FK/MTX |
| 52 | F | AML | active | MRD | BU/CY | FK/MTX |
| 62 | M | MDS | active | MRD | BU/CY | FK/MTX |
| 62 | M | MDS | active | MRD | BU/CY | FK/MTX |
| 40 | M | AML | CR 1 | MRD | BU/CY | FK/MTX |
| 44 | F | AML | CR 1 | MRD | FTBI/VP16 | FK/MTX |
| 54 | M | ALL | CR 1 | MRD | BU/CY | FK/MTX |
| 36 | M | AML | CR 1 | MRD | BU/CY | FK/MTX |
| 46 | F | CML | CR 2 | MRD | FTBI/VP16 | FK/MTX |
| 41 | M | MDS | active | MRD | BU/CY | FK/MTX |
| 43 | F | ALL | CR 2 | MRD | FTBI/VP16 | FK/MTX |
| 51 | F | MDS | active | MRD | BU/CY | FK/MTX |
| 54 | M | MDS | active | MRD | BU/CY | FK/MTX |
| 52 | F | CML | CR1 | MRD | BU/CY | FK/MTX |
| 56 | F | MDS | active | MRD | BU/CY | FK/MTX |
| 50 | M | AML | CR 1 | MRD | BU/CY | FK/MTX |
| 56 | M | MDS | active | MRD | BU/CY | FK/MTX |
| 57 | F | MDS | active | MRD | BU/CY | FK/MTX |
| 49 | F | ALL | CR 1 | MRD | FTBI/VP16 | FK/MTX |
| 34 | M | AML | CR 1 | MRD | BU/CY | FK/MTX |
| 59 | F | ALL | CR 1 | MRD | BU/CY | FK/MTX |
| 60 | M | MDS | active | MRD | BU/CY | FK/MTX |
| 32 | M | AML | CR 1 | MRD | FTBI/VP16 | FK/MTX |
| 57 | F | OAL | CR | MRD | BU/CY | FK/MTX |
| 43 | F | AML | CR 1 | MRD | BU/CY | FK/MTX |
| 47 | M | ALL | CR 1 | MRD | BU/CY | FK/MTX |
| 60 | M | MDS | active | MRD | BU/CY | FK/MTX |
| 63 | M | AML | CR 1 | MRD | BU/CY | FK/MTX |
| 49 | M | AML | CR 1 | MRD | BU/CY | FK/MTX |
| 51 | F | AML | CR 2 | MRD | BU/CY | FK/MTX |
| 49 | M | AML | CR 2 | MRD | BU/CY | FK/MTX |
| 40 | F | ALL | CR 1 | MRD | BU/CY | FK/MTX |
| 48 | M | NHL | CR 2 | MRD | BU/CY | FK/MTX |
| 52 | M | AML | CR 2 | MRD | BU/CY | FK/MTX |
| 36 | M | OAL | CR 1 | MRD | FTBI/VP16 | FK/MTX |
| 27 | M | NHL | active | MRD | FTBI/VP16 | FK/MTX |
| 57 | M | AML | CR 1 | MRD | BU/CY | FK/MTX |
| 56 | F | NHL | active | MRD | BU/CY | FK/MTX |
| 52 | F | AML | CR 1 | MRD | BU/CY | FK/MTX |
| 39 | M | AML | active | MRD | FTBI/VP16 | FK/MTX |
| 57 | F | MDS | active | MRD | BU/CY | FK/MTX |
| 26 | M | ALL | CR 1 | MRD | FTBI/VP16 | FK/MTX |
| 21 | F | ALL | CR3+ | MRD | FTBI/VP16 | FK/MTX |
| 55 | M | NHL | active | MRD | BU/CY | FK/MTX |
| 25 | M | ALL | CR 1 | MRD | FTBI/VP16 | FK/MTX |
| 34 | F | MDS | active | MRD | BU/CY | FK/MTX |
| 40 | F | AML | CR 1 | MRD | BU/CY | FK/MTX |
| 59 | M | ALL | CR 1 | MRD | BU/CY | FK/MTX |
| 42 | F | NHL | active | MRD | BU/CY | FK/MTX |
| 29 | M | ALL | CR 1 | MRD | FTBI/VP16 | FK/MTX |
| 26 | F | ALL | CR | MRD | FTBI/VP16 | FK/MTX |
| 40 | M | CML | CP1 | MRD | BU/CY | FK/MTX |
| 58 | F | MDS | CR | MRD | BU/CY | FK/MTX |
| 53 | M | ALL | CR 1 | MRD | BU/CY | FK/MTX |
| 21 | M | ALL | CR 2 | MRD | BU/CY | FK/MTX |

E. Donors

HLA-identical related or unrelated donors were used.

Donors were used that met all of the following inclusion criteria:
(1) Age ≥16 and ≤75 years at time of enrollment
(2) Matched to the patient as follows: Either one of: (i) matched related donor who is an 8/8 match for HLA-A, -B, -C, and -DRB1, all typed using DNA-based high resolution methods; (ii) matched unrelated donor who is an 8/8 match at HLA-A, -B, -C, and -DRB1, all typed using DNA-based high resolution methods.
(3) Able to donate at a site that will employ a Spectra Optia Apheresis System for post-mobilization apheresis;
(4) Meets federal eligibility criteria for donors of viable, leukocyte-rich cells or tissues as defined by 21 CFR § 1271 2018 and all relevant FDA Guidance for Industry (Eligibility Determination for Donors of Human Cells, Tissues, and Cellular and Tissue-Based Products, 2007; Use of Donor Screening Tests to Test Donors of Human Cells, Tissues and Cellular and Tissue-Based Products for Infection with Treponema pallidum (Syphilis), 2015; Use of Nucleic Acid Tests to Reduce the Risk of Transmission of Hepatitis B Virus from Donors of Human Cells, Tissues, and Cellular and Tissue-Based Products, 2016; Use of Nucleic Acid Tests to Reduce the Risk of Transmission of West Nile Virus from Living Donors of Human Cells, Tissues, and Cellular and Tissue-Based Products (HCT/Ps), 2016; Donor Screening Recommendations to Reduce the Risk of Transmission of Zika Virus by Human Cells, Tissues, and Cellular and Tissue-Based Products, 2018).
(5) Meets any other criteria for donation as specified by standard NMDP guidelines (NMDP donors) or institutional standards (non-NMDP donors).
(6) Female donors of child-bearing potential must have a negative serum or urine beta HCG test within 3 weeks of mobilization.
(7) Capable of undergoing leukapheresis, have adequate venous access, and be willing to undergo insertion of a central catheter should leukapheresis via peripheral vein be inadequate.

Donors determined to be ineligible, based on the results of required testing and/or screening, were nonetheless included if either apply, as per 21 CFR § 1271.65 2018: (a) the donor is a first-degree or second-degree blood relative of the recipient; or (b) Urgent medical need, meaning no comparable human cell product is available and the recipient is likely to suffer death or serious morbidity without the human cell product, as attested by the Investigator.

Donors meeting any of the following exclusion criteria were not eligible:
(1) Evidence of active infection
(2) Seropositive for HIV-1 or -2, HTLV-1 or -2
(3) Positive for anti-hepatitis C (HCV) antibody or HCV NAT.
(4) Positive serologic or PCR test results indicating acute or chronic HBV infection. Donors whose HBV infection status cannot be determined conclusively by serologic test results must be negative for HBV by PCR to be eligible for study participation.
(5) Potential for Zika virus infection as defined as any of the following: (i) Medical diagnosis of Zika virus infection in the past 6 months; (ii) Residence in, or travel to, an area with active Zika virus transmission within the past 6 months; (iii) Unprotected sex within the past 6 months with a person who is known to have either of the risk factors (i) or (ii). Donors determined to be ineligible based on the results of Zika virus screening may be determined to be eligible if: (a) the donor has no signs or symptoms consistent with active Zika virus infection; and (b) The donor is a first-degree or second-degree blood relative of the recipient, or ii) in cases of urgent medical need, meaning no comparable human cell product is available and the recipient is likely to suffer death or serious morbidity without the human cell product, as attested by the Investigator.
(6) Women who are pregnant or breastfeeding.

F. Generation of Cell Components

Donors received mobilization therapy with daily G-CSF. The recommended dose was 10 μg/kg/day SQ (rounded off to the nearest vial size of either 300 or 480 μg). The Mobilization Phase started on the first day of administration of G-CSF and continued until the final day of leukapheresis. A schematic of graft production and administration for the protocol is provided in FIG. 1C.

Large volume apheresis started on the 4th day of G-CSF administration, which was generally day −3 relative to CD34-enriched (HSPC) and Treg product infusions into the subject (defined as Day 0). Apheresis commenced with a target of ≥3×10⁶ CD34+ cells/kg recipient body weight post-selection. In order to have sufficient cells available for CD34 enrichment and Treg sorting procedures, donors underwent apheresis collections on 2 consecutive days (e.g., Days −3 and −2). To the degree possible, the 1st day's apheresis (Day −3) collection was scheduled for afternoon hours and the 2nd day's (Day −2) for early morning, thereby limiting the time from the end of the first collection to the infusion of the cellular products to less than 72 hours. Plerixafor (e.g., 0.24 mg/kg SC, once) was available prior to the second apheresis if recommended by the investigator and/or attending physician to achieve the HSPC dose target.

The CD34 reduced (flow-through) fractions were retained and used for isolation of donor Treg. For cell selection, clinical grade reagents were used under Good Manufacturing Practice (GMP) Conditions within the BMT Cellular Therapy Facility.

CD25+ cells were then selected from the CD34-depleted fraction using bead purification (Miltenyi). Tcons were obtained from the negative fraction and the positive fraction was used for Treg purification. CD4+CD25+CD127dim cells underwent further selection by FACS using a BD Influx cell sorter (BD Biosciences, San Jose CA). Enrichment of Tregs was following depletion of CD34+ cells by immunomagnetic selection, selection of CD25+ by immunomagnetic selection and purification by FACS sorting of CD4+CD127lowCD25+ cells. High purity of Tregs were obtained. These cells were highly suppressive in a mixed lymphocyte reaction (MLR).

When a lower than intended Treg dose level was achieved, e.g., a final Treg yield of <2×10⁶/kg body weight of the recipient, the recipient received a dose of 1-2×10⁶ Treg/kg if that dose could be achieved, and a reduced Tcon dose such that the ratio of administered Treg to Tcon was 1:1.

G. Treatments

Subjects received the cell components indicated in TABLE 9

TABLE 9

| | | Cell Population Target |
|---|---|---|
| | | Dose Range, per Recipient |
| Graft Component: | Dose Regimen | kg |
| HSPC (also described herein as | IV, Once, | ≥3 × 10⁶ |
| a “first composition of CD45+ | on Day 0 | |
| cells) | | |
| Treg (also described herein as a | IV, Once, | 2-3 × 10⁶ |
| “cell composition enriched for | on Day 0 | |
| Tregs) | | |
| Tcon (also described herein as a | IV, Once, | 3 × 10⁶ |
| “second composition of CD45+ | on Day +2 | |
| cells) | | |

In the Examples, term “HSPC” as a transplant, as a graft, as a cell dose, as a product, as a drug product, as a component, or as a graft component, and the like corresponds to the “first composition of CD45+ cells” and the like as disclosed elsewhere in the application, including the claims; the term “Treg” as a transplant, as a graft, as a cell dose, as a product, as a drug product, as a component, or as a graft component, and the like corresponds to the “cell composition enriched for Tregs) and the like as disclosed elsewhere in the application, including the claims; and the term “Tcon” as a transplant, as a graft, as a cell dose, as a product, as a drug product, as a component, or as a graft component, and the like corresponds to the “second composition of CD45+ cells) and the like as disclosed elsewhere in the application, including the claims.

Patients received Treg cells with purity higher than 90%, with a median Treg purity of 93.8%+/−3.1%. FIGS. 2B-D illustrates the cell dose of HSPCs and Treg cells administered to patients. Table 10 below illustrates an analysis of the HSPC drug product (e.g., the first population of CD45+ cells) from a representative subset of 20 subjects enrolled in this study. Table 11 below illustrates an analysis of the Treg drug product (e.g., the population of cells enriched for Tregs) from a representative subset of 20 subjects enrolled in this study. Cell components were provided as single dose transfer bags, with an approximate fill volume of 100 mL each for both the Treg and the HSPC components. For each one of Tables 10-11, samples from 20 subjects from the multicenter clinical trial were analyzed. Means and standard deviations (SD) shown in each table. The mean (SD) patient weight was 71.8 kg (11.2 kg). Impurity cell doses were calculated by multiplying the % impurity by the reported cell dose for each patient

The Tcon component (e.g., the second population of CD45+ cells) was provided frozen, after storage in a vapor phase liquid nitrogen tank, in an approximate volume of 15 mL. The pooled apheresis product was assessed for CD3+ Tcon cells and a volume of the apheresis frozen product comprising 3.0E+06 Tcons was calculated and administered to subjects. Table 12 below illustrates an analysis of the Tcon drug product from a representative subset of 20 subjects enrolled in this study. Analysis of the Tcon drug products from 20 grafts in the multicenter clinical trial (N=20). Means and standard deviations (SD) shown. Cell doses were calculated for the cell populations based on the targeted CD3+ T cell dose of 3M/kg. Biomarker data (BM), manufacturing data (mfg); mean (SD) patient weight was 71.8 kg (11.2 kg).

Each cellular therapy product comprises cells, Plasma-Lyte A, and human serum albumin, at a pH of approximately 7.4. The bags and/or primary bag containers had labels bearing the appropriate label text as required by governing regulatory agencies.

The amount of time from when the cell product was received from the donor to when it was administered to the recipient was under 60 hours as is shown for a subset of patients in TABLE 13.

Dosing was based on a subject's actual body weight rounded to the nearest tenth of a kilogram, as assessed during screening, or based on adjusted body weight (ABW) if the subject's actual weight is greater than 120% of the ideal body weight (IBW), calculated as ABW [in kg]=[(actual weight−IBW)×0.40]+IBW.

TABLE 14 illustrates endotoxin levels in each cell population for a subset of patients.

TABLE 10
HSPC product analysis

| | % of Live | | Cell Dose/ | |
|---|---|---|---|---|
| Cell population | CD45+ cells | SD | kg | SD |
| CD45+ live cells | 100 | 0.0 | 8.2E+06 | 3.6E+06 |
| HSPCs (purity) | 93.3 | 11.5 | 7.7E+06 | 3.8E+06 |
| Non-HSPCs (impurity) | 6.7 | 11.5 | 4.2E+05 | 7.3E+05 |
| Granulocytes | 3.2 | 6.8 | 1.9E+05 | 4.3E+05 |
| Monocytes | 1.2 | 2.2 | 7.2E+04 | 1.3E+05 |
| Lymphocytes | 1.1 | 0.8 | 7.8E+04 | 5.7E+04 |
| Non-T lymphs | 1.0 | 0.8 | 7.2E+04 | 5.6E+04 |
| T cells | 0.1 | 0.1 | 6.1E+03 | 9.5E+03 |

TABLE 11
Treg Product analysis

| | % of Live | | Cell Dose/ | |
|---|---|---|---|---|
| Cell population | CD45+ cells | SD | kg | SD |
| CD45+ live cells | 100.0 | 0.0 | 2.8E+06 | 3.6E+05 |
| Tregs (purity) | 96.5 | 2.2 | 2.7E+06 | 3.7E+05 |
| Non-Tregs (impurity) | 3.5 | 2.2 | 9.3E+04 | 5.4E+04 |
| Grans and Monos | 0.1 | 0.1 | 1.9E+03 | 1.4E+03 |
| Lymphocytes | 3.2 | 2.1 | 8.6E+04 | 5.2E+04 |
| T cells | 2.4 | 1.9 | 6.3E+04 | 4.6E+04 |
| CD4+ T cells | 2.3 | 1.9 | 6.2E+04 | 4.6E+04 |
| Non CD4+ T cells | 0.0 | 0.1 | 1.4E+03 | 1.6E+03 |
| Non-T cell lymphs | 0.8 | 1.2 | 2.2E+04 | 3.2E+04 |

TABLE 12
Tcon product analysis

| | % of Live | | Cell Dose/ | |
|---|---|---|---|---|
| Cell population | CD45+ cells | SD | kg | SD |
| CD45+ live cells (mfg) | 100.0 | 0.0 | 1.0E+07 | 2.4E+06 |
| CD45+ cell (BM) | 100.0 | 0.0 | 1.9E+07 | 7.1E+06 |
| Grans + Monos (mfg) | 52.2 | 8.1 | 6.2E+06 | 2.3E+06 |
| Grans + Monos (BM) | 56.7 | 8.8 | 1.1E+07 | 5.8E+06 |
| Granulocytes (BM) | 27.6 | 9.9 | 5.6E+06 | 3.9E+06 |
| Monocytes (BM) | 25.7 | 6.6 | 4.9E+06 | 2.1E+06 |
| Lymphocytes (mfg) | 41.1 | 7.8 | 4.6E+06 | 5.7E+05 |
| Lymphocytes (BM) | 29.8 | 9.4 | 5.1E+06 | 6.8E+05 |
| CD3+ (T) cells reported | 30.1 | 6.4 | 3.0E+06 | 0.0E+00 |
| CD3+ (T) cells (BM) | 17.7 | 5.7 | 3.0E+06 | 0.0E+00 |
| CD4+ T cells (BM) | 8.8 | 3.5 | 1.5E+06 | 3.2E+05 |
| CD8+ T cells (BM) | 7.4 | 3.2 | 1.2E+06 | 3.0E+05 |
| B cells (BM) | 5.2 | 2.5 | 9.1E+05 | 3.7E+05 |
| NK cells (BM) | 3.6 | 1.9 | 6.4E+05 | 3.3E+05 |
| HSPCs (mfg) | 0.8 | 0.4 | 9.0E+04 | 5.8E+04 |
| Tregs (BM) | 0.5 | 0.2 | 8.2E+04 | 3.8E+04 |

TABLE 13
Time duration between collection and administration

| Category | Activity | N | Time (hrs) |
|---|---|---|---|
| Transportation | Local collection site | N = 17 | 2.8 +/− 1.3 |
| | to sorting facility | | |
| Transportation | Out of State | N = 7 | 11.2 +/− 2.6 |
| | collection facility to | | |
| | sorting facility | | |
| Manufacturing | Product dwell time at | N = 24 | 25.6 +/− 5.0 |
| | sorting facility | | |
| Total | Vein-to-vein | N = 24 | 60.5 +/− 10.9 |

TABLE 14
Endotoxin levels

| | Endotoxin | Endotoxin | Endotoxin |
|---|---|---|---|
| | (EU/ml) in HSPC | (EU/ml) in Treg | (EU/ml) in Tcon |
| Patients | drug product | drug product | drug product |
| 1 | <0.025 | <0.05 | <0.5 |
| 2 | <0.025 | <0.05 | <0.5 |
| 3 | 0.043 | <0.061 | <0.5 |
| 4 | <0.025 | <0.061 | <0.5 |
| 5 | <0.026 | <0.05 | <0.5 |
| 6 | <0.025 | <0.05 | <0.5 |
| 7 | <0.025 | <0.05 | <0.5 |
| 8 | <0.025 | <0.05 | <0.5 |
| 9 | <0.025 | <0.05 | <0.5 |
| 10 | <0.025 | <0.05 | <0.717 |
| 11 | <0.025 | <0.05 | <0.5 |
| 12 | <0.025 | <0.05 | <0.5 |
| 13 | <0.025 | <0.05 | <0.5 |
| 14 | <0.025 | <0.05 | <0.5 |
| 15 | <0.025 | <0.05 | <0.5 |
| 16 | <0.025 | <0.05 | <0.5 |
| 17 | <0.025 | <0.05 | <0.5 |
| 18 | <0.025 | <0.05 | <0.5 |
| 19 | <0.025 | <0.05 | <0.5 |
| 20 | <0.025 | <0.05 | <0.5 |
| 21 | <0.025 | <0.05 | <0.5 |
| 22 | <0.025 | <0.05 | <0.5 |
| 23 | <0.025 | <0.05 | <0.5 |
| 24 | <0.025 | <0.05 | <0.5 |
| 25 | <0.025 | <0.05 | <0.5 |
| 26 | <0.025 | <0.05 | <0.5 |
| 27 | <0.025 | <0.05 | <0.5 |
| 28 | <0.025 | <0.05 | <0.5 |
| 29 | <0.5 | <0.5 | <0.5 |
| 30 | <0.025 | <0.05 | <0.5 |
| 31 | <0.025 | <0.05 | <0.5 |
| 32 | <0.025 | <0.05 | <0.5 |
| 33 | <0.025 | <0.05 | <0.5 |
| 34 | <0.025 | <0.063 | <0.5 |
| 35 | <0.025 | <0.05 | <0.5 |
| 36 | <0.025 | <0.05 | <0.5 |
| 37 | <0.025 | <0.05 | <0.5 |
| 38 | <0.025 | <0.05 | <0.5 |
| 39 | <0.025 | <0.05 | <0.5 |
| 40 | <0.025 | <0.05 | <0.5 |
| 41 | <0.025 | <0.05 | <0.5 |
| 42 | <0.025 | <0.05 | <0.5 |
| 43 | <0.025 | <0.05 | <0.5 |
| 44 | <0.025 | 0.066 | <0.636 |
| 45 | <0.025 | 0.05 | <0.5 |
| 46 | <0.025 | 0.05 | <0.5 |
| 47 | <0.025 | <0.05 | <0.5 |
| 48 | <0.025 | <0.025 | <0.5 |
| 49 | <0.025 | 0.075 | <0.5 |
| 50 | <0.025 | <0.05 | <0.5 |
| 51 | <0.025 | <0.05 | <0.5 |
| 52 | <0.025 | <0.05 | <0.5 |
| 53 | <0.025 | <0.05 | <0.5 |
| 54 | <0.0391 | <0.05 | <0.5 |
| 55 | <0.025 | <0.05 | <0.5 |
| 56 | <0.025 | <0.05 | <0.5 |
| 57 | <0.025 | <0.05 | <0.5 |
| 58 | <0.025 | <0.05 | <0.809 |
| 59 | <0.0294 | <0.05 | <0.5 |
| 60 | 0.0457 | <0.05 | <0.5 |
| 61 | 0.0427 | <0.063 | <0.5 |
| 62 | <0.025 | <0.05 | <0.5 |

Recipients had appropriate long-term central venous access placed prior to initiation of the conditioning regimen. Unless contraindicated, subjects were administered acetaminophen or paracetamol (e.g., 500-1000 mg) and diphenhydramine (e.g., 25-50 mg) prior to administration of Teach cell component.

The CD34+ HSPC cell component (e.g., the first population of CD4+ cells), then the Treg cell component (e.g., the population of cells enriched for Tregs), were intravenously (IV) through a central venous catheter on study Day 0.

Subjects received a myeloablative conditioning (MA) regimen prior to administration of the cell components. Examples of MA regimens are provided in TABLE 15. Busulfan could also be dosed to maintain an average daily AUC of 4,800-6,000 μM-min.

| MA regimen | Drugs and doses |
|---|---|
| Total Body Irradiation/Cyclophosphamide/ | TBI (1320-1400 cGy) |
| Thiotepa (TBI/Cy/Thiotepa) | Cyclophosphamide (120 mg/kg) |
| | Thiotepa (10 mg/kg) |
| Busulfan/Melphalan/Fludarabine | Busulfan (9.6 mg/kg IV) |
| (Bu/Mel/Flu) | Fludarabine (125 mg/m²) |
| | Melphalan (140 mg/m²) |
| Busulfan/Fludarabine (Bu/Flu) | Busulfan (16 mg/kg PO or 12.8 mg/kg IV) |
| | Fludarabine (160 mg/m²) or 120-180 mg/m² |
| | administered over 4 days |
| Busulfan/Cyclophosphamide (Bu/Cy) | Busulfan (16 mg/kg PO or 12.8 mg/kg IV) |
| | Cyclophosphamide (100 mg/kg) |
| Cyclophosphamide/Total Body Irradiation | Cyclophosphamide (100 mg/kg) |
| (Cy/TBI) | TBI (1200-1420 cGy) |
| Total Body Irradiation/Etoposide | TBI (1200-1320 cGy) |
| (TBI/Etoposide) | Etoposide (60 mg/kg) |
| Total Body Irradiation/Etoposide/ | TBI (1320 cGy) |
| Cyclophosphamide (TBI/Etoposide/Cy) | Etoposide (60 mg/kg, 1 dose) |
| | Cyclophosphamide (60 mg/kg, 1 dose) |
| Bu/Flu/TTP | Thiotepa 5 mg/kg × 2 days (days −7 and −6) |
| | Busulfan 3.2 mg/kg × 3 days (days −5 to −3), |
| | total dose 9.6 mg/kg |
| | Fludarabine 50/m² (meters squared − body |
| | surface area) × 3 days (days −5 to −3) |
| TBI/Flu/TTP | Total body irradiation: HFTBI administered in |
| | 11 fractions of 125 cGy (centigray) over 4 |
| | days, to a total dose of 1375 cGy. |
| | Thiotepa 5 mg/kg × 2 days (days −7 and −6) |
| | Fludarabine 25/m² × 5 days |
| TBI/Flu/TTP/Bu | Thiotepa 5 mg/kg × 2 days (days −7 and −6) |
| | Busulfan 3.2 mg/kg × 3 days (days −5 to −3), |
| | total dose 9.6 mg/kg |
| | Fludarabine 50/m² (meters squared − body |
| | surface area) × 3 days (days −5 to −3) |
| | Total body irradiation: HFTBI administered in |
| | 11 fractions of 125 cGy (centigray) over 4 |
| | days, to a total dose of 1375 cGy |

Subjects received either tacrolimus or sirolimus as a single-agent GVHD prophylaxis beginning on the day following Tcon infusion (typically Day +3), e.g., of the second population of CD45+ cells.

Tacrolimus was initiated at 0.03 mg/kg/day IV, with a target trough blood level of 5-10 ng/mL. Per os (PO) administration was permissible if the patient was able to tolerate food.

For subjects that underwent MA with TBI/Cy/Thiotepa, Cy/TBI, TBI/Etoposide, TBI/Etoposide/Cy (i.e., regimens that do not include busulfan), sirolimus was initiated as a single loading dose of 6 mg PO, followed by 2 mg daily for a target blood level of 3-8 ng/mL.

For subjects that did not show signs of ≥grade 2 acute GVHD prior to Day +60, the GVHD prophylaxis could be reduced, e.g., by approximately 20% of the dose per month. For subjects that showed signs of ≥grade 2 acute GVHD, GVHD prophylaxis could be tapered after no signs of GVHD were observed for a suitable period of time (e.g., a suitable period of time after ceasing administration of any GVHD therapeutic agents, and not observing ≥grade 2 GVHD).

H. Clinical Outcomes

The following paragraphs provide data and explanations related to clinical outcomes from the clinical studies described herein. As an introductory matter in the explanation of the data, it is to be recognized that graft failure can be a complication of HCT, and can be associated with significant mortality.

Neutrophil engraftment through Day +28: Neutrophil engraftment was defined as achieving an absolute neutrophil count (ANC) ≥500/mm³ for 3 consecutive days, by Day +28. The first of the three days was designated the day of engraftment. If ANC never dropped below 500/mm³, Day +1 was assigned as the day of engraftment. Study group patients showed earlier neutrophil (median of 11 days vs. 14 days, p<0.0001 by Mann-Whitney U).

Platelet engraftment through Day +50: Platelet engraftment was defined as achieving a platelet count >20,000/mm³ for 3 consecutive days without platelet transfusion in the preceding 7 days, by Day +50. The first of the three days was designated the day of engraftment. If platelet count never dropped below 20,000/mm³, Day +1 was assigned as the day of engraftment. Study group patients showed earlier platelet engraftment (11 vs 17 days, p<0.0001).

Secondary graft failure through Day +100: Secondary graft failure was defined as neutrophil engraftment followed by subsequent decline in absolute neutrophil counts <500 cells/μL, unresponsive to growth factor therapy, by Day +100.

A failure to achieve an absolute neutrophil count of >500 cells/μL after Day +30 can indicate primary graft failure. No subjects in the study group experienced primary graft failure.

A sustained loss of hematopoiesis after engraftment has occurred can indicate secondary graft failure. No subjects in the study group experienced secondary graft failure.

Peripheral blood samples from subjects were also analyzed for chimerism as part of the follow-up procedures. The blood samples were processed to select for various cell populations using standard markers for each cell population. Blood was drawn from subjects that received the composition of the disclosure on day +28, day +56, day +100, day +180 and day +365 post-transplant, and the frequency of peripheral blood cells were quantified by flow cytometry (Markers used for analysis are described in Table 16).

Achieving a platelet count >20,000/mm³ for 3 consecutive days without platelet transfusion can indicate platelet engraftment. As shown in FIG. 3A, subjects in the study group exhibited more rapid platelet engraftment than subjects in the SOC cohort, achieving platelet engraftment after a median of 11 days compared to 17 days for the SOC cohort (p<0.0001). FIG. 3B illustrates the platelet counts in donors before and after mobilization and in receiving patients before transplant and after transplants. Boxplots where shown: boxes show the 75th, 50^{th}, and 25^{th} percentiles; whiskers show the 90^{th} and 10^{th} percentiles. X-axes nomenclature: the leading number (e.g. 01, 02, 025, . . . ) are mentioned for ordering; following the underscore, Dscrn=healthy donor pre-G-CSF mobilization, Rscrn=recipient within 1 month prior to conditioning, apher=healthy donor blood draw at the time of apheresis, d028=recipient day 28 post transplant, d056-d365=recipient days post transplant. N's shown indicate the sample sizes for each timepoint. Symbols indicate values for individual measurements.

Achieving a sustained neutrophil count of >500 cells/μL can indicate neutrophil engraftment. FIG. 3C illustrates the platelet counts in donors before and after mobilization and in receiving patients before transplant and after transplants. Figure legends are similar to the legend described in FIG. 3B.

FIGS. 3D-E illustrate monocyte and lymphocyte engraftment in the patients. FIGS. 3M and N illustrate a comparison of lymphocyte and monocyte counts in representative patients compared to a representative SOC cohort.

FIG. 3F illustrates that B cells engraft in recipients of the composition of the disclosure, and that mature B cells are present by day +100 post-transplant. Superior engraftment and/or earlier functionality of engrafted B cells may represent a significant advantage over standard of care grafts, for example, enhancing immunity and allowing for vaccination post-transplant.

FIG. 3G illustrates CD3+ T cell engraftment; FIG. 3H demonstrates NK cell engraftment, FIG. 3I illustrates CD4+ T cell engraftment and FIG. 3J illustrates CD8+ T cell engraftment in recipients of the composition of the disclosure. FIG. 3K illustrates a ratio of CD4:CD8 T cells in the recipients.

FIG. 3L provides a comparison of the proportion of CD3+CD4+ T cells that were Tregs in healthy donors, compared to graft recipients on several days post-transplant. These data show that recipients of the composition of the disclosure exhibit high frequencies of circulating CD4+ Tregs. Without wishing to be bound by theory, a lower Treg frequency may contribute to prevention of GVHD, reduced risk of developing GVHD, reduced incidence of GVHD, reduced severity of GVHD, or a combination thereof.

FIG. 3O shows representative data from two subjects compared to a healthy control. In the healthy control, 3.72% of circulating CD3+CD4+ T cells were Tregs (CD25+CD127dim). In the two graft recipients, 28.10% and 23.7% of CD3+CD4+ T cells were Tregs on day +28, 32.3% and 17.8% on day +56, and 19.2% and 20.7% on day +100.

Blood drawn from a graft recipient on day +100 post-transplant the sample was processed for flow cytometric evaluation of various B cell markers including CD19, CD20, IgD, and CD27. FIG. 3P compares scatterplots from the graft recipient to a healthy control. In all cases, the Y axis is for CD19+ staining. The left panels show gating of lymphocytes to identify B cells (CD19+) and T cells (CD3+). 13.4% of lymphocytes in the graft recipient were B cells, compared to 9.84% in the healthy control. The following panels show that 98.3-100% of cells gates as CD19+ were also CD20+. The panels second from the right show the fraction of B cells that are IgD+, which can be used to identify mature B cells. 92.1% of B cells in the graft recipient were IgD+, and 89.5% in the healthy control. The right-most panels show staining for CD27, which can be used to identify memory B cells, late plasmablasts, and plasma cells, for example. 43.6% of B cells in the graft recipient were CD27+, and 67.10% in the healthy control.

These results demonstrate that B cells engraft in recipients of the composition of the disclosure, and that mature B cells are present by day +100 post-transplant. Superior engraftment and/or earlier functionality of engrafted B cells may represent a significant advantage over standard of care grafts, for example, enhancing immunity and allowing for vaccination post-transplant.

I. GVHD Evaluation

Acute GVHD was staged and graded per MAGIC Standardization criteria.

Chronic GVHD was diagnosed, staged and graded per the International NIH Chronic GVHD Diagnosis and Staging Consensus Working Group criteria.

Clinically significant manifestations of both acute and chronic GVHD were treated first by local, topical, and/or systemic corticosteroids (e.g. prednisone). Any patients who were refractory or resistant to, dependent upon, or intolerant of corticosteroids, per BMT-NIH-CIBMTR Task Force definition, were to be considered for second line therapy.

Treatment-emergent adverse events (TEAEs): TEAEs were categorized by System Organ Class and preferred term using MedDRA version 21.0 and were graded according to the CTCAE version 5.0.

Acute GVHD: Acute GVHD (aGVHD) is a significant driver of morbidity and mortality associated with alloHCT, reducing the severity and incidence of aGVHD has the potential to greatly benefit graft recipients. Acute GVHD was staged and graded per Mount Sinai Acute GvHD International Consortium (MAGIC) Standardization criteria.

Subjects were considered evaluable for aGVHD if they developed aGVHD symptoms before day +100 (100 days post-transplant), or were beyond day +100 without exhibiting aGVHD symptoms. Using these criteria, 17 patients are evaluable for aGVHD at the time of reporting.

The Grade ≥2 aGVHD rate observed was 16% at the time of reporting. This compares favorably to published rates of in similar populations, and was lower than patients in the SOC cohort. The onset of grade ≥2 aGVHD compared to the SOC cohort is shown in FIG. 4A. One subject developed aGVHD of the upper gastrointestinal (GI) tract, manifesting as nausea and cachexia. This subject's symptoms resolved with a short course of corticosteroids, which were subsequently weened.

Severe (Grade 3-4) aGVHD is a major contributor to non-relapse mortality post-alloHCT and can be observed in 10-20% of patients following an HLA-matched, related donor transplants. 5% of the patients developed grade 3-4 aGVHD in the study group, whereas 20% of patients in the SOC cohort developed Grade 3-4 aGVHD. The onset of grade ≥3 aGVHD in through Day +120 compared to the SOC cohort is shown in FIG. 4B.

As noted in TABLE 7, four patients in the study group did not receive any GVHD prophylaxis. Of those patients, only one has developed aGVHD symptoms (the case of upper GI tract aGVHD noted above). Results to the time of reporting suggest that the composition of the disclosure may be safely administered to patients without GVHD prophylaxis or with minimal prophylaxis. These strategies could significantly benefit patients due to, for example, increased graft versus tumor (GVT), increased graft versus infection (GVI), and reduced adverse effects that can be associated with immunosuppressive agents (e.g., renal toxicity and hepatotoxicity).

These results suggest a composition of the disclosure can reduce the incidence and severity of aGVHD in recipients compared to standard of care, for example, even in the absence of GVHD prophylactics or with reduced GVHD prophylaxis.

Steroid-refractory acute GVHD: Steroid-refractory acute GVHD was defined as per the EBMT-NIH-CIBMTR Task Force position statement.

Chronic GVHD: As chronic GVHD (cGVHD) is associated with significant morbidity and with decreased survival, reducing the severity or incidence of cGVHD has the potential to greatly benefit graft recipients. Chronic GVHD was diagnosed per 2014 International NIH Chronic GVHD Diagnosis and Staging Consensus Working Group criteria.

At the time of reporting, no subjects in the study group had developed moderate or severe cGVHD. One subject in the study group developed transient, steroid-responsive mild cGVHD of the skin.

Analysis was conducted using a cutoff of 365 days (i.e. cGVHD events that occur after Day +365 were not included). As shown in FIG. 4C, Subjects in the study group experienced significantly fewer moderate or severe cGVHD events than patients in the SOC cohort (5% vs. 43%, respectively; p<0.0001). Despite receiving less GVHD prophylaxis or no GVHD prophylaxis, data showed that a composition of the disclosure can significantly reduce the incidence of cGVHD compared to subjects in the SOC cohort. Additionally, several subjects in the study group have undergone greater than 2.5 years of follow-up with no cGVHD symptoms reported.

As cGVHD is associated with decreased overall survival and significant long-term morbidity, these results suggest that a composition of the disclosure can improve long-term survival and quality of life in recipient subjects compared to standard of care.

Post-Transplant Lymphoproliferative Disorder (PTLD): PTLD was defined as a biopsy consistent with the 2017 World Health Organization (WHO) classification of PTLD (nondestructive [plasmacytic hyperplasia, infectious mononucleosis-like, and florid follicular hyperplasia], polymorphic, monomorphic or Hodgkin lymphoma-like), along with lymphoma type-appropriate staging procedures such as computed tomography (CT) with or without 18F-fluorodeoxyglucose positron-emission tomography (FDG-PET).

Incidence of non-relapse mortality (NRM). NRM was defined as death without evidence of disease recurrence. Disease relapse/progression was considered a competing event.

Incidence of disease relapse: For acute leukemias, relapse was defined as any of the following (MRD+ alone was insufficient): (i) ≥5% blasts in the bone marrow or peripheral blood; or (ii) Reappearance of pre-transplant cytogenetic abnormality; or (iii) new evidence or redevelopment of extramedullary disease. For MDS, relapse was defined as any of the following: (i) satisfying criteria for evolution into acute leukemia; (ii) reappearance of pre-transplant morphologic abnormalities, detected in bone marrow specimens; or, (iii) reappearance of pre-transplant cytogenetic abnormality in at least one metaphase on each of two separate consecutive examinations at least one month apart, regardless of the number of metaphases analyzed.

Treatment related mortality includes deaths from complications or toxicities associated with therapy, such as infection, GVHD, or organ failure. At the time of reporting, the treatment-related mortality rate in the study group was 5% for one-year post-transplant, compared to 13% for the SOC cohort, as shown in FIG. 4D. No subjects in the study group had died from complications other than disease relapse.

Subjects were monitored for survival and relapse. For comparison of clinical outcomes, a standard of care (SOC) comparator cohort was identified retrospectively from subjects who received a SOC myeloablative alloHCT of mobilized peripheral blood from a fully HLA-matched donor at one of the same clinical sites during the same period. All subjects in the SOC cohort received myeloablative conditioning regimens and dual-agent GVHD prophylaxis with tacrolimus and methotrexate.

FIGS. 4E-H illustrate the relapse, GVHD and relapse free survival rates, chronic GVHD free survival rates and overall survival rates of subjects that were recipients of standard of care grafts compared to subjects that received grafts described in this example. These data suggest that the compositions described herein improve relapse-free survival in subjects undergoing myeloablative alloHCT.

GVHD and relapse-free survival is a composite readout that can refer to survival without relapse or Grade ≥3 acute or extensive chronic GVHD. At the time of reporting, the percent of GVHD-free and relapse-free survival to one-year post-transplant was 74% for the study group, compared to 34% for the SOC cohort, as shown in FIG. 4F. The difference was statistically significant (p=0.0001 by log-rank test).

Overall survival: OS was defined as the time from the date of transplant to the date of death from any cause or, for surviving patients, to the date of last follow-up. At the time of reporting, the percent of overall survival to one-year post-transplant was 90% for the study group, compared to 78% for the SOC cohort, as shown in FIG. 4H. The difference was statistically significant (p=0.0242 by log-rank test).

H. Hospitalization Time

Standard of care HCT can require a lengthy inpatient stay. Compared to patients in the SOC cohort, the median time from transplant (Day 0) to hospital discharge for patients in the study group was 2.5 days shorter (from 18.5 to 16 days, p<0.01 by Mann-Whitney U test). FIG. 4I illustrates hospitalization times for a representative subset of patients.

I. Disease Status after Therapy

Subjects in the study were first evaluated for relapse on day +90 post-transplant. At the time of reporting, only 16% patients relapsed compared to 19% patients in the SOC cohort.

TABLE 16: subject status for a representative subset of subjects past day +90. Abbreviations used: DLBCL, diffuse large B cell lymphoma; AML, acute myeloid leukemia; ALL, acute myeloid leukemia; MDS, myelodysplastic syndrome; MF, myelofibrosis; MPAL, mixed phenotype acute leukemia; CML, chronic myeloid leukemia; CR: complete remission; CR1, 1st complete remission; CR2, 2nd complete remission; active, active disease (not in CR as defined for given disease entity).

TABLE 16

| | Disease | |
|---|---|---|
| | status at | |
| Disease | transplant | Most recent follow up and status |
| DLBCL | Active | Relapsed, day +113 |
| AML | CR2 | Relapsed, day +367 |
| ALL | Active | Alive, in remission, day +1107 |
| ALL | CR2 | Alive, in remission, day +1079 |
| CML | Active | Relapsed, day +76, then responded to donor |
| | | lymphocyte infusion; Alive, in remission, day +1058 |
| AML | CR2 | Alive, in remission, day +981 |
| MF | Active | Alive, in remission, day +932 |
| AML | CR1 | Alive, in remission, day +260 |
| PTCL | Active | Alive, in remission, day +225 |
| AML | CR1 | Alive, in remission, day +211 |
| AML | CR1 | Alive, in remission, day +204 |
| MDS | Active | Alive, in remission, day +190 |
| ALL | CR1 | Relapsed, day +119 |
| ALL | CR2 | Alive, in remission, day +127 |
| MF | Active | Alive, in remission, day +119 |
| MPAL | CR1 | Alive, in remission, day +120 |
| AML | CR1 | Alive, in remission, day +99 |

The risk of relapse can be associated with disease status at the time of transplant. For example, the prognosis of AML or ALL subjects can be significantly worse if the subjects are not in complete remission at time of transplant (i.e., prognosis is worse if the subjects have active leukemia or detectable minimal residual disease at the time of transplant). In another example, prognosis is worse in subjects that have detectable minimal residual disease versus patients who do not have detectable minimal residual disease. Subjects with active disease or with minimal residual disease can represent a critical unmet medical need.

FIG. 5 summarizes the disease status of a subset of subjects in the study group before transplant and at day +90, +180, and +356 post-transplant. Of the 10 subjects that received the composition of the disclosure, 7 achieved durable remissions, demonstrating a graft-versus tumor effect. Two of the subjects that relapsed had active disease at the time of transplant, and one had detectable minimal residual disease (MRD) at the time of transplant

J. Characteristics of Transplanted Cells

A schematic of graft production and administration for the sorting protocol is provided in FIG. 1A. Cell product from apheresis collection Day 2 was given an assessment and a portion of the apheresis product comprising a heterogenous cell component (e.g., a second population of CD45+ cells) consisting of 3×10⁶ Tcon cells was administered to the subjects. A portion of the heterogenous cell component was analyzed for different cell components after staining for various cell populations as described in Table 17.

TABLE 17

| Cell population | Immunophenotype ((+) positive staining, (−) negative staining) |
|---|---|
| Granulocytes | CD45+, high side scatter, CD14− |
| Monocytes | CD45+, moderate side scatter, high forward scatter, CD14+ |
| Lymphocytes | CD45+, low forward and side scatter |
| PBMC | Defined as the total population of lymphocytes and monocytes |
| Lymphocyte subsets | |
| T cells | CD3+ lymphocytes |
| CD4+ T cells | CD4+, CD8- T cells |
| CD8+ T cells | CD8+, CD4- T cells |
| Naïve T cells | CD45RA+ CD45RO− T cells (can be subdivided based on CD4 and CD8 |
| | expression) |
| Memory T cells | CD45RA− CD45RO+ T cells (can be subdivided based on CD4 and CD8 |
| | expression) |
| Tregs | CD25+, CD127dim CD4+ T cells |
| NKT cells | CD56+ T cells |
| B cells | CD19+, CD3− lymphocytes |
| NK cells | CD56+, CD3− lymphocytes |
| CD34+ cells (HSPCs) | CD45dim, CD34+, CD3− cells that fall within the general lymphocyte |
| | scatter region |
| PBMC subsets | |
| Plasmacytoid dendritic cells | CD3−, CD19−, CD56−, CD14− (Lin−) cells that are CD4+, CD11b−, |
| (pDCs) | and BDCA2+ |
| Myeloid dendritic cells | CD3−, CD19−, CD56−, CD14− (Lin−) cells that are CD4+, CD11b+, |
| (mDCs) | and variably express CD16 |

Table 18 below illustrates data collected from a few patients from the study and identifies the various cell populations transplanted into a subject as part of the heterogenous cell component comprising 3×10⁶ Tcon cells.

TABLE 18
Heterogenous cell component

| | Parent | | Standard | | | | | |
|---|---|---|---|---|---|---|---|---|
| Cell subset | gate | Mean | Deviation | Median | Range | Minimum | Maximum | Count |
| Tcons | 45 + Lymph | 60.05 | 8.67 | 58.6 | 33.4 | 47.6 | 81 | 22 |
| CD4+ T | T cells | 48.59 | 10.07 | 46.55 | 36.1 | 32.7 | 68.8 | 22 |
| cells | | | | | | | | |
| CD8+ T | T cells | 42.45 | 10.27 | 42.1 | 32.7 | 24.7 | 57.4 | 22 |
| cells | | | | | | | | |
| B-cells | Lymph | 18.62 | 6.37 | 19.25 | 25.09 | 5.71 | 30.8 | 22 |
| NK cells | 45 + Lymph | 11.81 | 5.5 | 11.4 | 23.5 | 4.2 | 27.7 | 22 |
| NKT cells | T cells | 10.02 | 5.38 | 9.01 | 20.98 | 3.32 | 24.3 | 22 |
| Treg cells | T4 | 6.01 | 2.1 | 5.84 | 7.49 | 2.71 | 10.2 | 21 |
| Naïve T | T cells | 50.25 | 8.57 | 49.5 | 32.8 | 34.8 | 67.6 | 21 |
| cells | | | | | | | | |
| DRp38pT4 | T4 | 3.81 | 2.64 | 2.99 | 10.81 | 1.09 | 11.9 | 22 |
| DRp38pT8 | T8 | 7.39 | 4.67 | 5.72 | 18.24 | 1.96 | 20.2 | 22 |
| CD34+ | 45 + Lymp | 1.62 | 0.64 | 1.66 | 2.33 | 0.31 | 2.64 | 22 |
| HSPCs | h | | | | | | | |
| Granulocytes | 45+ | 58.48 | 9.12 | 58.7 | 34.8 | 38.3 | 73.1 | 20 |
| and | | | | | | | | |
| Monocytes | | | | | | | | |
| Lymphocytes | 45+ | 28.64 | 10.02 | 28.75 | 34.4 | 10.4 | 44.8 | 20 |
| CD14+ | Total | 26.28 | 6.59 | 26.5 | 25 | 16.4 | 41.4 | 20 |
| pDC | Lin- | 6.66 | 5.05 | 4.43 | 17.53 | 1.97 | 19.5 | 12 |
| mDC | Lin- | 14.15 | 9.2 | 10.95 | 27.7 | 3.3 | 31 | 12 |

### Example 2: Analysis of Patients Receiving Different Conditioning Regimens

The data was further quantified for clinical outcomes in subjects that received different conditioning regimens. FIG. 6A compares acute GVHD in patients that received a Bu/Cy conditioning (see Table 14) vs a conditioning regimen comprising Thiotepa (BFT—busulfan, fludarabine and thiotepa; regimen described in Table 14). The acute GVHD rates were similar for the two regimens but were lower in patients that received thiotepa. A higher difference was seen in patients that received thiotepa in chronic GVHD (FIG. 6B). Surprisingly, the relapse rate in patients dropped significantly (p<0.03) in patients with the BFT regimen where no patients who received the BFT regimen relapsed (see FIGS. 6C and D). The GVHD and Relapse Free survival rates (GRFS) were also significantly lower in the BFT regimen patients (see FIG. 6E). The overall survival was also improved in the BFT-receiving patients (see FIG. 6F). These data demonstrate that conditioning regimens comprising thiotepa provide a clear and surprising advantage.

### Example 3: Analysis of Patients Receiving Different GVHD Prophylactic Agent Regimens

The data from the clinical trial was further quantified for clinical outcomes in subjects that received different GVHD prophylactic agents—sirolimus or tacrolimus. FIG. 7A compares acute GVHD in patients that received sirolimus only or tacrolimus only versus standard of care (SOC) patients that received a combination of methotrexate and tacrolimus (see FIG. 1B for SOC regimen). The patients that received only tacrolimus showed higher GVHD rates (FIGS. 7A-C) than patients who received sirolimus. However, the survival (FIG. 7E), relapse rates (FIG. 7F), GRFS rates (FIG. 7G) and overall survival rates (FIG. 7H) were improved in tacrolimus-only patients relative to the sirolimus-only patients. As FIG. 7A shows, multiple drugs can be used as GHVD prophylaxis with significantly lower rates of acute GVHD versus standard of care.

Upon further analysis, it was observed that patients had different serum trough levels. Serum tacrolimus levels had direct effects on the clinical outcome in the patient populations as is shown in FIGS. 8A-9G). As illustrated in FIG. 8A, patients who maintained an average serum tacrolimus trough level higher than 4 ng/ml for the first 30 days post-transplant had lower GVHD rates. FIGS. 8B-C are derived from the data in FIG. 8A and further present data for acute GVHD and chronic GVHD rates. They illustrate a calculation of the probability of developing GVHD (y axis) if tacrolimus levels fall below the threshold value (x axis).

FIGS. 9A-9G illustrate direct comparison of clinical outcomes in patients that had a serum tacrolimus trough levels higher or lower than 4 ng/ml. Each figure legend describes the number of patients and the time periods where their serum tacrolimus trough levels were higher or lower than 4 ng/ml. Acute GVHD rates showed significant improvement with higher tacrolimus levels (FIG. 9A) and chronic GVHD rates also showed improvements (FIG. 9B).

Patients who received the same conditioning regimen (Bu/Cy for FIGS. 9C-D; TBI/BFT for FIGS. 9E-G) and but had different serum tacrolimus trough levels (higher or lower than 4 ng/ml) were also analyzed for GVHD rates and results are illustrated in FIGS. 9C-G. In all instances, a higher tacrolimus trough level showed improvement in GVHD rates.

When T cell chimerism was compared to the average trough tacrolimus level through day +30 post-transplant, a significant correlation was found; lower trough tacrolimus levels were associated with increased engraftment of donor T cells (p=0.0011). FIG. 9H shows the average trough tacrolimus level through day +30 post-transplant for a small subset of patients, plotted against the proportion of CD3+ cells of donor origin at day +30 (except that chimerism data is from day 90 where indicated by “D90”). These data suggest that maintaining lower circulating levels of tacrolimus can contribute to improved engraftment of donor T cells and improved donor T cell chimerism, which may contribute to improved relapse-free survival in allogeneic hematopoietic stem cell transplant recipients. Without wishing to be bound by a theory, it can be hypothesized that an improved chimerism due to higher tacrolimus levels may have led to the improved GVHD and survival outcomes in patients.

### CONCLUSION

While various embodiments of the present inventions have been shown and described herein, it will be obvious to those skilled in the art that such embodiments are provided by way of example only. Numerous variations, changes, and substitutions will be apparent to those skilled in the art without departing from the disclosure. For example, various embodiments of therapeutic compositions, methods of can be adapted for various pediatric, geriatric and veterinary applications, the latter including one or more of felines, canines and equines. Specific adaptions for such can include one or more of the cell types in the therapeutic composition administered to the patient and the dose of cells in the therapeutic composition. They can also be adapted for treatment of any number of hematologic and/or stem cell-based cancers including leukemia, lymphoma, myeloma, as well as a number of t-cell mediated and other autoimmune diseases including one more of multiple sclerosis, IBD, Celiac Disease, Crohn's disease, ulcerative colitis, ankylosing spondylosis, myasthenia gravis and diabetes. It should be understood that various alternatives to the embodiments of the invention described herein may be employed in practicing the invention.

Elements, characteristics, or acts from one embodiment can be readily recombined or substituted with one or more elements, characteristics or acts from other embodiments to form numerous additional embodiments within the scope of the invention. Moreover, elements that are shown or described as being combined with other elements, characteristics, steps or acts, can, in various embodiments, exist as stand-alone elements, characteristics, steps or acts. Further, various embodiments expressly contemplate the negative recitation of any element, characteristic, step or act etc. that is/are shown or described in one or more embodiments. Hence, the scope of the present invention is not limited to the specifics of the described embodiments, but is instead limited solely by the appended claims.

1-214. (canceled)

215. A multi-component pharmaceutical treatment to be administered to a human subject in need thereof, the multi-component treatment comprising:
(a) a solution comprising a first population of CD45+ cells comprising hematopoietic stem and progenitor cells (HSPCs) and granulocytes, wherein at most about 10% of the first population of CD45+ cells comprises granulocytes;
(b) a solution comprising a population of cells enriched for regulatory T cells (Tregs);
(c) a solution comprising a second population of CD45+ cells, wherein the second population of CD45+ cells comprises at least about 20% CD3+ conventional T cells (Tcons), at least about 10% monocytes, and at least about 10% granulocytes; and
(d) a solution comprising one or more doses of a graft vs host disease (GVHD) prophylactic agent.

216. The multi-component pharmaceutical treatment of claim 215, wherein the GVHD prophylactic agent is tacrolimus.

217. The multi-component pharmaceutical treatment of claim 216, wherein the tacrolimus is formulated for administration in an amount to maintain or that maintains a target blood level of at least about 3 ng/ml for at least about 20 days after administering the second population of CD45+ cells.

218. The multi-component pharmaceutical treatment of claim 215, wherein the first population of CD45+ cells comprises at most about 3% monocytes.

219. The multi-component pharmaceutical treatment of claim 215, wherein the first population of CD45+ cells comprises at most about 2% lymphocytes.

220. The multi-component pharmaceutical treatment of claim 215, wherein the second population of CD45+ cells comprises at least about 0.5% NK cells.

221. The multi-component pharmaceutical treatment of claim 215, wherein the second population of CD45+ cells comprises at least about 0.1% CD34+ cells or from about 0.2% to about 20% CD34+ cells.

222. The multi-component pharmaceutical treatment of claim 215, wherein the treatment further comprises a conditioning regimen.

223. The multi-component pharmaceutical treatment of claim 222, wherein the conditioning regimen is a myeloablative conditioning regimen.

224. The multi-component pharmaceutical treatment of claim 223, wherein the conditioning regimen comprises at least three conditioning reagents, wherein at least one conditioning reagent comprises thiotepa.

225. The multi-component pharmaceutical treatment of claim 224, wherein the conditioning regimen comprises one or more doses of busulfan, fludarabine and thiotepa.

226. The multi-component pharmaceutical treatment of claim 225, wherein the one or more doses of busulfan, fludarabine and thiotepa comprises from about 5 to about 12 mg of thiotepa per kg human subject's actual or ideal body weight, from about 7 to about 11 mg of busulfan per kg human subject actual or ideal body weight, and from about 100 to about 200 mg of fludarabine per meter² body surface area respectively.

227. The multi-component pharmaceutical treatment of claim 215, wherein the first population of CD45+ cells comprises less than about 5 EU of endotoxins per ml of the solution, the population of cells enriched for Tregs comprises less than about 5 EU of endotoxins per ml of the solution, and/or the second population of CD45+ cells comprises less than about 5 EU of endotoxins per ml of the solution.

228. The multi-component pharmaceutical treatment of claim 215, wherein said HSPCs are CD34+, and said Tregs are CD4+CD25+CD127dim or CD4+CD25+CD127dim or CD4+FOXP3+.

229. The multi-component pharmaceutical treatment of claim 215, wherein:
a) said first population of CD45+ cells comprising HSPCs comprises from about 5×10⁵ to about 2×10⁷ HSPCs per kilogram of ideal body of said human subject;
b) said population of cells enriched for Tregs comprises from about 1×10⁵ to about 1×10⁷ Tregs per kilogram of actual or ideal body weight of said human subject, or from about 5×10⁵ to about 4×10⁶ Tregs per kilogram of actual or ideal body weight of said human subject; and
c) said second population of CD45+ cells comprises from about 1×10⁵ to about 1×10⁷ Tcons per kilogram of actual or ideal body weight of said human subject, or from about 5×10⁵ to about 5×10⁶ Tcons per kilogram of actual or ideal body weight of said human subject.

230. The multi-component pharmaceutical treatment of claim 215, wherein a first dose of the one or more doses of the GVHD prophylactic agent is formulated for administration after administration of the second population of CD45+ cells.

231. A method of treating a human subject having a hematologic malignancy, the method comprising administering to the human subject the multi-component pharmaceutical treatment of claim 215.

232. The method of claim 231, wherein risk and/or severity of an adverse event associated with administration of the multi-component pharmaceutical treatment is reduced as compared to administration of a similar pharmaceutical treatment wherein a human subject receives Tcons but does not receive Tregs.

233. A method of transplanting a conventional T cell (Tcon) population into a human subject without eliciting a stage 2 or higher graft versus host disease (GVHD) response up to about 100 days after transplanting, the method comprising:
i. administering a heterogenous cell population comprising lymphocytes, granulocytes and monocytes, wherein at least about 30% of said lymphocytes comprises conventional T cells (Tcons); and
ii. administering a population of regulatory T cells (Tregs);
wherein the heterogenous cell component and/or the population of Tregs comprise less than about 5 EU/ml endotoxins.

234. A method of treating a human subject comprising:
a) administering a plurality of populations of cells, wherein the plurality of populations of cells comprises:
iii. a population of hematopoietic stem and progenitor cells (HSPCs);
iv. a population of cells comprising regulatory T cells (Tregs); and
v. a population of conventional T cells (Tcons); and
b) administering no more than one graft versus host disease (GVHD) prophylactic agent for less than about 120 days,
wherein the population of HSPCs comprises less than about 2% CD3+ cells.
